(19) United States
(12) Patent Application Publication
(10) Pub. No.: US 2025/0289853 A1
(43) Pub. Date: Sep. 18, 2025

(54) NUCLEIC ACID BINDING DOMAINS AND METHODS OF USE THEREOF

(71) Applicant: Altius Institute for Biomedical Sciences, Seattle, WA (US)

(72) Inventors: John A. Stamatoyannopoulos, Seattle, WA (US); Fyodor Urnov, Seattle, WA (US); Sean Thomas, Seattle, WA (US)

(21) Appl. No.: 19/063,769

(22) Filed: Feb. 26, 2025

Related U.S. Application Data
(63) Continuation of application No. 17/253,845, filed on Dec. 18, 2020, now Pat. No. 12,264,181.
(60) Provisional application No. 62/690,888, filed on Jun. 27, 2018.
(60) Provisional application No. 62/694,239, filed on Jul. 5, 2018.
(60) Provisional application No. 62/716,147, filed on Aug. 8, 2018.
(60) Provisional application No. 62/852,134, filed on May 23, 2019.

(51) Int. Cl. C07K 14/195; A61K 38/00; C12N 9/22; C12N 15/90

(52) U.S. Cl. CPC C07K 14/195; C12N 9/22; C12N 15/907; A61K 38/00; C07K 2319/80; C12N 2800/80

(57) ABSTRACT

Provided herein are polypeptides, compositions comprising the polypeptides and methods for genome editing and gene regulation (e.g., activation and/or repression) using the polypeptides or the compositions comprising the polypeptides, such as, DNA binding domains derived from the genus of Ralstonia. Also disclosed are DNA binding proteins that include a fragment of N-cap sequence of a TALE protein, such as, a Xanthomonas TALE protein. Also disclosed are DNA binding proteins that include a fragment of N-cap sequence of a DNA binding protein derived from bacteria of the genus Ralstonia.

### CROSS-REFERENCE TO RELATED APPLICATIONS

This application is a continuation of U.S. application Ser. No. 17/253,845, filed on Dec. 18, 2020, which application is a U.S. National Stage of International Application No. PCT/US2019/039318, filed on Jun. 26, 2019, which application claims the benefit U.S. Provisional Application No. 62/690,888, filed on Jun. 27, 2018, U.S. Provisional Application No. 62/694,239, filed on Jul. 5, 2018, U.S. Provisional Application No. 62/716,147, filed on Aug. 8, 2018 and U.S. Provisional Application No. 62/852,134, filed on May 23, 2019, which applications are incorporated herein by reference in its entirety for all purposes.

### REFERENCE TO AN ELECTRONIC SEQUENCE LISTING

The contents of the electronic sequence listing (ALTI-718CON_SEQ_LIST.xml; Size: 690,409 bytes; and Date of Creation: Feb. 19, 2025) is herein incorporated by reference in its entirety.

### INTRODUCTION

Genome editing and gene regulation techniques include the use of nucleic acid binding domains linked to a functional domain. Provided herein are polypeptides and methods for genome editing and gene regulation, wherein the nucleic acid binding domain is derived from DNA binding proteins from bacteria from the genus of Ralstonia or from Xanthomonas.

### SUMMARY

In various aspects, the present disclosure provides a polypeptide comprising a modular nucleic acid binding domain comprising a potency for a target site greater than 65% and a specificity ratio for the target site of at least 50:1; and a functional domain; wherein: the modular nucleic acid binding domain comprises a plurality of repeat units; at least one repeat unit of the plurality of repeat units comprises a binding region configured to bind to a target nucleic acid base within the target site; the potency comprises indel percentage at the target site, and wherein the specificity ratio comprises indel percentage at the target site over indel percentage at a top-ranked off-target site of the polypeptide.

In some aspects, the at least one repeat unit comprises a sequence of A₁₋₁₁X₁X₂B₁₄₋₃₅, wherein: each amino acid residue of A₁₋₁₁ comprises any amino acid residue; X₁X₂ comprises the binding region; each amino acid residue of B₁₄₋₃₅ comprises any amino acid; and a first repeat unit of the plurality of repeat units comprises at least one residue in A₁₋₁₁, B₁₄₋₃₅, or a combination thereof that differs from a corresponding residue in a second repeat unit of the plurality of repeat units.

In various aspects, the present disclosure provides a polypeptide comprising a modular nucleic acid binding domain and a functional domain, wherein: the modular nucleic acid binding domain comprises a plurality of repeat units; at least one repeat unit of the plurality comprises a sequence of A₁₋₁₁X₁X₂B₁₄₋₃₅; each amino acid residue of A₁₋₁₁ comprises any amino acid residue; X₁X₂ comprises a binding region configured to bind to a target nucleic acid base within a target sitc; each amino acid residue of B₁₄₋₃₅ comprises any amino acid; and a first repeat unit of the plurality of repeat units comprises at least one residue in A₁₋₁₁, B₁₄₋₃₅, or a combination thereof that differs from a corresponding residue in a second repeat unit of the plurality of repeat units.

In some aspects, the binding region comprises an amino acid residue at position 13 or an amino acid residue at position 12 and the amino acid residue at position 13. In further aspects, the amino acid residue at position 13 binds to the target nucleic acid base. In some aspects, the amino acid residue at position 12 stabilizes the configuration of the binding region.

In some aspects, the modular nucleic acid binding domain further comprises a potency for the target site greater than 65% and a specificity ratio for the target site of at least 50:1, wherein the potency comprises indel percentage at the target site and the specificity ratio comprises indel percentage at the target site over indel percentage at a top-ranked off-target site of the polypeptide. In further aspects, the indel percentage is measured by deep sequencing. In some aspects, the modular nucleic acid binding domain further comprises one or more properties selected from the following: (a) binds the target site, wherein the target site comprises a 5′ guanine; (b) comprises from 7 repeat units to 25 repeat units; (c) upon binding to the target site, the modular nucleic acid binding domain is separated from a second modular nucleic acid binding domain bound to a second target site by from 2 to 50 base pairs.

In some aspects, the modular nucleic acid binding domain comprises a Ralstonia repeat unit. In further aspects, the Ralstonia repeat unit is a Ralstonia solanacearum repeat unit. In still further aspects, the B₁₄₋₃₅ of at least one repeat unit of the plurality of repeat units has at least 92%, at least 93%, at least 94%, at least 95%, at least 96%, at least 97%, at least 98%, at least 99%, or a 100% sequence identity to GGKQALEAVRAQLLDLRAAPYG (SEQ ID NO: 280).

In some aspects, the binding region comprises HD binding to cytosine, NG binding to thymidine, NK binding to guanine, SI binding to adenosine, RS binding to adenosine, HN binding to guanine, or NT binds to adenosine. In some aspects, the at least one repeat unit comprises any one of SEQ ID NO: 267-SEQ ID NO: 279.

In further aspects, the at least one repeat unit comprises at least 80%, at least 85%, at least 90%, at least 95%, at least 96%, at least 97%, at least 98%, at least 99%, or a 100% sequence identity with any one of SEQ ID NO: 168-SEQ ID NO: 263. In further aspects, the at least one repeat unit comprises at least 80%, at least 85%, at least 90%, at least 95%, at least 96%, at least 97%, at least 98%, at least 99%, or a 100% sequence identity with SEQ ID NO: 209, SEQ ID NO: 197, SEQ ID NO: 233, SEQ ID NO: 253, SEQ ID NO: 203, or SEQ ID NO: 218. In some aspects, the at least one repeat unit comprises any one of SEQ ID NO: 168-SEQ ID NO: 263. In further aspects, the at least one repeat unit comprises SEQ ID NO: 209, SEQ ID NO: 197, SEQ ID NO: 233, SEQ ID NO: 253, SEQ ID NO: 203, or SEQ ID NO: 218.

In some aspects, the target nucleic acid base is cytosine, guanine, thymidine, adenosine, uracil or a combination thereof. In some aspects, the target site is a nucleic acid sequence within a PDCD1 gene, a CTLA4 gene, a LAG3 gene, a TET2 gene, a BTLA gene, a HAVCR2 gene, a CCR5 gene, a CXCR4 gene, a TRA gene, a TRB gene, a B2M gene, an albumin gene, a HBB gene, a HBA1 gene, a TTR gene, a NR3C1 gene, a CD52 gene, an crythroid specific enhancer of the BCL11A gene, a CBLB gene, a TGFBR1 gene, a SERPINA1 gene, a HBV genomic DNA in infected cells, a CEP290 gene, a DMD gene, a CFTR gene, an IL2RG gene, or a combination thereof.

In other aspects, a nucleic acid sequence encoding a chimeric antigen receptor (CAR), alpha-L iduronidase (IDUA), iduronate-2-sulfatase (IDS), or Factor 9 (F9), is inserted at the target site.

In some aspects, the modular nucleic acid binding domain comprises an N-terminus amino acid sequence, a C-terminus amino acid sequence, or a combination thereof. In further aspects, the N-terminus amino acid sequence is from Xanthomonas spp., Legionella quateirensis, or Ralstonia solanacearum. In still further aspects, the N-terminus amino acid sequence comprises at least 80%, at least 85%, at least 90%, at least 95%, at least 96%, at least 97%, at least 98%, at least 99%, or a 100% sequence identity to SEQ ID NO: 264, SEQ ID NO: 300, SEQ ID NO: 335, SEQ ID NO: 303, SEQ ID NO: 301, SEQ ID NO: 304, or SEQ ID NO: 320, SEQ ID NO: 321, or SEQ ID NO: 322. In still further aspects, the N-terminus amino acid sequence comprises SEQ ID NO: 264, SEQ ID NO: 300, SEQ ID NO: 335, SEQ ID NO: 303, SEQ ID NO: 301, SEQ ID NO: 304, or SEQ ID NO: 320, SEQ ID NO: 321, or SEQ ID NO: 322.

In some aspects, the C-terminus amino acid sequence is from Xanthomonas spp., Legionella quateirensis, or Ralstonia solanacearum. In further aspects, the C-terminus amino acid sequence comprises at least 80%, at least 85%, at least 90%, at least 95%, at least 96%, at least 97%, at least 98%, at least 99%, or a 100% sequence identity sequence identity to SEQ ID NO: 266, SEQ ID NO: 298, or SEQ ID NO: 306. In still further aspects, the C-terminus amino acid sequence comprises SEQ ID NO: 266, SEQ ID NO: 298, or SEQ ID NO: 306. In some aspects, the C-terminus amino acid sequence serves as a linker between the modular nucleic acid binding domain and the cleavage domain.

In some aspects, the modular nucleic acid binding domain comprises a half repeat. In further aspects, the half repeat comprises at least 80%, at least 85%, at least 90%, at least 95%, at least 96%, at least 97%, at least 98%, at least 99%, or a 100% sequence identity sequence identity to SEQ ID NO: 265, SEQ ID NO: 327-SEQ ID NO: 329, or SEQ ID NO: 290. In further aspects, the half repeat comprises SEQ ID NO: 265, SEQ ID NO: 327-SEQ ID NO: 329, or SEQ ID NO: 290.

In still further aspects, the functional domain is a cleavage domain or a repression domain. In some aspects, the cleavage domain comprises at least 33.3% divergence from SEQ ID NO: 163 and is immunologically orthogonal to SEQ ID NO: 163. In further aspects, the polypeptide comprises one or more of the following characteristics: (a) induces greater than 1% indels at a target site; (b) the cleavage domain comprises a molecular weight of less than 23 kDa; (c) the cleavage domain comprises less than 196 amino acids; (d) capable of cleaving across a spacer region greater than 24 base pairs.

In some aspects, the polypeptide induces greater than 5%, greater than 10%, greater than 20%, greater than 30%, greater than 40%, greater than 50%, greater than 60%, greater than 70%, greater than 80%, or greater than 90% indels at the target site. In some aspects, the cleavage domain comprises at least 35%, at least 40%, at least 45%, at least 50%, at least 55%, at least 60%, at least 65%, at least 70%, or at least 75% divergence from SEQ ID NO: 163. In some aspects, the cleavage domain comprises a sequence selected from SEQ ID NO: 316-SEQ ID NO: 319.

In further aspects, the cleavage domain comprises a nucleic acid sequence encoding for a sequence having at least 80% sequence identity with SEQ ID NO: 1-SEQ ID NO: 81. In still further aspects, the cleavage domain comprises a nucleic acid sequence encoding for a sequence selected from SEQ ID NO: 1-SEQ ID NO: 81. In some aspects, the nucleic acid sequence comprises at least 80% sequence identity with SEQ ID NO: 82-SEQ ID NO: 162. In further aspects, the nucleotide sequence encoding for the sequence comprises any one of SEQ ID NO: 82-SEQ ID NO: 162.

In some aspects, the repression domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-crbA, SID, MBD2, MBD3, Rb, or MeCP2.

In some aspects, the at least one repeat unit comprises 1-20 additional amino acid residues at the C-terminus. In some aspects, the at least repeat unit of the plurality of repeat units is separated from a neighboring repeat unit by a linker. In further aspects, the linker comprises a recognition site. In some aspects, the recognition site is for a small molecule, a protease, or a kinase. In some aspects, the recognition site serves as a localization signal. In some aspects, the plurality of repeat units comprises 3 to 60 repeat units.

In some aspects, a repeat unit of the plurality of repeat units recognizes a target nucleic acid base and wherein the plurality of repeat units has one or more of the following characteristics: (a) at least one repeat unit comprising greater than 39 amino acid residues; (b) at least one repeat unit comprising greater than 35 amino acid residues derived from the genus of Ralstonia; (c) at least one repeat unit comprising less than 32 amino acid residues; and (d) each repeat unit of the plurality of repeat units is separated from a neighboring repeat unit by a linker comprising a recognition site. In some aspects, the at least one repeat unit comprises an amino acid selected from glycine, alanine, threonine or histidine at a position after an amino acid residue at position 35. In some aspects, the at least one repeat unit comprises an amino acid selected from glycine, alanine, threonine or histidine at a position after an amino acid residue at position 39.

Also provided herein is a non-naturally occurring DNA binding polypeptide that includes from N- to C-terminus: a N-terminus region comprising at least residues N+110 to N+1 of a TALE protein, where the N-terminus region does not include residues N+288 to N+116 of the TALE protein; a plurality of TALE repeat units derived from a TALE protein; and C-terminus region of a TALE protein. The N-terminus region may not include at least amino acids N+288 to N+116 of the TALE protein. The N-terminus region may not include amino acids N+288 to up to N+116 of the TALE protein. The N-terminus region may not include at least amino acids N+288 to up to N+111 of the TALE protein. The N-terminus region may include residues N+1 to up to N+115 of the TALE protein. The N-terminus region may include residues N+1 to up to N+110 of the TALE protein. The C-terminus region may include full length C-terminus region of a TALE protein or a fragment thereof, e.g., residues C+1 to C+63 of the TALE protein. The DNA binding polypeptide may be fused to a heterologous functional domain, such as, enzyme, a transcriptional activator, a transcriptional repressor, or a DNA nucleotide modifier. The N-terminus region, the TALE repeat units, and the C-terminus region may be derived from the same TALE protein or from different TALE proteins. The TALE proteins from which the N-terminus region, the TALE repeat units, and the C-terminus region may be derived include Xanthomonas TALE proteins, such as, AvrBs3, AVRHAH1, AvrXa7, AVRB6, or AvrXa10.

In various aspects, the present disclosure provides a method of genome editing, the method comprising: administering any of the above polypeptides or compositions thereof and inducing a double stranded break.

In various aspects, the present disclosure provides method of gene repression, the method comprising administering any of the above polypeptides or compositions thereof and repressing gene expression.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

FIGS. 1A-1C show schematics of the domain structure of DNA binding proteins (not drawn to scale).

FIG. 2 shows nuclease activity mediated by DNA binding protein dimers that each include from N-terminus to C-terminus: a N-terminus region of a TALE protein, TALE repeat units, C-terminus region of a TALE protein, and a Fok1 endonuclease.

### DETAILED DESCRIPTION

The present disclosure provides modular nucleic acid binding domains (NBDs) derived from the genus of bacteria. For example, in some embodiments, the present disclosure provides NBDs derived from bacteria that serve as plant pathogens, such as from the genus of Xanthomonas spp. and Ralstonia. In particular embodiments, the present disclosure provides NBDs from the genus of Ralstonia. Also provided herein are NBDs from the animal pathogen, Legionella. Provided herein are sequences of repeat units derived from the genus of Ralstonia, which can be linked together to form non-naturally occurring modular nucleic acid binding domains (NBDs), capable of targeting and binding any target nucleic acid sequence (e.g., DNA sequence).

In some embodiments, “derived” indicates that a protein is from a particular source (e.g., Ralstonia), is a variant of a protein from a particular source (e.g., Ralstonia), is a mutated or modified form of the protein from a particular source (e.g., Ralstonia), and shares at least 30% sequence identity with, at least 40% sequence identity with, at least 50% sequence identity with, at least 60% sequence identity with, at least 70% sequence identity with, at least 80% sequence identity with, or at least 90% sequence identity with a protein from a particular source (e.g., Ralstonia).

In some embodiments, “modular” indicates that a particular polypeptide such as a nucleic acid binding domain, comprises a plurality of repeat units that can be switched and replaced with other repeat units. For example, any repeat unit in a modular nucleic acid binding domain can be switched with a different repeat unit. In some embodiments, modularity of the nucleic acid binding domains disclosed herein allows for switching the target nucleic acid base for a particular repeat unit by simply switching it out for another repeat unit. In some embodiments, modularity of the nucleic acid binding domains disclosed herein allows for swapping out a particular repeat unit for another repeat unit to increase the affinity of the repeat unit for a particular target nucleic acid. Overall, the modular nature of the nucleic acid binding domains disclosed herein enables the development of genome editing complexes that can precisely target any nucleic acid sequence of interest.

In particular embodiments, modular nucleic acid binding domains (NBDs), also referred to herein as “DNA binding polypeptides,” are provided herein from the genus of Ralstonia solanacearum. In some embodiments, modular nucleic acid binding domains derived from Ralstonia (RNBDs) can be engineered to bind to a target gene of interest for purposes of gene editing or gene regulation. An RNBD can be engineered to target and bind a specific nucleic acid sequence. The nucleic acid sequence can be DNA or RNA.

In some embodiments, the RNBD can comprise a plurality of repeat units, wherein each repeat unit recognizes and binds to a single nucleotide (in DNA or RNA) or base pair. Each repeat unit in the plurality of repeat units can be specifically selected to target and bind to a specific nucleic acid sequence, thus contributing to the modular nature of the DNA binding polypeptide. A non-naturally occurring Ralstonia-derived modular nucleic acid binding domain can comprise a plurality of repeat units, wherein each repeat unit of the plurality of repeat units recognizes a single target nucleotide, base pair, or both.

### Ralstonia-Derived DNA Binding Domains

In some embodiments, the repeat unit of a modular nucleic acid binding domain can be derived from a bacterial protein. For example, the bacterial protein can be a transcription activator like effector-like protein (TALE-like protein). The bacterial protein can be derived from Ralstonia solanacearum. Repeat units derived from Ralstonia solanacearum can be 33-35 amino acid residues in length. In some embodiments, the repeat unit can be derived from the naturally occurring Ralstonia solanacearum TALE-like protein.

TABLE 1 below shows exemplary repeat units derived from the genus of Ralstonia, which are capable of binding a target nucleic acid.

TABLE 1
Exemplary Ralstonia-derived Repeat Units

| SEQ ID NO | Sequence |
|---|---|
| SEQ ID | LDTEQVVAIASHNGGKQALEAVKADLLDLLGAPYV |
| NO: 168 | |
| SEQ ID | LDTEQVVAIASHNGGKQALEAVKADLLDLRGAPYA |
| NO: 169 | |
| SEQ ID | LDTEQVVAIASHNGGKQALEAVKADLLELRGAPYA |
| NO: 170 | |
| SEQ ID | LDTEQVVAIASHNGGKQALEAVKAHLLDLRGAPYA |
| NO: 171 | |
| SEQ ID | LNTEQVVAIASHNGGKQALEAVKADLLDLRGAPYA |
| NO: 172 | |
| SEQ ID | LNTEQVVAIASNNGGKQALEAVKTHLLDLRGARYA |
| NO: 173 | |
| SEQ ID | LNTEQVVAIASNPGGKQALEAVRALFPDLRAAPYA |
| NO: 174 | |
| SEQ ID | LNTEQVVAIASSHGGKQALEAVRALFPDLRAAPYA |
| NO: 175 | |
| SEQ ID | LNTEQVVAVASNKGGKQALEAVGAQLLALRAVPYA |
| NO: 176 | |
| SEQ ID | LNTEQVVAVASNKGGKQALEAVGAQLLALRAVPYE |
| NO: 177 | |
| SEQ ID | LSAAQVVAIASHDGGKQALEAVGTQLVALRAAPYA |
| NO: 178 | |
| SEQ ID | LSIAQVVAVASRSGGKQALEAVRAQLLALRAAPYG |
| NO: 179 | |
| SEQ ID | LSPEQVVAIASNHGGKQALEAVRALFRGLRAAPYG |
| NO: 180 | |
| SEQ ID | LSPEQVVAIASNNGGKQALEAVKAQLLELRAAPYE |
| NO: 181 | |
| SEQ ID | LSTAQLVAIASNPGGKQALEAIRALFRELRAAPYA |
| NO: 182 | |
| SEQ ID | LSTAQLVAIASNPGGKQALEAVRALFRELRAAPYA |
| NO: 183 | |
| SEQ ID | LSTAQLVAIASNPGGKQALEAVRAPFREVRAAPYA |
| NO: 184 | |
| SEQ ID | LSTAQLVSIASNPGGKQALEAVRALFRELRAAPYA |
| NO: 185 | |
| SEQ ID | LSTAQVAAIASHDGGKQALEAVGTQLVVLRAAPYA |
| NO: 186 | |
| SEQ ID | LSTAQVATIASSIGGRQALEALKVQLPVLRAAPYG |
| NO: 187 | |
| SEQ ID | LSTAQVATIASSIGGRQALEAVKVQLPVLRAAPYG |
| NO: 188 | |
| SEQ ID | LSTAQVVAIAANNGGKQALEAVRALLPVLRVAPYE |
| NO: 189 | |
| SEQ ID | LSTAQVVAIAGNGGGKQALEGIGEQLLKLRTAPYG |
| NO: 190 | |
| SEQ ID | LSTAQVVAIASHDGGKQALEAAGTQLVALRAAPYA |
| NO: 191 | |
| SEQ ID | LSTAQVVAIASHDGGKQALEAVGAQLVELRAAPYA |
| NO: 192 | |
| SEQ ID | LSTAQVVAIASHDGGKQALEAVGTQLVALRAAPYA |
| NO: 193 | |
| SEQ ID | LSTAQVVAIASHDGGNQALEAVGTQLVALRAAPYA |
| NO: 194 | |
| SEQ ID | LSTAQVVAIASHNGGKQALEAVKAQLLDLRGAPYA |
| NO: 195 | |
| SEQ ID | LSTAQVVAIASNDGGKQALEEVEAQLLALRAAPYE |
| NO: 196 | |
| SEQ ID | LSTAQVVAIASNGGGKQALEGIGEQLLKLRTAPYG |
| NO: 197 | |
| SEQ ID | LSTAQVVAIASNGGGKQALEGIGEQLRKLRTAPYG |
| NO: 198 | |
| SEQ ID | LSTAQVVAIASNPGGKQALEAVRALFRELRAAPYA |
| NO: 199 | |
| SEQ ID | LSTAQVVAIASQNGGKQALEAVKAQLLDLRGAPYA |
| NO: 200 | |
| SEQ ID | LSTAQVVAIASSHGGKQALEAVRALFRELRAAPYG |
| NO: 201 | |
| SEQ ID | LSTAQVVAIASSNGGKQALEAVWALLPVLRATPYD |
| NO: 202 | |
| SEQ ID | LSTAQVVAIATRSGGKQALEAVRAQLLDLRAAPYG |
| NO: 203 | |
| SEQ ID | LSTAQVVAVAGRNGGKQALEAVRAQLPALRAAPYG |
| NO: 204 | |
| SEQ ID | LSTAQVVAVASSNGGKQALEAVWALLPVLRATPYD |
| NO: 205 | |
| SEQ ID | LSTAQVVTIASSNGGKQALEAVWALLPVLRATPYD |
| NO: 206 | |
| SEQ ID | LSTEQVVAIAGHDGGKQALEAVGAQLVALRAAPYA |
| NO: 207 | |
| SEQ ID | LSTEQVVAIASHDGGKQALEAVGAQLVALLAAPYA |
| NO: 208 | |
| SEQ ID | LSTEQVVAIASHDGGKQALEAVGAQLVALRAAPYA |
| NO: 209 | |
| SEQ ID | LSTEQVVAIASHDGGKQALEAVGGQLVALRAAPYA |
| NO: 210 | |
| SEQ ID | LSTEQVVAIASHDGGKQALEAVGTQLVALRAAPYA |
| NO: 211 | |
| SEQ ID | LSTEQVVAIASHDGGKQALEAVGVQLVALRAAPYA |
| NO: 212 | |
| SEQ ID | LSTEQVVAIASHDGGKQALEAVVAQLVALRAAPYA |
| NO: 213 | |
| SEQ ID | LSTEQVVAIASHDGGKQPLEAVGAQLVALRAAPYA |
| NO: 214 | |
| SEQ ID | LSTEQVVAIASHGGGKQVLEGIGEQLLKLRAAPYG |
| NO: 215 | |
| SEQ ID | LSTEQVVAIASHKGGKQALEGIGEQLLKLRAAPYG |
| NO: 216 | |
| SEQ ID | LSTEQVVAIASHNGGKQALEAVKADLLDLRGAPYA |
| NO: 217 | |
| SEQ ID | LSTEQVVAIASHNGGKQALEAVKADLLELRGAPYA |
| NO: 218 | |
| SEQ ID | LSTEQVVAIASHNGGKQALEAVKAHLLDLRGAPYA |
| NO: 219 | |
| SEQ ID | LSTEQVVAIASHNGGKQALEAVKAHLLDLRGVPYA |
| NO: 220 | |
| SEQ ID | LSTEQVVAIASHNGGKQALEAVKAHLLELRGAPYA |
| NO: 221 | |
| SEQ ID | LSTEQVVAIASHNGGKQALEAVKAQLLDLRGAPYA |
| NO: 222 | |
| SEQ ID | LSTEQVVAIASHNGGKQALEAVKAQLLELRGAPYA |
| NO: 223 | |
| SEQ ID | LSTEQVVAIASHNGGKQALEAVKAQLPVLRRAPYG |
| NO: 224 | |
| SEQ ID | LSTEQVVAIASHNGGKQALEAVKTQLLELRGAPYA |
| NO: 225 | |
| SEQ ID | LSTEQVVAIASHNGGKQALEAVRAQLPALRAAPYG |
| NO: 226 | |
| SEQ ID | LSTEQVVAIASHNGSKQALEAVKAQLLDLRGAPYA |
| NO: 227 | |
| SEQ ID | LSTEQVVAIASNGGGKQALEGIGKQLQELRAAPHG |
| NO: 228 | |
| SEQ ID | LSTEQVVAIASNGGGKQALEGIGKQLQELRAAPYG |
| NO: 229 | |
| SEQ ID | LSTEQVVAIASNHGGKQALEAVRALFRELRAAPYA |
| NO: 230 | |
| SEQ ID | LSTEQVVAIASNHGGKQALEAVRALFRGLRAAPYG |
| NO: 231 | |
| SEQ ID | LSTEQVVAIASNKGGKQALEAVKADLLDLRGAPYV |
| NO: 232 | |
| SEQ ID | LSTEQVVAIASNKGGKQALEAVKAHLLDLLGAPYV |
| NO: 233 | |
| SEQ ID | LSTEQVVAIASNKGGKQALEAVKAQLLALRAAPYA |
| NO: 234 | |
| SEQ ID | LSTEQVVAIASNKGGKQALEAVKAQLLELRGAPYA |
| NO: 235 | |
| SEQ ID | LSTEQVVAIASNNGGKQALEAVKALLLELRAAPYE |
| NO: 236 | |
| SEQ ID | LSTEQVVAIASNNGGKQALEAVKAQLLALRAAPYE |
| NO: 237 | |
| SEQ ID | LSTEQVVAIASNNGGKQALEAVKAQLLDLRGAPYA |
| NO: 238 | |
| SEQ ID | LSTEQVVAIASNNGGKQALEAVKAQLLVLRAAPYG |
| NO: 239 | |
| SEQ ID | LSTEQVVAIASNNGGKQALEAVKAQLPALRAAPYE |
| NO: 240 | |
| SEQ ID | LSTEQVVAIASNNGGKQALEAVKAQLPVLRRAPCG |
| NO: 241 | |
| SEQ ID | LSTEQVVAIASNNGGKQALEAVKAQLPVLRRAPYG |
| NO: 242 | |
| SEQ ID | LSTEQVVAIASNNGGKQALEAVKARLLDLRGAPYA |
| NO: 243 | |
| SEQ ID | LSTEQVVAIASNNGGKQALEAVKTQLLALRTAPYE |
| NO: 244 | |
| SEQ ID | LSTEQVVAIASNPGGKQALEAVRALFPDLRAAPYA |
| NO: 245 | |
| SEQ ID | LSTEQVVAIASSHGGKQALEAVRALFPDLRAAPYA |
| NO: 246 | |
| SEQ ID | LSTEQVVAIASSHGGKQALEAVRALLPVLRATPYD |
| NO: 247 | |
| SEQ ID | LSTEQVVAVASHNGGKQALEAVRAQLLDLRAAPYE |
| NO: 248 | |
| SEQ ID | LSTEQVVAVASNKGGKQALAAVEAQLLRLRAAPYE |
| NO: 249 | |
| SEQ ID | LSTEQVVAVASNKGGKQALEEVEAQLLRLRAAPYE |
| NO: 250 | |
| SEQ ID | LSTEQVVAVASNKGGKQVLEAVGAQLLALRAVPYE |
| NO: 251 | |
| SEQ ID | LSTEQVVAVASNNGGKQALKAVKAQLLALRAAPYE |
| NO: 252 | |
| SEQ ID | LSTEQVVVIANSIGGKQALEAVKVQLPVLRAAPYE |
| NO: 253 | |
| SEQ ID | LSTGQVVAIASNGGGRQALEAVREQLLALRAVPYE |
| NO: 254 | |
| SEQ ID | LSPEQVVTIASNNGGKQALEAVRAQLLALRAAPYG |
| NO: 255 | |
| SEQ ID | LTIAQVVAVASHNGGKQALEAIGAQLLALRAAPYA |
| NO: 256 | |
| SEQ ID | LTIAQVVAVASHNGGKQALEVIGAQLLALRAAPYA |
| NO: 257 | |
| SEQ ID | LTPQQVVAIAANTGGKQALGAITTQLPILRAAPYE |
| NO: 258 | |
| SEQ ID | LTPQQVVAIASNTGGKQALEAVTVQLRVLRGARYG |
| NO: 259 | |
| SEQ ID | LTPQQVVAIASNTGGKRALEAVCVQLPVLRAAPYR |
| NO: 260 | |
| SEQ ID | LTPQQVVAIASNTGGKRALEAVRVQLPVLRAAPYE |
| NO: 261 | |
| SEQ ID | LTTAQVVAIASNDGGKQALEAVGAQLLVLRAVPYE |
| NO: 262 | |
| SEQ ID | LTTAQVVAIASNDGGKQTLEVAGAQLLALRAVPYE |
| NO: 263 | |
| SEQ ID | LSTAQVVAVASGSGGKPALEAVRAQLLALRAAPYG |
| NO: 336 | |
| SEQ ID | LSTAQVVAVASGSGGKPALEAVRAQLLALRAAPYG |
| NO: 337 | |
| SEQ ID | LNTAQIVAIASHDGGKPALEAVWAKLPVLRGAPYA |
| NO: 338 | |
| SEQ ID | LNTAQVVAIASHDGGKPALEAVRAKLPVLRGVPYA |
| NO: 339 | |
| SEQ ID | LNTAQVVAIASHDGGKPALEAVWAKLPVLRGVPYA |
| NO: 340 | |
| SEQ ID | LNTAQVVAIASHDGGKPALEAVWAKLPVLRGVPYE |
| NO: 341 | |
| SEQ ID | LSTAQVVAIASHDGGKPALEAVWAKLPVLRGAPYA |
| NO: 342 | |
| SEQ ID | LSTAQVVAVASHDGGKPALEAVRKQLPVLRGVPHQ |
| NO: 343 | |
| SEQ ID | LSTAQVVAVASHDGGKPALEAVRKQLPVLRGVPHQ |
| NO: 344 | |
| SEQ ID | LNTAQVVAIASHDGGKPALEAVWAKLPVLRGVPYA |
| NO: 345 | |
| SEQ ID | LSTEQVVAIASHNGGKLALEAVKAHLLDLRGAPYA |
| NO: 346 | |
| SEQ ID | LSTEQVVAIASHNGGKPALEAVKAHLLALRAAPYA |
| NO: 347 | |
| SEQ ID | LNTAQVVAIASHYGGKPALEAVWAKLPVLRGVPYA |
| NO: 348 | |
| SEQ ID | LNTEQVVAIASNNGGKPALEAVKAQLLELRAAPYE |
| NO: 349 | |
| SEQ ID | LSPEQVVAIASNNGGKPALEAVKALLLALRAAPYE |
| NO: 350 | |
| SEQ ID | LSPEQVVAIASNNGGKPALEAVKAQLLELRAAPYE |
| NO: 351 | |
| SEQ ID | LSTEQVVAIASNNGGKPALEAVKALLLALRAAPYE |
| NO: 352 | |
| SEQ ID | LSTEQVVAIASNNGGKPALEAVKALLLELRAAPYE |
| NO: 353 | |
| SEQ ID | LSPEQVVAIASNNGGKPALEAVKALLLALRAAPYE |
| NO: 354 | |
| SEQ ID | LSPEQVVAIASNNGGKPALEAVKAQLLELRAAPYE |
| NO: 355 | |
| SEQ ID | LSTEQVVAIASNNGGKPALEAVKALLLELRAAPYE |
| NO: 356 | |

In some embodiments, an RNBD of the present disclosure can comprise between 1 to 50 Ralstonia solanacearum-derived repeat units. In some embodiments, an RNBD of the present disclosure can comprise between 9 and 36 Ralstonia solanacearum-derived repeat units. Preferably, in some embodiments, an RNBD of the present disclosure can comprise between 12 and 30 Ralstonia solanacearum-derived repeat units. A RNBD described herein can comprise between 5 to 10 Ralstonia solanacearum-derived repeat units, between 10 to 15 Ralstonia solanacearum-derived repeat units, between 15 to 20 Ralstonia solanacearum-derived repeat units, between 20 to 25 Ralstonia solanacearum-derived repeat units, between 25 to 30 Ralstonia solanacearum-derived repeat units, or between 30 to 35 Ralstonia solanacearum-derived repeat units, between 35 to 40 Ralstonia solanacearum-derived repeat units. A RNBD described herein can comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or more Ralstonia solanacearum-derived repeat units.

A Ralstonia solanacearum-derived repeat unit can be derived from a wild-type repeat unit, such as any one of SEQ ID NO: 168-SEQ ID NO: 263 or SEQ ID NO: 336-SEQ ID NO: 356. A Ralstonia solanacearum-repeat unit can have at least 80% sequence identity with any one of SEQ ID NO: 168-SEQ ID NO: 263 or SEQ ID NO: 336-SEQ ID NO: 356. A Ralstonia solanacearum-derived repeat unit can also comprise a modified Ralstonia solanacearum-derived repeat unit enhanced for specific recognition of a nucleotide or base pair. An RNBD described herein can comprise one or more wild-type Ralstonia solanacearum-derived repeat units, one or more modified Ralstonia solanacearum-derived repeat units, or a combination thereof. In some embodiments, a modified Ralstonia solanacearum-derived repeat unit can comprise 1, 2, 3, 4, 5, 6, 7, 8, 9, 10, 11, 12, 13, 14, 15, 16, 17, 18, 19, 20, 21, 22, 23, 24, 25, 26, 27, 28, or 29 mutations that can enhance recognition of a specific nucleotide or base pair. In some embodiments, a modified Ralstonia solanacearum-derived repeat unit can comprise more than 1 modification, for example 1 to 5 modifications, 5 to 10 modifications, 10 to 15 modifications, 15 to 20 modifications, 20 to 25 modification, or 25-29 modifications. In some embodiments, An RNBD can comprise more than one modified Ralstonia solanacearum-derived repeat units, wherein each of the modified Ralstonia solanacearum-derived repeat units can have a different number of modifications.

The Ralstonia solanacearum-derived repeat units comprise amino acid residues at positions 12 and 13, what is referred to herein as, a repeat variable diresidue (RVD). The RVD can modulate binding affinity of the repeat unit for a particular nucleic acid base (e.g., adenosine, guanine, cytosine, thymidine, or uracil (in RNA sequences)). In some embodiments, a single amino acid residue can modulate binding to the target nucleic acid base. In some embodiments, two amino acid residues (RVD) can modulate binding to the target nucleic acid base. In some embodiments, any repeat unit disclosed herein can have an RVD selected from HD, HG, HK, HN, ND, NG, NH, NK, NN, NP, NT, QN, RN, RS, SH, SI, or SN. In some embodiments, an RVD of HD can bind to cytosinc. In some embodiments, an RVD of NG can bind to thymidine. In some embodiments, an RVD of NK can bind to guanine. In some embodiments, an RVD of SI can bind to adenosine. In some embodiments, an RVD of RS can bind to adenosine. In some embodiments, an RVD of HN can bind to guanine. In some embodiments, an RVD of NT can bind to adenosinc.

In some embodiments, a repeat unit having at least 80% sequence identity with SEQ ID NO: 209 can be included in a DNA binding domain of the present disclosure to bind to cytosine. In some embodiments, a repeat unit having at least 80% sequence identity with SEQ ID NO: 197 can be included in a DNA binding domain of the present disclosure to bind to thymidine. In some embodiments, a repeat unit having at least 80% sequence identity with SEQ ID NO: 233 can be included in a DNA binding domain of the present disclosure to bind to guanine. In some embodiments, a repeat unit having at least 80% sequence identity with SEQ ID NO: 253 can be included in a DNA binding domain of the present disclosure to bind to adenosine. In some embodiments, a repeat unit having at least 80% sequence identity with SEQ ID NO: 203 can be included in a DNA binding domain of the present disclosure to bind to adenosine. In some embodiments, a repeat unit having at least 80% sequence identity with SEQ ID NO: 218 can be included in a DNA binding domain of the present disclosure to bind to guanine. In some embodiments, the repeat unit of SEQ ID NO: 209 can be included in a DNA binding domain of the present disclosure to bind to cytosine. In some embodiments, the repeat unit of SEQ ID NO: 197 can be included in a DNA binding domain of the present disclosure to bind to thymidine. In some embodiments, the repeat unit of SEQ ID NO: 233 can be included in a DNA binding domain of the present disclosure to bind to guanine. In some embodiments, the repeat unit of SEQ ID NO: 253 can be included in a DNA binding domain of the present disclosure to bind to adenosine. In some embodiments, the repeat unit of SEQ ID NO: 203 can be included in a DNA binding domain of the present disclosure to bind to adenosine. In some embodiments, the repeat unit of SEQ ID NO: 218 can be included in a DNA binding domain of the present disclosure to bind to guanine.

In some embodiments, the present disclosure provides repeat units as set forth in SEQ ID NO: 267-SEQ ID NO: SEQ ID NO: 279. Unspecified amino acid residues in SEQ ID NO: 267-SEQ ID NO: SEQ ID NO: 279 can be any amino acid residues. In particular embodiments, unspecified amino acid residues in SEQ ID NO: 267-SEQ ID NO: SEQ ID NO: 279 can be those set forth in the Variable Definition column of TABLE 2.

TABLE 2 shows consensus sequences of Ralstonia-derived repeat units.

TABLE 2
Consensus Sequences of Ralstonia-derived Repeat Units

| RVD | Consensus Sequence | Variable Definition |
|---|---|---|
| HN | LX₁X₂X₃QVVX₄X₅ASHNGX₆KQALEX₇X₈X₉ | X₁: D\|N\|S\|T, X₂: I\|T\|V, X₃: A\|E, X₄: A\|T, X₅: |
| | X₁₀X₁₁LX₁₂X₁₃LX₁₄X₁₅X₁₆PYX₁₇ | I\|V, X₆: G\|S, X₇: A\|V, X₈: I\|V, X₉: G\|K\|R, X₁₀: |
| | (SEQ ID NO: 267) | A\|T, X₁₁: D\|H\|Q, X₁₂: L\|P, X₁₃: A\|D\|E\|V, X₁₄: |
| | | L\|R, X₁₅: A\|G\|R, X₁₆: A\|V, X₁₇: A\|E\|G\|V |
| NN | LX₁X₂X₃QVVAX₄AX₅NNGGKQALX₆AVX₇X₈ | X₁: N\|S, X₂: P\|T, X₃: A\|E, X₄: I\|V, X₅: A\|S, X₆: |
| | X₉LX₁₀X₁₁LRX₁₂AX₁₃X₁₄X₁₅ | E\|K, X₇: K\|R, X₈: A\|T, X₉: H\|L\|Q\|R, X₁₀: L\|P, |
| | (SEQ ID NO: 268) | X₁₁: A\|D\|E\|V, X₁₂: A\|G\|R\|T\|V, X₁₃: P\|R, X₁₄: |
| | | C\|Y, X₁₅: A\|E\|G |
| NP | LX₁TX₂QX₃VX₄IASNPGGKQALEAX₅RAX₆F | X₁: N\|S, X₂: A\|E, X₃: L\|V, X₄: A\|S, X₅: I\|V, X₆: |
| | X₇X₈X₉RAAPYA (SEQ ID NO: 269) | L\|P, X₇: P\|R, X₈: D\|E, X₉: L\|V |
| SH | LX₁TX₂QVVAIASSHGGKQALEAVRALX₃X₄ | X₁: N\|S, X₂: A\|E, X₃: F\|L, X₄: P\|R, X₅: D\|E\|V, |
| | X₅LRAX₆PYX₇ (SEQ ID NO: 270) | X₆: A\|T, X₇: A\|D\|G |
| NK | LX₁TEQVVAX₂ASNKGGKQX₃LX₄X₅VX₆AX₇ | X₁: N\|S, X₁₀: A\|G, X₁₁: A\|V, X₁₂: A\|E\|V, X₂: |
| | LLX₈LX₉X₁₀X₁₁PYX₁₂ | I\|V, X₃: A\|V, X₄: A\|E, X₅: A\|E, X₆: E\|G\|K, X₇: |
| | (SEQ ID NO: 271) | D\|H\|Q, X₈: A\|D\|E\|R, X₉: L\|R |
| HD | LSX₁X₂QVX₃AIAX₄HDGGX₅QX₆LEAX₇X₈X₉ | X₁: A\|T, X₂: A\|E, X₃: A\|V, X₄: G\|S, X₅: K\|N, |
| | QLVX₁₀LX₁₁AAPYA | X₆: A\|P, X₇: A\|V, X₈: G\|V, X₉: A\|G\|T\|V, X₁₀: |
| | (SEQ ID NO: 272) | A\|E\|V, X₁₁: L\|R |
| RS | LSX₁AQVVAX₂AX₃RSGGKQALEAVRAQLL | X₁: I\|T, X₂: I\|V, X₃: S\|T, X₄: A\|D |
| | X₄LRAAPYG (SEQ ID NO: 273) | |
| NH | LSX₁EQVVAIASNHGGKQALEAVRALFRX₂L | X₁: P\|T, X₂: E\|G, X₃: A\|G |
| | RAAPYX (SEQ ID NO: 274) | |
| SI | LSTX₁QVX₂X₃IAX₄SIGGX₅QALEAX₆KVQLP | X₁: A\|E, X₂: A\|V, X₃: T\|V, X₄: N\|S, X₅: K\|R, |
| | VLRAAPYX₇ (SEQ ID NO: 275) | X₆: L\|V, X₇: E\|G |
| ND | LX₁TAQVVAIASNDGGKQX₂LEX₃X₄X₅AQLL | X₁: S\|T, X₂: A\|T, X₃: A\|E\|V, X₄: A\|V, X₅: E\|G, |
| | X₆LRAX₇PYE (SEQ ID NO: 276) | X₆: A\|V, X₇: A\|V |
| SN | LSTAQVVX₁X₂ASSNGGKQALEAVWALLPV | X₁: A\|T, X₂: I\|V |
| | LRATPYD (SEQ ID NO: 277) | |
| NG | LSTX₁QVVAIAX₂NGGGX₃QALEX₄X₅X₆X₇QL | X₁: A\|E\|G, X₂: G\|S, X₃: K\|R, X₄: A\|G, X₅: I\|V, |
| | X₈X₉LRX₁₀X₁₁PX₁₂X₁₃ | X₆: G\|R, X₇: E\|K, X₈: L\|Q\|R, X₉: A\|E\|K, X₁₀: |
| | (SEQ ID NO: 278) | A\|T, X₁₁: A\|V, X₁₂: H\|Y, X₁₃: E\|G |
| NT | LTPQQVVAIAX₁NTGGKX₂ALX₃AX₄X₅X₆QL | X₁: A\|S, X₁₀: P\|R, X₁₁: E\|G\|R, X₂: Q\|R, X₃: |
| | X₇X₈LRX₉AX₁₀YX₁₁ | E\|G, X₄: I\|V, X₅: C\|R\|T, X₆: T\|V, X₇: P\|R, X₈: |
| | (SEQ ID NO: 279) | I\|V, X₉: A\|G |

In some aspects, the at least one repeat unit comprises any one of SEQ ID NO: 267-SEQ ID NO: 279. In some embodiments, the present disclosure provides a modular nucleic acid binding domain (e.g., RNBD or MAP-NBD), wherein the modular nucleic acid binding domain comprises a repeat unit with a sequence of A₁₋₁X₁X₂B₁₄₋₃₅, wherein A₁₋₁₁ comprises 11 amino acid residues and wherein each amino acid residue of A₁₋₁ can be any amino acid. In some embodiments, A₁₋₁ can be any amino acids in position 1 through position 11 of any one of SEQ ID NO: 168-SEQ ID NO: 263 or SEQ ID NO: 336-SEQ ID NO: 356. X₁X₂ comprises any repeat variable diresidue (RVD) disclosed herein and comprises at least one amino acid at position 12 or position 13. As described herein, this RVD contacts and binds to a target nucleic acid base of a target site. Said RVD can be the RVD of any repeat unit disclosed herein, such as position 12 and position 13 of any one of SEQ ID NO: 168-SEQ ID NO: 263 or SEQ ID NO: 336-SEQ ID NO: 356. B₁₄₋₃₅ can comprise 22 amino acid residues and each amino acid residue of B₁₄₋₃₅ can be any amino acid. In some embodiments, B₁₄₋₃₅ can be any amino acid in position 14 through position 35 of any one of SEQ ID NO: 168-SEQ ID NO: 263 or SEQ ID NO: 336-SEQ ID NO: 356. In particular embodiments, a modular nucleic acid binding domain (e.g., RNBD or MAP-NBD) having the above sequence of A₁₋₁X₁X₂B₁₄₋₃₅ can have a first repeat unit with at least one residue in A₁₋₁₁, B₁₄₋₃₅, or a combination thereof that differs from a corresponding residue in a second repeat unit in the modular nucleic acid binding domain (e.g., RNBD or MAP-NBD). In other words, at least two repeat units in a modular nucleic acid binding domain (e.g., RNBD or MAP-NBD) described herein can have different amino acid residues with respect to each other, at the same position outside the RVD region. Thus, in some embodiments, a modular nucleic acid binding domain (e.g., RNBD or MAP-NBD) described herein can have variant backbones with respect to each repeat unit in the plurality of repeat units that make up the modular nucleic acid binding domain. In some embodiments, an RNBD of the present disclosure can have a sequence of GGKQALEAVRAQLLDLRAAPYG (SEQ ID NO: 280) at B₁₄₋₃₅.

In some embodiments, the present disclosure provides a polypeptide comprising a modular nucleic acid binding domain and a functional domain, wherein: the modular nucleic acid binding domain comprises a plurality of repeat units; at least one repeat unit of the plurality comprises a sequence of A₁₋₁₁X₁X₂B₁₄₋₃₅; each amino acid residue of A₁₋₁₁ comprises any amino acid residue; X₁X₂ comprises a binding region configured to bind to a target nucleic acid base within a target site; each amino acid residue of B 14-35 comprises any amino acid; and a first repeat unit of the plurality of repeat units comprises at least one residue in A₁₋₁₁, B₁₄₋₃₅, or a combination thereof that differs from a corresponding residue in a second repeat unit of the plurality of repeat units. In some embodiments, the binding region comprises an amino acid residue at position 13 or an amino acid residue at position 12 and the amino acid residue at position 13. In further aspects, the amino acid residue at position 13 binds to the target nucleic acid base. In some aspects, the amino acid residue at position 12 stabilizes the configuration of the binding region.

In some embodiments, the modular nucleic acid binding domain comprises a Ralstonia repeat unit. In further aspects, the Ralstonia repeat unit is a Ralstonia solanacearum repeat unit. In still further aspects, the B₁₄₋₃₅ of at least one repeat unit of the plurality of repeat units has at least 92% sequence identity to GGKQALEAVRAQLLDLRAAPYG (SEQ ID NO: 280).

In some embodiments, a modular nucleic acid binding sequence (e.g., RNBD) can comprise one or more of the following characteristics: the modular nucleic acid binding sequence (e.g., RNBD) can bind a nucleic acid sequence, wherein the target site comprises a 5′ guanine, the modular nucleic acid binding domain (e.g., RNBD) can comprise 7 repeat units to 25 repeat units, a first modular nucleic acid binding sequence (e.g., RNBD) can bind a target nucleic acid sequence and be separated from a second modular nucleic acid binding domain (e.g., RNBD) from 2 to 50 base pairs, or any combination thereof.

In some embodiments, an RNBD of the present disclosure can have the full length naturally occurring N-terminus of a naturally occurring Ralstonia solanacearum-derived protein. In some embodiments, any truncation of the full length naturally occurring N-terminus of a naturally occurring Ralstonia solanacearum-derived protein can be used at the N-terminus of an RNBD of the present disclosure. For example, in some embodiments, amino acid residues at positions 1 (H) to position 137 (F) of the naturally occurring Ralstonia solanacearum-derived protein N-terminus can be used. In particular embodiments, said truncated N-terminus from position 1 (H) to position 137 (F) can have a sequence as follows: FGKLVALGYSREQIRKLKQESLSEIAKYHTTLTGQGFTHADICRISRRRQSLRVVARNYPELA AALPELTRAHIVDIARQRSGDLALQALLPVATALTAAPLRLSASQIATVAQYGERPAIQALY RLRRKLTRAPLH (SEQ ID NO: 264). In some embodiments, the naturally occurring N-terminus of Ralstonia solanacearum can be truncated to any length and used at the N-terminus of the engineered DNA binding domain. For example, the naturally occurring N-terminus of Ralstonia solanacearum can be truncated to amino acid residues at position 1 (H) to position 120 (K) as follows: KQESLSEIAKYHTTLTGQGFTHADICRISRRRQSLRVVARNYPELAAALPELTRAHIVDIARQ RSGDLALQALLPVATALTAAPLRLSASQIATVAQYGERPAIQALYRLRRKLTRAPLH (SEQ ID NO: 303) and used at the N-terminus of the RNBD. The naturally occurring N-terminus of Ralstonia solanacearum can be truncated such that it includes amino acid residues at positions 1 to 115 and used as the N-terminus of the engineered DNA binding domain. In certain aspects, the truncated N-terminus sequence may be at least 80%, 85%, 90%, 95%, 98%, 99%, or more identical to the amino acid sequence set forth in SEQ ID NO: 320. The naturally occurring N-terminus of Ralstonia solanacearum can be truncated to amino acid residues at positions 1 to 50, 1 to 70, 1 to 100, 1 to 120, 1 to 130, 10 to 40, 60 to 100, or 100 to 120 and used at the N-terminus of the engineered DNA binding domain. Truncation of the N-termini can be particularly advantageous for obtaining DNA binding domains, which are smaller in size including number of amino acids and overall molecular weight. A reduced number of amino acids can allow for more efficient packaging into a viral vector and a smaller molecular weight can result in more efficient loading of the DNA binding domains in non-viral vectors for delivery.

In some embodiments, the N-terminus, referred to as the amino terminus or the “NH2” domain, can recognize a guanine. In some embodiments, the N-terminus can be engineered to bind a cytosine, adenosine, thymidine, guanine, or uracil.

In some embodiments, an RNBD of the present disclosure can have a DNA binding domain, in which the final full length repeat unit of 33-35 amino acid residues is followed by a half repeat also derived from Ralstonia solanacearum. The half repeat can have 15 to 23 amino acid residues, for example, the half repeat can have 19 amino acid residues. In particular embodiments, the half repeat can have a sequence as follows: LSTAQVVAIACISGQQALE (SEQ ID NO: 265).

In some embodiments, an RNBD of the present disclosure can have the full length naturally occurring C-terminus of a naturally occurring Ralstonia solanacearum-derived protein. In some embodiments, any truncation of the full length naturally occurring C-terminus of a naturally occurring Ralstonia solanacearum-derived protein can be used at the C-terminus of an RNBD of the present disclosure. For example, in some embodiments, the RNBD can comprise amino acid residues at position 1 (A) to position 63 (S) as follows: AIEAHMPTLRQASHSLSPERVAAIACIGGRSAVEAVRQGLPVKAIRRIRREKAPVAGPPPAS (SEQ ID NO: 266) of the naturally occurring Ralstonia solanacearum-derived protein C-terminus. In some embodiments, the naturally occurring C-terminus of Ralstonia solanacearum can be truncated to any length and used at the C-terminus of the RNBD. For example, the naturally occurring C-terminus of Ralstonia solanacearum can be truncated to amino acid residues at positions 1 to 63 and used at the C-terminus of the RNBD. The naturally occurring C-terminus of Ralstonia solanacearum can be truncated amino acid residues at positions 1 to 50 and used at the C-terminus of the RNBD. The naturally occurring C-terminus of Ralstonia solanacearum can be truncated to amino acid residues at positions 1 to 63, 1 to 50, 1 to 70, 1 to 100, 1 to 120, 1 to 130, 10 to 40, 60 to 100, or 100 to 120 and used at the C-terminus of the RNBD.

TABLE 3 shows N-termini, C-termini, and half repeats derived from Ralstonia.

TABLE 3
Ralstonia-Derived N-terminus, C-terminus, and Half-Repeat

| SEQ ID NO | Description | Sequence |
|---|---|---|
| SEQ ID NO: 320 | Truncated N-terminus; positions 1 | SEIAKYHTTLTGQGFTHADICRISRRRQS |
| | (H) to 115 (S) of the naturally | LRVVARNYPELAAALPELTRAHIVDIAR |
| | occurring Ralstonia solanacearum- | QRSGDLALQALLPVATALTAAPLRLSAS |
| | derived protein N-terminus | QIATVAQYGERPAIQALYRLRRKLTRAP |
| | | LH |
| SEQ ID NO: 264 | Truncated N-terminus; positions 1 | FGKLVALGYSREQIRKLKQESLSEIAKYH |
| | (H) to 137 (F) of the naturally | TTLTGQGFTHADICRISRRRQSLRVVARN |
| | occurring Ralstonia solanacearum- | YPELAAALPELTRAHIVDIARQRSGDLAL |
| | derived protein N-terminus | QALLPVATALTAAPLRLSASQIATVAQY |
| | | GERPAIQALYRLRRKLTRAPLH |
| SEQ ID NO: 303 | Truncated N-terminus; positions 1 | KQESLSEIAKYHTTLTGQGFTHADICRIS |
| | (H) to 120 (K) of the naturally | RRRQSLRVVARNYPELAAALPELTRAHI |
| | occurring Ralstonia solanacearum- | VDIARQRSGDLALQALLPVATALTAAPL |
| | derived protein N-terminus | RLSASQIATVAQYGERPAIQALYRLRRK |
| | | LTRAPLH |
| SEQ ID NO: 265 | Half-repeat | LSTAQVVAIACISGQQALE |
| SEQ ID NO: 266 | Truncated C-terminus; positions 1 | AIEAHMPTLRQASHSLSPERVAAIACIGG |
| | (A) to 63 (S) of the naturally | RSAVEAVRQGLPVKAIRRIRREKAPVAG |
| | occurring Ralstonia solanacearum- | PPPAS |
| | derivedprotein C-terminus | |

In some embodiments, an RNBD can be engineered to target and bind to a site in the PDCD1 gene. For example, an RNBD with the sequence FGKLVALGYSREQIRKLKQESLSEIAKYHTTLTGQGFTHADICRISRRRQSLRVVARNYPELA AALPELTRAHIVDIARQRSGDLALQALLPVATALTAAPLRLSASQIATVAQYGERPAIQALY RLRRKLTRAPLHLTPQQVVAIASNTGGKRALEAVCVQLPVLRAAPYRLSTEQVVAIASHDG GKQALEAVGAQLVALRAAPYALSTEQVVAIASHDGGKQALEAVGAQLVALRAAPYALST AQVVAIASNGGGKQALEGIGEQLLKLRTAPYGLSTEQVVAIASNKGGKQALEAVKAHLLDL LGAPYVLSTEQVVAIASNKGGKQALEAVKAHLLDLLGAPYVLSTEQVVAIASNKGGKQAL EAVKAHLLDLLGAPYVLSTEQVVVIANSIGGKQALEAVKVQLPVLRAAPYELSTEQVVAIA SHDGGKQALEAVGAQLVALRAAPYALSTEQVVVIANSIGGKQALEAVKVQLPVLRAAPYE LSTEQVVAIASNKGGKQALEAVKAHLLDLLGAPYVLSTAQVVAIASNGGGKQALEGIGEQL LKLRTAPYGLSTAQVVAIASNGGGKQALEGIGEQLLKLRTAPYGLSTAQVVAIASNGGGKQ ALEGIGEQLLKLRTAPYGLSTEQVVAIASHDGGKQALEAVGAQLVALRAAPYALSTEQVVA IASHDGGKQALEAVGAQLVALRAAPYALSTEQVVAIASHDGGKQALEAVGAQLVALRAAP YALSTAQVVAIASNGGGKQALEGIGEQLLKLRTAPYGLSTAQVVAIASNGGGKQALEGIGE QLLKLRTAPYGLSTAQVVAIACISGQQALEAIEAHMPTLRQASHSLSPERVAAIACIGGRSAV EAVRQGLPVKAIRRIRREKAPVAGPPPAS (SEQ ID NO: 311) can bind to the GACCTGGGACAGTTTCCCTT (SEQ ID NO: 312) nucleic acid sequence in the PDCD1 gene. As another example, an RNBD with the sequence FGKLVALGYSREQIRKLKQESLSEIAKYHTTLTGQGFTHADICRISRRRQSLRVVARNYPELA AALPELTRAHIVDIARQRSGDLALQALLPVATALTAAPLRLSASQIATVAQYGERPAIQALY RLRRKLTRAPLHLTPQQVVAIASNTGGKRALEAVCVQLPVLRAAPYRLSTAQVVAIASNGG GKQALEGIGEQLLKLRTAPYGLSTEQVVAIASHDGGKQALEAVGAQLVALRAAPYALSTA QVVAIASNGGGKQALEGIGEQLLKLRTAPYGLSTEQVVAIASHNGGKQALEAVKADLLELR GAPYALSTEQVVAIASHDGGKQALEAVGAQLVALRAAPYALSTEQVVVIANSIGGKQALEA VKVQLPVLRAAPYELSTAQVVAIASNGGGKQALEGIGEQLLKLRTAPYGLSTEQVVAIASH NGGKQALEAVKADLLELRGAPYALSTEQVVAIASHDGGKQALEAVGAQLVALRAAPYALS TEQVVAIASHDGGKQALEAVGAQLVALRAAPYALSTAQVVAIASNGGGKQALEGIGEQLL KLRTAPYGLSTEQVVAIASHNGGKQALEAVKADLLELRGAPYALSTEQVVAIASHNGGKQ ALEAVKADLLELRGAPYALSTEQVVVIANSIGGKQALEAVKVQLPVLRAAPYELSTEQVVA IASHNGGKQALEAVKADLLELRGAPYALSTEQVVAIASHDGGKQALEAVGAQLVALRAAP YALSTAQVVAIACISGQQALEAIEAHMPTLRQASHSLSPERVAAIACIGGRSAVEAVRQGLP VKAIRRIRREKAPVAGPPPAS (SEQ ID NO: 313) can bind to the GATCTGCATGCCTGGAGC (SEQ ID NO: 314) nucleic acid sequence in the PDCD1 gene. As yet another example, an RNBD with the sequence FGKLVALGYSREQIRKLKQESLSEIAKYHTTLTGQGFTHADICRISRRRQSLRVVARNYPELA AALPELTRAHIVDIARQRSGDLALQALLPVATALTAAPLRLSASQIATVAQYGERPAIQALY RLRRKLTRAPLHLTPQQVVAIASNTGGKRALEAVCVQLPVLRAAPYRLSTAQVVAIASNGG GKQALEGIGEQLLKLRTAPYGLSTEQVVAIASHDGGKQALEAVGAQLVALRAAPYALSTA QVVAIASNGGGKQALEGIGEQLLKLRTAPYGLSTEQVVAIASHNGGKQALEAVKADLLELR GAPYALSTEQVVAIASHDGGKQALEAVGAQLVALRAAPYALSTAQVVAIATRSGGKQALE AVRAQLLDLRAAPYGLSTAQVVAIASNGGGKQALEGIGEQLLKLRTAPYGLSTEQVVAIAS HNGGKQALEAVKADLLELRGAPYALSTEQVVAIASHDGGKQALEAVGAQLVALRAAPYA LSTEQVVAIASHDGGKQALEAVGAQLVALRAAPYALSTAQVVAIASNGGGKQALEGIGEQ LLKLRTAPYGLSTEQVVAIASHNGGKQALEAVKADLLELRGAPYALSTEQVVAIASHNGGK QALEAVKADLLELRGAPYALSTAQVVAIATRSGGKQALEAVRAQLLDLRAAPYGLSTEQV VAIASHNGGKQALEAVKADLLELRGAPYALSTEQVVAIASHDGGKQALEAVGAQLVALRA APYALSTAQVVAIACISGQQALEAIEAHMPTLRQASHSLSPERVAAIACIGGRSAVEAVRQG LPVKAIRRIRREKAPVAGPPPAS (SEQ ID NO: 315) can bind to the GATCTGCATGCCTGGAGC (SEQ ID NO: 314) nucleic acid sequence in the PDCD1 gene. Any one of SEQ ID NO: 311, SEQ ID NO; 313, or SEQ ID NO: 315 can be fused to any repression domain described herein (e.g., KRAB) to yield a gene repressor capable of repressing expression of the target gene.

### Xanthomonas Derived Transcription Activator Like Effector (TALE)

The present disclosure provides a modular nucleic acid binding domain derived from Xanthomonas spp., also referred to herein as a transcription activator-like effector (TALE) protein, can comprise a plurality of repeat units. A repeat unit of the plurality of repeat units recognizes a single target nucleotide, base pair, or both. A repeat unit from Xanthomonas spp. can comprise 33-35 amino acid residues. In some embodiments, a repeat unit can be from Xanthomonas spp. protein having the sequence:

(SEQ ID NO: 299)
MDPIRSRTPSPARELLPGPQPDGVQPTADRGVSPPAGGPLDGLPARR
TMSRTRLPSPPAPSPAFSAGSFSDLLRQFDPSLFNTSLFDSLPPFGA
HHTEAATGEWDEVQSGLRAADAPPPTMRVAVTAARPPRAKPAPRRRA
AQPSDASPAAQVDLRTLGYSQQQQEKIKPKVRSTVAQHHEALVGHGF
THAHIVALSQHPAALGTVAVKYQDMIAALPEATHEAIVGVGKQWSGA
RALEALLTVAGELRGPPLQLDTGQLLKIAKRGGVTAVEAVHAWRNAL
TGAPLNLTPEQVVAIASHDGGKQALETVQRLLPVLCQAHGLTPQQVV
AIASNGGGKQALETVQRLLPVLCQAHGLTPQQVVAIASNSGGKQALE
TVQRLLPVLCQAHGLTPEQVVAIASNGGGKQALETVQRLLPVLCQAH
GLTPEQVVAIASNIGGKQALETVQALLPVLCQAHGLTPEQVVAIASN
IGGKQALETVQALLPVLCQAHGLTPEQVVAIASNIGGKQALETVQAL
LPVLCQAHGLTPEQVVAIASHDGGKQALETVQRLLPVLCQAHGLTPE
QVVAIASHDGGKQALETVQRLLPVLCQAHGLTPQQVVAIASNGGGKQ
ALETVQRLLPVLCQAHGLTPEQVVAIASNSGGKQALETVQALLPVLC
QAHGLTPEQVVAIASNSGGKQALETVQRLLPVLCQAHGLTPEQVVAI
ASHDGGKQALETVQRLLPVLCQAHGLTPEQVVAIASHDGGKQALETV
QRLLPVLCQAHGLTPEQVVAIASHDGGKQALETVQRLLPVLCQAHGL
TPQQVVAIASNGGGRPALETVQRLLPVLCQAHGLTPEQVVAIASHDG
GKQALETVQRLLPVLCQAHGLTPQQVVAIASNGGGRPALESIVAQLS
RPDPALAALTNDHLVALACLGGRPALDAVKKGLPHAPALIKRTNRRI
PERTSHRVADHAQVVRVLGFFQCHSHPAQAFDDAMTQFGMSRHGLLQ
LFRRVGVTELEARSGTLPPASQRWDRILQASGMKRAKPSPTSTQTPD
QASLHAFADSLERDLDAPSPMHEGDQTRASSRKRSRSDRAVTGPSAQ
QSFEVRVPEQRDALHLPLSWRVKRPRTSIGGGLPDPGTPTAADLAAS
STVMREQDEDPFAGAADDFPAFNEEELAWLMELLPQ.

In some embodiments, a TALE of the present disclosure can comprise between 1 to 50 Xanthomonas spp.-derived repeat units. In some embodiments, a TALE of the present disclosure can comprise between 9 and 36 Xanthomonas spp.-derived repeat units. Preferably, in some embodiments, a TALE of the present disclosure can comprise between 12 and 30 Xanthomonas spp.-derived repeat units. A TALE described herein can comprise between 5 to 10 Xanthomonas spp.-derived repeat units, between 10 to 15 Xanthomonas spp.-derived repeat units, between 15 to 20 Xanthomonas spp.-derived repeat units, between 20 to 25 Xanthomonas spp.-derived repeat units, between 25 to 30 Xanthomonas spp.-derived repeat units, or between 30 to 35 Xanthomonas spp.-derived repeat units, between 35 to 40 Xanthomonas spp.-derived repeat units. A TALE described herein can comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or more Xanthomonas spp.-derived repeat units, such as, repeat units derived from Xanthomonas spp. protein having the amino acid sequence set forth in SEQ ID NO:299.

A Xanthomonas spp.-derived repeat units can be derived from a wild-type repeat unit, such as any one of SEQ ID NO: 323-SEQ ID NO: 326. For example, a Xanthomonas spp.-derived repeat units can have a sequence of LTPDQVVAIASNHGGKQALETVQRLLPVLCQDHG (SEQ ID NO: 323) comprising an RVD of NH, which recognizes guanine. A Xanthomonas spp.-derived repeat units can have a sequence of LTPDQVVAIASNGGGKQALETVQRLLPVLCQDHG (SEQ ID NO: 324) comprising an RVD of NG, which recognizes thymidine. A Xanthomonas spp.-derived repeat units can have a sequence of LTPDQVVAIASNIGGKQALETVQRLLPVLCQDHG (SEQ ID NO: 325) comprising an RVD of NI, which recognizes adenosine. A Xanthomonas spp.-derived repeat units can have a sequence of LTPDQVVAIASHDGGKQALETVQRLLPVLCQDHG (SEQ ID NO: 326) comprising an RVD of HD, which recognizes cytosinc.

A Xanthomonas spp.-derived repeat unit can also comprise a modified Xanthomonas spp.-derived repeat units enhanced for specific recognition of a nucleotide or base pair. A TALE described herein can comprise one or more wild-type Xanthomonas spp.-derived repeat units, one or more modified Xanthomonas spp.-derived repeat units, or a combination thereof. In some embodiments, a modified Xanthomonas spp.-derived repeat units can comprise 1, 2, 3, 4, 5, 6, 7, 8, 9, 10, 11, 12, 13, 14, 15, 16, 17, 18, 19, 20, 21, 22, 23, 24, 25, 26, 27, 28, or 29 mutations that can enhance recognition of a specific nucleotide or base pair. In some embodiments, a modified Xanthomonas spp.-derived repeat unit can comprise more than 1 modification, for example 1 to 5 modifications, 5 to 10 modifications, 10 to 15 modifications, 15 to 20 modifications, 20 to 25 modification, or 25-29 modifications. In some embodiments, A TALE can comprise more than one modified Xanthomonas spp.-derived repeat units, wherein each of the modified Xanthomonas spp.-derived repeat units can have a different number of modifications.

In some embodiments, a TALE of the present disclosure can have the full length naturally occurring N-terminus of a naturally occurring Xanthomonas spp.-derived protein, such as the N-terminus of SEQ ID NO: 299. The N-terminus sequence in SEQ ID NO:299 is indicated by underlining.

In some embodiments, a TALE of the present disclosure can comprise the amino acid residues at position 1 (N) through position 137 (M) of the naturally occurring Xanthomonas spp.-derived protein as follows:

(SEQ ID NO: 300)
MVDLRTLGYSQQQQEKIKPKVRSTVAQHHEALVGHGFTHAHIVALS
QHPAALGTVAVKYQDMIAALPEATHEAIVGVGKQWSGARALEALLT
VAGELRGPPLQLDTGQLLKIAKRGGVTAVEAVHAWRNALTGAPLN

The amino acid sequence set forth in SEQ ID NO:300 includes a M added to the N-terminus which is not present in the wild type N-terminus region of a TALE protein. The N-terminus fragment sequence set out in SEQ ID NO:300 is generated by deleting amino acids N+288 through N+137 of the N-terminus region of a TALE protein, adding a M, such that amino acids N+136 through N+1 of the N-terminus region of the TALE protein are present.

In some embodiments, the N-terminus can be truncated such that the fragment of the N-terminus includes amino acids from position 1 (N) through position 120 (K) of the naturally occurring Xanthomonas spp.-derived protein as follows:

(SEQ ID NO: 301)
KPKVRSTVAQHHEALVGHGFTHAHIVALSQHPAALGTVAVKYQDMI
AALPEATHEAIVGVGKQWSGARALEALLTVAGELRGPPLQLDTGQL
LKIAKRGGVTAVEAVHAWRNALTGAPLN

In some embodiments, the N-terminus can be truncated such that the fragment of the N-terminus includes amino acids from position 1 (N) through position 115 (S) of the naturally occurring Xanthomonas spp.-derived protein as follows:

(SEQ ID NO: 321)
STVAQHHEALVGHGFTHAHIVALSQHPAALGTVAVKYQDMIAALPE
ATHEAIVGVGKQWSGARALEALLTVAGELRGPPLQLDTGQLLKIAK
RGGVTAVEAVHAWRNALTGAPLN .

In some embodiments, the N-terminus can be truncated such that the fragment of the N-terminus includes amino acids from position 1 (N) through position 110 (H) of the naturally occurring Xanthomonas spp.-derived protein as follows:

(SEQ ID NO: 447)
HHEALVGHGFTHAHIVALSQHPAALGTVAVKYQDMIAALPEATHEA
IVGVGKQWSGARALEALLTVAGELRGPPLQLDTGQLLKIAKRGGVT
AVEAVHAWRNALTGAPLN .

In some embodiments, a truncation of the naturally occurring Xanthomonas spp.-derived protein can be used at the N-terminus of a TALE disclosed herein. In some embodiments, a truncation of the naturally occurring Xanthomonas spp.-derived protein can be used at the N-terminus of a TALE disclosed herein and may include an amino acid sequence at least 85%, 90%, 95%, 96%, 97%, 98%, or 99% identical to the amino acid sequences set forth in one of SEQ ID NOs: 300, 301, 321, and 447. The naturally occurring N-terminus of Xanthomonas spp. can be truncated to amino acid residues at positions 1 to 50, 1 to 70, 1 to 100, 1 to 120, 1 to 130, 10 to 40, 60 to 100, or 100 to 120 and used at the N-terminus of the TALE.

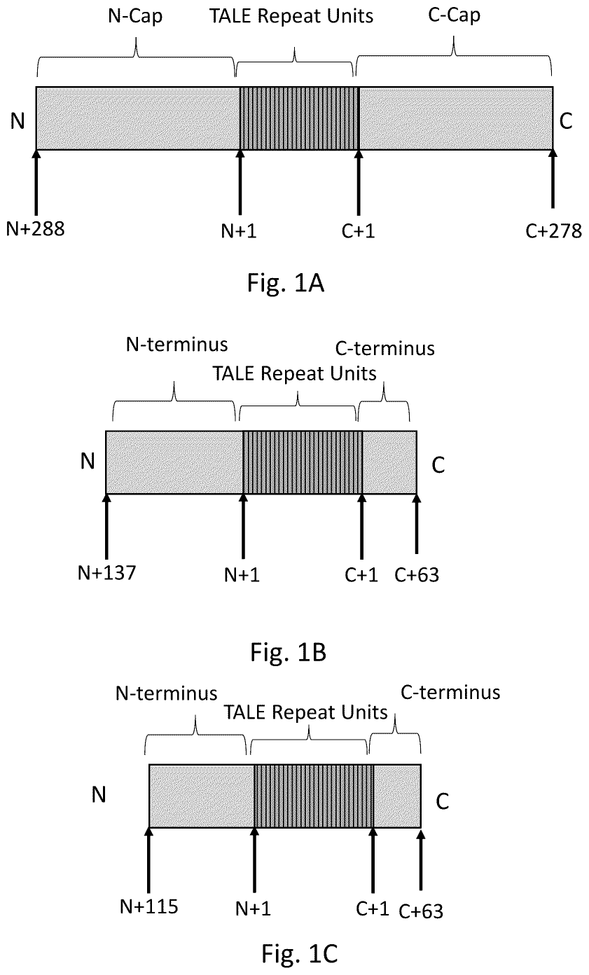

FIGS. 1A-1C show schematics of the domain structure of a TALE protein (not drawn to scale). ‘N’ and C′ indicate the amino and carboxy termini, respectively. The TALE repeat domain comprising TALE repeat units, N-Cap and C-Cap regions are labeled and the residue numbering scheme for the N-Cap and C-Cap regions and the N-terminus and C-terminus fragments are indicated. FIG. 1A includes the full-length N-cap region that extends from amino acid position N+1 to N+288 and full-length C-cap region that extends from amino acid position C+1 through C+278. FIG. 1B provides a schematic of a DNA binding protein comprising TALE repeat units and a truncated N-terminus that extends from amino acid position N+1 to N+136 (the notation N+137 indicates that a methionine added to the N-terminus increases the length to 137) and a truncated C-terminus that extends from amino acid position C+1 through C+63. FIG. 1C provides a schematic of a DNA binding protein comprising TALE repeat units and a truncated N-terminus that extends from amino acid position N+1 to N+115 and a truncated C-terminus that extends from amino acid position C+1 through C+63. In certain cases, the last repeat domain may be a half-repeat or a partial repeat as disclosed herein.

In some embodiments, a TALE of the present disclosure can have a DNA binding domain, in which the final full length repeat unit of 33-35 amino acid residues is followed by a half repeat also derived from Xanthomonas spp. The half repeat can have 15 to 23 amino acid residues, for example, the half repeat can have 19 amino acid residues. In particular embodiments, the half repeat can have a sequence as set forth in LTPQQVVAIASNGGGRPALE (SEQ ID NO: 297). In some embodiments, the half repeat can have a sequence as set forth in SEQ ID NO: 327, 328, 329, 330, 331, 332, 333, or 334).

TABLE 4
Xanthomonas Repeat Sequences

| SEQ ID | | |
|---|---|---|
| NO | Amino Acid Sequence | Description |
| 323 | LTPDQVVAIASNHGGKQALETVQRLLPVLCQDHG | RVD NH recognizing G |
| 324 | LTPDQVVAIASNGGGKQALETVQRLLPVLCQDHG | RVD NG recognizing T |
| 325 | LTPDQVVAIASNIGGKQALETVQRLLPVLCQDHG | RVD NI recognizing A |
| 326 | LTPDQVVAIASHDGGKQALETVQRLLPVLCQDHG | RVD HD recognizing C |
| 297 | LTPQQVVAIASNGGGRPALE | Half repeat |
| 327 | LTPEQVVAIASNGGGRPALE | Half repeat |
| 328 | LTPDQVVAIASNGGGRPALE | Half repeat |
| 329 | LTPEQVVAIASNIGGRPALE | Half repeat |
| 330 | LTPDQVVAIASNIGGRPALE | Half repeat |
| 331 | LTPEQVVAIASHDGGRPALE | Half repeat |
| 332 | LTPDQVVAIASHDGGRPALE | Half repeat |
| 333 | LTPEQVVAIASNHGGRPALE | Half repeat |
| 334 | LTPDQVVAIASNHGGRPALE | Half repeat |

In some embodiments, a TALE of the present disclosure can have the full length naturally occurring C-terminus of a naturally occurring Xanthomonas spp.-derived protein, such as the C-terminus of SEQ ID NO: 299. The C-terminus of the TALE protein sequence set forth in SEQ ID NO: 299 is italicized. In some embodiments, the C-terminus can be a fragment of the full length naturally occurring C-terminus of a naturally occurring Xanthomonas spp.-derived protein. In some embodiments, the C-terminus can be less than 250 amino acids long. In some embodiments, the C-terminus can be positions 1 (S) through position 278 (Q) of the naturally occurring Xanthomonas spp.-derived protein as follows: SIVAQLSRPDPALAALTNDHLVALACLGGRPALDAVKKGLPHAPALIKRTNRRIPERTSHRV ADHAQVVRVLGFFQCHSHPAQAFDDAMTQFGMSRHGLLQLFRRVGVTELEARSGTLPPAS QRWDRILQASGMKRAKPSPTSTQTPDQASLHAFADSLERDLDAPSPTHEGDQRRASSRKRS RSDRAVTGPSAQQSFEVRAPEQRDALHLPLSWRVKRPRTSIGGGLPDPGTPTAADLAASSTV MREQDEDPFAGAADDFPAFNEEELAWLMELLPQ (SEQ ID NO: 302). In some embodiments, any truncation of the full length naturally occurring C-terminus of a naturally occurring Xanthomonas spp.-derived protein can be used at the C-terminus of a TALE of the present disclosure. For example, in some embodiments, the naturally occurring N-terminus of Xanthomonas spp. can be truncated to amino acid residues at position 1 (S) to position 63 (X) as follows: SIVAQLSRPDPALAALTNDHLVALACLGGRPALDAVKKGLPHAPALIKRTNRRIPERTSHRV A (SEQ ID NO: 298). The naturally occurring C-terminus of Xanthomonas spp. can be truncated amino acid residues at positions 1 to 50 and used at the C-terminus of the engineered DNA binding domain. The naturally occurring C-terminus of Xanthomonas spp. can be truncated to amino acid residues at positions 1 to 63, 1 to 50, 1 to 70, 1 to 100, 1 to 120, 1 to 130, 10 to 40, 60 to 100, or 100 to 120 and used at the C-terminus of the engineered DNA binding domain.

The terms “N-cap” polypeptide and “N-terminal sequence” are used to refer to an amino acid sequence (polypeptide) that flanks the N-terminal portion of the first TALE repeat unit. The N-cap sequence can be of any length (including no amino acids), so long as the TALE-repeat unit(s) function to bind DNA. An N-terminal fragment and grammatical equivalents thereof refers to a shortened sequence of an N-terminal sequence which fragment is sufficient for the TALE repeat units to bind to DNA.

The term “C-cap” or “C-terminal region” refers to optionally present amino acid sequences that may be flanking the C-terminal portion of the last TALE repeat unit. The C-cap can also comprise any part of a terminal C-terminal TALE repeat, including 0 residues, truncations of a TALE repeat or a full TALE repeat. A C-terminal fragment and grammatical equivalents thereof refers to a shortened sequence of a C-terminal sequence which fragment is sufficient for the TALE repeat units to bind to DNA.

### Animal Pathogen Derived Modular Nucleic Acid Binding Domains

The present disclosure provides a modular nucleic acid binding domain derived from an animal pathogen protein (MAP-NBD) can comprise a plurality of repeat units, wherein a repeat unit of the plurality of repeat units recognizes a single target nucleotide, base pair, or both.

In some embodiments, the repeat unit can be derived from an animal pathogen, and can be referred to as a non-naturally occurring modular nucleic acid binding domain derived from an animal pathogen protein (MAP-NBD), or “modular animal pathogen-nucleic acid binding domain” (MAP-NBD). For example, in some cases, the animal pathogen can be from the Gram-negative bacterium genus, Legionella. In other cases, the animal pathogen can be from Burkholderia. In some cases, the animal pathogen can be from Paraburkholderia. In other cases, the animal pathogen can be from Francisella.

In particular embodiments, the repeat unit can be derived from a species of the genus of Legionella, such as Legionella quateirensis, the genus of Burkholderia, the genus of Paraburkholderia, or the genus of Francisella. In some embodiments, the repeat unit can comprise from 19 amino acid residues to 35 amino acid residues. In particular embodiments, the repeat unit can comprise 33 amino acid residues. In other embodiments, the repeat unit can comprise 35 amino acid residues. In some embodiments, the MAP-NBD is non-naturally occurring, and comprises a plurality of repeat units and wherein a repeat unit of the plurality of repeat units recognizes a single target nucleic acid.

In some embodiments, a repeat unit can be derived from a Legionella quateirensis protein with the following sequence:

(SEQ ID NO: 281)
MPDLELNFAIPLHLFDDETVFTHDATNDNSQASSSYSSKSSPASAN
ARKRTSRKEMSGPPSKEPANTKSRRANSQNNKLSLADRLTKYNIDE
EFYQTRSDSLLSLNYTKKQIERLILYKGRTSAVQQLLCKHEELLNL
ISPDGLGHKELIKIAARNGGGNNLIAVLSCYAKLKEMGFSSQQIIR
MVSHAGGANNLKAVTANHDDLQNMGFNVEQIVRMVSHNGGSKNLKA
VTDNHDDLKNMGFNAEQIVRMVSHGGGSKNLKAVTDNHDDLKNMGF
NAEQIVSMVSNNGGSKNLKAVTDNHDDLKNMGFNAEQIVSMVSNGG
GSLNLKAVKKYHDALKDRGENTEQIVRMVSHDGGSLNLKAVKKYHD
ALRERKFNVEQIVSIVSHGGGSLNLKAVKKYHDVLKDREFNAEQIV
RMVSHDGGSLNLKAVTDNHDDLKNMGFNAEQIVRMVSHKGGSKNLA
LVKEYFPVFSSFHFTADQIVALICQSKQCFRNLKKNHQQWKNKGLS
AEQIVDLILQETPPKPNFNNTSSSTPSPSAPSFFQGPSTPIPTPVL
DNSPAPIFSNPVCFFSSRSENNTEQYLQDSTLDLDSQLGDPTKNFN
VNNFWSLFPFDDVGYHPHSNDVGYHLHSDEESPFFDF.

In some embodiments, a repeat from a Legionella quateirensis protein can comprise a repeat with a canonical RVD or a non-canonical RVD. In some embodiments, a canonical RVD can comprise NN, NG, HD, or HD. In some embodiments, a non-canonical RVD can comprise RN, HA, HN, HG, HG, or HK.

In some embodiments, a repeat of SEQ ID NO: 282 comprises an RVD of HA and primarily recognizes a base of adenine (A). In some embodiments, a repeat of SEQ ID NO: 283 comprises an RVD of HN and recognizes a base comprising guanine (G). In some embodiments, a repeat of SEQ ID NO: 284 comprises an RVD of HG and recognizes a base comprising thymine (T). In some embodiments, a repeat of SEQ ID NO: 285 comprises an RVD of NN and recognizes a base comprising guanine (G). In some embodiments, a repeat of SEQ ID NO: 286 comprises an RVD of NG and recognizes a base comprising thymine (T). In some embodiments, a repeat of SEQ ID NO: 287 comprises an RVD of HD and recognizes a base comprising cytosine (C). In some embodiments, a repeat of SEQ ID NO: 288 comprises an RVD of HG and recognizes a base comprising thymine (T). In some embodiments, a repeat of SEQ ID NO: 289 comprises an RVD of HD and recognizes a base comprising cytosine (C). In some embodiments, a half-repeat of SEQ ID NO: 290 comprises an RVD of HK and recognizes a base comprising guanine (G). In some embodiments, a repeat of SEQ ID NO: 357 comprises an RVD of RN and recognizes a base comprising guanine (G).

TABLE 5 illustrates exemplary repeats from Legionella quateirensis, Burkholderia, Paraburkholderia, or Francisella that can make up a MAP-NBD of the present disclosure and the RVD at position 12 and 13 of the particular repeat. A MAP-NBD of the present disclosure can comprise at least one of the repeats disclosed in TABLE 5 including any one of SEQ ID NO: 357, SEQ ID NO: 282-SEQ ID NO: 290, or SEQ ID NO: 358-SEQ ID NO: 446. A MAP-NBD of the present disclosure can comprise any combination of repeats disclosed in TABLE 5 including any one of SEQ ID NO: 357, SEQ ID NO: 282-SEQ ID NO: 290, or SEQ ID NO: 358-SEQ ID NO: 446.

TABLE 5
Animal Pathogen Derived Repeat Units

| SEQ | | | |
|---|---|---|---|
| ID NO | Organism | Repeat Unit Sequence | RVD |
| 357 | L. quateirensis | LGHKELIKIAARNGGGNNLIAVLSCYAKLKEMG | RN |
| 282 | L. quateirensis | FSSQQIIRMVSHAGGANNLKAVTANHDDLQNMG | HA |
| 283 | L. quateirensis | FNVEQIVRMVSHNGGSKNLKAVTDNHDDLKNMG | HN |
| 284 | L. quateirensis | FNAEQIVRMVSHGGGSKNLKAVTDNHDDLKNMG | HG |
| 285 | L. quateirensis | FNAEQIVSMVSNNGGSKNLKAVTDNHDDLKNMG | NN |
| 286 | L. quateirensis | FNAEQIVSMVSNGGGSLNLKAVKKYHDALKDRG | NG |
| 287 | L. quateirensis | FNTEQIVRMVSHDGGSLNLKAVKKYHDALRERK | HD |
| 288 | L. quateirensis | FNVEQIVSIVSHGGGSLNLKAVKKYHDVLKDRE | HG |
| 289 | L. quateirensis | FNAEQIVRMVSHDGGSLNLKAVTDNHDDLKNMG | HD |
| 290 | L. quateirensis | FNAEQIVRMVSHKGGSKNL | HK |
| (half | | | |
| repeat) | | | |
| 358 | L. quateirensis | FSAEQIVRIAAHDGGSRNIEAVQQAQHVLKELG | HD |
| 359 | L. quateirensis | FSAEQIVSIVAHDGGSRNIEAVQQAQHILKELG | HD |
| 360 | L. quateirensis | FSRQQILRIASHDGGSKNIAAVQKFLPKLMNFGFN | HD |
| 361 | L. quateirensis | FSAEQIVRIAAHDGGSLNIDAVQQAQQALKELG | HD |
| 362 | L. quateirensis | FSTEQIVCIAGHGGGSLNIKAVLLAQQALKDLG | HG |
| 363 | L. quateirensis | FSSEQIVRVAAHGGGSLNIKAVLQAHQALKELD | HG |
| 364 | L. quateirensis | FSAEQIVHIAAHGGGSLNIKAILQAHQTLKELN | HG |
| 365 | L. quateirensis | FSAEQIVRIAAHIGGSRNIEAIQQAHHALKELG | HI |
| 366 | L. quateirensis | FSAEQIVRIAAHIGGSHNLKAVLQAQQALKELD | HI |
| 367 | L. quateirensis | FSAKHIVRIAAHIGGSLNIKAVQQAQQALKELG | HI |
| 368 | L. quateirensis | FNAEQIVRMVSHKGGSKNLALVKEYFPVFSSFH | HK |
| 369 | L. quateirensis | FNAEQIVRMVSHKGGSKNLALVKEYFPVFSSFHFT | HK |
| 370 | L. quateirensis | FSADQIVRIAAHKGGSHNIVAVQQAQQALKELD | HK |
| 371 | L. quateirensis | FNVEQIVRMVSHNGGSKNLKAVTDNHDDLKNMGFN | HN |
| 372 | L. quateirensis | FSADQVVKIAGHSGGSNNIAVMLA VFPRLRDFGFK | HS |
| 373 | L. quateirensis | FSAEQIVSIAAHVGGSHNIEAVQKAHQALKELD | HV |
| 374 | L. quateirensis | FNAEQIVSMVSNNGGSKNLKAVTDNHDDLKNMGFN | NN |
| 375 | L. quateirensis | FSHKELIKIAARNGGGNNLIAVLSCYAKLKEMG | RN |
| 376 | L. quateirensis | FSHKELIKIAARNGGGNNLIAVLSCYAKLKEMGFS | RN |
| 377 | Burkholderia | FSSGETVGATVGAGGTETVAQGGTASNTTVSSGGY | GA |
| 378 | Burkholderia | FSGGMATSTTVGSGGTQDVLAGGAAVGGTVGTGGV | GS |
| 379 | Burkholderia | FSAADIVKIAGKIGGAQALQAFITHRAALIQAGFS | KI |
| 380 | Burkholderia | FNPTDIVKIAGNDGGAQALQAVLELEPALRERGFS | ND |
| 381 | Burkholderia | FNPTDIVRMAGNDGGAQALQAVFELEPAFRERSFS | ND |
| 382 | Burkholderia | FNPTDIVRMAGNDGGAQALQAVLELEPAFRERGFS | ND |
| 383 | Burkholderia | FSQVDIVKIASNDGGAQALYSVLDVEPTFRERGFS | ND |
| 384 | Burkholderia | FSRADIVKIAGNDGGAQALYSVLDVEPPLRERGFS | ND |
| 385 | Burkholderia | FSRGDIVKIAGNDGGAQALYSVLDVEPPLRERGFS | ND |
| 386 | Burkholderia | FNRADIVRIAGNGGGAQALYSVRDAGPTLGKRGFS | NG |
| 387 | Burkholderia | FRQADIVKIASNGGSAQALNAVIKLGPTLRQRGFS | NG |
| 388 | Burkholderia | FRQADIVKMASNGGSAQALNAVIKLGPTLRQRGFS | NG |
| 389 | Burkholderia | FSRADIVKIAGNGGGAQALQAVLELEPTFRERGFS | NG |
| 390 | Burkholderia | FSRADIVRIAGNGGGAQALYSVLDVGPTLGKRGFS | NG |
| 391 | Burkholderia | FSRGDIVRIAGNGGGAQALQAVLELEPTLGERGFS | NG |
| 392 | Burkholderia | FSRADIVKIAGNGGGAQALQA VITHRAALTQAGFS | NG |
| 393 | Burkholderia | FSRGDTVKIAGNIGGAQALQAVLELEPTLRERGFS | NI |
| 394 | Burkholderia | FNPTDIVKIAGNIGGAQALQAVLELEPAFRERGFS | NI |
| 395 | Burkholderia | FSAADIVKIAGNIGGAQALQAIFTHRAALIQAGFS | NI |
| 396 | Burkholderia | FSAADIVKIAGNIGGAQALQA VITHRATLTQAGFS | NI |
| 397 | Burkholderia | FSATDIVKIASNIGGAQALQA VISRRAALIQAGFS | NI |
| 398 | Burkholderia | FSQPDIVKIAGNIGGAQALQAVLELEPAFRERGFS | NI |
| 399 | Burkholderia | FSRADIVKIAGNIGGAQALQAVLELESTFRERSFN | NI |
| 400 | Burkholderia | FSRADIVKIAGNIGGAQALQAVLELESTLRERSFN | NI |
| 401 | Burkholderia | FSRGDIVKMAGNIGGAQALQAGLELEPAFRERGFS | NI |
| 402 | Burkholderia | FSRGDIVKMAGNIGGAQALQAVLELEPAFHERSFC | NI |
| 403 | Burkholderia | FTLTDIVKMAGNIGGAQALKAVLEHGPTLRQRDLS | NI |
| 404 | Burkholderia | FTLTDIVKMAGNIGGAQALKVVLEHGPTLRQRDLS | NI |
| 405 | Burkholderia | FNPTDIVKIAGNNGGAQALQAVLELEPALRERGFS | NN |
| 406 | Burkholderia | FNPTDIVKIAGNNGGAQALQAVLELEPALRERSFS | NN |
| 407 | Burkholderia | FNPTDMVKIAGNNGGAQALQAVLELEPALRERGFS | NN |
| 408 | Burkholderia | FSAADIVKIASNNGGAQALQALIDHWSTLSGKTKA | NN |
| 409 | Burkholderia | FSAADIVKIASNNGGAQALQAVISRRAALIQAGFS | NN |
| 410 | Burkholderia | FSAADIVKIASNNGGAQALQAVITHRAALAQAGFS | NN |
| 411 | Burkholderia | FSAADIVKIASNNGGARALQALIDHWSTLSGKTKA | NN |
| 412 | Burkholderia | FTLTDIVEMAGNNGGAQALKAVLEHGSTLDERGFT | NN |
| 413 | Burkholderia | FTLTDIVKMAGNNGGAQALKAVLEHGPTLDERGFT | NN |
| 414 | Burkholderia | FTLTDIVKMAGNNGGAQALKVVLEHGPTLRQRGFS | NN |
| 415 | Burkholderia | FTLTDIVKMASNNGGAQALKAVLEHGPTLDERGFT | NN |
| 416 | Burkholderia | FSAADIVKIAGNSGGAQALQAVISHRAALTQAGFS | NS |
| 417 | Burkholderia | FSGGDAVSTVVRSGGAQSVASGGTASGTTVSAGAT | RS |
| 418 | Burkholderia | FRQTDIVKMAGSGGSAQALNAVIKHGPTLRQRGFS | SG |
| 419 | Burkholderia | FSLIDIVEIASNGGAQALKAVLKYGPVLTQAGRS | SN |
| 420 | Burkholderia | FSGGDAAGTVVSSGGAQNVTGGLASGTTVASGGAA | SS |
| 421 | Paraburkholderia | FNLTDIVEMAANSGGAQALKAVLEHGPTLRQRGLS | NS |
| 422 | Paraburkholderia | FNRASIVKIAGNSGGAQALQAVLKHGPTLDERGEN | NS |
| 423 | Paraburkholderia | FSQANIVKMAGNSGGAQALQAVLDLELVFRERGFS | NS |
| 424 | Paraburkholderia | FSQPDIVKMAGNSGGAQALQAVLDLELAFRERGFS | NS |
| 425 | Paraburkholderia | FSLIDIVEIASNGGAQALKAVLKYGPVLMQAGRS | SN |
| 426 | Francisella | YKSEDIIRLASHDGGSVNLEAVLRLHSQLTRLG | HD |
| 427 | Francisella | YKPEDIIRLASHGGGSVNLEAVLRLNPQLIGLG | HG |
| 428 | Francisella | YKSEDIIRLASHGGGSVNLEAVLRLHSQLTRLG | HG |
| 429 | Francisella | YKSEDIIRLASHGGGSVNLEAVLRLNPQLIGLG | HG |
| 430 | Paraburkholderia | FNLTDIVEMAGKGGGAQALKAVLEHGPTLRQRGEN | KG |
| 431 | Paraburkholderia | FRQADIIKIAGNDGGAQALQA VIEHGPTLRQHGFN | ND |
| 432 | Paraburkholderia | FSQADIVKIAGNDGGTQALHAVLDLERMLGERGFS | ND |
| 433 | Paraburkholderia | FSRADIVKIAGNGGGAQALKAVLEHEATLDERGFS | NG |
| 434 | Paraburkholderia | FSRADIVRIAGNGGGAQALYSVLDVEPTLGKRGFS | NG |
| 435 | Paraburkholderia | FSQPDIVKMASNIGGAQALQAVLELEPALRERGFS | NI |
| 436 | Paraburkholderia | FSQPDIVKMAGNIGGAQALQAVLSLGPALRERGFS | NI |
| 437 | Paraburkholderia | FSQPEIVKIAGNIGGAQALHTVLELEPTLHKRGFN | NI |
| 438 | Paraburkholderia | FSQSDIVKIAGNIGGAQALQAVLDLESMLGKRGFS | NI |
| 439 | Paraburkholderia | FSQSDIVKIAGNIGGAQALQAVLELEPTLRESDFR | NI |
| 440 | Paraburkholderia | FNPTDIVKIAGNKGGAQALQAVLELEPALRERGFN | NK |
| 441 | Paraburkholderia | FSPTDIIKIAGNNGGAQALQAVLDLELMLRERGFS | NN |
| 442 | Paraburkholderia | FSQADIVKIAGNNGGAQALYSVLDVEPTLGKRGFS | NN |
| 443 | Paraburkholderia | FSRGDIVTIAGNNGGAQALQAVLELEPTLRERGEN | NN |
| 444 | Paraburkholderia | FSRIDIVKIAANNGGAQALHAVLDLGPTLRECGFS | NN |
| 445 | Paraburkholderia | FSQADIVKIVGNNGGAQALQAVFELEPTLRERGEN | NN |
| 446 | Paraburkholderia | FSQPDIVRITGNRGGAQALQAVLALELTLRERGFS | NR |

In any one of the animal pathogen-derived repeat domains of SEQ ID NO: 357, SEQ ID NO: 282-SEQ ID NO: 290, and SEQ ID NO: 358-SEQ ID NO: 446, there can be considerable sequence divergence between repeats of a MAP-NBD outside of the RVD.

In some embodiments, a MAP-NBD of the present disclosure can comprise between 1 to 50 animal pathogen-derived repeat units. In some embodiments, a MAP-NBD of the present disclosure can comprise between 9 and 36 animal pathogen-derived repeat units. In some embodiments, a MAP-NBD of the present disclosure can comprise between 12 and 30 animal pathogen-derived repeat units. A MAP-NBD described herein can comprise between 5 to 10, 10 to 15, 15-20, 20 to 25, 25 to 30, 30 to 35, or 35 to 40, e.g., 15-25 animal pathogen-derived repeat units. A MAP-NBD described herein can comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 animal pathogen-derived repeat units.

A MAP-NBD described herein can comprise 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 animal pathogen-derived repeat units.

An animal pathogen-derived repeat units can be derived from a wild-type repeat unit, such as any one of SEQ ID NO: 357, SEQ ID NO: 282-SEQ ID NO: 290, and SEQ ID NO: 358-SEQ ID NO: 446. An animal pathogen-derived repeat unit can also comprise a modified animal pathogen-derived repeat units enhanced for specific recognition of a nucleotide or base pair. A MAP-NBD described herein can comprise one or more wild-type animal pathogen-derived repeat units, one or more modified animal pathogen-derived repeat units, or a combination thereof. In some embodiments, a modified animal pathogen-derived repeat units can comprise 1, 2, 3, 4, 5, 6, 7, 8, 9, 10, 11, 12, 13, 14, 15, 16, 17, 18, 19, 20, 21, 22, 23, 24, 25, 26, 27, 28, or 29 mutations that can enhance recognition of a specific nucleotide or base pair. In some embodiments, a modified animal pathogen-derived repeat unit can comprise more than 1 modification, for example 1 to 5 modifications, 5 to 10 modifications, 10 to 15 modifications, 15 to 20 modifications, 20 to 25 modification, or 25-29 modifications. In some embodiments, a MAP-NBD can comprise more than one modified animal pathogen-derived repeat units, wherein each of the modified animal pathogen-derived repeat units can have a different number of modifications.

In some embodiments, a MAP-NBD of the present disclosure can have the full length naturally occurring N-terminus of a naturally occurring Legionella quateirensis-derived protein, such as the N-terminus of SEQ ID NO: 281. A N-terminus can be the full length N-terminus sequence and can have a sequence of MPDLELNFAIPLHLFDDETVFTHDATNDNSQASSSYSSKSSPASANARKRTSRKEMSGPPSK EPANTKSRRANSQNNKLSLADRLTKYNIDEEFYQTRSDSLLSLNYTKKQIERLILYKGRTSA VQQLLCKHEELLNLISPDG (SEQ ID NO: 291). In some embodiments, any truncation of SEQ ID NO: 291 can be used as the N-terminus in a MAP-NBD of the present disclosure. For example, in some embodiments, a MAP-NBD comprises a truncated N-terminus including amino acid residues at position 1 (G) to position 137 (S) of the naturally occurring Legionella quateirensis N-terminus as follows: NFAIPLHLFDDETVFTHDATNDNSQASSSYSSKSSPASANARKRTSRKEMSGPPSKEPANTK SRRANSQNNKLSLADRLTKYNIDEEFYQTRSDSLLSLNYTKKQIERLILYKGRTSAVQQLLC KHEELLNLISPDG (SEQ ID NO: 335). For example, in some embodiments, a MAP-NBD comprises a truncated N-terminus including amino acid residues at position 1 (G) to position 120 (S) of the naturally occurring Legionella quateirensis N-terminus as follows: DATNDNSQASSSYSSKSSPASANARKRTSRKEMSGPPSKEPANTKSRRANSQNNKLSLADR LTKYNIDEEFYQTRSDSLLSLNYTKKQIERLILYKGRTSAVQQLLCKHEELLNLISPDG (SEQ ID NO: 304). In some embodiments, a MAP-NBD comprises a truncated N-terminus including amino acid residues at position 1 (G) to position 115 (K) of the naturally occurring Legionella quateirensis N-terminus as follows: NSQASSSYSSKSSPASANARKRTSRKEMSGPPSKEPANTKSRRANSQNNKLSLADRLTKYNI DEEFYQTRSDSLLSLNYTKKQIERLILYKGRTSAVQQLLCKHEELLNLISPDG (SEQ ID NO: 322). In some embodiments, any truncation of the naturally occurring Legionella quateirensis-derived protein can be used at the N-terminus of a DNA binding domain disclosed herein. The naturally occurring N-terminus of Legionella quateirensis can be truncated to amino acid residues at positions 1 to 50, 1 to 70, 1 to 100, 1 to 120, 1 to 130, 10 to 40, 60 to 100, or 100 to 120 and used at the N-terminus of the MAP-NBD.

In some embodiments, a MAP-NBD of the present disclosure can have the full length naturally occurring C-terminus of a naturally occurring Legionella quateirensis-derived protein. In some embodiments, A MAP-NBD of the present disclosure can have at its C-terminus amino acid residues at position 1 (A) to position 176 (F) of the naturally occurring Legionella quateirensis-derived protein as follows:

(SEQ ID NO: 305)
ALVKEYFPVFSSFHFTADQIVALICQSKQCFRNLKKNHQQWKNKGL
SAEQIVDLILQETPPKPNFNNTSSSTPSPSAPSFFQGPSTPIPTPV
LDNSPAPIFSNPVCFFSSRSENNTEQYLQDSTLDLDSQLGDPTKNF
NVNNFWSLFPFDDVGYHPHSNDVGYHLHSDEESPFFDF.

In some embodiments, a MAP-NBD of the present disclosure can have at its C-terminus amino acid residues at position 1 (A) to position 63 (P) of the naturally occurring Legionella quateirensis-derived protein as follows:

(SEQ ID NO: 306)
ALVKEYFPVFSSFHFTADQIVALICQSKQCFRNLKKNHQQWKNKGL
SAEQIVDLILQETPPKP.

In some embodiments, the present disclosure provides methods for identifying an animal pathogen-derived repeat unit. For example, a consensus sequence can be defined comprising a first repeat motif, a spacer, and a second repeat motif. The consensus sequence can be

```
  (SEQ ID NO: 292)
1xxx211x1xxx33x2x1xxxxxxxxxxxxxxxx211x1xxx33x
2x1xxxxxxxxx1,
  (SEQ ID NO: 293)
1xxx211x1xxx33x2x1xxxxxxxxxxxxxx1xxx211x1xxx3
3x2x1xxxxxxxxx1,
  (SEQ ID NO: 294)
1xxx211x1xxx33x2x1xxxxxxxxx1xxxxxx1xxx211x1xx
x33x2x1xxxxxxxxx1,
  (SEQ ID NO: 295)
1xxx211x1xxx33x2x1xxxxxxxxxxxxxxxx1xxx211x1xx
x33x2x1xxxxxxxxx1,
  (SEQ ID NO: 296)
1xxx211x1xxx33x2x1xxxxxxxxx1xxxxxxxx1xxx211x1
xxx33x2x1xxxxxxxxx1.
```

For any one of SEQ ID NO: 292-SEQ ID NO: 296, x can be any amino acid residue, 1, 2, and 3 are flexible residues that are defined as follows: 1 can be selected from any one of A, F, I, L, M, T, or V, 2 can be selected from any one of D, E, K, N, M, S, R, or Q, and 3 can be selected from any one of A, G, N, or S. Thus, in some embodiments, a MAP-NBD can be derived from an animal pathogen comprising the consensus sequence of SEQ ID NO: 292, SEQ ID NO: 293, SEQ ID NO: 294, SEQ ID NO: 295, or SEQ ID NO: 296. Any one of consensus sequences of SEQ ID NO: 292-SEQ ID NO: 296 can be compared against all sequences downloaded from NCBI, MGRast, JGI, and EBI databases to identify matches corresponding to animal pathogen proteins containing repeat units of a DNA-binding repeat unit.

In some embodiments, a MAP-NBD repeat unit can itself have a consensus sequence of 1xxx211x1xxx33x2x1xxxxxxxxx1 (SEQ ID NO: 293), wherein x can be any amino acid residue, 1, 2, and 3 are flexible residues that are defined as follows: 1 can be selected from any one of A, F, I, L, M, T, or V, 2 can be selected from any one of D, E, K, N, M, S, R, or Q, and 3 can be selected from any one of A, G, N, or S.

### Mixed DNA Binding Domains

In some embodiments, the present disclosure provides DNA binding domains in which the repeat units, the N-terminus, and the C-terminus can be derived from any one of Ralstonia solanacearum, Xanthomonas spp., Legionella quateirensis, Burkholderia, Paraburkholderia, or Francisella. For example, the present disclosure provides a DNA binding domain wherein the plurality of repeat units are selected from any one of SEQ ID NO: 168-SEQ ID NO: 263 or SEQ ID NO: 336-SEQ ID NO: 356 and can further comprise an N-terminus and/or C-terminus from Xanthomonas spp., (N-termini: SEQ ID NO: 298, SEQ ID NO: 300, SEQ ID NO: 301, and SEQ ID NO: 321; C-termini: SEQ ID NO: 302 and SEQ ID NO: 298) or Legionella quateirensis (N-termini: SEQ ID NO: 304 or SEQ ID NO: 322; C-termini: SEQ ID NO: 305 and SEQ ID NO: 306). In some embodiments, the present disclosure provides modular DNA binding domains in which the repeat units can be from Ralstonia solanacearum (e.g., any one of SEQ ID NO: 168-SEQ ID NO: 263 or SEQ ID NO: 336-SEQ ID NO: 356), Xanthomonas spp. (e.g., any one of SEQ ID NO: 323-SEQ ID NO: 334), an animal pathogen such as Legionella quateirensis, Burkholderia, Paraburkholderia, or Francisella (e.g., any one of SEQ ID NO: 357, SEQ ID NO: 282-SEQ ID NO: 290, or SEQ ID NO: 358-SEQ ID NO: 446), or any combination thereof.

### Nucleases for Genome Editing

Genome editing can include the process of modifying a DNA of a cell in order to introduce or knock out a target gene or a target gene region. In some instances, a subject may have a disease in which a protein is aberrantly expressed or completely lacking. One therapeutic strategy for treating this disease can be introduction of a target gene or a target gene region to correct the aberrant or missing protein. For example, genome editing can be used to modify the DNA of a cell in the subject in order to introduce a functional gene, which gives rise to a functional protein. Introduction of this functional gene and expression of the functional protein can relieve the disease state of the subject.

In other instances, a subject may have a disease in which protein is overexpressed or is targeted by a virus for infection of a cell. Alternatively, a therapy such as a cell therapy for cancer can be ineffective due to repression of certain processes by tumor cells (e.g., checkpoint inhibition). Still alternatively, it may be desirable to eliminate a particular protein expressed at the surface of a cell in order to generate a universal, off-the-shelf cell therapy for a subject in need thereof (e.g., TCR). In such cases, it can be desirable to partially or completely knock out the gene encoding for such a protein. Genome editing can be used to modify the DNA of a cell in the subject in order to partially or completely knock out the target gene, thus reducing or eliminating expression of the protein of interest.

Genome editing can include the use of any nuclease as described herein in combination with any DNA binding domain disclosed herein in order to bind to a target gene or target gene region and induce a double strand break, mediated by the nuclease. Genes can be introduced during this process, or DNA binding domains can be designed to cut at regions of the DNA such that after non-homologous end joining, the target gene or target gene region is removed. Genome editing systems that are further disclosed and described in detail herein can include DNA binding domains from Xanthomonas, Ralstonia, Legionella, Burkholderia, Paraburkholderia, or Francisella fused to nucleases.

The specificity and efficiency of genome editing can be dependent on the nuclease responsible for cleavage. More than 3,000 type II restriction endonucleases have been identified. They recognize short, usually palindromic, sequences of 4-8 bp and, in the presence of Mg2+, cleave the DNA within or in close proximity to the recognition sequence. Naturally, type IIs restriction enzymes themselves have a DNA recognition domain that can be separated from the catalytic, or cleavage, domain. As such, since cleavage occurs at a site adjacent to the DNA sequence bound by the recognition domain, these enzymes can be referred to as exhibiting “shifted” cleavage. These type IIs restriction enzymes having both the recognition domain and the cleavage domain can be 400-600 amino acids. The main criterion for classifying a restriction endonuclease as a type II enzyme is that it cleaves specifically within or close to its recognition site and that it does not require ATP hydrolysis for its nucleolytic activity. An example of a type II restriction endonucleases is FokI, which consists of a DNA recognition domain and a non-specific DNA cleavage domain. FokI cleaves DNA nine and thirteen bases downstream of an asymmetric sequence (recognizing a DNA sequence of GGATG).

In some embodiments, the DNA cleavage domain at the C-terminus of FokI itself can be combined with a variety of DNA-binding domains (e.g., RNBDs, TALEs, MAP-NBDs) of other molecules for genome editing purposes. This cleavage domain can be 180 amino acids in length and can be directly linked to a DNA binding domain (e.g., RNBDs, TALEs, MAP-NBDs). In some embodiments, the FokI cleavage domain only comprises a single catalytic site. Thus, in order to cleave phosphodiester bonds, these enzymes form transient homodimers, providing two catalytic sites capable of cleaving double stranded DNA. In some embodiments, a single DNA-binding domains (e.g., RNBDs, TALEs, MAP-NBDs) linked to a Type IIS cleaving domain may not nick the double stranded DNA at the targeted site. In some embodiments, cleaving of target DNA only occurs when a pair of DNA-binding domains (e.g., RNBDs, TALEs, MAP-NBDs), each linked to a Type IIS cleaving domain (e.g., any one of SEQ ID NO: 1-SEQ ID NO: 81 (nucleotide sequences of SEQ ID NO: 82-SEQ ID NO: 162)) bind to opposing strands of DNA and allow for formation of a transient homodimer in the spacer region (the base pairs between the C-terminus of the DNA binding domain on a top strand of DNA and the C-terminus of the DNA binding domain on a bottom strand of DNA). Said spacer region can be greater than 2 base pairs, greater than 5 base pairs, greater than 10 base pairs, greater than 15 base pairs, greater than 24 base pairs, greater than 25 base pairs, greater than 30 base pairs, greater than 35 base pairs, greater than 40 base pairs, greater than 45 base pairs, or greater than 50 base pairs. In some embodiments, the spacer region can be anywhere from 2 to 50 base pairs, 5 to 40 base pairs, 10 to 30 base pairs, 14 to 40 base pairs, 24 to 30 base pairs, 24 to 40 base pairs, or 24 to 50 base pairs. In some embodiments, the nuclease disclosed herein (e.g., any one of SEQ ID NO: 1-SEQ ID NO: 81 (nucleotide sequences of SEQ ID NO: 82-SEQ ID NO: 162) can be capable of cleaving over a spacer region of greater than 24 base pairs upon formation of a transient homodimer.

Comparative analyses showed that FokI phylogenetic groupings can largely be at least partially explained by a combination of local gene duplication, and the whole-genome duplication event that predates their speciation, however enzymes vary significantly in their activities. In some aspects, the disclosure provides enzymes identified in a phylogenetic, molecular, and comparative analyses of sequences from various proteins related to FokI in various sequenced species. In some instances, such enzymes can comprise one or more mutations relative to SEQ ID NO: 1-SEQ ID NO: 81 (nucleotide sequences of SEQ ID NO: 82-SEQ ID NO: 162). In some cases, the non-naturally occurring enzymes described herein can comprise about 1, 2, 3, 4, 5, 6, 7, 8, 9, 10, or more mutations. A mutation can be engineered to enhance cleavage efficiency. A mutation can abolish cleavage activity. In some cases, a mutation can enhance homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization, and similar mutations can be designed based on the phylogenetic analysis of SEQ ID NO: 1-SEQ ID NO: 81 (nucleotide sequences of SEQ ID NO: 82-SEQ ID NO: 162).

TABLE 6 shows exemplary amino acid sequences (SEQ ID NO: 1-SEQ ID NO: 81) of endonucleases for genome editing and the corresponding back-translated nucleic acid sequences (SEQ ID NO: 82-SEQ ID NO: 162) of the endonucleases, which were obtained using Genius software and selecting for human codon optimization.

TABLE 6
Amino Acid and Nucleic Acid Sequences of Endonucleases

| SEQ | | SEQ | |
|---|---|---|---|
| ID | | ID | Back Translated Nucleic |
| NO | Amino Acid Sequence | NO | Acid Sequences |
| 1 | FLVKGAMEIKKSELRHKLRHVPHEYIEL | 82 | TTCCTGGTGAAGGGCGCCATGGAGATCA |
| | IEIAQDSKQNRLLEFKVVEFFKKIYGYR | | AGAAGAGCGAGCTGAGGCACAAGCTGAG |
| | GKHLGGSRKPDGALFTDGLVLNHGIILD | | GCACGTGCCCCACGAGTACATCGAGCTG |
| | TKAYKDGYRLPISQADEMQRYVDENNKR | | ATCGAGATCGCCCAGGACAGCAAGCAGA |
| | SQVINPNEWWEIYPTSITDFKFLFVSGF | | ACAGGCTGCTGGAGTTCAAGGTGGTGGA |
| | FQGDYRKQLERVSHLTKCQGAVMSVEQL | | GTTCTTCAAGAAGATCTACGGCTACAGG |
| | LLGGEKIKEGSLTLEEVGKKFKNDEIVF | | GGCAAGCACCTGGGCGGCAGCAGGAAGC |
| | | | CCGACGGCGCCCTGTTCACCGACGGCCT |
| | | | GGTGCTGAACCACGGCATCATCCTGGAC |
| | | | ACCAAGGCCTACAAGGACGGCTACAGGC |
| | | | TGCCCATCAGCCAGGCCGACGAGATGCA |
| | | | GAGGTACGTGGACGAGAACAACAAGAGG |
| | | | AGCCAGGTGATCAACCCCAACGAGTGGT |
| | | | GGGAGATCTACCCCACCAGCATCACCGA |
| | | | CTTCAAGTTCCTGTTCGTGAGCGGCTTC |
| | | | TTCCAGGGCGACTACAGGAAGCAGCTGG |
| | | | AGAGGGTGAGCCACCTGACCAAGTGCCA |
| | | | GGGCGCCGTGATGAGCGTGGAGCAGCTG |
| | | | CTGCTGGGCGGCGAGAAGATCAAGGAGG |
| | | | GCAGCCTGACCCTGGAGGAGGTGGGCAA |
| | | | GAAGTTCAAGAACGACGAGATCGTGTTC |
| 2 | QIVKSSIEMSKANMRDNLQMLPHDYIEL | 83 | CAGATCGTGAAGAGCAGCATCGAGATGA |
| | IEISQDPYQNRIFEMKVMDLFINEYGFS | | GCAAGGCCAACATGAGGGACAACCTGCA |
| | GSHLGGSRKPDGAMYAHGFGVIVDTKAY | | GATGCTGCCCCACGACTACATCGAGCTG |
| | KDGYNLPISQADEMERYVRENIDRNEHV | | ATCGAGATCAGCCAGGACCCCTACCAGA |
| | NSNRWWNIFPEDTNEYKFLFVSGFFKGN | | ACAGGATCTTCGAGATGAAGGTGATGGA |
| | FEKQLERISIDTGVQGGALSVEHLLLGA | | CCTGTTCATCAACGAGTACGGCTTCAGC |
| | EYIKRGILTLYDFKNSFLNKEIQF | | GGCAGCCACCTGGGCGGCAGCAGGAAGC |
| | | | CCGACGGCGCCATGTACGCCCACGGCTT |
| | | | CGGCGTGATCGTGGACACCAAGGCCTAC |
| | | | AAGGACGGCTACAACCTGCCCATCAGCC |
| | | | AGGCCGACGAGATGGAGAGGTACGTGAG |
| | | | GGAGAACATCGACAGGAACGAGCACGTG |
| | | | AACAGCAACAGGTGGTGGAACATCTTCC |
| | | | CCGAGGACACCAACGAGTACAAGTTCCT |
| | | | GTTCGTGAGCGGCTTCTTCAAGGGCAAC |
| | | | TTCGAGAAGCAGCTGGAGAGGATCAGCA |
| | | | TCGACACCGGCGTGCAGGGCGGCGCCCT |
| | | | GAGCGTGGAGCACCTGCTGCTGGGCGCC |
| | | | GAGTACATCAAGAGGGGCATCCTGACCC |
| | | | TGTACGACTTCAAGAACAGCTTCCTGAA |
| | | | CAAGGAGATCCAGTTC |
| 3 | QTIKSSIEELKSELRTQLNVISHDYLQL | 84 | CAGACCATCAAGAGCAGCATCGAGGAGC |
| | VDISQDSQQNRLFEMKVMDLFINEFGYN | | TGAAGAGCGAGCTGAGGACCCAGCTGAA |
| | GSHLGGSRKPDGILYTEGLSKDYGIIVD | | CGTGATCAGCCACGACTACCTGCAGCTG |
| | TKAYKDGYNLPIAQADEMERYIRENIDR | | GTGGACATCAGCCAGGACAGCCAGCAGA |
| | NEVVNPNRWWEVFPSKINDYKFLFVSAY | | ACAGGCTGTTCGAGATGAAGGTGATGGA |
| | FKGNFKEQLERISINTGILGGAISVEHL | | CCTGTTCATCAACGAGTTCGGCTACAAC |
| | LLGAEYFKRGILSLEDVRDKFCNTEIEF | | GGCAGCCACCTGGGCGGCAGCAGGAAGC |
| | | | CCGACGGCATCCTGTACACCGAGGGCCT |
| | | | GAGCAAGGACTACGGCATCATCGTGGAC |
| | | | ACCAAGGCCTACAAGGACGGCTACAACC |
| | | | TGCCCATCGCCCAGGCCGACGAGATGGA |
| | | | GAGGTACATCAGGGAGAACATCGACAGG |
| | | | AACGAGGTGGTGAACCCCAACAGGTGGT |
| | | | GGGAGGTGTTCCCCAGCAAGATCAACGA |
| | | | CTACAAGTTCCTGTTCGTGAGCGCCTAC |
| | | | TTCAAGGGCAACTTCAAGGAGCAGCTGG |
| | | | AGAGGATCAGCATCAACACCGGCATCCT |
| | | | GGGCGGCGCCATCAGCGTGGAGCACCTG |
| | | | CTGCTGGGCGCCGAGTACTTCAAGAGGG |
| | | | GCATCCTGAGCCTGGAGGACGTGAGGGA |
| | | | CAAGTTCTGCAACACCGAGATCGAGTTC |
| 4 | GKSEVETIKEQMRGELTHLSHEYLGLLD | 85 | GGCAAGAGCGAGGTGGAGACCATCAAGG |
| | LAYDSKQNRLFELKTMQLLTEECGFEGL | | AGCAGATGAGGGGCGAGCTGACCCACCT |
| | HLGGSRKPDGIVYTKDENEQVGKENYGI | | GAGCCACGAGTACCTGGGCCTGCTGGAC |
| | IIDTKAYSGGYSLPISQADEMERYIGEN | | CTGGCCTACGACAGCAAGCAGAACAGGC |
| | QTRDIRINPNEWWKNFGDGVTEYYYLFV | | TGTTCGAGCTGAAGACCATGCAGCTGCT |
| | AGHFKGKYQEQIDRINCNKNIKGAAVSI | | GACCGAGGAGTGCGGCTTCGAGGGCCTG |
| | QQLLRIVNDYKAGKLTHEDMKLKIFHY | | CACCTGGGCGGCAGCAGGAAGCCCGACG |
| | | | GCATCGTGTACACCAAGGACGAGAACGA |
| | | | GCAGGTGGGCAAGGAGAACTACGGCATC |
| | | | ATCATCGACACCAAGGCCTACAGCGGCG |
| | | | GCTACAGCCTGCCCATCAGCCAGGCCGA |
| | | | CGAGATGGAGAGGTACATCGGCGAGAAC |
| | | | CAGACCAGGGACATCAGGATCAACCCCA |
| | | | ACGAGTGGTGGAAGAACTTCGGCGACGG |
| | | | CGTGACCGAGTACTACTACCTGTTCGTG |
| | | | GCCGGCCACTTCAAGGGCAAGTACCAGG |
| | | | AGCAGATCGACAGGATCAACTGCAACAA |
| | | | GAACATCAAGGGCGCCGCCGTGAGCATC |
| | | | CAGCAGCTGCTGAGGATCGTGAACGACT |
| | | | ACAAGGCCGGCAAGCTGACCCACGAGGA |
| | | | CATGAAGCTGAAGATCTTCCACTAC |
| 5 | MKILELLINECGYKGLHLGGARKPDGII | 86 | ATGAAGATCCTGGAGCTGCTGATCAACG |
| | YTEKEKYNYGVIIDTKAYSKGYNLPIGQ | | AGTGCGGCTACAAGGGCCTGCACCTGGG |
| | IDEMIRYIIENNERNIKRNTNCWWNNFE | | CGGCGCCAGGAAGCCCGACGGCATCATC |
| | KNVNEFYFSFISGEFTGNIEEKLNRIFI | | TACACCGAGAAGGAGAAGTACAACTACG |
| | STNIKGNAMSVKTLLYLANEIKANRISY | | GCGTGATCATCGACACCAAGGCCTACAG |
| | IELLNYFDNKV | | CAAGGGCTACAACCTGCCCATCGGCCAG |
| | | | ATCGACGAGATGATCAGGTACATCATCG |
| | | | AGAACAACGAGAGGAACATCAAGAGGAA |
| | | | CACCAACTGCTGGTGGAACAACTTCGAG |
| | | | AAGAACGTGAACGAGTTCTACTTCAGCT |
| | | | TCATCAGCGGCGAGTTCACCGGCAACAT |
| | | | CGAGGAGAAGCTGAACAGGATCTTCATC |
| | | | AGCACCAACATCAAGGGCAACGCCATGA |
| | | | GCGTGAAGACCCTGCTGTACCTGGCCAA |
| | | | CGAGATCAAGGCCAACAGGATCAGCTAC |
| | | | ATCGAGCTGCTGAACTACTTCGACAACA |
| | | | AGGTG |
| 6 | AKSSQSETKEKLREKLRNLPHEYLSLVD | 87 | GCCAAGAGCAGCCAGAGCGAGACCAAGG |
| | LAYDSKQNRLFEMKVIELLTEECGFQGL | | AGAAGCTGAGGGAGAAGCTGAGGAACCT |
| | HLGGSRRPDGVLYTAGLTDNYGIILDTK | | GCCCCACGAGTACCTGAGCCTGGTGGAC |
| | AYSSGYSLPIAQADEMERYVRENQTRDE | | CTGGCCTACGACAGCAAGCAGAACAGGC |
| | LVNPNQWWENFENGLGTFYFLFVAGHEN | | TGTTCGAGATGAAGGTGATCGAGCTGCT |
| | GNVQAQLERISRNTGVLGAAASISQLLL | | GACCGAGGAGTGCGGCTTCCAGGGCCTG |
| | LADAIRGGRMDRERLRHLMFQNEEFL | | CACCTGGGCGGCAGCAGGAGGCCCGACG |
| | | | GCGTGCTGTACACCGCCGGCCTGACCGA |
| | | | CAACTACGGCATCATCCTGGACACCAAG |
| | | | GCCTACAGCAGCGGCTACAGCCTGCCCA |
| | | | TCGCCCAGGCCGACGAGATGGAGAGGTA |
| | | | CGTGAGGGAGAACCAGACCAGGGACGAG |
| | | | CTGGTGAACCCCAACCAGTGGTGGGAGA |
| | | | ACTTCGAGAACGGCCTGGGCACCTTCTA |
| | | | CTTCCTGTTCGTGGCCGGCCACTTCAAC |
| | | | GGCAACGTGCAGGCCCAGCTGGAGAGGA |
| | | | TCAGCAGGAACACCGGCGTGCTGGGCGC |
| | | | CGCCGCCAGCATCAGCCAGCTGCTGCTG |
| | | | CTGGCCGACGCCATCAGGGGCGGCAGGA |
| | | | TGGACAGGGAGAGGCTGAGGCACCTGAT |
| | | | GTTCCAGAACGAGGAGTTCCTG |
| 7 | NSEKSEFTQEKDNLREKLDTLSHEYLSL | 88 | AACAGCGAGAAGAGCGAGTTCACCCAGG |
| | VDLAFDSQQNRLFEMKTVELLTKECNYK | | AGAAGGACAACCTGAGGGAGAAGCTGGA |
| | GVHLGGSRKPDGIIYTENSTDNYGVIID | | CACCCTGAGCCACGAGTACCTGAGCCTG |
| | TKAYSNGYNLPISQVDEMVRYVEENNKR | | GTGGACCTGGCCTTCGACAGCCAGCAGA |
| | EKERNSNEWWKEFGDNINKFYFSFISGK | | ACAGGCTGTTCGAGATGAAGACCGTGGA |
| | FIGNIEEKLQRITIFTNVYGNAMTIITL | | GCTGCTGACCAAGGAGTGCAACTACAAG |
| | LYLANEIKANRLKTMEVVKYFDNKV | | GGCGTGCACCTGGGCGGCAGCAGGAAGC |
| | | | CCGACGGCATCATCTACACCGAGAACAG |
| | | | CACCGACAACTACGGCGTGATCATCGAC |
| | | | ACCAAGGCCTACAGCAACGGCTACAACC |
| | | | TGCCCATCAGCCAGGTGGACGAGATGGT |
| | | | GAGGTACGTGGAGGAGAACAACAAGAGG |
| | | | GAGAAGGAGAGGAACAGCAACGAGTGGT |
| | | | GGAAGGAGTTCGGCGACAACATCAACAA |
| | | | GTTCTACTTCAGCTTCATCAGCGGCAAG |
| | | | TTCATCGGCAACATCGAGGAGAAGCTGC |
| | | | AGAGGATCACCATCTTCACCAACGTGTA |
| | | | CGGCAACGCCATGACCATCATCACCCTG |
| | | | CTGTACCTGGCCAACGAGATCAAGGCCA |
| | | | ACAGGCTGAAGACCATGGAGGTGGTGAA |
| | | | GTACTTCGACAACAAGGTG |
| 8 | NLTCSDLTEIKEEVRNALTHLSHEYLAL | 89 | AACCTGACCTGCAGCGACCTGACCGAGA |
| | IDLAYDSTQNRLFEMKTLQLLVEECGYQ | | TCAAGGAGGAGGTGAGGAACGCCCTGAC |
| | GTHLGGSRKPDGICYSEEAKSEGLEANY | | CCACCTGAGCCACGAGTACCTGGCCCTG |
| | GIIIDTKSYSGGYGLPISQADEMERYIR | | ATCGACCTGGCCTACGACAGCACCCAGA |
| | ENQTRDAEVNRNKWWEAFPETIDIFYFM | | ACAGGCTGTTCGAGATGAAGACCCTGCA |
| | FVAGHFKGNYFNQLERLQRSTGIKGAAV | | GCTGCTGGTGGAGGAGTGCGGCTACCAG |
| | DIKTLLLTANRCKTGELDHAGIESCFFN | | GGCACCCACCTGGGCGGCAGCAGGAAGC |
| | NCRL | | CCGACGGCATCTGCTACAGCGAGGAGGC |
| | | | CAAGAGCGAGGGCCTGGAGGCCAACTAC |
| | | | GGCATCATCATCGACACCAAGAGCTACA |
| | | | GCGGCGGCTACGGCCTGCCCATCAGCCA |
| | | | GGCCGACGAGATGGAGAGGTACATCAGG |
| | | | GAGAACCAGACCAGGGACGCCGAGGTGA |
| | | | ACAGGAACAAGTGGTGGGAGGCCTTCCC |
| | | | CGAGACCATCGACATCTTCTACTTCATG |
| | | | TTCGTGGCCGGCCACTTCAAGGGCAACT |
| | | | ACTTCAACCAGCTGGAGAGGCTGCAGAG |
| | | | GAGCACCGGCATCAAGGGCGCCGCCGTG |
| | | | GACATCAAGACCCTGCTGCTGACCGCCA |
| | | | ACAGGTGCAAGACCGGCGAGCTGGACCA |
| | | | CGCCGGCATCGAGAGCTGCTTCTTCAAC |
| | | | AACTGCAGGCTG |
| 9 | DNVKSNFNQEKDELREKLDTLSHEYLYL | 90 | GACAACGTGAAGAGCAACTTCAACCAGG |
| | LDLAYDSKQNKLFEMKILELLINECGYR | | AGAAGGACGAGCTGAGGGAGAAGCTGGA |
| | GLHLGGVRKPDGIIYTEKEKYNYGVIID | | CACCCTGAGCCACGAGTACCTGTACCTG |
| | TKAYSKGYNLPIGQIDEMIRYIIENNER | | CTGGACCTGGCCTACGACAGCAAGCAGA |
| | NIKRNTNCWWNNFEKNVNEFYFSFISGE | | ACAAGCTGTTCGAGATGAAGATCCTGGA |
| | FTGNIEEKLNRIFISTNIKGNAMSVKTL | | GCTGCTGATCAACGAGTGCGGCTACAGG |
| | LYLANEIKANRISFLEMEKYFDNKV | | GGCCTGCACCTGGGCGGCGTGAGGAAGC |
| | | | CCGACGGCATCATCTACACCGAGAAGGA |
| | | | GAAGTACAACTACGGCGTGATCATCGAC |
| | | | ACCAAGGCCTACAGCAAGGGCTACAACC |
| | | | TGCCCATCGGCCAGATCGACGAGATGAT |
| | | | CAGGTACATCATCGAGAACAACGAGAGG |
| | | | AACATCAAGAGGAACACCAACTGCTGGT |
| | | | GGAACAACTTCGAGAAGAACGTGAACGA |
| | | | GTTCTACTTCAGCTTCATCAGCGGCGAG |
| | | | TTCACCGGCAACATCGAGGAGAAGCTGA |
| | | | ACAGGATCTTCATCAGCACCAACATCAA |
| | | | GGGCAACGCCATGAGCGTGAAGACCCTG |
| | | | CTGTACCTGGCCAACGAGATCAAGGCCA |
| | | | ACAGGATCAGCTTCCTGGAGATGGAGAA |
| | | | GTACTTCGACAACAAGGTG |
| 10 | EGIKSNISLLKDELRGQISHISHEYLSL | 91 | GAGGGCATCAAGAGCAACATCAGCCTGC |
| | IDLAFDSKQNRLFEMKVLELLVNEYGFK | | TGAAGGACGAGCTGAGGGGCCAGATCAG |
| | GRHLGGSRKPDGIVYSTTLEDNFGIIVD | | CCACATCAGCCACGAGTACCTGAGCCTG |
| | TKAYSEGYSLPISQADEMERYVRENSNR | | ATCGACCTGGCCTTCGACAGCAAGCAGA |
| | DEEVNPNKWWENFSEEVKKYYFVFISGS | | ACAGGCTGTTCGAGATGAAGGTGCTGGA |
| | FKGKFEEQLRRLSMTTGVNGSAVNVVNL | | GCTGCTGGTGAACGAGTACGGCTTCAAG |
| | LLGAEKIRSGEMTIEELERAMFNNSEFI | | GGCAGGCACCTGGGCGGCAGCAGGAAGC |
| | | | CCGACGGCATCGTGTACAGCACCACCCT |
| | | | GGAGGACAACTTCGGCATCATCGTGGAC |
| | | | ACCAAGGCCTACAGCGAGGGCTACAGCC |
| | | | TGCCCATCAGCCAGGCCGACGAGATGGA |
| | | | GAGGTACGTGAGGGAGAACAGCAACAGG |
| | | | GACGAGGAGGTGAACCCCAACAAGTGGT |
| | | | GGGAGAACTTCAGCGAGGAGGTGAAGAA |
| | | | GTACTACTTCGTGTTCATCAGCGGCAGC |
| | | | TTCAAGGGCAAGTTCGAGGAGCAGCTGA |
| | | | GGAGGCTGAGCATGACCACCGGCGTGAA |
| | | | CGGCAGCGCCGTGAACGTGGTGAACCTG |
| | | | CTGCTGGGCGCCGAGAAGATCAGGAGCG |
| | | | GCGAGATGACCATCGAGGAGCTGGAGAG |
| | | | GGCCATGTTCAACAACAGCGAGTTCATC |
| 11 | ISKTNVLELKDKVRDKLKYVDNRYLALI | 92 | ATCAGCAAGACCAACGTGCTGGAGCTGA |
| | DLAYDGTANRDFEIQTIDLLINELKFKG | | AGGACAAGGTGAGGGACAAGCTGAAGTA |
| | VRLGESRKPDGIISYDINGVIIDNKAYS | | CGTGGACAACAGGTACCTGGCCCTGATC |
| | SGYNLPINQADEMIRYIEENQTRDKKIN | | GACCTGGCCTACGACGGCACCGCCAACA |
| | PNKWWESFDDKVKDFNYLFVSSFFKGNF | | GGGACTTCGAGATCCAGACCATCGACCT |
| | KNNLKHIANRTGVNGGVINVENLLYFAE | | GCTGATCAACGAGCTGAAGTTCAAGGGC |
| | ELKSGRLSYVDLFKMYDNDEINI | | GTGAGGCTGGGCGAGAGCAGGAAGCCCG |
| | | | ACGGCATCATCAGCTACGACATCAACGG |
| | | | CGTGATCATCGACAACAAGGCCTACAGC |
| | | | AGCGGCTACAACCTGCCCATCAACCAGG |
| | | | CCGACGAGATGATCAGGTACATCGAGGA |
| | | | GAACCAGACCAGGGACAAGAAGATCAAC |
| | | | CCCAACAAGTGGTGGGAGAGCTTCGACG |
| | | | ACAAGGTGAAGGACTTCAACTACCTGTT |
| | | | CGTGAGCAGCTTCTTCAAGGGCAACTTC |
| | | | AAGAACAACCTGAAGCACATCGCCAACA |
| | | | GGACCGGCGTGAACGGCGGCGTGATCAA |
| | | | CGTGGAGAACCTGCTGTACTTCGCCGAG |
| | | | GAGCTGAAGAGCGGCAGGCTGAGCTACG |
| | | | TGGACCTGTTCAAGATGTACGACAACGA |
| | | | CGAGATCAACATC |
| 12 | ISKTNVLELKDKVRDKLKYVDHRYLALI | 93 | ATCAGCAAGACCAACGTGCTGGAGCTGA |
| | DLAYDGTANRDFEIQTIDLLINELKFKG | | AGGACAAGGTGAGGGACAAGCTGAAGTA |
| | VRLGESRKPDGIISYDINGVIIDNKAYS | | CGTGGACCACAGGTACCTGGCCCTGATC |
| | TGYNLPINQADEMIRYIEENQTRDKKIN | | GACCTGGCCTACGACGGCACCGCCAACA |
| | SNKWWESFDDKVKNFNYLFVSSFFKGNF | | GGGACTTCGAGATCCAGACCATCGACCT |
| | KNNLKHIANRTGVNGGAINVENLLYFAE | | GCTGATCAACGAGCTGAAGTTCAAGGGC |
| | ELKAGRLSYVDSFTMYDNDEIYV | | GTGAGGCTGGGCGAGAGCAGGAAGCCCG |
| | | | ACGGCATCATCAGCTACGACATCAACGG |
| | | | CGTGATCATCGACAACAAGGCCTACAGC |
| | | | ACCGGCTACAACCTGCCCATCAACCAGG |
| | | | CCGACGAGATGATCAGGTACATCGAGGA |
| | | | GAACCAGACCAGGGACAAGAAGATCAAC |
| | | | AGCAACAAGTGGTGGGAGAGCTTCGACG |
| | | | ACAAGGTGAAGAACTTCAACTACCTGTT |
| | | | CGTGAGCAGCTTCTTCAAGGGCAACTTC |
| | | | AAGAACAACCTGAAGCACATCGCCAACA |
| | | | GGACCGGCGTGAACGGCGGCGCCATCAA |
| | | | CGTGGAGAACCTGCTGTACTTCGCCGAG |
| | | | GAGCTGAAGGCCGGCAGGCTGAGCTACG |
| | | | TGGACAGCTTCACCATGTACGACAACGA |
| | | | CGAGATCTACGTG |
| 13 | KAEKSEFLIEKDKLREKLDTLPHDYLSM | 94 | AAGGCCGAGAAGAGCGAGTTCCTGATCG |
| | VDLAYDSKQNRLFEMKTIELLINECNYK | | AGAAGGACAAGCTGAGGGAGAAGCTGGA |
| | GLHLGGTRKPDGIVYTNNEVENYGIIID | | CACCCTGCCCCACGACTACCTGAGCATG |
| | TKAYSKGYNLPISQVDEMTRYVEENNKR | | GTGGACCTGGCCTACGACAGCAAGCAGA |
| | EKKRNPNEWWNNFDSNVKKFYFSFISGK | | ACAGGCTGTTCGAGATGAAGACCATCGA |
| | FVGNIEEKLQRITLFTEIYGNAITVTTL | | GCTGCTGATCAACGAGTGCAACTACAAG |
| | LYIANEIKANRMKKSDIMEYFNDKV | | GGCCTGCACCTGGGCGGCACCAGGAAGC |
| | | | CCGACGGCATCGTGTACACCAACAACGA |
| | | | GGTGGAGAACTACGGCATCATCATCGAC |
| | | | ACCAAGGCCTACAGCAAGGGCTACAACC |
| | | | TGCCCATCAGCCAGGTGGACGAGATGAC |
| | | | CAGGTACGTGGAGGAGAACAACAAGAGG |
| | | | GAGAAGAAGAGGAACCCCAACGAGTGGT |
| | | | GGAACAACTTCGACAGCAACGTGAAGAA |
| | | | GTTCTACTTCAGCTTCATCAGCGGCAAG |
| | | | TTCGTGGGCAACATCGAGGAGAAGCTGC |
| | | | AGAGGATCACCCTGTTCACCGAGATCTA |
| | | | CGGCAACGCCATCACCGTGACCACCCTG |
| | | | CTGTACATCGCCAACGAGATCAAGGCCA |
| | | | ACAGGATGAAGAAGAGCGACATCATGGA |
| | | | GTACTTCAACGACAAGGTG |
| 14 | ISKTNVLELKDKVRDKLKYVDHRYLALI | 95 | ATCAGCAAGACCAACGTGCTGGAGCTGA |
| | DLAYDGTANRDFEIQTIDLLINELKFKG | | AGGACAAGGTGAGGGACAAGCTGAAGTA |
| | VRLGESRKPDGIISYNINGVIIDNKAYS | | CGTGGACCACAGGTACCTGGCCCTGATC |
| | TGYNLPINQADEMIRYIEENQTRDEKIN | | GACCTGGCCTACGACGGCACCGCCAACA |
| | SNKWWESFDDEVKDFNYLFVSSFFKGNF | | GGGACTTCGAGATCCAGACCATCGACCT |
| | KNNLKHIANRTGVNGGAINVENLLYFAE | | GCTGATCAACGAGCTGAAGTTCAAGGGC |
| | ELKAGRLSYVDSFTMYDNDEIYV | | GTGAGGCTGGGCGAGAGCAGGAAGCCCG |
| | | | ACGGCATCATCAGCTACAACATCAACGG |
| | | | CGTGATCATCGACAACAAGGCCTACAGC |
| | | | ACCGGCTACAACCTGCCCATCAACCAGG |
| | | | CCGACGAGATGATCAGGTACATCGAGGA |
| | | | GAACCAGACCAGGGACGAGAAGATCAAC |
| | | | AGCAACAAGTGGTGGGAGAGCTTCGACG |
| | | | ACGAGGTGAAGGACTTCAACTACCTGTT |
| | | | CGTGAGCAGCTTCTTCAAGGGCAACTTC |
| | | | AAGAACAACCTGAAGCACATCGCCAACA |
| | | | GGACCGGCGTGAACGGCGGCGCCATCAA |
| | | | CGTGGAGAACCTGCTGTACTTCGCCGAG |
| | | | GAGCTGAAGGCCGGCAGGCTGAGCTACG |
| | | | TGGACAGCTTCACCATGTACGACAACGA |
| | | | CGAGATCTACGTG |
| 15 | ISKTNILELKDKVRDKLKYVDHRYLALI | 96 | ATCAGCAAGACCAACATCCTGGAGCTGA |
| | DLAYDGTANRDFEIQTIDLLINELKFKG | | AGGACAAGGTGAGGGACAAGCTGAAGTA |
| | VRLGESRKPDGIISYNINGVIIDNKAYS | | CGTGGACCACAGGTACCTGGCCCTGATC |
| | TGYNLPINQADEMIRYIEENQTRDEKIN | | GACCTGGCCTACGACGGCACCGCCAACA |
| | SNKWWESFDEKVKDFNYLFVSSFFKGNF | | GGGACTTCGAGATCCAGACCATCGACCT |
| | KNNLKHIANRTGVNGGAINVENLLYFAE | | GCTGATCAACGAGCTGAAGTTCAAGGGC |
| | ELKAGRISYLDSFKMYNNDEIYL | | GTGAGGCTGGGCGAGAGCAGGAAGCCCG |
| | | | ACGGCATCATCAGCTACAACATCAACGG |
| | | | CGTGATCATCGACAACAAGGCCTACAGC |
| | | | ACCGGCTACAACCTGCCCATCAACCAGG |
| | | | CCGACGAGATGATCAGGTACATCGAGGA |
| | | | GAACCAGACCAGGGACGAGAAGATCAAC |
| | | | AGCAACAAGTGGTGGGAGAGCTTCGACG |
| | | | AGAAGGTGAAGGACTTCAACTACCTGTT |
| | | | CGTGAGCAGCTTCTTCAAGGGCAACTTC |
| | | | AAGAACAACCTGAAGCACATCGCCAACA |
| | | | GGACCGGCGTGAACGGCGGCGCCATCAA |
| | | | CGTGGAGAACCTGCTGTACTTCGCCGAG |
| | | | GAGCTGAAGGCCGGCAGGATCAGCTACC |
| | | | TGGACAGCTTCAAGATGTACAACAACGA |
| | | | CGAGATCTACCTG |
| 16 | ISKTNVLELKDKVRDKLKYVDHRYLALI | 97 | ATCAGCAAGACCAACGTGCTGGAGCTGA |
| | DLAYDGTANRDFEIQTIDLLINELKFKG | | AGGACAAGGTGAGGGACAAGCTGAAGTA |
| | VRLGESRKPDGIISYNINGVIIDNKAYS | | CGTGGACCACAGGTACCTGGCCCTGATC |
| | TGYNLPINQADEMIRYIEENQTRDEKIN | | GACCTGGCCTACGACGGCACCGCCAACA |
| | SNKWWESFDDKVKDFNYLFVSSFFKGNF | | GGGACTTCGAGATCCAGACCATCGACCT |
| | KNNLKHIANRTGVSGGAINVENLLYFAE | | GCTGATCAACGAGCTGAAGTTCAAGGGC |
| | ELKAGRLSYVDSFKMYDNDEIYV | | GTGAGGCTGGGCGAGAGCAGGAAGCCCG |
| | | | ACGGCATCATCAGCTACAACATCAACGG |
| | | | CGTGATCATCGACAACAAGGCCTACAGC |
| | | | ACCGGCTACAACCTGCCCATCAACCAGG |
| | | | CCGACGAGATGATCAGGTACATCGAGGA |
| | | | GAACCAGACCAGGGACGAGAAGATCAAC |
| | | | AGCAACAAGTGGTGGGAGAGCTTCGACG |
| | | | ACAAGGTGAAGGACTTCAACTACCTGTT |
| | | | CGTGAGCAGCTTCTTCAAGGGCAACTTC |
| | | | AAGAACAACCTGAAGCACATCGCCAACA |
| | | | GGACCGGCGTGAGCGGCGGCGCCATCAA |
| | | | CGTGGAGAACCTGCTGTACTTCGCCGAG |
| | | | GAGCTGAAGGCCGGCAGGCTGAGCTACG |
| | | | TGGACAGCTTCAAGATGTACGACAACGA |
| | | | CGAGATCTACGTG |
| 17 | ISKTNVLELKDKVRNKLKYVDHRYLALI | 98 | ATCAGCAAGACCAACGTGCTGGAGCTGA |
| | DLAYDGTANRDFEIQTIDLLINELKFKG | | AGGACAAGGTGAGGAACAAGCTGAAGTA |
| | VRLGESRKPDGIISYDINGVIIDNKSYS | | CGTGGACCACAGGTACCTGGCCCTGATC |
| | TGYNLPINQADEMIRYIEENQTRDEKIN | | GACCTGGCCTACGACGGCACCGCCAACA |
| | SNKWWESFDEKVKDFNYLFVSSFFKGNF | | GGGACTTCGAGATCCAGACCATCGACCT |
| | KNNLKHIANRTGVNGGAINVENLLYFAE | | GCTGATCAACGAGCTGAAGTTCAAGGGC |
| | ELKSGRLSYVDSFTMYDNDEIYV | | GTGAGGCTGGGCGAGAGCAGGAAGCCCG |
| | | | ACGGCATCATCAGCTACGACATCAACGG |
| | | | CGTGATCATCGACAACAAGAGCTACAGC |
| | | | ACCGGCTACAACCTGCCCATCAACCAGG |
| | | | CCGACGAGATGATCAGGTACATCGAGGA |
| | | | GAACCAGACCAGGGACGAGAAGATCAAC |
| | | | AGCAACAAGTGGTGGGAGAGCTTCGACG |
| | | | AGAAGGTGAAGGACTTCAACTACCTGTT |
| | | | CGTGAGCAGCTTCTTCAAGGGCAACTTC |
| | | | AAGAACAACCTGAAGCACATCGCCAACA |
| | | | GGACCGGCGTGAACGGCGGCGCCATCAA |
| | | | CGTGGAGAACCTGCTGTACTTCGCCGAG |
| | | | GAGCTGAAGAGCGGCAGGCTGAGCTACG |
| | | | TGGACAGCTTCACCATGTACGACAACGA |
| | | | CGAGATCTACGTG |
| 18 | ISKTNVLELKDKVRDKLKYVDHRYLSLI | 99 | ATCAGCAAGACCAACGTGCTGGAGCTGA |
| | DLAYDGNANRDFEIQTIDLLINELNFKG | | AGGACAAGGTGAGGGACAAGCTGAAGTA |
| | VRLGESRKPDGIISYNINGVIIDNKAYS | | CGTGGACCACAGGTACCTGAGCCTGATC |
| | TGYNLPINQADEMIRYIEENQTRDEKIN | | GACCTGGCCTACGACGGCAACGCCAACA |
| | SNKWWESFDDKVKDFNYLFVSSFFKGNF | | GGGACTTCGAGATCCAGACCATCGACCT |
| | KNNLKHIANRTGVSGGAINVENLLYFAE | | GCTGATCAACGAGCTGAACTTCAAGGGC |
| | ELKAGRLSYADSFTMYDNDEIYV | | GTGAGGCTGGGCGAGAGCAGGAAGCCCG |
| | | | ACGGCATCATCAGCTACAACATCAACGG |
| | | | CGTGATCATCGACAACAAGGCCTACAGC |
| | | | ACCGGCTACAACCTGCCCATCAACCAGG |
| | | | CCGACGAGATGATCAGGTACATCGAGGA |
| | | | GAACCAGACCAGGGACGAGAAGATCAAC |
| | | | AGCAACAAGTGGTGGGAGAGCTTCGACG |
| | | | ACAAGGTGAAGGACTTCAACTACCTGTT |
| | | | CGTGAGCAGCTTCTTCAAGGGCAACTTC |
| | | | AAGAACAACCTGAAGCACATCGCCAACA |
| | | | GGACCGGCGTGAGCGGCGGCGCCATCAA |
| | | | CGTGGAGAACCTGCTGTACTTCGCCGAG |
| | | | GAGCTGAAGGCCGGCAGGCTGAGCTACG |
| | | | CCGACAGCTTCACCATGTACGACAACGA |
| | | | CGAGATCTACGTG |
| 19 | IAKTNVLGLKDKVRDRLKYVDHRYLALI | 100 | ATCGCCAAGACCAACGTGCTGGGCCTGA |
| | DLAYDGTANRDFEIQTIDLLINELKFKG | | AGGACAAGGTGAGGGACAGGCTGAAGTA |
| | VRLGESRKPDGIISYNVNGVIIDNKAYS | | CGTGGACCACAGGTACCTGGCCCTGATC |
| | KGYNLPINQADEMIRYIEENQTRDEKIN | | GACCTGGCCTACGACGGCACCGCCAACA |
| | ANKWWESFDDKVEEFSYLFVSSFFKGNF | | GGGACTTCGAGATCCAGACCATCGACCT |
| | KNNLKHIANRTGVNGGAINVENLLYFAE | | GCTGATCAACGAGCTGAAGTTCAAGGGC |
| | ELKSGRLSYMDSFSLYDNDEICV | | GTGAGGCTGGGCGAGAGCAGGAAGCCCG |
| | | | ACGGCATCATCAGCTACAACGTGAACGG |
| | | | CGTGATCATCGACAACAAGGCCTACAGC |
| | | | AAGGGCTACAACCTGCCCATCAACCAGG |
| | | | CCGACGAGATGATCAGGTACATCGAGGA |
| | | | GAACCAGACCAGGGACGAGAAGATCAAC |
| | | | GCCAACAAGTGGTGGGAGAGCTTCGACG |
| | | | ACAAGGTGGAGGAGTTCAGCTACCTGTT |
| | | | CGTGAGCAGCTTCTTCAAGGGCAACTTC |
| | | | AAGAACAACCTGAAGCACATCGCCAACA |
| | | | GGACCGGCGTGAACGGCGGCGCCATCAA |
| | | | CGTGGAGAACCTGCTGTACTTCGCCGAG |
| | | | GAGCTGAAGAGCGGCAGGCTGAGCTACA |
| | | | TGGACAGCTTCAGCCTGTACGACAACGA |
| | | | CGAGATCTGCGTG |
| 20 | ELKDEQSEKRKAKFLKETKLPMKYIELL | 101 | GAGCTGAAGGACGAGCAGAGCGAGAAGA |
| | DIAYDGKRNRDFEIVTMELFREVYRLNS | | GGAAGGCCAAGTTCCTGAAGGAGACCAA |
| | KLLGGGRKPDGLIYTDDFGVIVDTKAYG | | GCTGCCCATGAAGTACATCGAGCTGCTG |
| | EGYSKSINQADEMIRYIEDNKRRDEKRN | | GACATCGCCTACGACGGCAAGAGGAACA |
| | PIKWWESFPSSISQNNFYFLWVSSKFVG | | GGGACTTCGAGATCGTGACCATGGAGCT |
| | KFQEQLAYTANETQTKGGAINVEQILIG | | GTTCAGGGAGGTGTACAGGCTGAACAGC |
| | ADLIMQKMLDINTIPSFFENQEIIF | | AAGCTGCTGGGCGGCGGCAGGAAGCCCG |
| | | | ACGGCCTGATCTACACCGACGACTTCGG |
| | | | CGTGATCGTGGACACCAAGGCCTACGGC |
| | | | GAGGGCTACAGCAAGAGCATCAACCAGG |
| | | | CCGACGAGATGATCAGGTACATCGAGGA |
| | | | CAACAAGAGGAGGGACGAGAAGAGGAAC |
| | | | CCCATCAAGTGGTGGGAGAGCTTCCCCA |
| | | | GCAGCATCAGCCAGAACAACTTCTACTT |
| | | | CCTGTGGGTGAGCAGCAAGTTCGTGGGC |
| | | | AAGTTCCAGGAGCAGCTGGCCTACACCG |
| | | | CCAACGAGACCCAGACCAAGGGCGGCGC |
| | | | CATCAACGTGGAGCAGATCCTGATCGGC |
| | | | GCCGACCTGATCATGCAGAAGATGCTGG |
| | | | ACATCAACACCATCCCCAGCTTCTTCGA |
| | | | GAACCAGGAGATCATCTTC |
| 21 | IFKTNVLELKDSIREKLDYIDHRYLSLV | 102 | ATCTTCAAGACCAACGTGCTGGAGCTGA |
| | DLAYDSKANRDFEIQTIDLLINELDFKG | | AGGACAGCATCAGGGAGAAGCTGGACTA |
| | LRLGESRKPDGIISYDINGVIIDNKAYS | | CATCGACCACAGGTACCTGAGCCTGGTG |
| | KGYNLPINQADEMIRYIQENQSRNEKIN | | GACCTGGCCTACGACAGCAAGGCCAACA |
| | PNKWWENFEDKVIKFNYLFISSLFVGGF | | GGGACTTCGAGATCCAGACCATCGACCT |
| | KKNLQHIANRTGVNGGAIDVENLLYFAE | | GCTGATCAACGAGCTGGACTTCAAGGGC |
| | EIKSGRLTYKDSFSRYINDEIKM | | CTGAGGCTGGGCGAGAGCAGGAAGCCCG |
| | | | ACGGCATCATCAGCTACGACATCAACGG |
| | | | CGTGATCATCGACAACAAGGCCTACAGC |
| | | | AAGGGCTACAACCTGCCCATCAACCAGG |
| | | | CCGACGAGATGATCAGGTACATCCAGGA |
| | | | GAACCAGAGCAGGAACGAGAAGATCAAC |
| | | | CCCAACAAGTGGTGGGAGAACTTCGAGG |
| | | | ACAAGGTGATCAAGTTCAACTACCTGTT |
| | | | CATCAGCAGCCTGTTCGTGGGCGGCTTC |
| | | | AAGAAGAACCTGCAGCACATCGCCAACA |
| | | | GGACCGGCGTGAACGGCGGCGCCATCGA |
| | | | CGTGGAGAACCTGCTGTACTTCGCCGAG |
| | | | GAGATCAAGAGCGGCAGGCTGACCTACA |
| | | | AGGACAGCTTCAGCAGGTACATCAACGA |
| | | | CGAGATCAAGATG |
| 22 | LPVKSEVSVFKDYLRTHLTHVDHRYLIL | 103 | CTGCCCGTGAAGAGCGAGGTGAGCGTGT |
| | VDLGFDGSSDRDYEMKTAELFTAELGFM | | TCAAGGACTACCTGAGGACCCACCTGAC |
| | GARLGDTRKPDVCVYHGANGLIIDNKAY | | CCACGTGGACCACAGGTACCTGATCCTG |
| | GKGYSLPIKQADEIYRYIEENKERDARL | | GTGGACCTGGGCTTCGACGGCAGCAGCG |
| | NPNQWWKVFDESVTHFRFAFISGSFTGG | | ACAGGGACTACGAGATGAAGACCGCCGA |
| | FKDRIELISMRSGICGAAVNSVNLLLMA | | GCTGTTCACCGCCGAGCTGGGCTTCATG |
| | EELKSGRLDYEEWFQYFDCNDEISF | | GGCGCCAGGCTGGGCGACACCAGGAAGC |
| | | | CCGACGTGTGCGTGTACCACGGCGCCAA |
| | | | CGGCCTGATCATCGACAACAAGGCCTAC |
| | | | GGCAAGGGCTACAGCCTGCCCATCAAGC |
| | | | AGGCCGACGAGATCTACAGGTACATCGA |
| | | | GGAGAACAAGGAGAGGGACGCCAGGCTG |
| | | | AACCCCAACCAGTGGTGGAAGGTGTTCG |
| | | | ACGAGAGCGTGACCCACTTCAGGTTCGC |
| | | | CTTCATCAGCGGCAGCTTCACCGGCGGC |
| | | | TTCAAGGACAGGATCGAGCTGATCAGCA |
| | | | TGAGGAGCGGCATCTGCGGCGCCGCCGT |
| | | | GAACAGCGTGAACCTGCTGCTGATGGCC |
| | | | GAGGAGCTGAAGAGCGGCAGGCTGGACT |
| | | | ACGAGGAGTGGTTCCAGTACTTCGACTG |
| | | | CAACGACGAGATCAGCTTC |
| 23 | ISVKSDMAVVKDSVRERLAHVSHEYLIL | 104 | ATCAGCGTGAAGAGCGACATGGCCGTGG |
| | IDLGFDGTSDRDYEIQTAELFTRELDFL | | TGAAGGACAGCGTGAGGGAGAGGCTGGC |
| | GGRLGDTRKPDVCIYYGKDGMIIDNKAY | | CCACGTGAGCCACGAGTACCTGATCCTG |
| | GKGYSLPIKQADEMYRYLEENKERNEKI | | ATCGACCTGGGCTTCGACGGCACCAGCG |
| | NPNRWWKVFDEGVTDYRFAFVSGSFTGG | | ACAGGGACTACGAGATCCAGACCGCCGA |
| | FKDRLENIHMRSGLCGGAIDSVTLLLLA | | GCTGTTCACCAGGGAGCTGGACTTCCTG |
| | EELKAGRMEYSEFFRLFDCNDEVTF | | GGCGGCAGGCTGGGCGACACCAGGAAGC |
| | | | CCGACGTGTGCATCTACTACGGCAAGGA |
| | | | CGGCATGATCATCGACAACAAGGCCTAC |
| | | | GGCAAGGGCTACAGCCTGCCCATCAAGC |
| | | | AGGCCGACGAGATGTACAGGTACCTGGA |
| | | | GGAGAACAAGGAGAGGAACGAGAAGATC |
| | | | AACCCCAACAGGTGGTGGAAGGTGTTCG |
| | | | ACGAGGGCGTGACCGACTACAGGTTCGC |
| | | | CTTCGTGAGCGGCAGCTTCACCGGCGGC |
| | | | TTCAAGGACAGGCTGGAGAACATCCACA |
| | | | TGAGGAGCGGCCTGTGCGGCGGCGCCAT |
| | | | CGACAGCGTGACCCTGCTGCTGCTGGCC |
| | | | GAGGAGCTGAAGGCCGGCAGGATGGAGT |
| | | | ACAGCGAGTTCTTCAGGCTGTTCGACTG |
| | | | CAACGACGAGGTGACCTTC |
| 24 | ELKDKAADAVKAKFLKLTGLSMKYIELL | 105 | GAGCTGAAGGACAAGGCCGCCGACGCCG |
| | DIAYDSSRNRDFEILTADLFKNVYGLDA | | TGAAGGCCAAGTTCCTGAAGCTGACCGG |
| | MHLGGGRKPDAIAQTSHFGIIIDTKAYG | | CCTGAGCATGAAGTACATCGAGCTGCTG |
| | NGYSKSISQEDEMVRYIEDNQQRSITRN | | GACATCGCCTACGACAGCAGCAGGAACA |
| | SVEWWKNFNSSIPSTAFYFLWVSSKFVG | | GGGACTTCGAGATCCTGACCGCCGACCT |
| | KFDDQLLATYNRTNTCGGALNVEQLLIG | | GTTCAAGAACGTGTACGGCCTGGACGCC |
| | AYKVKAGLLGIGQIPSYFKNKEIAW | | ATGCACCTGGGCGGCGGCAGGAAGCCCG |
| | | | ACGCCATCGCCCAGACCAGCCACTTCGG |
| | | | CATCATCATCGACACCAAGGCCTACGGC |
| | | | AACGGCTACAGCAAGAGCATCAGCCAGG |
| | | | AGGACGAGATGGTGAGGTACATCGAGGA |
| | | | CAACCAGCAGAGGAGCATCACCAGGAAC |
| | | | AGCGTGGAGTGGTGGAAGAACTTCAACA |
| | | | GCAGCATCCCCAGCACCGCCTTCTACTT |
| | | | CCTGTGGGTGAGCAGCAAGTTCGTGGGC |
| | | | AAGTTCGACGACCAGCTGCTGGCCACCT |
| | | | ACAACAGGACCAACACCTGCGGCGGCGC |
| | | | CCTGAACGTGGAGCAGCTGCTGATCGGC |
| | | | GCCTACAAGGTGAAGGCCGGCCTGCTGG |
| | | | GCATCGGCCAGATCCCCAGCTACTTCAA |
| | | | GAACAAGGAGATCGCCTGG |
| 25 | ISVKSDMAVVKDSVRERLAHVSHEYLLL | 106 | ATCAGCGTGAAGAGCGACATGGCCGTGG |
| | IDLGFDGTSDRDYEIQTAELLTRELDFL | | TGAAGGACAGCGTGAGGGAGAGGCTGGC |
| | GGRLGDTRKPDVCIYYGKDGMIIDNKAY | | CCACGTGAGCCACGAGTACCTGCTGCTG |
| | GKGYSLPIKQADEMYRYLEENKERNEKI | | ATCGACCTGGGCTTCGACGGCACCAGCG |
| | NPNRWWKVFDEGVTDYRFAFVSGSFTGG | | ACAGGGACTACGAGATCCAGACCGCCGA |
| | FKDRLENIHMRSGLCGGAIDSVTLLLLA | | GCTGCTGACCAGGGAGCTGGACTTCCTG |
| | EELKAGRMEYSEFFRLFDCNDEVTF | | GGCGGCAGGCTGGGCGACACCAGGAAGC |
| | | | CCGACGTGTGCATCTACTACGGCAAGGA |
| | | | CGGCATGATCATCGACAACAAGGCCTAC |
| | | | GGCAAGGGCTACAGCCTGCCCATCAAGC |
| | | | AGGCCGACGAGATGTACAGGTACCTGGA |
| | | | GGAGAACAAGGAGAGGAACGAGAAGATC |
| | | | AACCCCAACAGGTGGTGGAAGGTGTTCG |
| | | | ACGAGGGCGTGACCGACTACAGGTTCGC |
| | | | CTTCGTGAGCGGCAGCTTCACCGGCGGC |
| | | | TTCAAGGACAGGCTGGAGAACATCCACA |
| | | | TGAGGAGCGGCCTGTGCGGCGGCGCCAT |
| | | | CGACAGCGTGACCCTGCTGCTGCTGGCC |
| | | | GAGGAGCTGAAGGCCGGCAGGATGGAGT |
| | | | ACAGCGAGTTCTTCAGGCTGTTCGACTG |
| | | | CAACGACGAGGTGACCTTC |
| 26 | ELKDEQAEKRKAKFLKETNLPMKYIELL | 107 | GAGCTGAAGGACGAGCAGGCCGAGAAGA |
| | DIAYDGKRNRDFEIVTMELFRNVYRLHS | | GGAAGGCCAAGTTCCTGAAGGAGACCAA |
| | KLLGGGRKPDGLLYQDRFGVIVDTKAYG | | CCTGCCCATGAAGTACATCGAGCTGCTG |
| | KGYSKSINQADEMIRYIEDNKRRDENRN | | GACATCGCCTACGACGGCAAGAGGAACA |
| | PIKWWEAFPDTIPQEEFYFMWVSSKFIG | | GGGACTTCGAGATCGTGACCATGGAGCT |
| | KFQEQLDYTSNETQIKGAALNVEQLLLG | | GTTCAGGAACGTGTACAGGCTGCACAGC |
| | ADLVLKGQLHISDLPSYFQNKEIEF | | AAGCTGCTGGGCGGCGGCAGGAAGCCCG |
| | | | ACGGCCTGCTGTACCAGGACAGGTTCGG |
| | | | CGTGATCGTGGACACCAAGGCCTACGGC |
| | | | AAGGGCTACAGCAAGAGCATCAACCAGG |
| | | | CCGACGAGATGATCAGGTACATCGAGGA |
| | | | CAACAAGAGGAGGGACGAGAACAGGAAC |
| | | | CCCATCAAGTGGTGGGAGGCCTTCCCCG |
| | | | ACACCATCCCCCAGGAGGAGTTCTACTT |
| | | | CATGTGGGTGAGCAGCAAGTTCATCGGC |
| | | | AAGTTCCAGGAGCAGCTGGACTACACCA |
| | | | GCAACGAGACCCAGATCAAGGGCGCCGC |
| | | | CCTGAACGTGGAGCAGCTGCTGCTGGGC |
| | | | GCCGACCTGGTGCTGAAGGGCCAGCTGC |
| | | | ACATCAGCGACCTGCCCAGCTACTTCCA |
| | | | GAACAAGGAGATCGAGTTC |
| 27 | RNLDNVERDNRKAEFLAKTSLPPRFIEL | 108 | AGGAACCTGGACAACGTGGAGAGGGACA |
| | LSIAYESKSNRDFEMITAELFKDVYGLG | | ACAGGAAGGCCGAGTTCCTGGCCAAGAC |
| | AVHLGNAKKPDALAFNDDFGIIIDTKAY | | CAGCCTGCCCCCCAGGTTCATCGAGCTG |
| | SNGYSKNINQEDEMVRYIEDNQIRSPDR | | CTGAGCATCGCCTACGAGAGCAAGAGCA |
| | NNNEWWLSFPPSIPENDFHFLWVSSYFT | | ACAGGGACTTCGAGATGATCACCGCCGA |
| | GRFEEQLQETSARTGGTTGGALDVEQLL | | GCTGTTCAAGGACGTGTACGGCCTGGGC |
| | IGGSLIQEGSLAPHEVPAYMQNRVIHF | | GCCGTGCACCTGGGCAACGCCAAGAAGC |
| | | | CCGACGCCCTGGCCTTCAACGACGACTT |
| | | | CGGCATCATCATCGACACCAAGGCCTAC |
| | | | AGCAACGGCTACAGCAAGAACATCAACC |
| | | | AGGAGGACGAGATGGTGAGGTACATCGA |
| | | | GGACAACCAGATCAGGAGCCCCGACAGG |
| | | | AACAACAACGAGTGGTGGCTGAGCTTCC |
| | | | CCCCCAGCATCCCCGAGAACGACTTCCA |
| | | | CTTCCTGTGGGTGAGCAGCTACTTCACC |
| | | | GGCAGGTTCGAGGAGCAGCTGCAGGAGA |
| | | | CCAGCGCCAGGACCGGCGGCACCACCGG |
| | | | CGGCGCCCTGGACGTGGAGCAGCTGCTG |
| | | | ATCGGCGGCAGCCTGATCCAGGAGGGCA |
| | | | GCCTGGCCCCCCACGAGGTGCCCGCCTA |
| | | | CATGCAGAACAGGGTGATCCACTTC |
| 28 | SPVKSEVSVFKDYLRTHLTHVDHRYLIL | 109 | AGCCCCGTGAAGAGCGAGGTGAGCGTGT |
| | VDLGFDGSSDRDYEMKTAELFTAELGFM | | TCAAGGACTACCTGAGGACCCACCTGAC |
| | GARLGDTRKPDVCVYHGAHGLIIDNKAY | | CCACGTGGACCACAGGTACCTGATCCTG |
| | GKGYSLPIKQADEIYRYIEENKERAVRL | | GTGGACCTGGGCTTCGACGGCAGCAGCG |
| | NPNQWWKVFDESVAHFRFAFISGSFTGG | | ACAGGGACTACGAGATGAAGACCGCCGA |
| | FKDRIELISMRSGICGAAVNSVNLLLMA | | GCTGTTCACCGCCGAGCTGGGCTTCATG |
| | EELKSGRLNYEEWFQYFDCNDEISL | | GGCGCCAGGCTGGGCGACACCAGGAAGC |
| | | | CCGACGTGTGCGTGTACCACGGCGCCCA |
| | | | CGGCCTGATCATCGACAACAAGGCCTAC |
| | | | GGCAAGGGCTACAGCCTGCCCATCAAGC |
| | | | AGGCCGACGAGATCTACAGGTACATCGA |
| | | | GGAGAACAAGGAGAGGGCCGTGAGGCTG |
| | | | AACCCCAACCAGTGGTGGAAGGTGTTCG |
| | | | ACGAGAGCGTGGCCCACTTCAGGTTCGC |
| | | | CTTCATCAGCGGCAGCTTCACCGGCGGC |
| | | | TTCAAGGACAGGATCGAGCTGATCAGCA |
| | | | TGAGGAGCGGCATCTGCGGCGCCGCCGT |
| | | | GAACAGCGTGAACCTGCTGCTGATGGCC |
| | | | GAGGAGCTGAAGAGCGGCAGGCTGAACT |
| | | | ACGAGGAGTGGTTCCAGTACTTCGACTG |
| | | | CAACGACGAGATCAGCCTG |
| 29 | TLVDIEKERKKAYFLKETSLSPRYIELL | 110 | ACCCTGGTGGACATCGAGAAGGAGAGGA |
| | EIAFDPKRNRDFEVITAELLKAGYGLKA | | AGAAGGCCTACTTCCTGAAGGAGACCAG |
| | KVLGGGRRPDGIAYTKDYGLIVDTKAYS | | CCTGAGCCCCAGGTACATCGAGCTGCTG |
| | NGYGKNIGQADEMIRYIEDNQKRDNKRN | | GAGATCGCCTTCGACCCCAAGAGGAACA |
| | PIEWWREFEVQIPANSYYYLWVSGRFTG | | GGGACTTCGAGGTGATCACCGCCGAGCT |
| | RFDEQLVYTSSQTNTRGGALEVEQLLWG | | GCTGAAGGCCGGCTACGGCCTGAAGGCC |
| | ADAVMKGKLNVSDLPKYMNNSIIKL | | AAGGTGCTGGGCGGCGGCAGGAGGCCCG |
| | | | ACGGCATCGCCTACACCAAGGACTACGG |
| | | | CCTGATCGTGGACACCAAGGCCTACAGC |
| | | | AACGGCTACGGCAAGAACATCGGCCAGG |
| | | | CCGACGAGATGATCAGGTACATCGAGGA |
| | | | CAACCAGAAGAGGGACAACAAGAGGAAC |
| | | | CCCATCGAGTGGTGGAGGGAGTTCGAGG |
| | | | TGCAGATCCCCGCCAACAGCTACTACTA |
| | | | CCTGTGGGTGAGCGGCAGGTTCACCGGC |
| | | | AGGTTCGACGAGCAGCTGGTGTACACCA |
| | | | GCAGCCAGACCAACACCAGGGGCGGCGC |
| | | | CCTGGAGGTGGAGCAGCTGCTGTGGGGC |
| | | | GCCGACGCCGTGATGAAGGGCAAGCTGA |
| | | | ACGTGAGCGACCTGCCCAAGTACATGAA |
| | | | CAACAGCATCATCAAGCTG |
| 30 | ELRDKVIEEQKAIFLQKTKLPLSYIELL | 111 | GAGCTGAGGGACAAGGTGATCGAGGAGC |
| | EIARDGKRSRDFELITIELFKNIYKINA | | AGAAGGCCATCTTCCTGCAGAAGACCAA |
| | RILGGARKPDGVLYMPEFGVIVDTKAYA | | GCTGCCCCTGAGCTACATCGAGCTGCTG |
| | DGYSKSIAQADEMIRYIEDNKRRDPSRN | | GAGATCGCCAGGGACGGCAAGAGGAGCA |
| | STKWWEHFPTSIPANNFYFLWVSSVFVN | | GGGACTTCGAGCTGATCACCATCGAGCT |
| | KFHEQLSYTAQETQTVGAALSVEQLLLG | | GTTCAAGAACATCTACAAGATCAACGCC |
| | ADSVLKGNLTTEKFIDSFKNQEIVF | | AGGATCCTGGGCGGCGCCAGGAAGCCCG |
| | | | ACGGCGTGCTGTACATGCCCGAGTTCGG |
| | | | CGTGATCGTGGACACCAAGGCCTACGCC |
| | | | GACGGCTACAGCAAGAGCATCGCCCAGG |
| | | | CCGACGAGATGATCAGGTACATCGAGGA |
| | | | CAACAAGAGGAGGGACCCCAGCAGGAAC |
| | | | AGCACCAAGTGGTGGGAGCACTTCCCCA |
| | | | CCAGCATCCCCGCCAACAACTTCTACTT |
| | | | CCTGTGGGTGAGCAGCGTGTTCGTGAAC |
| | | | AAGTTCCACGAGCAGCTGAGCTACACCG |
| | | | CCCAGGAGACCCAGACCGTGGGCGCCGC |
| | | | CCTGAGCGTGGAGCAGCTGCTGCTGGGC |
| | | | GCCGACAGCGTGCTGAAGGGCAACCTGA |
| | | | CCACCGAGAAGTTCATCGACAGCTTCAA |
| | | | GAACCAGGAGATCGTGTTC |
| 31 | GATKSDLSLLKDDIRKKLNHINHKYLVL | 112 | GGCGCCACCAAGAGCGACCTGAGCCTGC |
| | IDLGFDGTADRDYELQTADLLTSELAFK | | TGAAGGACGACATCAGGAAGAAGCTGAA |
| | GARLGDSRKPDVCVYHDKNGLIIDNKAY | | CCACATCAACCACAAGTACCTGGTGCTG |
| | GSGYSLPIKQADEMLRYIEENQKRDKAL | | ATCGACCTGGGCTTCGACGGCACCGCCG |
| | NPNEWWTIFDDAVSKFNFAFVSGEFTGG | | ACAGGGACTACGAGCTGCAGACCGCCGA |
| | FKDRLENISRRSYTNGAAINSVNLLLLA | | CCTGCTGACCAGCGAGCTGGCCTTCAAG |
| | EEIKSGRISYGDAFTKFECNDEIII | | GGCGCCAGGCTGGGCGACAGCAGGAAGC |
| | | | CCGACGTGTGCGTGTACCACGACAAGAA |
| | | | CGGCCTGATCATCGACAACAAGGCCTAC |
| | | | GGCAGCGGCTACAGCCTGCCCATCAAGC |
| | | | AGGCCGACGAGATGCTGAGGTACATCGA |
| | | | GGAGAACCAGAAGAGGGACAAGGCCCTG |
| | | | AACCCCAACGAGTGGTGGACCATCTTCG |
| | | | ACGACGCCGTGAGCAAGTTCAACTTCGC |
| | | | CTTCGTGAGCGGCGAGTTCACCGGCGGC |
| | | | TTCAAGGACAGGCTGGAGAACATCAGCA |
| | | | GGAGGAGCTACACCAACGGCGCCGCCAT |
| | | | CAACAGCGTGAACCTGCTGCTGCTGGCC |
| | | | GAGGAGATCAAGAGCGGCAGGATCAGCT |
| | | | ACGGCGACGCCTTCACCAAGTTCGAGTG |
| | | | CAACGACGAGATCATCATC |
| 32 | ELRNAALDKQKVNFINKTGLPMKYIELL | 113 | GAGCTGAGGAACGCCGCCCTGGACAAGC |
| | EIAFDGSRNRDFEMVTADLFKNVYGENS | | AGAAGGTGAACTTCATCAACAAGACCGG |
| | ILLGGGRKPDGLIFTDRFGVIIDTKAYG | | CCTGCCCATGAAGTACATCGAGCTGCTG |
| | NGYSKSIGQEDEMVRYIEDNQLRDSNRN | | GAGATCGCCTTCGACGGCAGCAGGAACA |
| | SVEWWKNFDEKIESENFYFMWISSKFIG | | GGGACTTCGAGATGGTGACCGCCGACCT |
| | QFSDQLQSTSDRTNTKGAALNVEQLLLG | | GTTCAAGAACGTGTACGGCTTCAACAGC |
| | AAAARDGKLDINSLPIYMNNKEILW | | ATCCTGCTGGGCGGCGGCAGGAAGCCCG |
| | | | ACGGCCTGATCTTCACCGACAGGTTCGG |
| | | | CGTGATCATCGACACCAAGGCCTACGGC |
| | | | AACGGCTACAGCAAGAGCATCGGCCAGG |
| | | | AGGACGAGATGGTGAGGTACATCGAGGA |
| | | | CAACCAGCTGAGGGACAGCAACAGGAAC |
| | | | AGCGTGGAGTGGTGGAAGAACTTCGACG |
| | | | AGAAGATCGAGAGCGAGAACTTCTACTT |
| | | | CATGTGGATCAGCAGCAAGTTCATCGGC |
| | | | CAGTTCAGCGACCAGCTGCAGAGCACCA |
| | | | GCGACAGGACCAACACCAAGGGCGCCGC |
| | | | CCTGAACGTGGAGCAGCTGCTGCTGGGC |
| | | | GCCGCCGCCGCCAGGGACGGCAAGCTGG |
| | | | ACATCAACAGCCTGCCCATCTACATGAA |
| | | | CAACAAGGAGATCCTGTGG |
| 33 | ELKDEQSEKRKAYFLKETNLPLKYIELL | 114 | GAGCTGAAGGACGAGCAGAGCGAGAAGA |
| | DIAYDGKRNRDFEIVTMELFRNVYRLQS | | GGAAGGCCTACTTCCTGAAGGAGACCAA |
| | KLLGGVRKPDGLLYKHRFGIIVDTKAYG | | CCTGCCCCTGAAGTACATCGAGCTGCTG |
| | EGYSKSISQADEMIRYIEDNKRRDENRN | | GACATCGCCTACGACGGCAAGAGGAACA |
| | STKWWEHFPDCIPKQSFYFMWVSSKFVG | | GGGACTTCGAGATCGTGACCATGGAGCT |
| | KFQEQLDYTANETKTNGAALNVEQLLWG | | GTTCAGGAACGTGTACAGGCTGCAGAGC |
| | ADLVAKGKLDISQLPSYFQNKEIEF | | AAGCTGCTGGGCGGCGTGAGGAAGCCCG |
| | | | ACGGCCTGCTGTACAAGCACAGGTTCGG |
| | | | CATCATCGTGGACACCAAGGCCTACGGC |
| | | | GAGGGCTACAGCAAGAGCATCAGCCAGG |
| | | | CCGACGAGATGATCAGGTACATCGAGGA |
| | | | CAACAAGAGGAGGGACGAGAACAGGAAC |
| | | | AGCACCAAGTGGTGGGAGCACTTCCCCG |
| | | | ACTGCATCCCCAAGCAGAGCTTCTACTT |
| | | | CATGTGGGTGAGCAGCAAGTTCGTGGGC |
| | | | AAGTTCCAGGAGCAGCTGGACTACACCG |
| | | | CCAACGAGACCAAGACCAACGGCGCCGC |
| | | | CCTGAACGTGGAGCAGCTGCTGTGGGGC |
| | | | GCCGACCTGGTGGCCAAGGGCAAGCTGG |
| | | | ACATCAGCCAGCTGCCCAGCTACTTCCA |
| | | | GAACAAGGAGATCGAGTTC |
| 34 | HNNKFKNYLRENSELSFKFIELIDIAYD | 115 | CACAACAACAAGTTCAAGAACTACCTGA |
| | GNRNRDMEIITAELLKEIYGLNVKLLGG | | GGGAGAACAGCGAGCTGAGCTTCAAGTT |
| | GRKPDILAYTDDIGIIIDTKAYKDGYGK | | CATCGAGCTGATCGACATCGCCTACGAC |
| | QINQADEMIRYIEDNQRRDLIRNPNEWW | | GGCAACAGGAACAGGGACATGGAGATCA |
| | RYFPKSISKEKIYFMWISSYFKNNFYEQ | | TCACCGCCGAGCTGCTGAAGGAGATCTA |
| | VQYTAQETKSIGAALNVRQLLLCADAIQ | | CGGCCTGAACGTGAAGCTGCTGGGCGGC |
| | KEVLSLDTFLGSFRNEEINLL | | GGCAGGAAGCCCGACATCCTGGCCTACA |
| | | | CCGACGACATCGGCATCATCATCGACAC |
| | | | CAAGGCCTACAAGGACGGCTACGGCAAG |
| | | | CAGATCAACCAGGCCGACGAGATGATCA |
| | | | GGTACATCGAGGACAACCAGAGGAGGGA |
| | | | CCTGATCAGGAACCCCAACGAGTGGTGG |
| | | | AGGTACTTCCCCAAGAGCATCAGCAAGG |
| | | | AGAAGATCTACTTCATGTGGATCAGCAG |
| | | | CTACTTCAAGAACAACTTCTACGAGCAG |
| | | | GTGCAGTACACCGCCCAGGAGACCAAGA |
| | | | GCATCGGCGCCGCCCTGAACGTGAGGCA |
| | | | GCTGCTGCTGTGCGCCGACGCCATCCAG |
| | | | AAGGAGGTGCTGAGCCTGGACACCTTCC |
| | | | TGGGCAGCTTCAGGAACGAGGAGATCAA |
| | | | CCTG |
| 35 | PVKSEVSILKDYLRSHLTHIDHKYLILV | 116 | CTGCCCGTGAAGAGCGAGGTGAGCATCC |
| | DLGYDGTSDRDYEIQTAQLLTAELSFLG | | TGAAGGACTACCTGAGGAGCCACCTGAC |
| | GRLGDTRKPDVCIYYEDNGLIIDNKAYG | | CCACATCGACCACAAGTACCTGATCCTG |
| | KGYSLPMKQADEMYRYIEENKERSELLN | | GTGGACCTGGGCTACGACGGCACCAGCG |
| | PNCWWNIFDKDVKTFHFAFLSGEFTGGF | | ACAGGGACTACGAGATCCAGACCGCCCA |
| | RDRLNHISMRSGMRGAAVNSANLLIMAE | | GCTGCTGACCGCCGAGCTGAGCTTCCTG |
| | KLKAGTMEYEEFFRLFDTNDEILF | | GGCGGCAGGCTGGGCGACACCAGGAAGC |
| | | | CCGACGTGTGCATCTACTACGAGGACAA |
| | | | CGGCCTGATCATCGACAACAAGGCCTAC |
| | | | GGCAAGGGCTACAGCCTGCCCATGAAGC |
| | | | AGGCCGACGAGATGTACAGGTACATCGA |
| | | | GGAGAACAAGGAGAGGAGCGAGCTGCTG |
| | | | AACCCCAACTGCTGGTGGAACATCTTCG |
| | | | ACAAGGACGTGAAGACCTTCCACTTCGC |
| | | | CTTCCTGAGCGGCGAGTTCACCGGCGGC |
| | | | TTCAGGGACAGGCTGAACCACATCAGCA |
| | | | TGAGGAGCGGCATGAGGGGCGCCGCCGT |
| | | | GAACAGCGCCAACCTGCTGATCATGGCC |
| | | | GAGAAGCTGAAGGCCGGCACCATGGAGT |
| | | | ACGAGGAGTTCTTCAGGCTGTTCGACAC |
| | | | CAACGACGAGATCCTGTTC |
| 36 | LPVKSQVSILKDYLRSYLSHVDHKYLIL | 117 | CTGCCCGTGAAGAGCCAGGTGAGCATCC |
| | LDLGFDGTSDRDYEIWTAQLLTAELSFL | | TGAAGGACTACCTGAGGAGCTACCTGAG |
| | GGRLGDTRKPDVCIYYEDNGLIIDNKAY | | CCACGTGGACCACAAGTACCTGATCCTG |
| | GKGYSLPIKQADEMYRYIEENKERSDLL | | CTGGACCTGGGCTTCGACGGCACCAGCG |
| | NPNCWWNIFGEGVKTFRFAFLSGEFTGG | | ACAGGGACTACGAGATCTGGACCGCCCA |
| | FKDRLNHISMRSGIKGAAVNSANLLIMA | | GCTGCTGACCGCCGAGCTGAGCTTCCTG |
| | EQLKSGTMSYEEFFQLFDYNDEIIF | | GGCGGCAGGCTGGGCGACACCAGGAAGC |
| | | | CCGACGTGTGCATCTACTACGAGGACAA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAGCGACCTGCTG |
| | | | AACCCCAACTGCTGGTGGAACATCTTCG |
| | | | GCGAGGGCGTGAAGACCTTCAGGTTCGC |
| | | | CTTCCTGAGCGGCGAGTTCACCGGCGGC |
| | | | TTCAAGGACAGGCTGAACCACATCAGCA |
| | | | TGAGGAGCGGCATCAAGGGCGCCGCCGT |
| | | | GAACAGCGCCAACCTGCTGATCATGGCC |
| | | | GAGCAGCTGAAGAGCGGCACCATGAGCT |
| | | | ACGAGGAGTTCTTCCAGCTGTTCGACTA |
| | | | CAACGACGAGATCATCTTC |
| 37 | VSKTNILELKDNTREKLVYLDHRYLSLF | 118 | GTGAGCAAGACCAACATCCTGGAGCTGA |
| | DLAYDDKASRDFEIQTIDLLINELQFKG | | AGGACAACACCAGGGAGAAGCTGGTGTA |
| | LRLGERRKPDGIISYGVNGVIIDNKAYS | | CCTGGACCACAGGTACCTGAGCCTGTTC |
| | KGYNLPIRQADEMIRYIQENQSRDEKLN | | GACCTGGCCTACGACGACAAGGCCAGCA |
| | PNKWWENFEEETSKFNYLFISSKFISGF | | GGGACTTCGAGATCCAGACCATCGACCT |
| | KKNLQYIADRTGVNGGAINVENLLCFAE | | GCTGATCAACGAGCTGCAGTTCAAGGGC |
| | MLKSGKLEYNDFFNQYNNDEIIM | | CTGAGGCTGGGCGAGAGGAGGAAGCCCG |
| | | | ACGGCATCATCAGCTACGGCGTGAACGG |
| | | | CGTGATCATCGACAACAAGGCCTACAGC |
| | | | AAGGGCTACAACCTGCCCATCAGGCAGG |
| | | | CCGACGAGATGATCAGGTACATCCAGGA |
| | | | GAACCAGAGCAGGGACGAGAAGCTGAAC |
| | | | CCCAACAAGTGGTGGGAGAACTTCGAGG |
| | | | AGGAGACCAGCAAGTTCAACTACCTGTT |
| | | | CATCAGCAGCAAGTTCATCAGCGGCTTC |
| | | | AAGAAGAACCTGCAGTACATCGCCGACA |
| | | | GGACCGGCGTGAACGGCGGCGCCATCAA |
| | | | CGTGGAGAACCTGCTGTGCTTCGCCGAG |
| | | | ATGCTGAAGAGCGGCAAGCTGGAGTACA |
| | | | ACGACTTCTTCAACCAGTACAACAACGA |
| | | | CGAGATCATCATG |
| 38 | LPVKSQVSILKDYLRSCLSHVDHKYLIL | 119 | CTGCCCGTGAAGAGCCAGGTGAGCATCC |
| | LDLGFDGTSDRDYEIQTAQLLTAELSFL | | TGAAGGACTACCTGAGGAGCTGCCTGAG |
| | GGRLGDTRKPDVCIYYEDNGLIIDNKAY | | CCACGTGGACCACAAGTACCTGATCCTG |
| | GKGYSLPIKQADEMYRYIEENKERSELL | | CTGGACCTGGGCTTCGACGGCACCAGCG |
| | NPNCWWNIFDEGVKTFRFAFLSGEFTGG | | ACAGGGACTACGAGATCCAGACCGCCCA |
| | FKDRLNHISMRSGIKGAAVNSANLLIIA | | GCTGCTGACCGCCGAGCTGAGCTTCCTG |
| | EQLKSGTMSYEEFFQLFDQNDEITV | | GGCGGCAGGCTGGGCGACACCAGGAAGC |
| | | | CCGACGTGTGCATCTACTACGAGGACAA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAGCGAGCTGCTG |
| | | | AACCCCAACTGCTGGTGGAACATCTTCG |
| | | | ACGAGGGCGTGAAGACCTTCAGGTTCGC |
| | | | CTTCCTGAGCGGCGAGTTCACCGGCGGC |
| | | | TTCAAGGACAGGCTGAACCACATCAGCA |
| | | | TGAGGAGCGGCATCAAGGGCGCCGCCGT |
| | | | GAACAGCGCCAACCTGCTGATCATCGCC |
| | | | GAGCAGCTGAAGAGCGGCACCATGAGCT |
| | | | ACGAGGAGTTCTTCCAGCTGTTCGACCA |
| | | | GAACGACGAGATCACCGTG |
| 39 | MSSKSEISVIKDNIRKRLNHINHKYLVL | 120 | ATGAGCAGCAAGAGCGAGATCAGCGTGA |
| | IDLGFDGTADRDYELQTADLLTSELSFK | | TCAAGGACAACATCAGGAAGAGGCTGAA |
| | GARLGDTRKPDVCVYHGTNGLIIDNKAY | | CCACATCAACCACAAGTACCTGGTGCTG |
| | GKGYSLPIKQADEMLRYIEENQKRDKSL | | ATCGACCTGGGCTTCGACGGCACCGCCG |
| | NPNEWWTIFDDAVSKFNFAFVSGEFTGG | | ACAGGGACTACGAGCTGCAGACCGCCGA |
| | FKDRLENISRRSSVNGAAINSVNLLLLA | | CCTGCTGACCAGCGAGCTGAGCTTCAAG |
| | EEIKSGRMSYSDAFKNFDCNKEITI | | GGCGCCAGGCTGGGCGACACCAGGAAGC |
| | | | CCGACGTGTGCGTGTACCACGGCACCAA |
| | | | CGGCCTGATCATCGACAACAAGGCCTAC |
| | | | GGCAAGGGCTACAGCCTGCCCATCAAGC |
| | | | AGGCCGACGAGATGCTGAGGTACATCGA |
| | | | GGAGAACCAGAAGAGGGACAAGAGCCTG |
| | | | AACCCCAACGAGTGGTGGACCATCTTCG |
| | | | ACGACGCCGTGAGCAAGTTCAACTTCGC |
| | | | CTTCGTGAGCGGCGAGTTCACCGGCGGC |
| | | | TTCAAGGACAGGCTGGAGAACATCAGCA |
| | | | GGAGGAGCAGCGTGAACGGCGCCGCCAT |
| | | | CAACAGCGTGAACCTGCTGCTGCTGGCC |
| | | | GAGGAGATCAAGAGCGGCAGGATGAGCT |
| | | | ACAGCGACGCCTTCAAGAACTTCGACTG |
| | | | CAACAAGGAGATCACCATC |
| 40 | RNLDKVERDSRKAEFLAKTSLPPRFIEL | 121 | AGGAACCTGGACAAGGTGGAGAGGGACA |
| | LSIAYESKSNRDFEMITAEFFKDVYGLG | | GCAGGAAGGCCGAGTTCCTGGCCAAGAC |
| | AVHLGNARKPDALAFTDNFGIVIDTKAY | | CAGCCTGCCCCCCAGGTTCATCGAGCTG |
| | SNGYSKNINQEDEMVRYIEDNQIRSPER | | CTGAGCATCGCCTACGAGAGCAAGAGCA |
| | NKNEWWLSFPPSIPENNFHFLWVSSYFT | | ACAGGGACTTCGAGATGATCACCGCCGA |
| | GYFEEQLQETSDRAGGMTGGALDIEQLL | | GTTCTTCAAGGACGTGTACGGCCTGGGC |
| | IGGSLVQEGKLAPHDIPEYMQNRVIHF | | GCCGTGCACCTGGGCAACGCCAGGAAGC |
| | | | CCGACGCCCTGGCCTTCACCGACAACTT |
| | | | CGGCATCGTGATCGACACCAAGGCCTAC |
| | | | AGCAACGGCTACAGCAAGAACATCAACC |
| | | | AGGAGGACGAGATGGTGAGGTACATCGA |
| | | | GGACAACCAGATCAGGAGCCCCGAGAGG |
| | | | AACAAGAACGAGTGGTGGCTGAGCTTCC |
| | | | CCCCCAGCATCCCCGAGAACAACTTCCA |
| | | | CTTCCTGTGGGTGAGCAGCTACTTCACC |
| | | | GGCTACTTCGAGGAGCAGCTGCAGGAGA |
| | | | CCAGCGACAGGGCCGGCGGCATGACCGG |
| | | | CGGCGCCCTGGACATCGAGCAGCTGCTG |
| | | | ATCGGCGGCAGCCTGGTGCAGGAGGGCA |
| | | | AGCTGGCCCCCCACGACATCCCCGAGTA |
| | | | CATGCAGAACAGGGTGATCCACTTC |
| 41 | APVKSEVSLCKDILRSHLTHVDHKYLIL | 122 | GCCCCCGTGAAGAGCGAGGTGAGCCTGT |
| | LDLGFDGTSDRDYEIQTAQLLTAELDFK | | GCAAGGACATCCTGAGGAGCCACCTGAC |
| | GARLGDTRKPDVCVYYGEDGLILDNKAY | | CCACGTGGACCACAAGTACCTGATCCTG |
| | GKGYSLPIKQADEMYRYIEENKERNERL | | CTGGACCTGGGCTTCGACGGCACCAGCG |
| | NPNKWWEIFDKDVVRYHFAFVSGTFTGG | | ACAGGGACTACGAGATCCAGACCGCCCA |
| | FKERLDNIRMRSGICGAAVNSMNLLLMA | | GCTGCTGACCGCCGAGCTGGACTTCAAG |
| | EELKSGRLGYKECFALFDCNDEIAF | | GGCGCCAGGCTGGGCGACACCAGGAAGC |
| | | | CCGACGTGTGCGTGTACTACGGCGAGGA |
| | | | CGGCCTGATCCTGGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAACGAGAGGCTG |
| | | | AACCCCAACAAGTGGTGGGAGATCTTCG |
| | | | ACAAGGACGTGGTGAGGTACCACTTCGC |
| | | | CTTCGTGAGCGGCACCTTCACCGGCGGC |
| | | | TTCAAGGAGAGGCTGGACAACATCAGGA |
| | | | TGAGGAGCGGCATCTGCGGCGCCGCCGT |
| | | | GAACAGCATGAACCTGCTGCTGATGGCC |
| | | | GAGGAGCTGAAGAGCGGCAGGCTGGGCT |
| | | | ACAAGGAGTGCTTCGCCCTGTTCGACTG |
| | | | CAACGACGAGATCGCCTTC |
| 42 | SCVKDEVNDIVDRVRVKLKNIDHKYLIL | 123 | AGCTGCGTGAAGGACGAGGTGAACGACA |
| | ISLAYSDETERTKKNSDARDFEIQTAEL | | TCGTGGACAGGGTGAGGGTGAAGCTGAA |
| | FTKELGENGIRLGESNKPDVLISFGANG | | GAACATCGACCACAAGTACCTGATCCTG |
| | TIIDNKSYKDGFNIPRVTSDQMIRYINE | | ATCAGCCTGGCCTACAGCGACGAGACCG |
| | NNQRTTQLNPNEWWKNFDSSVSNYTFLF | | AGAGGACCAAGAAGAACAGCGACGCCAG |
| | VTSFLKGSFKNQIEYISNATNGTRGAAI | | GGACTTCGAGATCCAGACCGCCGAGCTG |
| | NVESLLYISEDIKSGKIKQSDFYSEFKN | | TTCACCAAGGAGCTGGGCTTCAACGGCA |
| | DEIVY | | TCAGGCTGGGCGAGAGCAACAAGCCCGA |
| | | | CGTGCTGATCAGCTTCGGCGCCAACGGC |
| | | | ACCATCATCGACAACAAGAGCTACAAGG |
| | | | ACGGCTTCAACATCCCCAGGGTGACCAG |
| | | | CGACCAGATGATCAGGTACATCAACGAG |
| | | | AACAACCAGAGGACCACCCAGCTGAACC |
| | | | CCAACGAGTGGTGGAAGAACTTCGACAG |
| | | | CAGCGTGAGCAACTACACCTTCCTGTTC |
| | | | GTGACCAGCTTCCTGAAGGGCAGCTTCA |
| | | | AGAACCAGATCGAGTACATCAGCAACGC |
| | | | CACCAACGGCACCAGGGGCGCCGCCATC |
| | | | AACGTGGAGAGCCTGCTGTACATCAGCG |
| | | | AGGACATCAAGAGCGGCAAGATCAAGCA |
| | | | GAGCGACTTCTACAGCGAGTTCAAGAAC |
| | | | GACGAGATCGTGTAC |
| 43 | SQGDKAREQLKAKFLAKTNLLPRYVELL | 124 | AGCCAGGGCGACAAGGCCAGGGAGCAGC |
| | DIAYDSKRNRDFEMVTAELFNFAYLLPA | | TGAAGGCCAAGTTCCTGGCCAAGACCAA |
| | VHLGGVRKPDALVATKKFGIIVDTKAYA | | CCTGCTGCCCAGGTACGTGGAGCTGCTG |
| | NGYSRNANQADEMARYITENQKRDPKTN | | GACATCGCCTACGACAGCAAGAGGAACA |
| | PNRWWDNFDARIPPNAYYFLWVSSFFTG | | GGGACTTCGAGATGGTGACCGCCGAGCT |
| | QFDDQLSYTAHRTNTHGGALNVEQLLIG | | GTTCAACTTCGCCTACCTGCTGCCCGCC |
| | ANMIQTGQLDRNKLPEYMQDKEITF | | GTGCACCTGGGCGGCGTGAGGAAGCCCG |
| | | | ACGCCCTGGTGGCCACCAAGAAGTTCGG |
| | | | CATCATCGTGGACACCAAGGCCTACGCC |
| | | | AACGGCTACAGCAGGAACGCCAACCAGG |
| | | | CCGACGAGATGGCCAGGTACATCACCGA |
| | | | GAACCAGAAGAGGGACCCCAAGACCAAC |
| | | | CCCAACAGGTGGTGGGACAACTTCGACG |
| | | | CCAGGATCCCCCCCAACGCCTACTACTT |
| | | | CCTGTGGGTGAGCAGCTTCTTCACCGGC |
| | | | CAGTTCGACGACCAGCTGAGCTACACCG |
| | | | CCCACAGGACCAACACCCACGGCGGCGC |
| | | | CCTGAACGTGGAGCAGCTGCTGATCGGC |
| | | | GCCAACATGATCCAGACCGGCCAGCTGG |
| | | | ACAGGAACAAGCTGCCCGAGTACATGCA |
| | | | GGACAAGGAGATCACCTTC |
| 44 | KVQKSNILDVIEKCREKINNIPHEYLAL | 125 | AAGGTGCAGAAGAGCAACATCCTGGACG |
| | IPMSFDENESTMFEIKTIELLTEHCKFD | | TGATCGAGAAGTGCAGGGAGAAGATCAA |
| | GLHCGGASKPDGLIYSEDYGVIIDTKSY | | CAACATCCCCCACGAGTACCTGGCCCTG |
| | KDGENIQTPERDKMKRYIEENQNRNPQH | | ATCCCCATGAGCTTCGACGAGAACGAGA |
| | NKTRWWDEFPHNISNFLFLFVSGKFGGN | | GCACCATGTTCGAGATCAAGACCATCGA |
| | FKEQLRILSEQTNNTLGGALSSYVLLNI | | GCTGCTGACCGAGCACTGCAAGTTCGAC |
| | AEQIAINKIDHCDFKTRISCLDEVA | | GGCCTGCACTGCGGCGGCGCCAGCAAGC |
| | | | CCGACGGCCTGATCTACAGCGAGGACTA |
| | | | CGGCGTGATCATCGACACCAAGAGCTAC |
| | | | AAGGACGGCTTCAACATCCAGACCCCCG |
| | | | AGAGGGACAAGATGAAGAGGTACATCGA |
| | | | GGAGAACCAGAACAGGAACCCCCAGCAC |
| | | | AACAAGACCAGGTGGTGGGACGAGTTCC |
| | | | CCCACAACATCAGCAACTTCCTGTTCCT |
| | | | GTTCGTGAGCGGCAAGTTCGGCGGCAAC |
| | | | TTCAAGGAGCAGCTGAGGATCCTGAGCG |
| | | | AGCAGACCAACAACACCCTGGGCGGCGC |
| | | | CCTGAGCAGCTACGTGCTGCTGAACATC |
| | | | GCCGAGCAGATCGCCATCAACAAGATCG |
| | | | ACCACTGCGACTTCAAGACCAGGATCAG |
| | | | CTGCCTGGACGAGGTGGCC |
| 45 | VPVKSEVSLCKDYLRSYLTHVDHKYLIL | 126 | GTGCCCGTGAAGAGCGAGGTGAGCCTGT |
| | LDLGFDGTSDRDYEIQTAQLLTAELDFK | | GCAAGGACTACCTGAGGAGCTACCTGAC |
| | GARLGDTRKPDVCVYYGEDGLIIDNKAY | | CCACGTGGACCACAAGTACCTGATCCTG |
| | GKGYSLPIKQADEIYRYIEENKKRDEKL | | CTGGACCTGGGCTTCGACGGCACCAGCG |
| | NPNKWWEIFDKGVVRYHFAFVSGAFTGG | | ACAGGGACTACGAGATCCAGACCGCCCA |
| | FKERLDNIRMRSGICGAAINSMNLLLMA | | GCTGCTGACCGCCGAGCTGGACTTCAAG |
| | EELKSGRLGYEECFALFDCNDEITF | | GGCGCCAGGCTGGGCGACACCAGGAAGC |
| | | | CCGACGTGTGCGTGTACTACGGCGAGGA |
| | | | CGGCCTGATCATCGACAACAAGGCCTAC |
| | | | GGCAAGGGCTACAGCCTGCCCATCAAGC |
| | | | AGGCCGACGAGATCTACAGGTACATCGA |
| | | | GGAGAACAAGAAGAGGGACGAGAAGCTG |
| | | | AACCCCAACAAGTGGTGGGAGATCTTCG |
| | | | ACAAGGGCGTGGTGAGGTACCACTTCGC |
| | | | CTTCGTGAGCGGCGCCTTCACCGGCGGC |
| | | | TTCAAGGAGAGGCTGGACAACATCAGGA |
| | | | TGAGGAGCGGCATCTGCGGCGCCGCCAT |
| | | | CAACAGCATGAACCTGCTGCTGATGGCC |
| | | | GAGGAGCTGAAGAGCGGCAGGCTGGGCT |
| | | | ACGAGGAGTGCTTCGCCCTGTTCGACTG |
| | | | CAACGACGAGATCACCTTC |
| 46 | VPVKSEVSLCKDYLRSHLNHVDHRYLIL | 127 | GTGCCCGTGAAGAGCGAGGTGAGCCTGT |
| | LDLGFDGTSDRDYEIQTAQLLTGELNFK | | GCAAGGACTACCTGAGGAGCCACCTGAA |
| | GARLGDTRKPDVCVYYGEDGLIIDNKAY | | CCACGTGGACCACAGGTACCTGATCCTG |
| | GKGYSLPIKQADEMYRYIEENKERNEKL | | CTGGACCTGGGCTTCGACGGCACCAGCG |
| | NPNKWWEIFDKDVIHYHFAFVSGAFTGG | | ACAGGGACTACGAGATCCAGACCGCCCA |
| | FKERLENIRMRSGIYGAAVNSMNLLLMA | | GCTGCTGACCGGCGAGCTGAACTTCAAG |
| | EELKSGRLDYKECFKLFDCNDEIVL | | GGCGCCAGGCTGGGCGACACCAGGAAGC |
| | | | CCGACGTGTGCGTGTACTACGGCGAGGA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAACGAGAAGCTG |
| | | | AACCCCAACAAGTGGTGGGAGATCTTCG |
| | | | ACAAGGACGTGATCCACTACCACTTCGC |
| | | | CTTCGTGAGCGGCGCCTTCACCGGCGGC |
| | | | TTCAAGGAGAGGCTGGAGAACATCAGGA |
| | | | TGAGGAGCGGCATCTACGGCGCCGCCGT |
| | | | GAACAGCATGAACCTGCTGCTGATGGCC |
| | | | GAGGAGCTGAAGAGCGGCAGGCTGGACT |
| | | | ACAAGGAGTGCTTCAAGCTGTTCGACTG |
| | | | CAACGACGAGATCGTGCTG |
| 47 | VPVKSEVSLLKDYLRSHLVHVDHKYLVL | 128 | GTGCCCGTGAAGAGCGAGGTGAGCCTGC |
| | LDLGFDGTSDRDYEIQTAQLLTGELNFK | | TGAAGGACTACCTGAGGAGCCACCTGGT |
| | GARLGDTRKPDVCVYYGEDGLIIDNKAY | | GCACGTGGACCACAAGTACCTGGTGCTG |
| | GKGYSLPIKQADEMYRYIEENKERNEKL | | CTGGACCTGGGCTTCGACGGCACCAGCG |
| | NPNKWWEIFGNDVIHYHFAFVSGAFTGG | | ACAGGGACTACGAGATCCAGACCGCCCA |
| | FKERLDNIRMRSGIYGAAVNSMNLLLLA | | GCTGCTGACCGGCGAGCTGAACTTCAAG |
| | EELKSGRLGYKECFKLFDCNDEIVL | | GGCGCCAGGCTGGGCGACACCAGGAAGC |
| | | | CCGACGTGTGCGTGTACTACGGCGAGGA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAACGAGAAGCTG |
| | | | AACCCCAACAAGTGGTGGGAGATCTTCG |
| | | | GCAACGACGTGATCCACTACCACTTCGC |
| | | | CTTCGTGAGCGGCGCCTTCACCGGCGGC |
| | | | TTCAAGGAGAGGCTGGACAACATCAGGA |
| | | | TGAGGAGCGGCATCTACGGCGCCGCCGT |
| | | | GAACAGCATGAACCTGCTGCTGCTGGCC |
| | | | GAGGAGCTGAAGAGCGGCAGGCTGGGCT |
| | | | ACAAGGAGTGCTTCAAGCTGTTCGACTG |
| | | | CAACGACGAGATCGTGCTG |
| 48 | ECVKDNVVDIKDRVRNKLIHLDHKYLAL | 129 | GAGTGCGTGAAGGACAACGTGGTGGACA |
| | IDLAYSDAASRAKKNADAREFEIQTADL | | TCAAGGACAGGGTGAGGAACAAGCTGAT |
| | FTKELSFNGQRLGDSRKPDVIISYGLDG | | CCACCTGGACCACAAGTACCTGGCCCTG |
| | TIVDNKSYKDGFNISRTCADEMSRYINE | | ATCGACCTGGCCTACAGCGACGCCGCCA |
| | NNLRQKSLNPNEWWKNFDSTITAYTFLF | | GCAGGGCCAAGAAGAACGCCGACGCCAG |
| | ITSYLKGQFEDQLEYVSNANGGIKGAAI | | GGAGTTCGAGATCCAGACCGCCGACCTG |
| | GVESLLYLSEGIKAGRISHADFYSNFNN | | TTCACCAAGGAGCTGAGCTTCAACGGCC |
| | KEMIY | | AGAGGCTGGGCGACAGCAGGAAGCCCGA |
| | | | CGTGATCATCAGCTACGGCCTGGACGGC |
| | | | ACCATCGTGGACAACAAGAGCTACAAGG |
| | | | ACGGCTTCAACATCAGCAGGACCTGCGC |
| | | | CGACGAGATGAGCAGGTACATCAACGAG |
| | | | AACAACCTGAGGCAGAAGAGCCTGAACC |
| | | | CCAACGAGTGGTGGAAGAACTTCGACAG |
| | | | CACCATCACCGCCTACACCTTCCTGTTC |
| | | | ATCACCAGCTACCTGAAGGGCCAGTTCG |
| | | | AGGACCAGCTGGAGTACGTGAGCAACGC |
| | | | CAACGGCGGCATCAAGGGCGCCGCCATC |
| | | | GGCGTGGAGAGCCTGCTGTACCTGAGCG |
| | | | AGGGCATCAAGGCCGGCAGGATCAGCCA |
| | | | CGCCGACTTCTACAGCAACTTCAACAAC |
| | | | AAGGAGATGATCTAC |
| 49 | IAKSDFSIIKDNIRRKLQYVNHKYLLLI | 130 | ATCGCCAAGAGCGACTTCAGCATCATCA |
| | DLGFDSDSNRDYEIQTAELLTTELAFKG | | AGGACAACATCAGGAGGAAGCTGCAGTA |
| | ARLGDTRKPDVCVYYGENGLIIDNKAYS | | CGTGAACCACAAGTACCTGCTGCTGATC |
| | KGYSLPMSQADEMVRYIEENKARQSSIN | | GACCTGGGCTTCGACAGCGACAGCAACA |
| | PNQWWKIFEDTVCNFNYAFVSGEFTGGF | | GGGACTACGAGATCCAGACCGCCGAGCT |
| | KDRLNNICERTRVSGGAINTINLLLLAE | | GCTGACCACCGAGCTGGCCTTCAAGGGC |
| | ELKSGRMSYPKCFSYFDTNDEVHI | | GCCAGGCTGGGCGACACCAGGAAGCCCG |
| | | | ACGTGTGCGTGTACTACGGCGAGAACGG |
| | | | CCTGATCATCGACAACAAGGCCTACAGC |
| | | | AAGGGCTACAGCCTGCCCATGAGCCAGG |
| | | | CCGACGAGATGGTGAGGTACATCGAGGA |
| | | | GAACAAGGCCAGGCAGAGCAGCATCAAC |
| | | | CCCAACCAGTGGTGGAAGATCTTCGAGG |
| | | | ACACCGTGTGCAACTTCAACTACGCCTT |
| | | | CGTGAGCGGCGAGTTCACCGGCGGCTTC |
| | | | AAGGACAGGCTGAACAACATCTGCGAGA |
| | | | GGACCAGGGTGAGCGGCGGCGCCATCAA |
| | | | CACCATCAACCTGCTGCTGCTGGCCGAG |
| | | | GAGCTGAAGAGCGGCAGGATGAGCTACC |
| | | | CCAAGTGCTTCAGCTACTTCGACACCAA |
| | | | CGACGAGGTGCACATC |
| 50 | LKYLGIKKQNRAFEIITAELFNTSYKLS | 131 | CTGAAGTACCTGGGCATCAAGAAGCAGA |
| | ATHLGGGRRPDVLVYNDNFGIIVDTKAY | | ACAGGGCCTTCGAGATCATCACCGCCGA |
| | KDGYGRNVNQEDEMVRYITENNIRKQDI | | GCTGTTCAACACCAGCTACAAGCTGAGC |
| | NKNDWWKYFSKSIPSTSYYHLWISSQFV | | GCCACCCACCTGGGCGGCGGCAGGAGGC |
| | GMFSDQLRETSSRTGENGGAMNVEQLLI | | CCGACGTGCTGGTGTACAACGACAACTT |
| | GANQVLNNVLDPNCLPKYMENKEIIF | | CGGCATCATCGTGGACACCAAGGCCTAC |
| | | | AAGGACGGCTACGGCAGGAACGTGAACC |
| | | | AGGAGGACGAGATGGTGAGGTACATCAC |
| | | | CGAGAACAACATCAGGAAGCAGGACATC |
| | | | AACAAGAACGACTGGTGGAAGTACTTCA |
| | | | GCAAGAGCATCCCCAGCACCAGCTACTA |
| | | | CCACCTGTGGATCAGCAGCCAGTTCGTG |
| | | | GGCATGTTCAGCGACCAGCTGAGGGAGA |
| | | | CCAGCAGCAGGACCGGCGAGAACGGCGG |
| | | | CGCCATGAACGTGGAGCAGCTGCTGATC |
| | | | GGCGCCAACCAGGTGCTGAACAACGTGC |
| | | | TGGACCCCAACTGCCTGCCCAAGTACAT |
| | | | GGAGAACAAGGAGATCATCTTC |
| 51 | VPVKSEVSLCKDYLRSHLNHVDHKYLIL | 132 | GTGCCCGTGAAGAGCGAGGTGAGCCTGT |
| | LDLGFDGTSDRDYEIQTAQLLTGELNFK | | GCAAGGACTACCTGAGGAGCCACCTGAA |
| | GARLGDTRKPDVCVYYGEDGLIIDNKAY | | CCACGTGGACCACAAGTACCTGATCCTG |
| | GKGYSLPIKQADEMYRYIEENKERNEKL | | CTGGACCTGGGCTTCGACGGCACCAGCG |
| | NPNKWWEIFDKDVIHYHFAFVSGAFTGG | | ACAGGGACTACGAGATCCAGACCGCCCA |
| | FRERLENIRMRSGIYGAAVNSMNLLLMA | | GCTGCTGACCGGCGAGCTGAACTTCAAG |
| | EELKSGRLGYKECFKLFDCNDEIVL | | GGCGCCAGGCTGGGCGACACCAGGAAGC |
| | | | CCGACGTGTGCGTGTACTACGGCGAGGA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAACGAGAAGCTG |
| | | | AACCCCAACAAGTGGTGGGAGATCTTCG |
| | | | ACAAGGACGTGATCCACTACCACTTCGC |
| | | | CTTCGTGAGCGGCGCCTTCACCGGCGGC |
| | | | TTCAGGGAGAGGCTGGAGAACATCAGGA |
| | | | TGAGGAGCGGCATCTACGGCGCCGCCGT |
| | | | GAACAGCATGAACCTGCTGCTGATGGCC |
| | | | GAGGAGCTGAAGAGCGGCAGGCTGGGCT |
| | | | ACAAGGAGTGCTTCAAGCTGTTCGACTG |
| | | | CAACGACGAGATCGTGCTG |
| 52 | VPVKSEVSLLKDYLRTHLLHVDHRYLIL | 133 | GTGCCCGTGAAGAGCGAGGTGAGCCTGC |
| | LDLGFDGTSDRDYEIQTAQLLTGELNFK | | TGAAGGACTACCTGAGGACCCACCTGCT |
| | GARLGDTRKPDVCVYYGEDGLIIDNKAY | | GCACGTGGACCACAGGTACCTGATCCTG |
| | GKGYSLPIKQADEMYRYIEENKERNEKL | | CTGGACCTGGGCTTCGACGGCACCAGCG |
| | NPNKWWEIFDNDVIHYHFAFISGAFTGG | | ACAGGGACTACGAGATCCAGACCGCCCA |
| | FKERLDNIRMRSGIYGAAVNSMNLLLMA | | GCTGCTGACCGGCGAGCTGAACTTCAAG |
| | EELKSGRLGYKECFKLFDCNDEIVL | | GGCGCCAGGCTGGGCGACACCAGGAAGC |
| | | | CCGACGTGTGCGTGTACTACGGCGAGGA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAACGAGAAGCTG |
| | | | AACCCCAACAAGTGGTGGGAGATCTTCG |
| | | | ACAACGACGTGATCCACTACCACTTCGC |
| | | | CTTCATCAGCGGCGCCTTCACCGGCGGC |
| | | | TTCAAGGAGAGGCTGGACAACATCAGGA |
| | | | TGAGGAGCGGCATCTACGGCGCCGCCGT |
| | | | GAACAGCATGAACCTGCTGCTGATGGCC |
| | | | GAGGAGCTGAAGAGCGGCAGGCTGGGCT |
| | | | ACAAGGAGTGCTTCAAGCTGTTCGACTG |
| | | | CAACGACGAGATCGTGCTG |
| 53 | VPVKSEVSLCKDYLRSHLNHVDHKYLIL | 134 | GTGCCCGTGAAGAGCGAGGTGAGCCTGT |
| | LDLGFDGTSDRDYEIQTAQLLTGELNFK | | GCAAGGACTACCTGAGGAGCCACCTGAA |
| | GARLGDTRKPDVCVYYGEDGLIIDNKAY | | CCACGTGGACCACAAGTACCTGATCCTG |
| | GKGYSLPIKQADEMYRYIEENKERNEKL | | CTGGACCTGGGCTTCGACGGCACCAGCG |
| | NPNKWWEIFDNDVIHYHFAFVSGAFTGG | | ACAGGGACTACGAGATCCAGACCGCCCA |
| | FRERLENIRMRSGIYGAAVNSMNLLLMA | | GCTGCTGACCGGCGAGCTGAACTTCAAG |
| | EELKSGRLGYKECFKLFDCNDEIVL | | GGCGCCAGGCTGGGCGACACCAGGAAGC |
| | | | CCGACGTGTGCGTGTACTACGGCGAGGA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAACGAGAAGCTG |
| | | | AACCCCAACAAGTGGTGGGAGATCTTCG |
| | | | ACAACGACGTGATCCACTACCACTTCGC |
| | | | CTTCGTGAGCGGCGCCTTCACCGGCGGC |
| | | | TTCAGGGAGAGGCTGGAGAACATCAGGA |
| | | | TGAGGAGCGGCATCTACGGCGCCGCCGT |
| | | | GAACAGCATGAACCTGCTGCTGATGGCC |
| | | | GAGGAGCTGAAGAGCGGCAGGCTGGGCT |
| | | | ACAAGGAGTGCTTCAAGCTGTTCGACTG |
| | | | CAACGACGAGATCGTGCTG |
| 54 | VPVKSEMSLLKDYLRTHLLHVDHRYLIL | 135 | GTGCCCGTGAAGAGCGAGATGAGCCTGC |
| | LDLGFDGASDRDYEIQTAQLLTGELNFK | | TGAAGGACTACCTGAGGACCCACCTGCT |
| | GARLGDTRKPDVCVYYGEDGLIIDNKAY | | GCACGTGGACCACAGGTACCTGATCCTG |
| | GKGYSLPIKQADEMYRYIEENKERNEKL | | CTGGACCTGGGCTTCGACGGCGCCAGCG |
| | NPNKWWEIFDNDVIHYHFAFVSGAFTGG | | ACAGGGACTACGAGATCCAGACCGCCCA |
| | FKERLDNIRMRSGIYGAAVNSMNLLLMA | | GCTGCTGACCGGCGAGCTGAACTTCAAG |
| | EELKSGRLGYKECFKLFDCNDEIVL | | GGCGCCAGGCTGGGCGACACCAGGAAGC |
| | | | CCGACGTGTGCGTGTACTACGGCGAGGA |
| | | | CGGCCTGATCATCGACAACAAGGCCTAC |
| | | | GGCAAGGGCTACAGCCTGCCCATCAAGC |
| | | | AGGCCGACGAGATGTACAGGTACATCGA |
| | | | GGAGAACAAGGAGAGGAACGAGAAGCTG |
| | | | AACCCCAACAAGTGGTGGGAGATCTTCG |
| | | | ACAACGACGTGATCCACTACCACTTCGC |
| | | | CTTCGTGAGCGGCGCCTTCACCGGCGGC |
| | | | TTCAAGGAGAGGCTGGACAACATCAGGA |
| | | | TGAGGAGCGGCATCTACGGCGCCGCCGT |
| | | | GAACAGCATGAACCTGCTGCTGATGGCC |
| | | | GAGGAGCTGAAGAGCGGCAGGCTGGGCT |
| | | | ACAAGGAGTGCTTCAAGCTGTTCGACTG |
| | | | CAACGACGAGATCGTGCTG |
| 55 | ILVDKEREMRKAKFLKETVLDSKFISLL | 136 | ATCCTGGTGGACAAGGAGAGGGAGATGA |
| | DLAADATKSRDFEIVTAELFKEAYNLNS | | GGAAGGCCAAGTTCCTGAAGGAGACCGT |
| | VLLGGSNKPDGLVFTDDFGILLDTKAYK | | GCTGGACAGCAAGTTCATCAGCCTGCTG |
| | NGFSIYAKDRDQMIRYVDDNNKRDKIRN | | GACCTGGCCGCCGACGCCACCAAGAGCA |
| | PNEWWKSFSPLIPNDKFYYLWVSNFFKG | | GGGACTTCGAGATCGTGACCGCCGAGCT |
| | QFKNQIEYVNRETNTYGAVLNVEQLLYG | | GTTCAAGGAGGCCTACAACCTGAACAGC |
| | ADAVIKGIINPNKLHEYFSNDEIKF | | GTGCTGCTGGGCGGCAGCAACAAGCCCG |
| | | | ACGGCCTGGTGTTCACCGACGACTTCGG |
| | | | CATCCTGCTGGACACCAAGGCCTACAAG |
| | | | AACGGCTTCAGCATCTACGCCAAGGACA |
| | | | GGGACCAGATGATCAGGTACGTGGACGA |
| | | | CAACAACAAGAGGGACAAGATCAGGAAC |
| | | | CCCAACGAGTGGTGGAAGAGCTTCAGCC |
| | | | CCCTGATCCCCAACGACAAGTTCTACTA |
| | | | CCTGTGGGTGAGCAACTTCTTCAAGGGC |
| | | | CAGTTCAAGAACCAGATCGAGTACGTGA |
| | | | ACAGGGAGACCAACACCTACGGCGCCGT |
| | | | GCTGAACGTGGAGCAGCTGCTGTACGGC |
| | | | GCCGACGCCGTGATCAAGGGCATCATCA |
| | | | ACCCCAACAAGCTGCACGAGTACTTCAG |
| | | | CAACGACGAGATCAAGTTC |
| 56 | TVDEKERLELKEYFISNTRIPSKYITLL | 137 | ACCGTGGACGAGAAGGAGAGGCTGGAGC |
| | DLAYDGNANRDFEIVTAELFKDIFKLQS | | TGAAGGAGTACTTCATCAGCAACACCAG |
| | KHMGGTRKPDILIWTDKFGVIADTKAYS | | GATCCCCAGCAAGTACATCACCCTGCTG |
| | KGYKKNISEADKMVRYVNENTNRNKVDN | | GACCTGGCCTACGACGGCAACGCCAACA |
| | TNEWWNSFDSRIPKDAYYFLWISSEFVG | | GGGACTTCGAGATCGTGACCGCCGAGCT |
| | KFDEQLTETSSRTGRNGASINVYQLLRG | | GTTCAAGGACATCTTCAAGCTGCAGAGC |
| | ADLVQKSKFNIHDLPNLMQNNEIKF | | AAGCACATGGGCGGCACCAGGAAGCCCG |
| | | | ACATCCTGATCTGGACCGACAAGTTCGG |
| | | | CGTGATCGCCGACACCAAGGCCTACAGC |
| | | | AAGGGCTACAAGAAGAACATCAGCGAGG |
| | | | CCGACAAGATGGTGAGGTACGTGAACGA |
| | | | GAACACCAACAGGAACAAGGTGGACAAC |
| | | | ACCAACGAGTGGTGGAACAGCTTCGACA |
| | | | GCAGGATCCCCAAGGACGCCTACTACTT |
| | | | CCTGTGGATCAGCAGCGAGTTCGTGGGC |
| | | | AAGTTCGACGAGCAGCTGACCGAGACCA |
| | | | GCAGCAGGACCGGCAGGAACGGCGCCAG |
| | | | CATCAACGTGTACCAGCTGCTGAGGGGC |
| | | | GCCGACCTGGTGCAGAAGAGCAAGTTCA |
| | | | ACATCCACGACCTGCCCAACCTGATGCA |
| | | | GAACAACGAGATCAAGTTC |
| 57 | TLQKSDIEKFKNQLRTELTNIDHSYLKG | 138 | ACCCTGCAGAAGAGCGACATCGAGAAGT |
| | IDIASKKTTTNVENTEFEAISTKVFTDE | | TCAAGAACCAGCTGAGGACCGAGCTGAC |
| | LGFFGEHLGGSNKPDGLIWDNDCAIILD | | CAACATCGACCACAGCTACCTGAAGGGC |
| | SKAYSEGFPLTASHTDAMGRYLRQFKER | | ATCGACATCGCCAGCAAGAAGACCACCA |
| | KEEIKPTWWDIAPDNLANTYFAYVSGSF | | CCAACGTGGAGAACACCGAGTTCGAGGC |
| | SGNYKAQLQKFRQDTNHMGGALEFVKLL | | CATCAGCACCAAGGTGTTCACCGACGAG |
| | LLANNYKAHKMSINEVKESILDYNISY | | CTGGGCTTCTTCGGCGAGCACCTGGGCG |
| | | | GCAGCAACAAGCCCGACGGCCTGATCTG |
| | | | GGACAACGACTGCGCCATCATCCTGGAC |
| | | | AGCAAGGCCTACAGCGAGGGCTTCCCCC |
| | | | TGACCGCCAGCCACACCGACGCCATGGG |
| | | | CAGGTACCTGAGGCAGTTCAAGGAGAGG |
| | | | AAGGAGGAGATCAAGCCCACCTGGTGGG |
| | | | ACATCGCCCCCGACAACCTGGCCAACAC |
| | | | CTACTTCGCCTACGTGAGCGGCAGCTTC |
| | | | AGCGGCAACTACAAGGCCCAGCTGCAGA |
| | | | AGTTCAGGCAGGACACCAACCACATGGG |
| | | | CGGCGCCCTGGAGTTCGTGAAGCTGCTG |
| | | | CTGCTGGCCAACAACTACAAGGCCCACA |
| | | | AGATGAGCATCAACGAGGTGAAGGAGAG |
| | | | CATCCTGGACTACAACATCAGCTAC |
| 58 | VKEKTDAALVKERVRLQLHNINHKYLAL | 139 | GTGAAGGAGAAGACCGACGCCGCCCTGG |
| | IDYAFSGKNNSRDFEVYTIDLLVNELTF | | TGAAGGAGAGGGTGAGGCTGCAGCTGCA |
| | GGLHLGGTRKPDGIFYHGSNGIIIDNKA | | CAACATCAACCACAAGTACCTGGCCCTG |
| | YAKGFVITRNMADEMIRYVQENNDRNPE | | ATCGACTACGCCTTCAGCGGCAAGAACA |
| | RNPNCWWKGFPHDVTRYNYVFISSMFKG | | ACAGCAGGGACTTCGAGGTGTACACCAT |
| | EVEHMLDNIRQSTGIDGCVLTIENLLYY | | CGACCTGCTGGTGAACGAGCTGACCTTC |
| | ADAIKGGTLSKATFINGENANKEMVF | | GGCGGCCTGCACCTGGGCGGCACCAGGA |
| | | | AGCCCGACGGCATCTTCTACCACGGCAG |
| | | | CAACGGCATCATCATCGACAACAAGGCC |
| | | | TACGCCAAGGGCTTCGTGATCACCAGGA |
| | | | ACATGGCCGACGAGATGATCAGGTACGT |
| | | | GCAGGAGAACAACGACAGGAACCCCGAG |
| | | | AGGAACCCCAACTGCTGGTGGAAGGGCT |
| | | | TCCCCCACGACGTGACCAGGTACAACTA |
| | | | CGTGTTCATCAGCAGCATGTTCAAGGGC |
| | | | GAGGTGGAGCACATGCTGGACAACATCA |
| | | | GGCAGAGCACCGGCATCGACGGCTGCGT |
| | | | GCTGACCATCGAGAACCTGCTGTACTAC |
| | | | GCCGACGCCATCAAGGGCGGCACCCTGA |
| | | | GCAAGGCCACCTTCATCAACGGCTTCAA |
| | | | CGCCAACAAGGAGATGGTGTTC |
| 59 | VKETTDSVIIKDRVRLKLHHVNHKYLTL | 140 | GTGAAGGAGACCACCGACAGCGTGATCA |
| | IDYAFSGKNNCMDFEVYTIDLLVNELAF | | TCAAGGACAGGGTGAGGCTGAAGCTGCA |
| | NGVHLGGTRKPDGIFYHNRNGIIIDNKA | | CCACGTGAACCACAAGTACCTGACCCTG |
| | YSHGFTLSRAMADEMIRYIQENNDRNPE | | ATCGACTACGCCTTCAGCGGCAAGAACA |
| | RNPNKWWENFDKGVNQFNFVFISSLFKG | | ACTGCATGGACTTCGAGGTGTACACCAT |
| | EIEHMLTNIKQSTDGVEGCVLSAENLLY | | CGACCTGCTGGTGAACGAGCTGGCCTTC |
| | FAEAMKSGVMPKTEFISYFGAGKEIQF | | AACGGCGTGCACCTGGGCGGCACCAGGA |
| | | | AGCCCGACGGCATCTTCTACCACAACAG |
| | | | GAACGGCATCATCATCGACAACAAGGCC |
| | | | TACAGCCACGGCTTCACCCTGAGCAGGG |
| | | | CCATGGCCGACGAGATGATCAGGTACAT |
| | | | CCAGGAGAACAACGACAGGAACCCCGAG |
| | | | AGGAACCCCAACAAGTGGTGGGAGAACT |
| | | | TCGACAAGGGCGTGAACCAGTTCAACTT |
| | | | CGTGTTCATCAGCAGCCTGTTCAAGGGC |
| | | | GAGATCGAGCACATGCTGACCAACATCA |
| | | | AGCAGAGCACCGACGGCGTGGAGGGCTG |
| | | | CGTGCTGAGCGCCGAGAACCTGCTGTAC |
| | | | TTCGCCGAGGCCATGAAGAGCGGCGTGA |
| | | | TGCCCAAGACCGAGTTCATCAGCTACTT |
| | | | CGGCGCCGGCAAGGAGATCCAGTTC |
| 60 | SACKADITELKDKIRKSLKVLDHKYLVL | 141 | AGCGCCTGCAAGGCCGACATCACCGAGC |
| | VDLAYSDASTKSKKNSDAREFEIQTADL | | TGAAGGACAAGATCAGGAAGAGCCTGAA |
| | FTKELKFDGMRLGDSNRPDVIISHDNFG | | GGTGCTGGACCACAAGTACCTGGTGCTG |
| | TIIDNKSYKDGFNIDKKCADEMSRYINE | | GTGGACCTGGCCTACAGCGACGCCAGCA |
| | NQRRIPELPKNEWWKNFDVNVDIFTFLF | | CCAAGAGCAAGAAGAACAGCGACGCCAG |
| | ITSYLKGNFKDQLEYISKSQSDIKGAAI | | GGAGTTCGAGATCCAGACCGCCGACCTG |
| | SVEHLLYISEKVKNGSMDKADFFKLFNN | | TTCACCAAGGAGCTGAAGTTCGACGGCA |
| | DEIRV | | TGAGGCTGGGCGACAGCAACAGGCCCGA |
| | | | CGTGATCATCAGCCACGACAACTTCGGC |
| | | | ACCATCATCGACAACAAGAGCTACAAGG |
| | | | ACGGCTTCAACATCGACAAGAAGTGCGC |
| | | | CGACGAGATGAGCAGGTACATCAACGAG |
| | | | AACCAGAGGAGGATCCCCGAGCTGCCCA |
| | | | AGAACGAGTGGTGGAAGAACTTCGACGT |
| | | | GAACGTGGACATCTTCACCTTCCTGTTC |
| | | | ATCACCAGCTACCTGAAGGGCAACTTCA |
| | | | AGGACCAGCTGGAGTACATCAGCAAGAG |
| | | | CCAGAGCGACATCAAGGGCGCCGCCATC |
| | | | AGCGTGGAGCACCTGCTGTACATCAGCG |
| | | | AGAAGGTGAAGAACGGCAGCATGGACAA |
| | | | GGCCGACTTCTTCAAGCTGTTCAACAAC |
| | | | GACGAGATCAGGGTG |
| 61 | VLKDKHLEKIKEKFLENTSLDPRFISLI | 142 | GTGCTGAAGGACAAGCACCTGGAGAAGA |
| | EISRDKKQNRAFEIITAELFNTSYNLSA | | TCAAGGAGAAGTTCCTGGAGAACACCAG |
| | IHLGGGRRPDVLAYNDNFGIIVDTKAYK | | CCTGGACCCCAGGTTCATCAGCCTGATC |
| | NGYGRNVNQEDEMVRYITENKIRKQDIS | | GAGATCAGCAGGGACAAGAAGCAGAACA |
| | KNNWWKYFSKSIPSTSYYHLWISSEFVG | | GGGCCTTCGAGATCATCACCGCCGAGCT |
| | MFSDQLRETSSRTGENGGAMNVEQLLIG | | GTTCAACACCAGCTACAACCTGAGCGCC |
| | ANQVLNNVLDPNRLPEYMENKEIIF | | ATCCACCTGGGCGGCGGCAGGAGGCCCG |
| | | | ACGTGCTGGCCTACAACGACAACTTCGG |
| | | | CATCATCGTGGACACCAAGGCCTACAAG |
| | | | AACGGCTACGGCAGGAACGTGAACCAGG |
| | | | AGGACGAGATGGTGAGGTACATCACCGA |
| | | | GAACAAGATCAGGAAGCAGGACATCAGC |
| | | | AAGAACAACTGGTGGAAGTACTTCAGCA |
| | | | AGAGCATCCCCAGCACCAGCTACTACCA |
| | | | CCTGTGGATCAGCAGCGAGTTCGTGGGC |
| | | | ATGTTCAGCGACCAGCTGAGGGAGACCA |
| | | | GCAGCAGGACCGGCGAGAACGGCGGCGC |
| | | | CATGAACGTGGAGCAGCTGCTGATCGGC |
| | | | GCCAACCAGGTGCTGAACAACGTGCTGG |
| | | | ACCCCAACAGGCTGCCCGAGTACATGGA |
| | | | GAACAAGGAGATCATCTTC |
| 62 | ALKDKHLEKIKEKFLENTSLDPRFISLI | 143 | GCCCTGAAGGACAAGCACCTGGAGAAGA |
| | EISRDKKQNRAFEIITAELFNTSYKLSA | | TCAAGGAGAAGTTCCTGGAGAACACCAG |
| | THLGGGRRPDVLVYNDNFGIIVDTKAYK | | CCTGGACCCCAGGTTCATCAGCCTGATC |
| | DGYGRNVNQEDEMVRYITENNIRKQDIN | | GAGATCAGCAGGGACAAGAAGCAGAACA |
| | KNDWWKYFSKSIPSTSYYHLWISSQFVG | | GGGCCTTCGAGATCATCACCGCCGAGCT |
| | MFSDQLRETSSRTGENGGAMNVEQLLIG | | GTTCAACACCAGCTACAAGCTGAGCGCC |
| | ANQVLNNVLDPNCLPKYMENKEIIF | | ACCCACCTGGGCGGCGGCAGGAGGCCCG |
| | | | ACGTGCTGGTGTACAACGACAACTTCGG |
| | | | CATCATCGTGGACACCAAGGCCTACAAG |
| | | | GACGGCTACGGCAGGAACGTGAACCAGG |
| | | | AGGACGAGATGGTGAGGTACATCACCGA |
| | | | GAACAACATCAGGAAGCAGGACATCAAC |
| | | | AAGAACGACTGGTGGAAGTACTTCAGCA |
| | | | AGAGCATCCCCAGCACCAGCTACTACCA |
| | | | CCTGTGGATCAGCAGCCAGTTCGTGGGC |
| | | | ATGTTCAGCGACCAGCTGAGGGAGACCA |
| | | | GCAGCAGGACCGGCGAGAACGGCGGCGC |
| | | | CATGAACGTGGAGCAGCTGCTGATCGGC |
| | | | GCCAACCAGGTGCTGAACAACGTGCTGG |
| | | | ACCCCAACTGCCTGCCCAAGTACATGGA |
| | | | GAACAAGGAGATCATCTTC |
| 63 | VLEKSDIEKFKNQLRTELTNIDHSYLKG | 144 | GTGCTGGAGAAGAGCGACATCGAGAAGT |
| | IDIASKKKTSNVENTEFEAISTKIFTDE | | TCAAGAACCAGCTGAGGACCGAGCTGAC |
| | LGFSGKHLGGSNKPDGLLWDDDCAIILD | | CAACATCGACCACAGCTACCTGAAGGGC |
| | SKAYSEGFPLTASHTDAMGRYLRQFTER | | ATCGACATCGCCAGCAAGAAGAAGACCA |
| | KEEIKPTWWDIAPEHLDNTYFAYVSGSF | | GCAACGTGGAGAACACCGAGTTCGAGGC |
| | SGNYKEQLQKFRQDTNHLGGALEFVKLL | | CATCAGCACCAAGATCTTCACCGACGAG |
| | LLANNYKTQKMSKKEVKKSILDYNISY | | CTGGGCTTCAGCGGCAAGCACCTGGGCG |
| | | | GCAGCAACAAGCCCGACGGCCTGCTGTG |
| | | | GGACGACGACTGCGCCATCATCCTGGAC |
| | | | AGCAAGGCCTACAGCGAGGGCTTCCCCC |
| | | | TGACCGCCAGCCACACCGACGCCATGGG |
| | | | CAGGTACCTGAGGCAGTTCACCGAGAGG |
| | | | AAGGAGGAGATCAAGCCCACCTGGTGGG |
| | | | ACATCGCCCCCGAGCACCTGGACAACAC |
| | | | CTACTTCGCCTACGTGAGCGGCAGCTTC |
| | | | AGCGGCAACTACAAGGAGCAGCTGCAGA |
| | | | AGTTCAGGCAGGACACCAACCACCTGGG |
| | | | CGGCGCCCTGGAGTTCGTGAAGCTGCTG |
| | | | CTGCTGGCCAACAACTACAAGACCCAGA |
| | | | AGATGAGCAAGAAGGAGGTGAAGAAGAG |
| | | | CATCCTGGACTACAACATCAGCTAC |
| 64 | AEADVTSEKIKNHFRRVTELPERYLELL | 145 | GCCGAGGCCGACGTGACCAGCGAGAAGA |
| | DIAFDHKRNRDFEMVTAGLFKDVYGLES | | TCAAGAACCACTTCAGGAGGGTGACCGA |
| | VHLGGANKPDGVVYNDNFGIILDTKAYE | | GCTGCCCGAGAGGTACCTGGAGCTGCTG |
| | NGYGKHISQIDEMVRYIDDNRLRDTTRN | | GACATCGCCTTCGACCACAAGAGGAACA |
| | PNKWWENFDADIPSDQFYYLWVSGKFLP | | GGGACTTCGAGATGGTGACCGCCGGCCT |
| | NFAEQLKQTNYRSHANGGGLEVQQLLLG | | GTTCAAGGACGTGTACGGCCTGGAGAGC |
| | ADAVKRRKLDVNTIPNYMKNEVITL | | GTGCACCTGGGCGGCGCCAACAAGCCCG |
| | | | ACGGCGTGGTGTACAACGACAACTTCGG |
| | | | CATCATCCTGGACACCAAGGCCTACGAG |
| | | | AACGGCTACGGCAAGCACATCAGCCAGA |
| | | | TCGACGAGATGGTGAGGTACATCGACGA |
| | | | CAACAGGCTGAGGGACACCACCAGGAAC |
| | | | CCCAACAAGTGGTGGGAGAACTTCGACG |
| | | | CCGACATCCCCAGCGACCAGTTCTACTA |
| | | | CCTGTGGGTGAGCGGCAAGTTCCTGCCC |
| | | | AACTTCGCCGAGCAGCTGAAGCAGACCA |
| | | | ACTACAGGAGCCACGCCAACGGCGGCGG |
| | | | CCTGGAGGTGCAGCAGCTGCTGCTGGGC |
| | | | GCCGACGCCGTGAAGAGGAGGAAGCTGG |
| | | | ACGTGAACACCATCCCCAACTACATGAA |
| | | | GAACGAGGTGATCACCCTG |
| 65 | AEADLNSEKIKNHYRKITNLPEKYIELL | 146 | GCCGAGGCCGACCTGAACAGCGAGAAGA |
| | DIAFDHRRHQDFEIVTAGLFKDCYGLSS | | TCAAGAACCACTACAGGAAGATCACCAA |
| | IHLGGQNKPDGVVENNKFGIILDTKAYE | | CCTGCCCGAGAAGTACATCGAGCTGCTG |
| | KGYGMHIGQIDEMCRYIDDNKKRDIVRQ | | GACATCGCCTTCGACCACAGGAGGCACC |
| | PNEWWKNFGDNIPKDQFYYLWISGKFLP | | AGGACTTCGAGATCGTGACCGCCGGCCT |
| | RFNEQLKQTHYRTSINGGGLEVSQLLLG | | GTTCAAGGACTGCTACGGCCTGAGCAGC |
| | ANAAMKGKLDVNTLPKHMNNQVIKL | | ATCCACCTGGGCGGCCAGAACAAGCCCG |
| | | | ACGGCGTGGTGTTCAACAACAAGTTCGG |
| | | | CATCATCCTGGACACCAAGGCCTACGAG |
| | | | AAGGGCTACGGCATGCACATCGGCCAGA |
| | | | TCGACGAGATGTGCAGGTACATCGACGA |
| | | | CAACAAGAAGAGGGACATCGTGAGGCAG |
| | | | CCCAACGAGTGGTGGAAGAACTTCGGCG |
| | | | ACAACATCCCCAAGGACCAGTTCTACTA |
| | | | CCTGTGGATCAGCGGCAAGTTCCTGCCC |
| | | | AGGTTCAACGAGCAGCTGAAGCAGACCC |
| | | | ACTACAGGACCAGCATCAACGGCGGCGG |
| | | | CCTGGAGGTGAGCCAGCTGCTGCTGGGC |
| | | | GCCAACGCCGCCATGAAGGGCAAGCTGG |
| | | | ACGTGAACACCCTGCCCAAGCACATGAA |
| | | | CAACCAGGTGATCAAGCTG |
| 66 | VLKDAALQKTKNTLLNELTEIDPADIEV | 147 | GTGCTGAAGGACGCCGCCCTGCAGAAGA |
| | IEMSWKKATTRSQNTLEATLFEVKVVEI | | CCAAGAACACCCTGCTGAACGAGCTGAC |
| | FKKYFELNGEHLGGQNRPDGAVYYNSTY | | CGAGATCGACCCCGCCGACATCGAGGTG |
| | GIILDTKAYSNGYNIPVDQQREMVDYIT | | ATCGAGATGAGCTGGAAGAAGGCCACCA |
| | DVIDKNQNVTPNRWWEAFPATLLKNNIY | | CCAGGAGCCAGAACACCCTGGAGGCCAC |
| | YLWVAGGFTGKYLDQLTRTHNQTNMDGG | | CCTGTTCGAGGTGAAGGTGGTGGAGATC |
| | AMTTEVLLRLANKVSSGNLKTTDIPKLM | | TTCAAGAAGTACTTCGAGCTGAACGGCG |
| | TNKLILS | | AGCACCTGGGCGGCCAGAACAGGCCCGA |
| | | | CGGCGCCGTGTACTACAACAGCACCTAC |
| | | | GGCATCATCCTGGACACCAAGGCCTACA |
| | | | GCAACGGCTACAACATCCCCGTGGACCA |
| | | | GCAGAGGGAGATGGTGGACTACATCACC |
| | | | GACGTGATCGACAAGAACCAGAACGTGA |
| | | | CCCCCAACAGGTGGTGGGAGGCCTTCCC |
| | | | CGCCACCCTGCTGAAGAACAACATCTAC |
| | | | TACCTGTGGGTGGCCGGCGGCTTCACCG |
| | | | GCAAGTACCTGGACCAGCTGACCAGGAC |
| | | | CCACAACCAGACCAACATGGACGGCGGC |
| | | | GCCATGACCACCGAGGTGCTGCTGAGGC |
| | | | TGGCCAACAAGGTGAGCAGCGGCAACCT |
| | | | GAAGACCACCGACATCCCCAAGCTGATG |
| | | | ACCAACAAGCTGATCCTGAGC |
| 67 | AEADLDSERIKNHYRKITNLPEKYIELL | 148 | GCCGAGGCCGACCTGGACAGCGAGAGGA |
| | DIAFDHHRHQDFEIITAGLFKDCYGLSS | | TCAAGAACCACTACAGGAAGATCACCAA |
| | IHLGGQNKPDGVVFNGKFGIILDTKAYE | | CCTGCCCGAGAAGTACATCGAGCTGCTG |
| | KGYGMHINQIDEMCRYIEDNKQRDKIRQ | | GACATCGCCTTCGACCACCACAGGCACC |
| | PNEWWNNFGDNIPENKFYYLWVSGKFLP | | AGGACTTCGAGATCATCACCGCCGGCCT |
| | KFNEQLKQTHYRTGINGGGLEVSQLLLG | | GTTCAAGGACTGCTACGGCCTGAGCAGC |
| | ADAVMKGALNVNILPTYMHNNVIQ | | ATCCACCTGGGCGGCCAGAACAAGCCCG |
| | | | ACGGCGTGGTGTTCAACGGCAAGTTCGG |
| | | | CATCATCCTGGACACCAAGGCCTACGAG |
| | | | AAGGGCTACGGCATGCACATCAACCAGA |
| | | | TCGACGAGATGTGCAGGTACATCGAGGA |
| | | | CAACAAGCAGAGGGACAAGATCAGGCAG |
| | | | CCCAACGAGTGGTGGAACAACTTCGGCG |
| | | | ACAACATCCCCGAGAACAAGTTCTACTA |
| | | | CCTGTGGGTGAGCGGCAAGTTCCTGCCC |
| | | | AAGTTCAACGAGCAGCTGAAGCAGACCC |
| | | | ACTACAGGACCGGCATCAACGGCGGCGG |
| | | | CCTGGAGGTGAGCCAGCTGCTGCTGGGC |
| | | | GCCGACGCCGTGATGAAGGGCGCCCTGA |
| | | | ACGTGAACATCCTGCCCACCTACATGCA |
| | | | CAACAACGTGATCCAG |
| 68 | EISDIALQKEKAYFYKNTALSKRHISIL | 149 | GAGATCAGCGACATCGCCCTGCAGAAGG |
| | EIAFDGSKNRDLEILSAEVFKDYYQLES | | AGAAGGCCTACTTCTACAAGAACACCGC |
| | IHLGGGLKPDGIAFNQNFGIIVDTKAYK | | CCTGAGCAAGAGGCACATCAGCATCCTG |
| | GVYSRSRAEADKMFRYIEDNKKRDPKRN | | GAGATCGCCTTCGACGGCAGCAAGAACA |
| | QSLWWRSFNEHIPANNFYFLWISGKFQR | | GGGACCTGGAGATCCTGAGCGCCGAGGT |
| | NFDTQINQLNYETGYRGGALSARQFLIG | | GTTCAAGGACTACTACCAGCTGGAGAGC |
| | ADAIQKGKIDINDLPSYFNNSVISF | | ATCCACCTGGGCGGCGGCCTGAAGCCCG |
| | | | ACGGCATCGCCTTCAACCAGAACTTCGG |
| | | | CATCATCGTGGACACCAAGGCCTACAAG |
| | | | GGCGTGTACAGCAGGAGCAGGGCCGAGG |
| | | | CCGACAAGATGTTCAGGTACATCGAGGA |
| | | | CAACAAGAAGAGGGACCCCAAGAGGAAC |
| | | | CAGAGCCTGTGGTGGAGGAGCTTCAACG |
| | | | AGCACATCCCCGCCAACAACTTCTACTT |
| | | | CCTGTGGATCAGCGGCAAGTTCCAGAGG |
| | | | AACTTCGACACCCAGATCAACCAGCTGA |
| | | | ACTACGAGACCGGCTACAGGGGCGGCGC |
| | | | CCTGAGCGCCAGGCAGTTCCTGATCGGC |
| | | | GCCGACGCCATCCAGAAGGGCAAGATCG |
| | | | ACATCAACGACCTGCCCAGCTACTTCAA |
| | | | CAACAGCGTGATCAGCTTC |
| 69 | TSREKSRLNLKEYFVSNTNLPNKFITLL | 150 | ACCAGCAGGGAGAAGAGCAGGCTGAACC |
| | DLAYDGKANRDFELITSELFREIYKLNT | | TGAAGGAGTACTTCGTGAGCAACACCAA |
| | RHLGGTRKPDILIWNENFGIIADTKAYS | | CCTGCCCAACAAGTTCATCACCCTGCTG |
| | KGYKKNISEEDKMVRYIDENIKRSKDYN | | GACCTGGCCTACGACGGCAAGGCCAACA |
| | PNEWWKVFDNEISSNNYFYLWISSEFIG | | GGGACTTCGAGCTGATCACCAGCGAGCT |
| | KFEEQLQETAQRTNVKGASINVYQLLMG | | GTTCAGGGAGATCTACAAGCTGAACACC |
| | AHKVQTKELNVNSIPKYMNNTEIKF | | AGGCACCTGGGCGGCACCAGGAAGCCCG |
| | | | ACATCCTGATCTGGAACGAGAACTTCGG |
| | | | CATCATCGCCGACACCAAGGCCTACAGC |
| | | | AAGGGCTACAAGAAGAACATCAGCGAGG |
| | | | AGGACAAGATGGTGAGGTACATCGACGA |
| | | | GAACATCAAGAGGAGCAAGGACTACAAC |
| | | | CCCAACGAGTGGTGGAAGGTGTTCGACA |
| | | | ACGAGATCAGCAGCAACAACTACTTCTA |
| | | | CCTGTGGATCAGCAGCGAGTTCATCGGC |
| | | | AAGTTCGAGGAGCAGCTGCAGGAGACCG |
| | | | CCCAGAGGACCAACGTGAAGGGCGCCAG |
| | | | CATCAACGTGTACCAGCTGCTGATGGGC |
| | | | GCCCACAAGGTGCAGACCAAGGAGCTGA |
| | | | ACGTGAACAGCATCCCCAAGTACATGAA |
| | | | CAACACCGAGATCAAGTTC |
| 70 | NCIKDSIIDIKDRVRTKLVHLDHKYLAL | 151 | AACTGCATCAAGGACAGCATCATCGACA |
| | IDLAFSDADTRTKKNSDAREFEIQTADL | | TCAAGGACAGGGTGAGGACCAAGCTGGT |
| | FTKELSFNGQRLGDSRKPDIIISFDKIG | | GCACCTGGACCACAAGTACCTGGCCCTG |
| | TIIDNKSYKDGFNISRPCADEMIRYINE | | ATCGACCTGGCCTTCAGCGACGCCGACA |
| | NNLRKKSLNANEWWNKFDPTITAYSFLF | | CCAGGACCAAGAAGAACAGCGACGCCAG |
| | ITSYLKGQFQEQLEYISNANGGIKGAAI | | GGAGTTCGAGATCCAGACCGCCGACCTG |
| | GIENLLYLSEALKSGKISHKDFYQNFNN | | TTCACCAAGGAGCTGAGCTTCAACGGCC |
| | KEITY | | AGAGGCTGGGCGACAGCAGGAAGCCCGA |
| | | | CATCATCATCAGCTTCGACAAGATCGGC |
| | | | ACCATCATCGACAACAAGAGCTACAAGG |
| | | | ACGGCTTCAACATCAGCAGGCCCTGCGC |
| | | | CGACGAGATGATCAGGTACATCAACGAG |
| | | | AACAACCTGAGGAAGAAGAGCCTGAACG |
| | | | CCAACGAGTGGTGGAACAAGTTCGACCC |
| | | | CACCATCACCGCCTACAGCTTCCTGTTC |
| | | | ATCACCAGCTACCTGAAGGGCCAGTTCC |
| | | | AGGAGCAGCTGGAGTACATCAGCAACGC |
| | | | CAACGGCGGCATCAAGGGCGCCGCCATC |
| | | | GGCATCGAGAACCTGCTGTACCTGAGCG |
| | | | AGGCCCTGAAGAGCGGCAAGATCAGCCA |
| | | | CAAGGACTTCTACCAGAACTTCAACAAC |
| | | | AAGGAGATCACCTAC |
| 71 | LPQKDQVQQQQDELRPMLKNVDHRYLQL | 152 | CTGCCCCAGAAGGACCAGGTGCAGCAGC |
| | VELALDSDQNSEYSQFEQLTMELVLKHL | | AGCAGGACGAGCTGAGGCCCATGCTGAA |
| | DFDGKPLGGSNKPDGIAWDNDGNFIIFD | | GAACGTGGACCACAGGTACCTGCAGCTG |
| | TKAYNKGYSLAGNTDKVKRYIDDVRDRD | | GTGGAGCTGGCCCTGGACAGCGACCAGA |
| | TSRTSTWWQLVPKSIDVHNLLRFVYVSG | | ACAGCGAGTACAGCCAGTTCGAGCAGCT |
| | NFTGNYMKLLDSLRSWSNAQGGLASVEK | | GACCATGGAGCTGGTGCTGAAGCACCTG |
| | LLLTSELYLRNMYSHQELIDSWTDNNVK | | GACTTCGACGGCAAGCCCCTGGGCGGCA |
| | H | | GCAACAAGCCCGACGGCATCGCCTGGGA |
| | | | CAACGACGGCAACTTCATCATCTTCGAC |
| | | | ACCAAGGCCTACAACAAGGGCTACAGCC |
| | | | TGGCCGGCAACACCGACAAGGTGAAGAG |
| | | | GTACATCGACGACGTGAGGGACAGGGAC |
| | | | ACCAGCAGGACCAGCACCTGGTGGCAGC |
| | | | TGGTGCCCAAGAGCATCGACGTGCACAA |
| | | | CCTGCTGAGGTTCGTGTACGTGAGCGGC |
| | | | AACTTCACCGGCAACTACATGAAGCTGC |
| | | | TGGACAGCCTGAGGAGCTGGAGCAACGC |
| | | | CCAGGGCGGCCTGGCCAGCGTGGAGAAG |
| | | | CTGCTGCTGACCAGCGAGCTGTACCTGA |
| | | | GGAACATGTACAGCCACCAGGAGCTGAT |
| | | | CGACAGCTGGACCGACAACAACGTGAAG |
| | | | CAC |
| 72 | TTDAVVVKDRARVRLHNINHKYLTLIDY | 153 | ACCACCGACGCCGTGGTGGTGAAGGACA |
| | AFSGKNNCTEFEIYTIDLLVNELAFNGI | | GGGCCAGGGTGAGGCTGCACAACATCAA |
| | HLGGTRKPDGIFDYNQQGIIIDNKAYSK | | CCACAAGTACCTGACCCTGATCGACTAC |
| | GFTITRSMADEMVRYVQENNDRNPERNK | | GCCTTCAGCGGCAAGAACAACTGCACCG |
| | TQWWLNFGDNVNHFNFVFISSMFKGEVR | | AGTTCGAGATCTACACCATCGACCTGCT |
| | HMLNNIKQSTGVDGCVLTAENLLYFADA | | GGTGAACGAGCTGGCCTTCAACGGCATC |
| | IKGGTVKRTDFINLFGKNDEL | | CACCTGGGCGGCACCAGGAAGCCCGACG |
| | | | GCATCTTCGACTACAACCAGCAGGGCAT |
| | | | CATCATCGACAACAAGGCCTACAGCAAG |
| | | | GGCTTCACCATCACCAGGAGCATGGCCG |
| | | | ACGAGATGGTGAGGTACGTGCAGGAGAA |
| | | | CAACGACAGGAACCCCGAGAGGAACAAG |
| | | | ACCCAGTGGTGGCTGAACTTCGGCGACA |
| | | | ACGTGAACCACTTCAACTTCGTGTTCAT |
| | | | CAGCAGCATGTTCAAGGGCGAGGTGAGG |
| | | | CACATGCTGAACAACATCAAGCAGAGCA |
| | | | CCGGCGTGGACGGCTGCGTGCTGACCGC |
| | | | CGAGAACCTGCTGTACTTCGCCGACGCC |
| | | | ATCAAGGGCGGCACCGTGAAGAGGACCG |
| | | | ACTTCATCAACCTGTTCGGCAAGAACGA |
| | | | CGAGCTG |
| 73 | LPKKDNVQRQQDELRPLLKHVDHRYLQL | 154 | CTGCCCAAGAAGGACAACGTGCAGAGGC |
| | VELALDSSQNSEYSMLESMTMELLLTHL | | AGCAGGACGAGCTGAGGCCCCTGCTGAA |
| | DFDGASLGGASKPDGIAWDKDGNFLIVD | | GCACGTGGACCACAGGTACCTGCAGCTG |
| | TKAYDNGYSLAGNTDKVARYIDDVRAKD | | GTGGAGCTGGCCCTGGACAGCAGCCAGA |
| | PNRASTWWTQVPESLNVDDNLSFMYVSG | | ACAGCGAGTACAGCATGCTGGAGAGCAT |
| | SFTGNYQRLLKDLRARTNARGGLTTVEK | | GACCATGGAGCTGCTGCTGACCCACCTG |
| | LLLTSEAYLAKSGYGHTQLLNDWTDDNI | | GACTTCGACGGCGCCAGCCTGGGCGGCG |
| | DH | | CCAGCAAGCCCGACGGCATCGCCTGGGA |
| | | | CAAGGACGGCAACTTCCTGATCGTGGAC |
| | | | ACCAAGGCCTACGACAACGGCTACAGCC |
| | | | TGGCCGGCAACACCGACAAGGTGGCCAG |
| | | | GTACATCGACGACGTGAGGGCCAAGGAC |
| | | | CCCAACAGGGCCAGCACCTGGTGGACCC |
| | | | AGGTGCCCGAGAGCCTGAACGTGGACGA |
| | | | CAACCTGAGCTTCATGTACGTGAGCGGC |
| | | | AGCTTCACCGGCAACTACCAGAGGCTGC |
| | | | TGAAGGACCTGAGGGCCAGGACCAACGC |
| | | | CAGGGGCGGCCTGACCACCGTGGAGAAG |
| | | | CTGCTGCTGACCAGCGAGGCCTACCTGG |
| | | | CCAAGAGCGGCTACGGCCACACCCAGCT |
| | | | GCTGAACGACTGGACCGACGACAACATC |
| | | | GACCAC |
| 74 | QIKDKYLEDLKLELYKKTNLPNKYYEMV | 155 | CAGATCAAGGACAAGTACCTGGAGGACC |
| | DIAYDGKRNREFEIYTSDLMQEIYGFKT | | TGAAGCTGGAGCTGTACAAGAAGACCAA |
| | TLLGGTRKPDVVSYSDAHGYIIDTKAYA | | CCTGCCCAACAAGTACTACGAGATGGTG |
| | NGYRKEIKQEDEMVRYIEDNQLKDVLRN | | GACATCGCCTACGACGGCAAGAGGAACA |
| | PNKWWECFDDAEHKKEYYFLWISSKFVG | | GGGAGTTCGAGATCTACACCAGCGACCT |
| | EFSSQLQDTSRRTGIKGGAVNIVQLLLG | | GATGCAGGAGATCTACGGCTTCAAGACC |
| | AHLVYSGEISKDQFAAYMNNTEINF | | ACCCTGCTGGGCGGCACCAGGAAGCCCG |
| | | | ACGTGGTGAGCTACAGCGACGCCCACGG |
| | | | CTACATCATCGACACCAAGGCCTACGCC |
| | | | AACGGCTACAGGAAGGAGATCAAGCAGG |
| | | | AGGACGAGATGGTGAGGTACATCGAGGA |
| | | | CAACCAGCTGAAGGACGTGCTGAGGAAC |
| | | | CCCAACAAGTGGTGGGAGTGCTTCGACG |
| | | | ACGCCGAGCACAAGAAGGAGTACTACTT |
| | | | CCTGTGGATCAGCAGCAAGTTCGTGGGC |
| | | | GAGTTCAGCAGCCAGCTGCAGGACACCA |
| | | | GCAGGAGGACCGGCATCAAGGGCGGCGC |
| | | | CGTGAACATCGTGCAGCTGCTGCTGGGC |
| | | | GCCCACCTGGTGTACAGCGGCGAGATCA |
| | | | GCAAGGACCAGTTCGCCGCCTACATGAA |
| | | | CAACACCGAGATCAACTTC |
| 75 | MNPRNEIVIAKHLSGGNRPEIVCYHPED | 156 | ATGAACCCCAGGAACGAGATCGTGATCG |
| | KPDHGLILDSKAYKSGFTIPSGERDKMV | | CCAAGCACCTGAGCGGCGGCAACAGGCC |
| | RYIEEYITKNQLQNPNEWWKNLKGAEYP | | CGAGATCGTGTGCTACCACCCCGAGGAC |
| | GIVGFGFISNSFLGHYRKQLDYIMRRTK | | AAGCCCGACCACGGCCTGATCCTGGACA |
| | IKGSSITTEHLLKTVEDVLSEKGNVIDF | | GCAAGGCCTACAAGAGCGGCTTCACCAT |
| | FKYFLE | | CCCCAGCGGCGAGAGGGACAAGATGGTG |
| | | | AGGTACATCGAGGAGTACATCACCAAGA |
| | | | ACCAGCTGCAGAACCCCAACGAGTGGTG |
| | | | GAAGAACCTGAAGGGCGCCGAGTACCCC |
| | | | GGCATCGTGGGCTTCGGCTTCATCAGCA |
| | | | ACAGCTTCCTGGGCCACTACAGGAAGCA |
| | | | GCTGGACTACATCATGAGGAGGACCAAG |
| | | | ATCAAGGGCAGCAGCATCACCACCGAGC |
| | | | ACCTGCTGAAGACCGTGGAGGACGTGCT |
| | | | GAGCGAGAAGGGCAACGTGATCGACTTC |
| | | | TTCAAGTACTTCCTGGAG |
| 76 | EIKNQEIEELKQIALNKYTALPSEWVEL | 157 | GAGATCAAGAACCAGGAGATCGAGGAGC |
| | IEISRDKDQSTIFEMKVAELFKTCYRIK | | TGAAGCAGATCGCCCTGAACAAGTACAC |
| | SLHLGGASKPDCLLWDDSFSVIVDAKAY | | CGCCCTGCCCAGCGAGTGGGTGGAGCTG |
| | KDGFPFQASEKDKMVRYLRECERKDKAE | | ATCGAGATCAGCAGGGACAAGGACCAGA |
| | NATEWWNNFPPELNSNQLFFMFASSFFS | | GCACCATCTTCGAGATGAAGGTGGCCGA |
| | STAEKHLESVSIASKFSGCAWDVDNLLS | | GCTGTTCAAGACCTGCTACAGGATCAAG |
| | GANFFLQNPQATLQYHLIRVFSNKVVD | | AGCCTGCACCTGGGCGGCGCCAGCAAGC |
| | | | CCGACTGCCTGCTGTGGGACGACAGCTT |
| | | | CAGCGTGATCGTGGACGCCAAGGCCTAC |
| | | | AAGGACGGCTTCCCCTTCCAGGCCAGCG |
| | | | AGAAGGACAAGATGGTGAGGTACCTGAG |
| | | | GGAGTGCGAGAGGAAGGACAAGGCCGAG |
| | | | AACGCCACCGAGTGGTGGAACAACTTCC |
| | | | CCCCCGAGCTGAACAGCAACCAGCTGTT |
| | | | CTTCATGTTCGCCAGCAGCTTCTTCAGC |
| | | | AGCACCGCCGAGAAGCACCTGGAGAGCG |
| | | | TGAGCATCGCCAGCAAGTTCAGCGGCTG |
| | | | CGCCTGGGACGTGGACAACCTGCTGAGC |
| | | | GGCGCCAACTTCTTCCTGCAGAACCCCC |
| | | | AGGCCACCCTGCAGTACCACCTGATCAG |
| | | | GGTGTTCAGCAACAAGGTGGTGGAC |
| 77 | LPHKDNVIKQQDELRPMLKHVNHKYLQL | 158 | CTGCCCCACAAGGACAACGTGATCAAGC |
| | VELAFESSRNSEYSQFETLTMELVLKYL | | AGCAGGACGAGCTGAGGCCCATGCTGAA |
| | DFSGKSLGGANKPDGIAWDPLGNFLIFD | | GCACGTGAACCACAAGTACCTGCAGCTG |
| | TKAYKHGYTLSNNTDRVARYINDVRDKD | | GTGGAGCTGGCCTTCGAGAGCAGCAGGA |
| | IQRISRWWQSIPTYIDVKNKLQFVYISG | | ACAGCGAGTACAGCCAGTTCGAGACCCT |
| | SFTGHYLRLLNDLRSRTRAKGGLVTVEK | | GACCATGGAGCTGGTGCTGAAGTACCTG |
| | LLLTTERYLAEADYTHKELFDDWMDDNI | | GACTTCAGCGGCAAGAGCCTGGGCGGCG |
| | EH | | CCAACAAGCCCGACGGCATCGCCTGGGA |
| | | | CCCCCTGGGCAACTTCCTGATCTTCGAC |
| | | | ACCAAGGCCTACAAGCACGGCTACACCC |
| | | | TGAGCAACAACACCGACAGGGTGGCCAG |
| | | | GTACATCAACGACGTGAGGGACAAGGAC |
| | | | ATCCAGAGGATCAGCAGGTGGTGGCAGA |
| | | | GCATCCCCACCTACATCGACGTGAAGAA |
| | | | CAAGCTGCAGTTCGTGTACATCAGCGGC |
| | | | AGCTTCACCGGCCACTACCTGAGGCTGC |
| | | | TGAACGACCTGAGGAGCAGGACCAGGGC |
| | | | CAAGGGCGGCCTGGTGACCGTGGAGAAG |
| | | | CTGCTGCTGACCACCGAGAGGTACCTGG |
| | | | CCGAGGCCGACTACACCCACAAGGAGCT |
| | | | GTTCGACGACTGGATGGACGACAACATC |
| | | | GAGCAC |
| 78 | RISPSNLEQTKQQLREELINLDHQYLDI | 159 | AGGATCAGCCCCAGCAACCTGGAGCAGA |
| | LDFSIAGNVGARQFEVRIVELLNEIIIA | | CCAAGCAGCAGCTGAGGGAGGAGCTGAT |
| | KHLSGGNRPEIIGENPKENPEDCIIMDS | | CAACCTGGACCACCAGTACCTGGACATC |
| | KAYKEGFNIPANERDKMIRYVEEYNAKD | | CTGGACTTCAGCATCGCCGGCAACGTGG |
| | NTLNNNKWWKNFESPNYPTNQVKFSFVS | | GCGCCAGGCAGTTCGAGGTGAGGATCGT |
| | SSFIGQFTNQLTYINNRTNVNGSAITAE | | GGAGCTGCTGAACGAGATCATCATCGCC |
| | TLLRKVENVMNVNTEYNLNNFFEELGSN | | AAGCACCTGAGCGGCGGCAACAGGCCCG |
| | TLVA | | AGATCATCGGCTTCAACCCCAAGGAGAA |
| | | | CCCCGAGGACTGCATCATCATGGACAGC |
| | | | AAGGCCTACAAGGAGGGCTTCAACATCC |
| | | | CCGCCAACGAGAGGGACAAGATGATCAG |
| | | | GTACGTGGAGGAGTACAACGCCAAGGAC |
| | | | AACACCCTGAACAACAACAAGTGGTGGA |
| | | | AGAACTTCGAGAGCCCCAACTACCCCAC |
| | | | CAACCAGGTGAAGTTCAGCTTCGTGAGC |
| | | | AGCAGCTTCATCGGCCAGTTCACCAACC |
| | | | AGCTGACCTACATCAACAACAGGACCAA |
| | | | CGTGAACGGCAGCGCCATCACCGCCGAG |
| | | | ACCCTGCTGAGGAAGGTGGAGAACGTGA |
| | | | TGAACGTGAACACCGAGTACAACCTGAA |
| | | | CAACTTCTTCGAGGAGCTGGGCAGCAAC |
| | | | ACCCTGGTGGCC |
| 79 | TFDSTVADNLKNLILPKLKELDHKYLQA | 160 | ACCTTCGACAGCACCGTGGCCGACAACC |
| | IDIAYKRSNTTNHENTLLEVLSADLFTK | | TGAAGAACCTGATCCTGCCCAAGCTGAA |
| | EMDYHGKHLGGANKPDGFVYDEETGWIL | | GGAGCTGGACCACAAGTACCTGCAGGCC |
| | DSKAYRDGFAVTAHTTDAMGRYIDQYRD | | ATCGACATCGCCTACAAGAGGAGCAACA |
| | RDDKSTWWEDFPKDLPQTYFAYVSGFYI | | CCACCAACCACGAGAACACCCTGCTGGA |
| | GKYQEQLQDFENRKHMKGGLIEVAKLIL | | GGTGCTGAGCGCCGACCTGTTCACCAAG |
| | LAEKYKENKITHDQITLQILNDHISQ | | GAGATGGACTACCACGGCAAGCACCTGG |
| | | | GCGGCGCCAACAAGCCCGACGGCTTCGT |
| | | | GTACGACGAGGAGACCGGCTGGATCCTG |
| | | | GACAGCAAGGCCTACAGGGACGGCTTCG |
| | | | CCGTGACCGCCCACACCACCGACGCCAT |
| | | | GGGCAGGTACATCGACCAGTACAGGGAC |
| | | | AGGGACGACAAGAGCACCTGGTGGGAGG |
| | | | ACTTCCCCAAGGACCTGCCCCAGACCTA |
| | | | CTTCGCCTACGTGAGCGGCTTCTACATC |
| | | | GGCAAGTACCAGGAGCAGCTGCAGGACT |
| | | | TCGAGAACAGGAAGCACATGAAGGGCGG |
| | | | CCTGATCGAGGTGGCCAAGCTGATCCTG |
| | | | CTGGCCGAGAAGTACAAGGAGAACAAGA |
| | | | TCACCCACGACCAGATCACCCTGCAGAT |
| | | | CCTGAACGACCACATCAGCCAG |
| 80 | PLDVVEQMKAELRPLLNHVNHRLLAIID | 161 | CCCCTGGACGTGGTGGAGCAGATGAAGG |
| | FSYNMSRGDDKRLEDYTAQIYKLISHDT | | CCGAGCTGAGGCCCCTGCTGAACCACGT |
| | HLLAGPSRPDVVSVINDLGIIIDSKAYK | | GAACCACAGGCTGCTGGCCATCATCGAC |
| | QGFNIPQAEEDKMVRYLDESIRRDPAIN | | TTCAGCTACAACATGAGCAGGGGCGACG |
| | PTKWWEYLGASTEYVFQFVSSSFSSGAS | | ACAAGAGGCTGGAGGACTACACCGCCCA |
| | AKLRQIHRRSSIEGSIITAKNLLLLAEN | | GATCTACAAGCTGATCAGCCACGACACC |
| | FLCTNTINIDLFRQNNEI | | CACCTGCTGGCCGGCCCCAGCAGGCCCG |
| | | | ACGTGGTGAGCGTGATCAACGACCTGGG |
| | | | CATCATCATCGACAGCAAGGCCTACAAG |
| | | | CAGGGCTTCAACATCCCCCAGGCCGAGG |
| | | | AGGACAAGATGGTGAGGTACCTGGACGA |
| | | | GAGCATCAGGAGGGACCCCGCCATCAAC |
| | | | CCCACCAAGTGGTGGGAGTACCTGGGCG |
| | | | CCAGCACCGAGTACGTGTTCCAGTTCGT |
| | | | GAGCAGCAGCTTCAGCAGCGGCGCCAGC |
| | | | GCCAAGCTGAGGCAGATCCACAGGAGGA |
| | | | GCAGCATCGAGGGCAGCATCATCACCGC |
| | | | CAAGAACCTGCTGCTGCTGGCCGAGAAC |
| | | | TTCCTGTGCACCAACACCATCAACATCG |
| | | | ACCTGTTCAGGCAGAACAACGAGATC |
| 81 | QLVPSYITQTKLRLSGLINYIDHSYFDL | 162 | CAGCTGGTGCCCAGCTACATCACCCAGA |
| | IDLGFDGRQNRLYELRIVELLNLINSLK | | CCAAGCTGAGGCTGAGCGGCCTGATCAA |
| | ALHLSGGNRPEIIAYSPDVNPINGVIMD | | CTACATCGACCACAGCTACTTCGACCTG |
| | SKSYRGGFNIPNSERDKMIRYINEYNQK | | ATCGACCTGGGCTTCGACGGCAGGCAGA |
| | NPTLNSNRWWENFRAPDYPQSPLKYSFV | | ACAGGCTGTACGAGCTGAGGATCGTGGA |
| | SGNFIGQFLNQIQYILTQTGINGGAITS | | GCTGCTGAACCTGATCAACAGCCTGAAG |
| | EKLIEKVNAVLNPNISYTINNFENDLGC | | GCCCTGCACCTGAGCGGCGGCAACAGGC |
| | NRLVQ | | CCGAGATCATCGCCTACAGCCCCGACGT |
| | | | GAACCCCATCAACGGCGTGATCATGGAC |
| | | | AGCAAGAGCTACAGGGGCGGCTTCAACA |
| | | | TCCCCAACAGCGAGAGGGACAAGATGAT |
| | | | CAGGTACATCAACGAGTACAACCAGAAG |
| | | | AACCCCACCCTGAACAGCAACAGGTGGT |
| | | | GGGAGAACTTCAGGGCCCCCGACTACCC |
| | | | CCAGAGCCCCCTGAAGTACAGCTTCGTG |
| | | | AGCGGCAACTTCATCGGCCAGTTCCTGA |
| | | | ACCAGATCCAGTACATCCTGACCCAGAC |
| | | | CGGCATCAACGGCGGCGCCATCACCAGC |
| | | | GAGAAGCTGATCGAGAAGGTGAACGCCG |
| | | | TGCTGAACCCCAACATCAGCTACACCAT |
| | | | CAACAACTTCTTCAACGACCTGGGCTGC |
| | | | AACAGGCTGGTGCAG |

In some embodiments, an endonuclease of the present disclosure can have a sequence of X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀X₃₁ X₃₂X₃₃X₃₄X₃₅X₃₆X₃₇X₃₈X₃₉X₄₀X₄₁X₄₂X₄₃KX₄₄X₄₅X₄₆X₄₇X₄₈X₄₉X₅₀X₅₁X₅₂X₅₃X₅₄X₅₅GX₅₆HLGGX₅₇ RX₅₈PDGX₅₉X₆₀X₆₁X₆₂X₆₃X₆₄X₆₅X₆₆X₆₇X₆₈X₆₉X₇₀X₇₁X₇₂X₇₃X₇₄GX₇₅IX₇₆DTKX₇₇YX₇₈X₇₉GYX₈₀L PIX₈₁QX₈₂DEMX₈₃RYX₈₄X₈₅ENX₈₆X₈₇RX₈₈X₈₉X₉₀X₉₁NX₉₂NX₉₃WWX₉₄X₉₅X₉₆X₉₇X₉₈X₉₉X₁₀₀X₁₀₁ X₁₀₂X₁₀₃X₁₀₄X₁₀₅X₁₀₆FX₁₀₇X₁₀₈X₁₀₉X₁₁₀FX₁₁₁GX₁₁₂X₁₁₃X₁₁₄X₁₁₅X₁₁₆X₁₁₇X₁₁₈RX₁₁₉X₁₂₀X₁₂₁X₁₂₂X₁₂₃ X₁₂₄X₁₂₅X₁₂₆GX₁₂₇X₁₂₈X₁₂₉X₁₃₀X₁₃₁X₁₃₂X₁₃₃LLX₁₃₄X₁₃₅X₁₃₆X₁₃₇X₁₃₈X₁₃₉X₁₄₀X₁₄₁X₁₄₂X₁₄₃X₁₄₄X₁₄₅ X₁₄₆X₁₄₇X₁₄₈X₁₄₉X₁₅₀X₁₅₁X₁₅₂X₁₅₃FX₁₅₄X₁₅₅X₁₅₆X₁₅₇X₁₅₈X₁₅₉X₁₆₀ (SEQ ID NO: 316), wherein X₁ is F, Q, N, D, or absent, X₂ is L, I, T, S, N, or absent, X₃ is V, I, G, A, E, T, or absent, X₄ is K, C, or absent, X₅ is G, S, or absent, X₆ is A, S, E, D, N, or absent, X₇ is M, I, V, Q, F, L, or absent, X₈ is E, S, T, N, or absent, X₉ is I, M, E, T, Q, or absent, X₁₀ is K, S, L, I, T, E, or absent, X₁₁ is K or absent, X₁₂ is S, A, E, D, or absent, X₁₃ is E, N, Q, K, or absent, X₁₄ is L, M, V, or absent, X₁₅ is R or absent, X₁₆ is H, D, T, G, E, N, or absent, X₁₇ is K, N, Q, E, A, or absent, X₁₈ is L or absent, X₁₉ is R, Q, N, T, D, or absent, X₂₀ is H, M, V, N, T, or absent, X₂₁ is V, L, I, or absent, X₂₂ is P, S, or absent, X₂₃ is H or absent, X₂₄ is E, D, or absent, X₂₅ is Y or absent, X₂₆ is I, L, or absent, X₂₇ is E, Q, G, S, A, Y, or absent, X₂₈ is L or absent, X₂₉ is I, V, L, or absent, X₃₀ is E, D, or absent, X₃₁ is I, L, or absent, X₃₂ is A, S, or absent, X₃₃ is Q, Y, F, or absent, X₃₄ is D or absent, X₃₅ is S, P, or absent, X₃₆ is K, Y, Q, T, or absent, X₃₇ is Q or absent, X₃₈ is N or absent, X₃₉ is R, K, or absent, X₄₀ is L, I, or absent, X₄₁ is L, F, or absent, X₄₂ is E or absent, X₄₃ is F, M, L, or absent, X₄₄ is V, T, or I, X₄₅ is V, M, L, or I, X₄₆ is E, D, or Q, X₄₇ is F or L, X₄₈ is F or L, X₄₉ is K, I, T, or V, X₅₀ is K, N, or E, X₅₁ is I or E, X₅₂ is Y, F, or C, X₅₃ is G, or N, X₅₄ is Y, or F, X₅₅ is R, S, N, E, K, or Q, X₅₆ is K, S, L, V, or T, X₅₇ is S, A, or V, X₅₈ is K or R, X₅₉ is A, I, or V, X₆₀ is L, M, V, I, or C, X₆₁ is F or Y, X₆₂ is T, A, or S, X₆₃ is K, E, or absent, X₆₄ is D, E, or absent, X₆₅ is E, A, or absent, X₆₆ is N, K, or absent, X₆₇ is E, S, or absent, X₆₈ is D, E, Q, A, or absent, X₆₉ is G, V, K, N, or absent, X₇₀ is L, G, E, S, or absent, X₇₁ is V, S, K, T, E, or absent, X₇₂ is L, H, K, E, Y, D, or A, X₇₃ is N, G, or D, X₇₄ is H, F, or Y, X₇₅ is I, or V, X₇₆ is L, V, or I, X₇₇ is A or S, X₇₈ is K or S, X₇₉ is D, G, K, S, or N, X₈₀ is R, N, S, or G, X₈₁ is S, A, or G, X₈₂ is A, I, or V, X₈₃ is Q, E, I, or V, X₈₄ is V or I, X₈₅ is D, R, G, I, or E, X₈₆ is N, I, or Q, X₈₇ is K, D, T, E, or K, X₈₈ is S, N, D, or E, X₈₉ is Q, E, I, K, or A, X₉₀ is V, H, R, K, L, or E, X₉₁ is I, V, or R, X₉₂ is P, S, T, or R, X₉₃ is E, R, C, Q, or K, X₉₄ is E, N, or K, X₉₅ is I, V, N, E, or A, X₉₆ is Y or F, X₉₇ is P, G, or E, X₉₈ is T, E, S, D, K, or N, X₉₉ is S, D, K, G, N, or T, X₁₀₀ is I, T, V, or L, X₁₀₁ is T, N, G, or D, X₁₀₂ is D, E, T, K, or I, X₁₀₃ is F or Y, X₁₀₄ is K or Y, X₁₀s is F or Y, X₁₀₆ is L, S, or M, X₁₀₇ is V or I, X₁₀₈ is S or A, X₁₀₉ is G or A, X₁₁₀ is F, Y, H, E, or K, Xin is Q, K, T, N, or I, X₁₁₂ is D, N, or K, X₁₃ is Y, F, I, or V, X₁₁₄ is R, E, K, Q, or F, X₁₁₅ is K, E, A, or N, X₁₁₆ is Q or K, X₁₇ is L or I, X₁₁₈ is E, D, N, or Q, X₁₁₉ is V, I, or L, X₁₂₀ is S, N, F, T, or Q, X₁₂₁ is H, I, C, or R, X₁₂₂ is L, D, N, S, or F, X₁₂₃ is T or K, X₁₂₄ is K, G, or N, X₁₂₅ is C, V, or I, X₁₂₆ is Q, L, K, or Y, X₁₂₇ is A, G, or N, X₁₂₈ is V or A, X₁₂₉ is M, L, I, V, or A, X₁₃₀ is S, T, or D, X₁₃₁ is V or I, X₁₃₂ is E, Q, K, S, or I, X₁₃₃ is Q, H, or T, X₁₃₄ is L, R, or Y, X₁₃₅ is G, I, L, or T, X₁₃₆ is G, A, or V, X₁₃₇ is E, N, or D, X₁₃₈ is K, Y, D, E, A, or R, X₁₃₉ is I, F, Y, or C, X₁₄₀ is K or R, X₁₄₁ is E, R, A, G, or T, X₁₄₂ is G or N, X₁₄₃ is S, I, K, R, or E, X₁₄₄ is L, I, or M, X₁₄₅ is T, S, D, or K, X₁₄₆ is L, H, Y, R, T, or F, X₁₄₇ is E, Y, I, M, A, or L, X₁₄₈ is E, D, R, or G, X₁₄₉ is V, F, M, L, or I, X₁₅₀ is G, K, R, L, V, or E, X₁₅₁ is K, N, D, L, H, or S, X₁₅₂ is K, L, C, or absent, X₁₅₃ is K, S, I, Y, M, or F, X₁₅₄ is K, L, C, H, D, Q, or N, X₁₅₅ is N or Y, X₁₅₆ is D, K, T, E, C, or absent, X₁₅₇ is E, V, R, or absent, X₁₅₈ is I, F, L, or absent, X₁₅₉ is V, Q, E, L, or absent, and X₁₆₀ is F or absent.

In some embodiments, an endonuclease of the present disclosure can have a sequence of X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀X₃₁ X₃₂X₃₃X₃₄X₃₅X₃₆X₃₇X₃₈X₃₉X₄₀X₄₁X₄₂X₄₃KX₄₄X₄₅X₄₆X₄₇X₄₈X₄₉X₅₀X₅₁X₅₂X₅₃X₅₄X₅₅GX₅₆HLGGX₅₇ RX₅₈PDGX₅₉X₆₀X₆₁X₆₂X₆₃X₆₄X₆₅X₆₆X₆₇X₆₈X₆₉X₇₀X₇₁X₇₂X₇₃X₇₄GX₇₅IX₇₆DTKX₇₇YX₇₈X₇₉GYX₈₀L PIX₈₁QX₈₂DEMX₈₃RYX₈₄X₈₅ENX₈₆X₈₇RX₈₈X₈₉X₉₀X₉₁NX₉₂NX₉₃WWX₉₄X₉₅X₉₆X₉₇X₉₈X₉₉X₁₀₀X₁₀₁ X₁₀₂X₁₀₃X₁₀₄X₁₀₅X₁₀₆FX₁₀₇X₁₀₈X₁₀₉X₁₁₀FX₁₁₁GX₁₁₂X₁₁₃X₁₁₄X₁₁₅X₁₁₆X₁₁₇X₁₁₈RX₁₁₉X₁₂₀X₁₂₁X₁₂₂X₁₂₃ X₁₂₄X₁₂₅X₁₂₆GX₁₂₇X₁₂₈X₁₂₉X₁₃₀X₁₃₁X₁₃₂X₁₃₃LLX₁₃₄X₁₃₅X₁₃₆X₁₃₇X₁₃₈X₁₃₉X₁₄₀X₁₄₁X₁₄₂X₁₄₃X₁₄₄X₁₄₅ X₁₄₆X₁₄₇X₁₄₈X₁₄₉X₁₅₀X₁₅₁X₁₅₂X₁₅₃FX₁₅₄X₁₅₅X₁₅₆X₁₅₇X₁₅₈X₁₅₉X₁₆₀ (SEQ ID NO: 317), wherein X₁ is F, Q, N, or absent, X₂ is L, I, T, S, or absent, X₃ is V, I, G, A, E, T, or absent, X₄ is K, C, or absent, X₅ is G, S, or absent, X₆ is A, S, E, D, or absent, X₇ is M, I, V, Q, F, L, or absent, X₈ is E, S, T, or absent, X₉ is I, M, E, T, Q, or absent, X₁₀ is K, S, L, I, T, E, or absent, X₁₁ is K or absent, X₁₂ is S, A, E, D, or absent, X₁₃ is E, N, Q, K, or absent, X₁₄ is L, M, V, or absent, X₁₅ is R or absent, X 16 is H, D, T, G, E, N, or absent, X₁₇ is K, N, Q, E, A, or absent, X₁₈ is L or absent, X₁₉ is R, Q, N, T, D, or absent, X₂₀ is H, M, V, N, T, or absent, X₂₁ is V, L, I, or absent, X₂₂ is P, S, or absent, X₂₃ is H or absent, X₂₄ is E, D, or absent, X₂₅ is Y or absent, X₂₆ is I, L, or absent, X₂₇ is E, Q, G, S, A, or absent, X₂₈ is L or absent, X₂₉ is I, V, L, or absent, X₃₀ is E, D, or absent, X₃₁ is I, L, or absent, X₃₂ is A, S, or absent, X₃₃ is Q, Y, F, or absent, X₃₄ is D or absent, X₃₅ is S, P, or absent, X₃₆ is K, Y, Q, T, or absent, X₃₇ is Q or absent, X₃₈ is N or absent, X₃₉ is R or absent, X₄₀ is L, I, or absent, X₄₁ is L, F, or absent, X₄₂ is E or absent, X₄₃ is F, M, L, or absent, X₄₄ is V, T, or I, X₄₅ is V, M, L, or I, X₄₆ is E, D, or Q, X₄₇ is F or L, X₄₈ is F or L, X₄₉ is K, I, T, or V, X₅₀ is K, N, or E, X₅₁ is I or E, X₅₂ is Y, F, or C, X₅₃ is G, or N, X₅₄ is Y, or F, X₅₅ is R, S, N, E, K, or Q, X₅₆ is K, S, L, V, or T, X₅₇ is S or A, X₅₈ is K or R, X₅₉ is A, I, or V, X₆₀ is L, M, V, I, or C, X₆₁ is F or Y, X₆₂ is T, A, or S, X₆₃ is K, E, or absent, X₆₄ is D, E, or absent, X₆₅ is E, A, or absent, X₆₆ is N, K, or absent, X₆₇ is E, S, or absent, X₆₈ is D, E, Q, A, or absent, X₆₉ is G, V, K, N, or absent, X₇₀ is L, G, E, S, or absent, X₇₁ is V, S, K, T, E, or absent, X₇₂ is L, H, K, E, Y, D, or A, X₇₃ is N, G, or D, X₇₄ is H, F, or Y, X₇₅ is I, or V, X₇₆ is L, V, or I, X₇₇ is A or S, X₇₈ is K or S, X₇₉ is D, G, K, S, or N, X₈₀ is R, N, S, or G, X₈₁ is S, A, or G, X₈₂ is A, I, or V, X₈₃ is Q, E, I, or V, X₈₄ is V or I, X₈₅ is D, R, G, I, or E, X₈₆ is N, I, or Q, X₈₇ is K, D, T, E, or K, X₈₈ is S, N, D, or E, X₈₉ is Q, E, I, K, or A, X₉₀ is V, H, R, K, L, or E, X₉₁ is I, V, or R, X₉₂ is P, S, T, or R, X₉₃ is E, R, C, Q, or K, X₉₄ is E, N, or K, X₉₅ is I, V, N, E, or A, X₉₆ is Y or F, X₉₇ is P, G, or E, X₉₈ is T, E, S, D, K, or N, X₉₉ is S, D, K, G, N, or T, X₁₀₀ is I, T, V, or L, X₁₀₁ is T, N, G, or D, X₁₀₂ is D, E, T, K, or I, X₁₀₃ is F or Y, X₁₀₄ is K or Y, X₁₀₅ is F or Y, X₁₀₆ is L, S, or M, X₁₀₇ is V or I, X₁₀₈ is S or A, X₁₀₉ is G or A, X₁₁₀ is F, Y, H, E, or K, Xin is Q, K, T, N, or I, X₁₁₂ is D, N, or K, X₁₁₃ is Y, F, I, or V, X₁₁₄ is R, E, K, Q, or F, X₁₁₅ is K, E, A, or N, X₁₁₆ is Q or K, X₁₁₇ is L or I, X₁₁₈ is E, D, N, or Q, X₁₁₉ is V, I, or L, X₁₂₀ is S, N, F, T, or Q, X₁₂₁ is H, I, C, or R, X₁₂₂ is L, D, N, S, or F, X₁₂₃ is T or K, X₁₂₄ is K, G, or N, X₁₂₅ is C, V, or I, X₁₂₆ is Q, L, K, or Y, X₁₂₇ is A, G, or N, X₁₂₈ is V or A, X₁₂₉ is M, L, I, V, or A, X₁₃₀ is S, T, or D, X₁₃₁ is V or I, X₁₃₂ is E, Q, K, S, or I, X₁₃₃ is Q, H, or T, X₁₃₄ is L, R, or Y, X₁₃₅ is G, I, L, or T, X₁₃₆ is G, A, or V, X₁₃₇ is E, N, or D, X₁₃₈ is K, Y, D, E, A, or R, X₁₃₉ is I, F, Y, or C, X₁₄₀ is K or R, X₁₄₁ is E, R, A, G, or T, X₁₄₂ is G or N, X₁₄₃ is S, I, K, R, or E, X₁₄₄ is L, I, or M, X₁₄₅ is T, S, D, or K, X₁₄₆ is L, H, Y, R, or T, X₁₄₇ is E, Y, I, M, or A, X₁₄₈ is E, D, R, or G, X₁₄₉ is V, F, M, L, or I, X₁₅₀ is G, K, R, L, V, or E, X₁₅₁ is K, N, D, L, H, or S, X₁₅₂ is K, L, C, or absent, X₁₅₃ is K, S, I, Y, M, or F, X₁₅₄ is K, L, C, H, D, Q, or N, X₁₅₅ is Nor Y, X₁₅₆ is D, K, T, E, C, or absent, X₁₅₇ is E, V, R, or absent, X₁₅₈ is I, F, L, or absent, X₁₅₉ is V, Q, E, L, or absent, and X₁₆₀ is F or absent.

In some embodiments, an endonuclease of the present disclosure can have a sequence of X₁LVKSSX₂EEX₃KEELREKLX₄HLSHEYLX₅LX₆DLAYDSKQNRLFEMKVX-ELLINECGYX₈G LHLGGSRKPDGIX₉YTEGLKX₁₀NYGIIIDTKAYSDGYNLPISQADEMERYIRENNTRNX₁₁X₁₂V NPNEWWENFPX₁₃NINEFYFLFVSGHFKGNX₁₄EEQLERISIX₁₅TX₁₆IKGAAMSVX₁₇TLLLLAN EIKAGRLX₁₈LEEVX₁₉KYFDNKEIX₂₀F (SEQ ID NO: 318), wherein X₁ is F, Q, N, D, or absent, X₂ is M, I, V, Q, F, L, or absent, X₃ is K, S, L, I, T, E, or absent, X₄ is R, Q, N, T, D, or absent, X₅ is E, Q, G, S, A, Y, or absent, X₆ is I, V, L, or absent, X₇ is V, M, L, or I, X₈ is R, S, N, E, K, or Q, X₉ is L, M, V, I, or C, X₁₀ is L, H, K, E, Y, D, or A, X₁₁ is Q, E, I, K, or A, X₁₂ is V, H, R, K, L, or E, X₁₃ is T, E, S, D, K, or N, X₁₄ is Y, F, I, or V, X₁₅ is L, D, N, S, or F, X₁₆ is K, G, or N, X₁₇ is E, Q, K, S, or I, X₁₈ is T, S, D, or K, X₁₉ is G, K, R, L, V, or E, and X₂₀ is V, Q, E, L, or absent.

In some embodiments, an endonuclease of the present disclosure can have a sequence of X₁LVKSSX₂EEX₃KEELREKLX₄HLSHEYLXSLX₆DLAYDSKQNRLFEMKVX₇ELLINECGYX₈G LHLGGSRKPDGIX₉YTEGLKX₁₀NYGIIIDTKAYSDGYNLPISQADEMERYIRENNTRNX₁₁X₁₂V NPNEWWENFPX₁₃NINEFYFLFVSGHFKGNX₁₄EEQLERISIX₁STX₁₆IKGAAMSVX₁₇TLLLLAN EIKAGRLX₁₈LEEVX₁₉KYFDNKEIX₂₀F (SEQ ID NO: 319), wherein X₁ is F, Q, N, or absent, X₂ is M, I, V, Q, F, L, or absent, X₃ is K, S, L, I, T, E, or absent, X₄ is R, Q, N, T, D, or absent, X₅ is E, Q, G, S, A, or absent, X₆ is I, V, L, or absent, X₇ is V, M, L, or I, X₈ is R, S, N, E, K, or Q, X₉ is L, M, V, I, or C, X₁₀ is L, H, K, E, Y, D, or A, X₁₁ is Q, E, I, K, or A, X₁₂ is V, H, R, K, L, or E, X₁₃ is T, E, S, D, K, or N, X₁₄ is Y, F, I, or V, X₁₅ is L, D, N, S, or F, X₁₆ is K, G, or N, X₁₇ is E, Q, K, S, or I, X₁₈ is T, S, D, or K, X₁₉ is G, K, R, L, V, or E, and X₂₀ is V, Q, E, L, or absent. In some embodiments, a cleavage domain disclosed herein comprises a sequence selected from SEQ ID NO: 316-SEQ ID NO: 319.

In some embodiments, an endonuclease of the present disclosure can have conserved amino acid residues at position 76 (D or E), position 98 (D), and position 100 (K), which together preserve catalytic function. In some embodiments, an endonuclease of the present disclosure can have conserved amino acid residues at position 114 (D) and position 118 (R), which together preserve dimerization of two cleavage domains.

In some embodiments, endonucleases disclosed herein (e.g., SEQ ID NO: 1-SEQ ID NO: 81 (nucleic acid sequences of SEQ ID NO: 82-SEQ ID NO: 162)) can have at least 33.3% divergence from SEQ ID NO: 163 (FokI) and, is immunologically orthogonal to SEQ ID NO: 163 (FokI). In some embodiments, an immunologically orthogonal endonuclease (e.g., SEQ ID NO: 1-SEQ ID NO: 81 (nucleic acid sequences of SEQ ID NO: 82-SEQ ID NO: 162)) can be administered to a patient that has already received, and is thus can have an adverse immune reaction to, FokI. In some embodiments, endonucleases disclosed herein (e.g., SEQ ID NO: 1-SEQ ID NO: 81 (nucleic acid sequences of SEQ ID NO: 82-SEQ ID NO: 162)) can have at least 35%, at least 40%, at least 45%, at least 50%, at least 55%, at least 60%, at least 65%, at least 70%, or at least 75% divergence from SEQ ID NO: 163 (FokI).

In some embodiments, an endonuclease disclosed herein (e.g., SEQ ID NO: 1-SEQ ID NO: 81 (nucleic acid sequences of SEQ ID NO: 82-SEQ ID NO: 162)) can be fused to any nucleic acid binding domain disclosed herein to form a non-naturally occurring fusion protein. This fusion protein can have one or more of the following characteristics: (a) induces greater than 1% indels (insertions/deletions) at a target site; (b) the cleavage domain comprises a molecular weight of less than 23 kDa; (c) the cleavage domain comprises less than 196 amino acids; and (d) capable of cleaving across a spacer region greater than 24 base pairs. In some embodiments, the non-naturally occurring fusion protein can induce greater than 5%, greater than 10%, greater than 20%, greater than 30%, greater than 40%, greater than 50%, greater than 60%, greater than 70%, greater than 80%, or greater than 90% indels at the target site. In some embodiments, indels are generated via the non-homologous end joining (NHEJ) pathway upon administration of a genome editing complex disclosed herein to a subject. Indels can be measured using deep sequencing.

In still various embodiments, the functional domain can be a cleavage domain or a repression domain. In some aspects, the cleavage domain comprises at least 33.3% divergence from SEQ ID NO: 163 and is immunologically orthogonal to SEQ ID NO: 163. In further aspects, the polypeptide can comprise one or more of the following characteristics: (a) induces greater than 1% indels at a target site; (b) the cleavage domain comprises a molecular weight of less than 23 kDa; (c) the cleavage domain comprises less than 196 amino acids; (d) capable of cleaving across a spacer region greater than 24 base pairs.

### Dna Binding Domains Fused to SEQ ID NO: 1-SEQ ID NO: 81 (Nucleic Acid Sequences of SEQ ID NO: 82-SEQ ID NO: 162)

The present disclosure provides for novel compositions of endonucleases with modular nucleic acid binding domains (e.g., TALEs, RNBDs, or MAP-NBDs) described herein. In some instances the novel endonucleases can be fused to a DNA binding domain from Xanthomonas spp. (TALE), Ralstonia (RNBD), or Legionella (MAP-NBD) resulting in genome editing complexes. A TALEN, RNBD-nuclease, or MAP-NBD-nuclease can include multiple components including the DNA binding domain, an optional linker, and a repressor domain. The genome editing complexes described herein can be used to selectively bind and cleave to a target gene sequence for genome editing purposes. For example, a DNA binding domain from Xanthomonas, Ralstonia, or Legionella of the present disclosure can be used to direct the binding of a genome editing complex to a desired genomic sequence.

The genome editing complexes described herein, comprising a DNA binding domain fused to an endonuclease, can be used to edit genomic loci of interest by binding to a target nucleic acid sequence via the DNA binding domain and cleaving phosphodiester bonds of target double stranded DNA via the endonuclease.

In some aspects, DNA binding domains fused to nucleases can create a site-specific double-stranded DNA break when fused to a nuclease. Such breaks can then be subsequently repaired by cellular machinery, through either homology-dependent repair or non-homologous end joining (NHEJ). Genome editing, using DNA binding domains fused to nucleases described herein, can thus be used to delete a sequence of interest (e.g., an aberrantly expressed or mutated gene) or to introduce a nucleic acid sequence of interest (e.g., a functional gene). DNA binding domains of the present disclosure can be programmed to delivery virtually any nuclease, including those disclosed herein, to any target site for therapeutic purposes, including ex vivo engineered cell therapies obtained using the compositions disclosed herein or gene therapy by direct in vivo administration of the compositions disclosed herein. In addition, the DNA binding domain can bind to specific DNA sequences and in some cases they can activate the expression of host genes. In some instances, the disclosure provides for enzymes, e.g., SEQ ID NO: 1-SEQ ID NO: 81 (or any one of nucleic acid sequences of SEQ ID NO: 82-SEQ ID NO: 162) that can be fused to the DNA binding domains of TALEs, RNBDs, and MAP-NBDs. In some instances, enzymes of the disclosure, including SEQ ID NO: 1 (nucleic acid sequence of SEQ ID NO: 82), SEQ ID NO: 4 (nucleic acid sequence of SEQ ID NO: 85), and SEQ ID NO: 8 (nucleic acid sequence of SEQ ID NO: 89), can achieve greater than 30% indels via the NHEJ pathway on a target gene when fused to a DNA binding domain of a TALE, RNBD, and MAP-NBD.

A non-naturally occurring fusion protein of the disclosure, e.g., any one of SEQ ID NO: 1-SEQ ID NO: 81 (or any one of nucleic acid sequences of SEQ ID NO: 82-SEQ ID NO: 162) fused to a DNA binding domain, can comprise a repeat unit. A repeat unit can be from a wild-type DNA-binding domain (Ralstonia solanacearum, Xanthomonas spp., Legionella quateirensis, Burkholderia, Paraburkholderia, or Francisella) or a modified repeat unit enhanced for specific recognition of a particular nucleic acid base. A modified repeat unit can comprise 1, 2, 3, 4, 5, 6, 7, 8, 9, 10, 15, 20, 25 or more mutations that can enhance the repeat module for specific recognition of a particular nucleic acid base. In some embodiments, a modified repeat unit is modified at amino acid position 2, 3, 4, 11, 12, 13, 21, 23, 24, 25, 26, 27, 28, 30, 31, 32, 33, 34, or 35. In some embodiments, a modified repeat unit is modified at amino acid positions 12 or 13.

As described in further detail below, a non-naturally occurring fusion protein of the disclosure, e.g., anyone of SEQ ID NO: 1-SEQ ID NO: 81 (or any one of nucleic acid sequences of SEQ ID NO: 82-SEQ ID NO: 162) fused to a plurality of repeat units (e.g., derived from Ralstonia solanacearum, Xanthomonas spp., Legionella quateirensis, Burkholderia, Paraburkholderia, or Francisella), can further comprise a C-terminal truncation, which can served as a linker between the DNA binding domain and the nuclease.

A non-naturally occurring fusion protein of the disclosure, e.g., anyone of SEQ ID NO: 1-SEQ ID NO: 81 (or any one of nucleic acid sequences of SEQ ID NO: 82-SEQ ID NO: 162) fused to a DNA binding domain, can further comprise an N-terminal cap as described in further detail below. An N-terminal cap can be a polypeptide portion flanking the DNA-binding repeat module. An N-terminal cap can be any length and can comprise from 0 to 136 amino acid residues in length. An N-terminal cap can be 5, 10, 15, 20, 25, 30, 35, 40, 45, 50, 60, 70, 80, 90, 100, 110, 120, or 130 amino acid residues in length. In some embodiments, an N-terminal cap can modulate structural stability of the DNA-binding repeat units. In some embodiments, an N-terminal cap can modulate nonspecific interactions. In some cases, an N-terminal cap can decrease nonspecific interaction. In some cases, an N-terminal cap can reduce off-target effect. As used here, off-target effect refers to the interaction of a genome editing complex with a sequence that is not the target binding site of interest. An N-terminal cap can further comprise a wild-type N-terminal cap sequence of a protein from Ralstonia solanacearum, Xanthomonas spp., Legionella quateirensis, Burkholderia, Paraburkholderia, or Francisella or can comprise a modified N-terminal cap sequence.

In some embodiments, a DNA binding domain comprises at least one repeat unit having a repeat variable diresidue (RVD), which contacts a target nucleic acid base. In some embodiments, a DNA binding domain comprises more than one repeat unit, each having an RVD, which contacts a target nucleic acid base. In some embodiments, the DNA binding domain comprises 1 to 50 RVDs. In some embodiments, the DNA binding domain components of the fusion proteins can be at least 14 RVDs, at least 15 RVDs, at least 16 RVDs, at least 17 RVDs, at least 18 RVDs, at least 19 RVDs, at least 20 RVDs in length, or at least 21 RVDs in length. In some embodiments, the DNA binding domains can be 16 to 21 RVDs in length.

In some embodiments, any one of the DNA binding domains described herein can bind to a region of interest of any gene. For example, the DNA binding domains described herein can bind upstream of the promoter region, upstream of the gene transcription start site, or downstream of the transcription start site. In certain embodiments, the DNA binding domain binding region is no farther than 50 base pairs downstream of the transcription start site. In some embodiments, the DNA binding domain is designed to bind in proximity to the transcription start site (TSS). In other embodiments, the TALE can be designed to bind in the 5′ UTR region.

A DNA binding domain described herein can comprise between 1 to 50 repeat units. A DNA binding domain described herein can comprise between 5 and 45, between 8 to 45, between 10 to 40, between 12 to 35, between 15 to 30, between 20 to 30, between 8 to 40, between 8 to 35, between 8 to 30, between 10 to 35, between 10 to 30, between 10 to 25, between 10 to 20, or between 15 to 25 repeat units.

A DNA binding domain described herein can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more repeat units. A DNA binding domain described herein can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, or 50 repeat units. A DNA binding domain described herein can comprise 5 repeat units. A DNA binding domain described herein can comprise 10 repeat units. A DNA binding domain described herein can comprise 11 repeat units. A DNA binding domain described herein can comprise 12 repeat units, or another suitable number.

A repeat unit of a DNA binding domain can be 18, 19, 20, 21, 22, 23, 24, 25, 26, 27, 28, 29, 30, 31, 32, 33, 34, 35, 36 37, 38, 39 or 40 residues in length.

In some embodiments, the effector can be a protein secreted from Xanthomonas or Ralstonia bacteria upon plant infection. In some embodiments, the effector can be a protein that is a mutated form of, or otherwise derived from, a protein secreted from Xanthomonas or Ralstonia bacteria. The effector can further comprise a DNA-binding module which includes a variable number of about 33-35 amino acid residue repeat units. Each amino acid repeat unit recognizes one base pair through two adjacent amino acids (e.g., at amino acid positions 12 and 13 of the repeat unit). As such, amino acid positions 12 and 13 of the repeat unit can also be referred to as repeat variable diresidue (RVD).

### Linkers

A nuclease, e.g., anyone of SEQ ID NO: 1-SEQ ID NO: 81 (or any one of nucleic acid sequences of SEQ ID NO: 82-SEQ ID NO: 162) fused to a DNA binding domain (e.g., an RNBD, a MAP-NBD, a TALE), can further include a linker connecting SEQ ID NO: 1-SEQ ID NO: 81 (or any one of nucleic acid sequences of SEQ ID NO: 82-SEQ ID NO: 162) to the DNA binding domain. A linker used herein can be a short flexible linker comprising 0 base pairs, 3 to 6 base pairs, 6 to 12 base pairs, 12 to 15 base pairs, 15 to 21 base pairs, 21 to 24 base pairs, 24 to 30 base pairs, 30 to 36 base pairs, 36 to 42 base pairs, 42 to 48 base pairs, or 1 to 48 base pairs. The nucleic acid sequence of the linker can encode for an amino acid sequence comprising 0 residues, 1-3 residues, 4-7 residues, 8-10 residues, 10-12 residues, 12-15 residues, or 1-15 residues. Linkers can include, but are not limited to, residues such as glycine, methionine, aspartic acid, alanine, lysine, serine, leucine, threonine, tryptophan, or any combination thereof.

When linking a repressor domain to an RNBD, MAP-NBD, or TALE, the linker can have a nucleic acid sequence of GGCGGTGGCGGAGGGATGGATGCTAAGTCACTAACTGCCTGGTCC (SEQ ID NO: 165) and an amino acid sequence of GGGGGMDAKSLTAWS (SEQ ID NO: 166).

A nuclease, e.g., anyone of SEQ ID NO: 1-SEQ ID NO: 81 (or any one of nucleic acid sequences of SEQ ID NO: 82-SEQ ID NO: 162) can be connected to a DNA binding domain via a linker, a linker can be between 1 to 70 amino acid residues in length. A linker can be from 5 to 45, from 5 to 40, from 5 to 35, from 5 to 30, from 5 to 25, from 5 to 20, from 5 to 15, from 10 to 40, from 10 to 35, from 10 to 30, from 10 to 25, from 10 to 20, from 12 to 40, from 12 to 35, from 12 to 30, from 12 to 25, from 12 to 20, from 14 to 40, from 14 to 35, from 14 to 30, from 14 to 25, from 14 to 20, from 14 to 16, from 15 to 40, from 15 to 35, from 15 to 30, from 15 to 25, from 15 to 20, from 15 to 18, from 18 to 40, from 18 to 35, from 18 to 30, from 18 to 25, from 18 to 24, from 20 to 40, from 20 to 35, from 20 to 30, from 25 to 30, from 25 to 70, from 30 to 70, from 5 to 70, from 35 to 70, from 40 to 70, from 45 to 70, from 50 to 70, from 55 to 70, from 60 to 70, or from 65 to 70 amino acid residues in length.

A linker for linking a nuclease, e.g., anyone of SEQ ID NO: 1-SEQ ID NO: 81 (or any one of nucleic acid sequences of SEQ ID NO: 82-SEQ ID NO: 162) to a DNA binding domain can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, or 70 amino acid residues in length.

In some embodiments, the linker can be the N-terminus of a naturally occurring Ralstonia solanacearum-derived protein, Xanthomonas spp.-derived protein, or animal pathogen-derived protein, wherein any functional domain disclosed herein is fused to the N-terminus of the engineered DNA binding domain. In some embodiments, the linker comprising the N-terminus can comprise the full length naturally occurring N-terminus of a naturally occurring Ralstonia solanacearum-derived protein, Xanthomonas spp.-derived protein, or animal pathogen-derived protein, or a truncation of the naturally occurring N-terminus, such as amino acid residues at positions 1 to 137 of the naturally occurring Ralstonia solanacearum-derived protein N-terminus (e.g., SEQ ID NO: 264), positions 1 (H) to 115 (S) of the naturally occurring Ralstonia solanacearum-derived protein N-terminus (SEQ ID NO: 320), positions 1 (N) to 115 (S) of the naturally occurring Xanthomonas spp.-derived protein N-terminus (SEQ ID NO: 321), or positions 1 (G) to 115 (K) of the naturally occurring Legionella quateirensis-derived protein N-terminus (SEQ ID NO: 322). In some embodiments, the linker can comprise amino acid residues at positions 1 to 120 of the naturally occurring Ralstonia solanacearum-derived protein (SEQ ID NO: 303), Xanthomonas spp.-derived protein (SEQ ID NO: 301), or Legionella quateirensis-derived protein (SEQ ID N): 304). In some embodiments, the linker can comprise the naturally occurring N-terminus of Ralstonia solanacearum truncated to any length. For example, the naturally occurring N-terminus of Ralstonia solanacearum can be truncated to amino acid residues at positions 1 to 120, 1 to 115, 1 to 50, 1 to 70, 1 to 100, 1 to 120, 1 to 130, 10 to 40, 60 to 100, or 100 to 120 and used at the N-terminus of the engineered DNA binding domain as a linker to a nuclease or a repressor.

In other embodiments, the linker can be the C-terminus of a naturally occurring Ralstonia solanacearum-derived protein, Xanthomonas spp.-derived protein, or animal pathogen-derived protein, wherein any functional domain disclosed herein is fused to the C-terminus of the engineered DNA binding domain. In some embodiments, the linker comprising the C-terminus can comprise the full length naturally occurring C-terminus of a naturally occurring Ralstonia solanacearum-derived protein, Xanthomonas spp.-derived protein, or animal pathogen-derived protein, or a truncation of the naturally occurring C-terminus, such as positions 1 to 63 of the naturally occurring Ralstonia solanacearum-derived protein (SEQ ID NO: 266), Xanthomonas spp.-derived protein (SEQ ID NO: 298), or Legionella quateirensis-derived protein (SEQ ID NO: 306). In some embodiments, the naturally occurring C-terminus of Ralstonia solanacearum-derived protein, Xanthomonas spp.-derived protein, or Legionella quateirensis-derived protein can be truncated to any length and used at the C-terminus of the engineered DNA binding domain and used as a linker to a nuclease or repressor. For example, the naturally occurring C-terminus of Ralstonia solanacearum-derived protein, Xanthomonas spp.-derived protein, or Legionella quateirensis-derived protein can be truncated to amino acid residues at positions 1 to 63, 1 to 50, 1 to 70, 1 to 100, 1 to 120, 1 to 130, 10 to 40, 60 to 100, or 100 to 120 and used at the C-terminus of the engineered DNA binding domain.

### Linkers Comprising Recognition Sites

In some embodiments, the present disclosure provides DNA binding domains (e.g., RNBDs, MAP-NBDs, TALEs) with gapped repeat units for use as gene editing complexes. A DNA binding domain (e.g., RNBDs, MAP-NBDs, TALEs) with gapped repeat can comprise of a plurality of repeat units in which each repeat unit of the plurality of repeat units is separated from a neighboring repeat unit by a linker. This linker can comprise a recognition site for additional functionality and activity. For example, the linker can comprise a recognition site for a small molecule. As another example, the linker can serve as a recognition site for a protease. In yet another example, the linker can serve as a recognition site for a kinase. In other embodiments, the recognition site can serve as a localization signal.

Each repeat unit of a DNA binding domain (e.g., RNBDs, MAP-NBDs, TALEs) comprises a secondary structure in which the RVD interfaces with and binds to a target nucleic acid base on double stranded DNA, while the remainder of the repeat unit protrudes from the surface of the DNA. Thus, the linkers comprising a recognition site between each repeat unit are removed from the surface of the DNA and are solvent accessible. In some embodiments, these solvent accessible linkers comprising recognition sites can have extra activity while mediating gene editing. In some embodiments, the at least one repeat unit comprises 1-20 additional amino acid residues at the C-terminus. In some aspects, the at least repeat unit of the plurality of repeat units is separated from a neighboring repeat unit by a linker. In further aspects, the linker comprises a recognition site. In some aspects, the recognition site is for a small molecule, a protease, or a kinase. In some aspects, the recognition site serves as a localization signal. In some aspects, the plurality of repeat units comprises 3 to 60 repeat units.

Examples of a left and a right DNA binding domain comprising repeat units derived from Xanthomonas spp. are shown below in TABLE 7 for AAVS1 and GA7. “X,” shown in bold and underlining, represents a linker comprising a recognition site and can comprise 1-40 amino acid residues. An amino acid residue of the linker can comprise a glycine, an alanine, a threonine, or a histidine.

TABLE 7
Exemplary Left or Right Gapped
DNA Binding Domains

| SEQ | | |
|---|---|---|
| ID | | |
| NO | Construct | Sequence |
| 307 | AAVS1_ | LTPDQVVAIASHDGGKQALETVQRLLPVLC |
| | Left | QDHGXLTPDQVVAIASHDGGKQALETVQRL |
| | | LPVLCQDHGXLTPDQVVAIASHDGGKQALE |
| | | TVQRLLPVLCQDHGXLTPDQVVAIASHDGG |
| | | KQALETVQRLLPVLCQDHGXLTPDQVVAIA |
| | | SNGGGKQALETVQRLLPVLCQDHGXLTPDQ |
| | | VVAIASHDGGKQALETVQRLLPVLCQDHGX |
| | | LTPDQVVAIASHDGGKQALETVQRLLPVLC |
| | | QDHGXLTPDQVVAIASNIGGKQALETVQRL |
| | | LPVLCQDHGXLTPDQVVAIASHDGGKQALE |
| | | TVQRLLPVLCQDHGXLTPDQVVAIASHDGG |
| | | KQALETVQRLLPVLCQDHGXLTPDQVVAIA |
| | | SHDGGKQALETVQRLLPVLCQDHGXLTPDQ |
| | | VVAIASHDGGKQALETVQRLLPVLCQDHGX |
| | | LTPDQVVAIASNIGGKQALETVQRLLPVLC |
| | | QDHGXLTPDQVVAIASHDGGKQALETVQRL |
| | | LPVLCQDHGXLTPDQVVAIASNIGGKQALE |
| | | TVQRLLPVLCQDHGXLTPDQVVAIASNHGG |
| | | KQALETVQRLLPVLCQDHGXLTPDQVVAIA |
| | | SNGGG |
| 308 | AAVS1_ | LTPDQVVAIASNGGGKQALETVQRLLPVLC |
| | Right | QDHGXLTPDQVVAIASNGGGKQALETVQRL |
| | | LPVLCQDHGXLTPDQVVAIASNGGGKQALE |
| | | TVQRLLPVLCQDHGXLTPDQVVAIASHDGG |
| | | KQALETVQRLLPVLCQDHGXLTPDQVVAIA |
| | | SNGGGKQALETVQRLLPVLCQDHGXLTPDQ |
| | | VVAIASNHGGKQALETVQRLLPVLCQDHGX |
| | | LTPDQVVAIASNGGGKQALETVQRLLPVLC |
| | | QDHGXLTPDQVVAIASHDGGKQALETVQRL |
| | | LPVLCQDHGXLTPDQVVAIASNIGGKQALE |
| | | TVQRLLPVLCQDHGXLTPDQVVAIASHDGG |
| | | KQALETVQRLLPVLCQDHGXLTPDQVVAIA |
| | | SHDGGKQALETVQRLLPVLCQDHGXLTPDQ |
| | | VVAIASNIGGKQALETVQRLLPVLCQDHGX |
| | | LTPDQVVAIASNIGGKQALETVQRLLPVLC |
| | | QDHGXLTPDQVVAIASNGGGKQALETVQRL |
| | | LPVLCQDHGXLTPDQVVAIASHDGGKQALE |
| | | TVQRLLPVLCQDHGXLTPDQVVAIASHDGG |
| | | KQALETVQRLLPVLCQDHGXLTPDQVVAIA |
| | | SNGGGKQALESIVAQLSRPDPALA |
| 309 | GA7.2 | LTPDQVVAIASNHGGKQALETVQRLLPVLC |
| | Left | QDHGXLTPDQVVAIASHDGGKQALETVQRL |
| | | LPVLCQDHGXLTPDQVVAIASNGGGKQALE |
| | | TVQRLLPVLCQDHGXLTPDQVVAIASHDGG |
| | | KQALETVQRLLPVLCQDHGXLTPDQVVAIA |
| | | SNIGGKQALETVQRLLPVLCQDHGXLTPDQ |
| | | VVAIASNHGGKQALETVQRLLPVLCQDHGX |
| | | LTPDQVVAIASHDGGKQALETVQRLLPVLC |
| | | QDHGXLTPDQVVAIASHDGGKQALETVQRL |
| | | LPVLCQDHGXLTPDQVVAIASHDGGKQALE |
| | | TVQRLLPVLCQDHGXLTPDQVVAIASNIGG |
| | | KQALETVQRLLPVLCQDHGXLTPDQVVAIA |
| | | SNHGGKQALETVQRLLPVLCQDHGXLTPDQ |
| | | VVAIASHDGGKQALETVQRLLPVLCQDHGX |
| | | LTPDQVVAIASNGGGKQALETVQRLLPVLC |
| | | QDHGXLTPDQVVAIASHDGGKQALETVQRL |
| | | LPVLCQDHGXLTPDQVVAIASNIGGKQALE |
| | | TVQRLLPVLCQDHGXLTPDQVVAIASNHGG |
| | | KQALETVQRLLPVLCQDHGXLTPDQVVAIA |
| | | SHDGGKQALETVQRLLPVLCQDHGXLTPDQ |
| | | VVAIASHDGGKQALETVQRLLPVLCQDHGX |
| | | LTPDQVVAIASNGGGK |
| 310 | GA7.2 | LTPDQVVAIASHDGGKQALETVQRLLPVLC |
| | Right | QDHGXLTPDQVVAIASHDGGKQALETVQRL |
| | | LPVLCQDHGXLTPDQVVAIASHDGGKQALE |
| | | TVQRLLPVLCQDHGXLTPDQVVAIASHDGG |
| | | KQALETVQRLLPVLCQDHGXLTPDQVVAIA |
| | | SHDGGKQALETVQRLLPVLCQDHGXLTPDQ |
| | | VVAIASNGGGKQALETVQRLLPVLCQDHGX |
| | | LTPDQVVAIASHDGGKQALETVQRLLPVLC |
| | | QDHGXLTPDQVVAIASNGGGKQALETVQRL |
| | | LPVLCQDHGXLTPDQVVAIASHDGGKQALE |
| | | TVQRLLPVLCQDHGXLTPDQVVAIASNIGG |
| | | KQALETVQRLLPVLCQDHGXLTPDQVVAIA |
| | | SNGGGKQALETVQRLLPVLCQDHGXLTPDQ |
| | | VVAIASNGGGKQALETVQRLLPVLCQDHGX |
| | | LTPDQVVAIASHDGGKQALETVQRLLPVLC |
| | | QDHGXLTPDQVVAIASNGGGKQALETVQRL |
| | | LPVLCQDHGXLTPDQVVAIASHDGGKQALE |
| | | TVQRLLPVLCQDHGXLTPDQVVAIASNGGG |
| | | KQALETVQRLLPVLCQDHGXLTPDQVVAIA |
| | | SNIGGKQALETVQRLLPVLCQDHGXLTPDQ |
| | | VVAIASHDGGKQALETVQRLLPVLCQDHGX |
| | | LTPDQVVAIASHDGGKQALETVQRLLPVLC |
| | | QDHGXLTPDQVVAIASNIGGKQALETVQRL |
| | | LPVLCQDHGXLTPDQVVASASNGGGKQALE |
| | | SIVAQLSRPDPALA |

### Tunable Repeat Units

In some embodiments, the present disclosure provides DNA binding domains (e.g., RNBDs, MAP-NBDs, TALEs) with expanded repeat units. For example, a DNA binding domain (e.g., RNBDs, MAP-NBDs, TALEs) comprises a plurality of repeat units in which each repeat unit is usually 33-35 amino acid residues in length. The present disclosure provides repeat units, which are greater than 35 amino acid residues in length. In some embodiments, the present disclosure provides repeat units, which are greater than 39 amino acid residues in length. In some embodiments, the present disclosure provides repeat units which are 35 to 40, 39 to 40, 35 to 45, 39 to 45, 35 to 50, 39 to 50, 35 to 50, 35 to 60, 39 to 60, 35 to 70, 39 to 70, 35 to 79, or 39 to 79 amino acid residues long.

In other embodiments, the present disclosure provides DNA binding domains (e.g., RNBDs, MAP-NBDs, TALEs) with contracted repeat units. For example, the present disclosure provides repeat units, which are less than 32 amino acid residues in length. In some embodiments, the present disclosure provides repeat units, which are 15 to 32, 16 to 32, 17 to 32, 18 to 32, 19 to 32, 20 to 32, 21 to 32, 22 to 32, 23 to 32, 24 to 32, 25 to 32, 26 to 32, 27 to 32, 28 to 32, 29 to 32, 30 to 32, or 31 to 32 amino acid residues in length.

In some embodiments, said expanded repeat units can be tuned to modulate binding of each repeat unit to its target nucleic acid, resulting in the ability to overall modulate binding of the DNA binding domain (e.g., RNBDs, MAP-NBDs, TALEs) to a target gene of interest. For example, expanding repeat units can improve binding affinity of the repeat unit to its target nucleic acid base and thereby increase binding affinity of the DNA binding domain (e.g., RNBDs, MAP-NBDs, TALEs) to a target gene. In other embodiments, contracting repeat units can improve binding affinity of the repeat unit to its target nucleic acid base and thereby increase binding affinity of the DNA binding domain (e.g., RNBDs, MAP-NBDs, TALEs) for a target gene.

### Functional Domains

An RNBD (e.g., Ralstonia solanacearum-derived), or another binding domain (e.g., MAP-NBD or TALE), can be linked to a functional domain. The functional domain can provide different types activity, such as genome editing, gene regulation (e.g., activation or repression), or visualization of a genomic locus via imaging.

### A. Genome Editing Domains

For example, an RNBD (e.g., Ralstonia solanacearum-derived), or another binding domain (e.g., MAP-NBD or TALE), can be linked to a nuclease, wherein the RNBD provides specificity and targeting and the nuclease provides genome editing functionality. In some embodiments, the nuclease can be a cleavage domain, which dimerizes with another copy of the same cleavage domain to form an active full domain capable of cleaving DNA. In other embodiments, the nuclease can be a cleavage domain, which is capable of cleaving DNA without needing to dimerize. For example, a nuclease comprising a cleavage domain can be an endonuclease, such as FokI or Bfil. In some embodiments, two cleavage domains (e.g., FokI or Bfil) can be fused together to form a fully functional single cleavage domain. When cleavage domains are used as the nuclease, two RNBDs can be engineered, the first RNBD binding to a top strand of a target nucleic acid sequence and comprising a first FokI cleavage domain and a second RNBD binding to a bottom strand of a target nucleic acid sequence and comprising a second FokI cleavage domain.

In some embodiments, a fully functional cleavage domain, capable of cleaving DNA without needing to dimerize include meganucleases, also referred to as homing endonucleases. For example, a meganuclease can include I-AniI or I-OnuI. In some embodiments, the nuclease can be a type IIS restriction enzyme, such as FokI or Bfil.

A nuclease domain fused to an RNBD (e.g., Ralstonia solanacearum-derived), or another binding domain (e.g., MAP-NBD or TALE), can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V, and VIII; DNA polymerase I, RNA exonuclease 2, and the like.

A nuclease domain fused to an RNBD (e.g., Ralstonia solanacearum-derived), or another binding domain (e.g., MAP-NBD or TALE), can be a restriction endonuclease (or restriction enzyme). In some instances, a restriction enzyme cleaves DNA at a site removed from the recognition site and has a separate binding and cleavage domains. In some instances, such restriction enzyme is a Type IIS restriction enzyme.

A nuclease domain fused to an RNBD (e.g., Ralstonia solanacearum-derived), or another binding domain (e.g., MAP-NBD or TALE), can be a Type IIS nuclease. A Type IIS nuclease can be FokI or Bfil. In some cases, a nuclease domain fused to an RNBD (e.g., Ralstonia solanacearum-derived) is FokI. In other cases, a nuclease domain fused to an RNBD (e.g., Ralstonia solanacearum-derived) is Bfil.

FokI can be a wild-type FokI or can comprise one or more mutations. In some cases, FokI can comprise 1, 2, 3, 4, 5, 6, 7, 8, 9, 10, or more mutations. A mutation can enhance cleavage efficiency. A mutation can abolish cleavage activity. In some cases, a mutation can modulate homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization.

In some instances, a FokI cleavage domain is, for example, as described in Kim et al. “Hybrid restriction enzymes: Zinc finger fusions to Fok I cleavage domain,” PNAS 93:1156-1160 (1996), which is incorporated herein by reference in its entirety. In some cases, a FokI cleavage domain described herein has a sequence as follows: QLVKSELEEKKSELRHKLKYVPHEYIELIEIARNSTQDRILEMKVMEFFMKVYGYRGKHLG GSRKPDGAIYTVGSPIDYGVIVDTKAYSGGYNLPIGQADEMQRYVEENQTRNKHINPNEWW KVRRKENNGEINF (SEQ ID NO: 163). In other instances, a FokI cleavage domain described herein is a FokI, for example, as described in U.S. Pat. No. 8,586,526, which is incorporated herein by reference in its entirety.

An RNBD (e.g., Ralstonia solanacearum-derived) can be linked to a functional group that modifies DNA nucleotides, for example an adenosine deaminase.

In some embodiments, an RNBD (e.g., Ralstonia solanacearum-derived) can be linked to any nuclease as set forth in TABLE 6 showing exemplary amino acid sequences (SEQ ID NO: 1-SEQ ID NO: 81) of endonucleases for genome editing and the corresponding back-translated nucleic acid sequences (SEQ ID NO: 82-SEQ ID NO: 162) of the endonucleases.

For purposes of gene editing, a first DNA binding domain (e.g., of a TALE, RNBD, or MAP-NBD) linked to a cleavage domain and a second DNA binding domain (e.g., of a TALE, RNBD, or MAP-NBD) linked to a cleavage domain can be provided. The first DNA binding domain (e.g., of a TALE, RNBD, or MAP-NBD) linked to a cleavage domain can recognize a top strand of double stranded DNA and bind to said region of double stranded DNA. The second DNA binding domain (e.g., of a TALE, RNBD, or MAP-NBD) linked to a cleavage domain can recognize a separate, non-overlapping bottom strand of double stranded DNA and bind to said region of double stranded DNA. The target nucleic acid sequence on the bottom strand can have its complementary nucleic acid sequence in the top strand positioned 10 to 20 nucleotides towards the 3′ end from the first region. In some embodiments this stretch of 10 to 20 nucleotides can be referred to as the spacer region. In some embodiments, this first DNA binding domain (e.g., of a TALE, RNBD, or MAP-NBD) linked to a cleavage domain and the second DNA binding domain (e.g., of a TALE, RNBD, or MAP-NBD) linked to a cleavage domain both bind at a target site, allowing for dimerization of the two cleavage domains in the spacer region and allowing for catalytic activity and cleaving of the target DNA.

a. Potency and Specificity of Genome Editing

In some embodiments, the efficiency of genome editing with a genome editing complex of the present disclosure (e.g., any one of an RNBD, MAP-NBD, or TALE fused to any nuclease disclosed herein) can be determined. Specifically, the potency and specificity of the genome editing complex can indicate whether a particular modular nucleic acid binding domain fused to a nuclease provides efficient editing. Potency can be defined as the percent indels (insertions/deletions) that are generated via the non-homologous end joining (NHEJ) pathway at a target site after administering a modular nucleic acid binding domain fused to a nuclease to a subject. A modular nucleic acid binding domain can have a potency of greater than 50%, greater than 55%, greater than 60%, greater than 65%, greater than 70%, greater than 75%, greater than 80%, greater than 85%, greater than 90%, greater than 95%, greater than 92%, greater than 95%, greater than 97%, or greater than 99%. A modular nucleic acid binding domain can have a potency of from 50% to 100%, 50% to 60%, 60% to 70%, 70% to 80%, 80% to 90%, or 90% to 100%.

Specificity can be defined as a specificity ratio, wherein the ratio is the percent indels at a target site of interest over the percent indels at the top-ranked off-target site for a particular genome editing complex (e.g., any DNA binding domain linked to a nuclease described herein) of interest. A high specificity ratio would indicate that a modular nucleic acid binding domain fused to a nuclease edits primarily at the desired target site and exhibits fewer instances of undesirable, off-target editing. A low specificity ratio would indicate that a modular nucleic acid binding domain fused to a nuclease does not edit efficiently at the desired target site and/or can indicate that the modular nucleic acid binding domain fused to a nuclease exhibits high off-target activity. A modular nucleic acid binding domain can have a specificity ratio for the target site of at least 50:1, 55:1, 60:1, 65:1, 70:1, 75:1, 80:1, 85:1, 90:1, 92:1, 95:1, 97:1, 99:1, 50:2, 55:2, 60:2, 65:2, 70:2, 75:2, 80:2, 85:2, 90:2, 92:2, 95:2, 97:2, 99:2, 50:3, 55:3, 60:3, 65:3, 70:3, 75:3, 80:3, 85:3, 90:3, 92:3, 95:3, 97:3, 99:3, 50:4, 55:4, 60:4, 65:4, 70:4, 75:4, 80:4, 85:4, 90:4, 92:4, 95:4, 97:4, 99:4, 50:5, 55:5, 60:5, 65:5, 70:5, 75:5, 80:5, 85:5, 90:5, 92:5, 95:5, 97:5, or 99:5. A modular nucleic acid binding domain can have a specificity ratio for the target site from 50:1 to 100:1, 99:5 to 50:1, or 99:5 to 100:1. Percent indels can be measured via deep sequencing techniques.

In some embodiments, the present disclosure provides a polypeptide comprising a modular nucleic acid binding domain comprising a potency for a target site greater than 65% and a specificity ratio for the target site of at least 50:1; and a functional domain; wherein: the modular nucleic acid binding domain comprises a plurality of repeat units; at least one repeat unit of the plurality of repeat units comprises a binding region configured to bind to a target nucleic acid base within the target site; the potency comprises indel percentage at the target site, and wherein the specificity ratio comprises indel percentage at the target site over indel percentage at a top-ranked off-target site of the polypeptide. Indel percentage can be measured by deep sequencing.

The top-ranked off-target site for a polypeptide (e.g., a modular nucleic acid binding domain linked to a cleavage domain) can be determined using the predicted report of genome-wide nuclease off-target sites (PROGNOS) ranking algorithms as described in Fine et al. (Nucleic Acids Res. 2014 April; 42 (6): c42. doi: 10.1093/nar/gkt1326. Epub 2013 Dec. 30.). As described in Fine et al, the PROGNOS algorithm TALEN v2.0 can use the DNA target sequence as input; prior construction and experimental characterization of the specific nucleases are not necessary. Based on the differences between the sequence of a potential off-target site in the genome and the intended target sequence, the algorithm can generate a score that is used to rank potential off-target sites. If two (or more) potential off-target sites have equal scores, they can be further ranked by the type of genomic region annotated for each site with the following order: Exon >Promoter>Intron>Intergenic. A final ranking by chromosomal location can be employed as a tie-breaker to ensure consistency in the ranking order. Thus, a score can be generated for each potential off-target site.

### B. Regulatory Domains

As another example, an RNBD (e.g., Ralstonia solanacearum-derived), or another binding domain (e.g., MAP-NBD or TALE), can be linked to a gene regulating domain. A gene regulation domain can be an activator or a repressor. For example, an RNBD (e.g., Ralstonia solanacearum-derived), or another binding domain (e.g., MAP-NBD or TALE), can be linked to an activation domain, such as VP16, VP64, p65, p300 catalytic domain, TET1 catalytic domain, TDG, Ldb1 self-associated domain, SAM activator (VP64, p65, HSF1), or VPR (VP64, p65, Rta). Alternatively, an RNBD (e.g., Ralstonia solanacearum-derived), or another binding domain (e.g., MAP-NBD or TALE), can be linked to a repressor, such as KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

In some embodiments, an RNBD (e.g., Ralstonia solanacearum-derived), or another binding domain (e.g., MAP-NBD or TALE), can be linked to a DNA modifying protein, such as DNMT3a. An RNBD (e.g., Ralstonia solanacearum-derived), or another binding domain (e.g., MAP-NBD or TALE), can be linked to a chromatin-modifying protein, such as lysine-specific histone demethylase 1 (LSD1). An RNBD (e.g., Ralstonia solanacearum-derived), or another binding domain (e.g., MAP-NBD or TALE), can be linked to a protein that is capable of recruiting other proteins, such as KRAB. The DNA modifying protein (e.g., DNMT3a) and proteins capable of recruiting other proteins (e.g., KRAB) can serve as repressors of transcription. Thus, RNBDs (e.g., Ralstonia solanacearum-derived), or another binding domain (e.g., MAP-NBD or TALE), linked to a DNA modifying protein (e.g., DNMT3a) or a domain capable of recruiting other proteins (e.g., KRAB, a domain found in transcriptional repressors, such as Kox1) can provide gene repression functionality, can serve as transcription factors, wherein the RNBD (e.g., Ralstonia solanacearum-derived), or another binding domain (e.g., MAP-NBD or TALE), provides specificity and targeting and the DNA modifying protein and the protein capable of recruiting other proteins provides gene repression functionality, which can be referred to as a TALE-transcription factor (TALE-TF), RNBD-transcription factor (RNBD-TF), or MAP-NBD-transcription factor (MAP-NBD-TF).

In some embodiments, expression of the target gene can be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 95%, at least 97%, or at least 99% by using a DNA binding domain fused to a repression domain (e.g., an RNBD-TF, a MAP-NBD-TF, or TALE-TF) of the present disclosure as compared to non-treated cells. In some embodiments, expression of the target gene can be reduced by 5% to 10%, 10% to 15%, 15% to 20%, 20%, to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, 90% to 95%, or 95% to 99% by using an RNBD-TF, a MAP-NBD-TF, or TALE-TF of the present disclosure as compared to non-treated cells. In some embodiments, expression of the checkpoint gene can be reduced by over 90% by using an RNBD-TF, a MAP-NBD-TF, or TALE-TF of the present disclosure as compared to non-treated cells.

In some embodiments, repression of the target gene with a DNA binding domain fused to a repression domain (e.g., an RNBD-TF, a MAP-NBD-TF, or TALE-TF) of the present disclosure and subsequent reduced expression of the target gene can last for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days. In some embodiments, repression of the target gene with an RNBD-TF, a MAP-NBD-TF, or TALE-TF of the present disclosure and subsequent reduced expression of the target gene can last for 1 days to 3 days, 3 days to 5 days, 5 days to 7 days, 7 days to 9 days, 9 days to 11 days, 11 days to 13 days, 13 days to 15 days, 15 days to 17 days, 17 days to 19 days, 19 days to 21 days, 21 days to 23 days, 23 days to 25 days, or 25 days to 28 days.

In various aspects, the present disclosure provides a method of identifying a target binding site in a target gene of a cell, the method comprising: (a) contacting a cell with an engineered genomic regulatory complex comprising a DNA binding domain, a repressor domain, and a linker; (b) measuring expression of the target gene; and (c) determining expression of the target gene is repressed by at least 50%, at least 60%, at least 70%, at least 80%, at least 85%, at least 90%, at least 92%, at least 95%, at least 97%, or at least 99% for at least 3 days, wherein the target gene is selected from: a checkpoint gene and a T cell surface receptor.

In some aspects, expression of the target gene is repressed in at least 75%, at least 80%, at least 85%, at least 90%, at least 95%, or at least 99% of a plurality of the cells. In some aspects, the engineered genomic regulatory complex is undetectable after at least 3 days. In some aspects, determining the engineered genomic regulatory complex is undetectable is measured by qPCR, imaging of a FLAG-tag, or a combination thereof. In some aspects, the measuring expression of the target gene comprises flow cytometry quantification of expression of the target gene.

In some embodiments, repression of the target gene with a DNA binding domain fused to a repression domain (e.g., an RNBD-TF, a MAP-NBD-TF, or TALE-TF) of the present disclosure can last even after the DNA binding domain-gene regulator becomes undetectable. The DNA binding domain fused to a repression domain (e.g., an RNBD-TF, a MAP-NBD-TF, or TALE-TF) can become undetectable after at least 3 days. In some embodiments, the DNA binding domain fused to a repression domain (e.g., an RNBD-TF, a MAP-NBD-TF, or TALE-TF) can become undetectable after at least 1 day, at least 2 days, at least 3 days, at least 4 days, at least 5 days, at least 6 days, at least 1 week, at least 2 weeks, at least 3 weeks, or at least 4 weeks. In some embodiments, qPCR or imaging via the FLAG-tag can be used to confirm that the DNA binding domain fused to a repression domain (e.g., an RNBD-TF, a MAP-NBD-TF, or TALE-TF) is no longer detectable.

### C. Imaging Moieties

An RNBD (e.g., Ralstonia solanacearum-derived), or another binding domain (e.g., MAP-NBD or TALE), can be linked to a fluorophore, such as Hydroxycoumarin, methoxycoumarin, Alexa fluor, aminocoumarin, Cy2, FAM, Alexa fluor 488, Fluorescein FITC, Alexa fluor 430, Alexa fluor 532, HEX, Cy3, TRITC, Alexa fluor 546, Alexa fluor 555, R-phycocrythrin (PE), Rhodamine Red-X, Tamara, Cy3.5, Rox, Alexa fluor 568, Red 613, Texas Red, Alexa fluor 594, Alexa fluor 633, Allophycocyanin, Alexa fluor 633, Cy5, Alexa fluor 660, Cy5.5, TruRed, Alexa fluor 680, Cy7, GFP, or mCHERRY. An RNBD (e.g., Ralstonia solanacearum-derived) can be linked to a biotinylation reagent.

### Genes and Indications of Interest

In some embodiments, genome editing can be performed by fusing a nuclease of the present disclosure with a DNA binding domain for a particular genomic locus of interest. Genetic modification can involve introducing a functional gene for therapeutic purposes, knocking out a gene for therapeutic gene, or engineering a cell ex vivo (e.g., HSCs or CAR T cells) to be administered back into a subject in need thereof. For example, the genome editing complex can have a target site within PDCD1, CTLA4, LAG3, TET2, BTLA, HAVCR2, CCR5, CXCR4, TRA, TRB, B2M, albumin, HBB, HBA1, TTR, NR3C1, CD52, crythroid specific enhancer of the BCL11A gene, CBLB, TGFBR1, SERPINA1, HBV genomic DNA in infected cells, CEP290, DMD, CFTR, IL2RG, CS-1, or any combination thereof. In some embodiments, a genome editing complex can cleave double stranded DNA at a target site in order to insert a chimeric antigen receptor (CAR), alpha-L iduronidase (IDUA), iduronate-2-sulfatase (IDS), or Factor 9 (F9). Cells, such as hematopoietic stem cells (HSCs) and T cells, can be engineered ex vivo with the genome editing complex. Alternatively, genome editing complexes can be directly administered to a subject in need thereof.

The subject receiving treatment can be suffering from a disease such as transthyretin amyloidosis (ATTR), HIV, glioblastoma multiforme, cancer, acute lymphoblastic leukemia, acute myeloid leukemia, beta-thalassemia, sickle cell disease, MPSI, MPSII, Hemophilia B, multiple myeloma, melanoma, sarcoma, Leber congenital amaurosis (LCA10), CD19 malignancies, BCMA-related malignancies, duchenne muscular dystrophy (DMD), cystic fibrosis, alpha-1 antitrypsin deficiency, X-linked severe combined immunodeficiency (X-SCID), or Hepatitis B.

### Samples for Analysis

In some aspects, described herein include methods of modifying the genetic material of a target cell utilizing an RNBD described herein. A sample described herein may be a fresh sample. The sample may be a live sample.

The sample may be a cell sample. The cell sample may be obtained from the cells or tissue of an animal. The animal cell may comprise a cell from an invertebrate, fish, amphibian, reptile, or mammal. The mammalian cell may be obtained from a primate, ape, equine, bovine, porcine, canine, feline, or rodent. The mammal may be a primate, ape, dog, cat, rabbit, ferret, or the like. The rodent may be a mouse, rat, hamster, gerbil, hamster, chinchilla, or guinea pig. The bird cell may be from a canary, parakeet, or parrot. The reptile cell may be from a turtle, lizard, or snake. The fish cell may be from a tropical fish. For example, the fish cell may be from a zebrafish (such as Danio rerio). The amphibian cell may be from a frog. An invertebrate cell may be from an insect, arthropod, marine invertebrate, or worm. The worm cell may be from a nematode (such as Caenorhabditis elegans). The arthropod cell may be from a tarantula or hermit crab.

The cell sample may be obtained from a mammalian cell. For example, the mammalian cell may be an epithelial cell, connective tissue cell, hormone secreting cell, a nerve cell, a skeletal muscle cell, a blood cell, an immune system cell, or a stem cell. A cell may be a fresh cell, live cell, fixed cell, intact cell, or cell lysate. Cell samples can be any primary cell, such as a hematopoctic stem cell (HSCs) or naïve or stimulated T cells (e.g., CD4+ T cells).

Cell samples may be cells derived from a cell line, such as an immortalized cell line. Exemplary cell lines include, but are not limited to, 293A cell line, 293 FT cell line, 293F cell line, 293 H cell line, HEK 293 cell line, CHO DG44 cell line, CHO-S cell line, CHO-K1 cell line, Expi293F™ cell line, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cell line, FreeStyle™ CHO-S cell line, GripTite™ 293 MSR cell line, GS-CHO cell line, HepaRG™ cell line, T-REx™ Jurkat cell line, Per.C6 cell line, T-REX™-293 cell line, T-REx™-CHO cell line, T-REX™-HeLa cell line, NC-HIMT cell line, PC12 cell line, A549 cells, and K562 cells.

In some embodiments, an RNBD of the present disclosure can be used to modify a target cell. The target cell can itself be unmodified or modified. For example, an unmodified cell can be edited with an RNBD of the present disclosure to introduce an insertion, deletion, or mutation in its genome. In some embodiments, a modified cell already having a mutation can be repaired with an RNBD of the present disclosure.

In some instances, a target cell is a cell comprising one or more single nucleotide polymorphism (SNP). In some instances, an RNBD-nuclease described herein is designed to target and edit a target cell comprising a SNP.

In some cases, a target cell is a cell that does not contain a modification. For example, a target cell can comprise a genome without genetic defect (e.g., without genetic mutation) and an RNBD-nuclease described herein can be used to introduce a modification (e.g., a mutation) within the genome.

The cell sample may be obtained from cells of a primate. The primate may be a human, or a non-human primate. The cell sample may be obtained from a human. For example, the cell sample may comprise cells obtained from blood, urine, stool, saliva, lymph fluid, cerebrospinal fluid, synovial fluid, cystic fluid, ascites, pleural effusion, amniotic fluid, chorionic villus sample, vaginal fluid, interstitial fluid, buccal swab sample, sputum, bronchial lavage, Pap smear sample, or ocular fluid. The cell sample may comprise cells obtained from a blood sample, an aspirate sample, or a smear sample.

The cell sample may be a circulating tumor cell sample. A circulating tumor cell sample may comprise lymphoma cells, fetal cells, apoptotic cells, epithelia cells, endothelial cells, stem cells, progenitor cells, mesenchymal cells, osteoblast cells, osteocytes, hematopoietic stem cells (HSC) (e.g., a CD34+HSC), foam cells, adipose cells, transcervical cells, circulating cardiocytes, circulating fibrocytes, circulating cancer stem cells, circulating myocytes, circulating cells from a kidney, circulating cells from a gastrointestinal tract, circulating cells from a lung, circulating cells from reproductive organs, circulating cells from a central nervous system, circulating hepatic cells, circulating cells from a spleen, circulating cells from a thymus, circulating cells from a thyroid, circulating cells from an endocrine gland, circulating cells from a parathyroid, circulating cells from a pituitary, circulating cells from an adrenal gland, circulating cells from islets of Langerhans, circulating cells from a pancreas, circulating cells from a hypothalamus, circulating cells from prostate tissues, circulating cells from breast tissues, circulating cells from circulating retinal cells, circulating ophthalmic cells, circulating auditory cells, circulating epidermal cells, circulating cells from the urinary tract, or combinations thereof.

The cell can be a T cell. For example, in some embodiments, the T cell can be an engineered T cell transduced to express a chimeric antigen receptor (CAR). The CAR T cell can be engineered to bind to BCMA, CD19, CD22, WT1, LICAM, MUC16, RORI, or LeY.

A cell sample may be a peripheral blood mononuclear cell sample.

A cell sample may comprise cancerous cells. The cancerous cells may form a cancer which may be a solid tumor or a hematologic malignancy. The cancerous cell sample may comprise cells obtained from a solid tumor. The solid tumor may include a sarcoma or a carcinoma. Exemplary sarcoma cell sample may include, but are not limited to, cell sample obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, plcomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma cell samples may include, but are not limited to, cell samples obtained from an anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

The cancerous cell sample may comprise cells obtained from a hematologic malignancy. Hematologic malignancy may comprise a leukemia, a lymphoma, a myeloma, a non-Hodgkin's lymphoma, or a Hodgkin's lymphoma. The hematologic malignancy may be a T-cell based hematologic malignancy. The hematologic malignancy may be a B-cell based hematologic malignancy. Exemplary B-cell based hematologic malignancy may include, but are not limited to, chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy may include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

A cell sample described herein may comprise a tumor cell line sample. Exemplary tumor cell line sample may include, but are not limited to, cell samples from tumor cell lines such as 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

A cell sample may comprise cells obtained from a biopsy sample, necropsy sample, or autopsy sample.

The cell samples (such as a biopsy sample) may be obtained from an individual by any suitable means of obtaining the sample using well-known and routine clinical methods. Procedures for obtaining tissue samples from an individual are well known. For example, procedures for drawing and processing tissue sample such as from a needle aspiration biopsy are well-known and may be employed to obtain a sample for use in the methods provided. Typically, for collection of such a tissue sample, a thin hollow needle is inserted into a mass such as a tumor mass for sampling of cells that, after being stained, will be examined under a microscope.

A cell may be a live cell. A cell may be a eukaryotic cell. A cell may be a yeast cell. A cell may be a plant cell. A cell may be obtained from an agricultural plant.

### EXAMPLES

These examples are provided for illustrative purposes only and not to limit the scope of the claims provided herein.

### Example 1

### Genome Editing Complexes and Gene Repressors

This example describes genome editing complexes and gene repressors. A Ralstonia-derived modular nucleic acid binding domain (RNBD) is engineered by encoding for a plurality of repeat units, wherein each repeat unit is selected from any combination of SEQ ID NO: 168-SEQ ID NO: 263 or SEQ ID NO: 336-SEQ ID NO: 356. RNBDs are engineered to have an N-terminus as set forth in SEQ ID NO: 264 of SEQ ID NO: 303 and a C-terminus as set forth in SEQ ID NO: 266. The RNBD is engineered to also include a half repeat as set forth in SEQ ID NO: 265, prior to the C-terminus of SEQ ID NO: 266.

Genome Editing. The RNBD is linked to a nuclease, such as FokI or any one of SEQ ID NO: 1-SEQ ID NO: 81 (nucleic acid Sequences of SEQ ID NO: 82-SEQ ID NO: 162).

Gene Regulation. The RNBD is linked to an activator (e.g., VP16, VP64, p65, p300 catalytic domain, TET1 catalytic domain, TDG, Ldb1 self-associated domain, SAM activator (VP64, p65, HSF1), or VPR (VP64, p65, Rta) or a repressor (e.g., KRAB, Sin3a, LSD1, SUV39H1, G9A (EHMT2), DNMT1, DNMT3A-DNMT3L, DNMT3B, KOX, TGF-beta-inducible early gene (TIEG), v-crbA, SID, MBD2, MBD3, Rb, or MeCP2).

### Example 2

### Mixed DNA Binding Domains

This example illustrates mixed DNA binding domains fused to nucleases to form genome editing complexes or fused to regulation domains to form gene activators or repressors. A Ralstonia-derived modular nucleic acid binding domain (RNBD) is engineered by encoding for a plurality of repeat units, wherein each repeat unit is selected from any combination of SEQ ID NO: 168-SEQ ID NO: 263 or SEQ ID NO: 336-SEQ ID NO: 356. RNBDs are engineered with an N-terminus as set forth in SEQ ID NO: 301 (Xanthomonas) or SEQ ID NO: 304 (Legionella). RNBDs are engineered with a C-terminus as set forth in SEQ ID NO: 298 (Xanthomonas) or SEQ ID NO: 306 (Legionella).

Genome Editing. The RNBD is linked to a nuclease, such as FokI or any one of SEQ ID NO: 1-SEQ ID NO: 81 (nucleic acid Sequences of SEQ ID NO: 82-SEQ ID NO: 162).

Gene Regulation. The RNBD is linked to an activator (e.g., VP16, VP64, p65, p300 catalytic domain, TET1 catalytic domain, TDG, Ldb1 self-associated domain, SAM activator (VP64, p65, HSF1), or VPR (VP64, p65, Rta) or a repressor (e.g., KRAB, Sin3a, LSD1, SUV39H1, G9A (EHMT2), DNMT1, DNMT3A-DNMT3L, DNMT3B, KOX, TGF-beta-inducible early gene (TIEG), v-crbA, SID, MBD2, MBD3, Rb, or MeCP2).

### Example 3

### Genome Editing with an RNBD Fused to a Nuclease

This example illustrates genome editing with an RNBD fused to a nuclease. A first modular Ralstonia nucleic acid binding domain (RNBD) described herein, is fused to a cleavage half domain, such as an nuclease and a second modular Ralstonia DNA binding domain (RNBD) described herein, is fused to another cleavage half domain. The nucleic acid binding domains are fused to the nuclease, optionally, via a naturally occurring linker, a variant or truncation of a naturally occurring linker, or a synthetic linker. The first RNBD-nuclease complex recognizes a target nucleic acid sequence on the top strand of double stranded DNA and binds said region of the double stranded DNA and the second RNBD-nuclease complex recognizes a target nucleic acid sequence on the bottom strand of double stranded DNA and binds said region of the double stranded DNA. The 3′ end of the target nucleic acid sequence on the top strand and the 3′ end of the target nucleic acid sequence on the bottom strand are spaced 2 to 50 base pairs apart, referred to herein as the “spacer region.” Gene editing is carried out by dimerization of the two cleavage half domains in the spacer region followed by cleaving of the DNA phosphodiester bonds. Gene editing allows for the insertion of a sequence or deletion of a sequence.

### Direct Administration to Introduce a Gene

The genome editing complex is administered directly to a subject in need thereof and is taken up by a cell. The subject has a disease. The DNA binding domain of the genome editing complex binds a region of DNA in a target cell and the cleavage domain induces a double strand break in the DNA of the target cell to introduce a gene. The introduced gene is a mutated gene or a functional gene.

Factor IX. The genome editing complex with a cleavage domain introduces a double strand break into the albumin gene locus (e.g., into intron 1) concomitant with delivery to the cell of an ectopic nucleic acid bearing a cDNA of the factor IX gene. The double strand break leads to the integration of the ectopic nucleic acid into intron 1 of the albumin gene; the factor IX protein is secreted by the cell into the circulation. The target cell is a hepatocyte and the subject in need thereof has Hemophilia B.

### Ex Vivo Engineering of a Cell to Introduce a Gene

The genome editing complex is transfected into cells ex vivo along with an ectopic nucleic acid bearing a gene. Upon transfection of cells ex vivo, the DNA binding domain of the genome editing complex binds a region of DNA in a target cell and the cleavage domain induces a double strand break in the DNA of the target cell to introduce an ectopically provided gene (also provided to the cell) into the region cleaved by the genome editing complex. The resulting engineered cells with modified DNA are administered to a subject in need thereof. The subject has a disease.

CAR. The genome editing complex with a cleavage domain introduces a chimeric antigen receptor (CAR) by editing the DNA of a target cell. The target cell is a T cell and the subject has cancer, such as a blood cancer. Upon administration of the engineered cells to a subject, the engineered CAR T cells effectively eliminate cancer in the subject.

### Direct Administration to Partially or Completely Knock Out a Gene

The genome editing complex is administered directly to a subject in need thereof and is taken up by a cell. The subject has a disease. The DNA binding domain of the genome editing complex binds a region of DNA in a target cell and the cleavage domain induces a double strand break in the DNA of the target cell to partially or completely knock out a gene.

TTR. The genome editing complex with a cleavage domain partially or completely knocks out the transthyretin (TTR) gene by editing the DNA of a target cell. The target cell is a liver cell and the subject in need thereof has transthyretin amyloidosis (ATTR).

SERPINA1. The genome editing complex with a cleavage domain partially or completely knocks out the SERPINA1 gene by editing the DNA of a target cell. The target cell is a liver cell and the subject in need thereof has alpha-1 antitrypsin deficiency (dA1AT def).

### Ex Vivo Engineering of a Cell to Partially or Completely Knock Out a Gene or a Gene Regulatory Region

The genome editing complex is transfected in cells ex vivo. Upon transfection of cells ex vivo, the DNA binding domain of the genome editing complex binds a region of DNA in a target cell and the cleavage domain induces a double strand break in the DNA of the target cell to partially or completely knock out a gene or a gene regulatory region. The subject has a disease.

BCL11A Enhancer. The genome editing complex with a cleavage domain partially or completely knocks out the BCL11A erythroid enhancer by editing the DNA of a target cell. The target cell is an HPSC and the subject in need thereof has b-thalassemia or sickle cell disease.

CCR5. The genome editing complex with a cleavage domain partially or completely knocks the CCR5 gene by editing the DNA of a target cell, thereby allowing for introduction of a mutated version of CCR5. Target cells, in which mutated versions of CCR5 are introduced via the action of the genome editing complex, are not infected by HIV via the modified CCR5 receptor. The target cell is a T cell or a hematopoietic stem cell (HPSC) and the subject has HIV.

Upon administration of the genome editing complex directly to a subject or upon administration of an engineered cell with DNA that has been modified with the genome editing complex, the disease symptoms are eliminated or reduced.

### Example 4

### Tale Protein with N-Terminus Fragment

A DNA binding protein engineered to have a shortened N-terminus derived from a TALE protein was generated. U.S. Pat. No. 8,586,526 shows that while the N-terminus region (referred to as N-cap) from a TALE protein can be shortened by deleting amino acids at the N-terminus, deleting amino acids beyond amino acid position N+134 decreased DNA binding affinity, with the decrease in DNA binding apparent even with deletion of amino acids beyond amino acid position N+137. U.S. Pat. No. 8,586,526 concluded that amino acid sequence from N+1 through N+137 are required for binding to DNA while the first 152 amino acids of the N-cap sequence are dispensable.

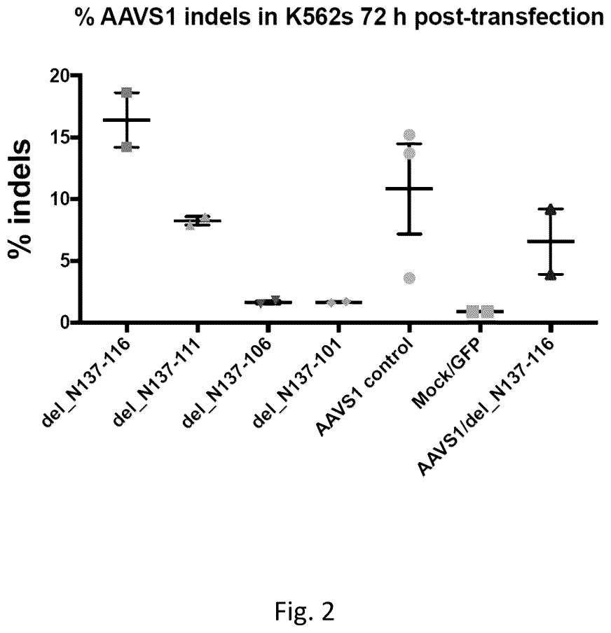

However, it has been discovered that further deleting amino acids till position N+116 surprising leads to recovery of DNA binding. Even shorter N-terminus regions such as a fragment having deletion till position N+111 also retains DNA binding activity. Deleting amino acids till position N+106 significantly decreases DNA binding. Further deletion of the N-terminus region, such as, deleting amino acids till position N+101 does not lead to recovery of DNA binding. See FIG. 2.

TALEN monomers recognizing 5′-TTTCTGTCACCAATCCT-3′ and 5′-TCCCCTCCACCCCACAGT-3′ in the human AAVS1 locus were engineered to harbor N-terminus regions that included deletions encompassing residues N137-116, N137-111, N137-106 and N137-101. While these residues are numbered with reference to the N+137 construct in U.S. Pat. No. 8,586,526, N137-116 refers to deletion of amino acids starting at the N-terminus of the N-cap sequence (N+228) and extending through amino acid residue 116 such that the resulting fragment retains amino acids residues from position N+115 to position N+1, and so on. The amino acid sequence of the N-terminal truncation del_N137-116 is set forth in SEQ ID NO:321. The amino acid sequence of the N-terminal truncation del_N137-111 is set forth in SEQ ID NO:447.

NK562 cells were transfected with 2 μg plasmid DNA for each TALEN monomer using an AMAXA™ Nucleofector™ 96-well Shuttle™ system as per the manufacturer's recommendations. Full length TALEN monomers were included (“AAVS1 control”), together with N137-116/full length and full length/N137-116 heterodimers. Cells were cold shocked at 30° C. and genomic DNA was harvested at 72 h using QuickExtract™ (Lucigen). Indel rates were determined by amplicon sequencing. The TALE repeats present in the TALE monomers have the sequence LTPDQVVAIAS (RVD) GGKQALETVQRLLPVLCQDHG, with a RVD selected based on the target sequence.

FIG. 2 represents DNA binding activity assayed by measuring nuclease activity of Fok1 fused to C-terminus of the polypeptides. AAVS1 control data set correspond to TALENS using the standard full-length N-terminus (N+288 to N+1). N-terminal truncation del_N137-116 (N-terminus extending from N+115 to N+1) showed higher activity than standard full-length N-terminus (N+288 to N+1). N-terminal truncation del_N137-111 (N-terminus extending from N+110 to N+1) was also active. Further truncation del_N137-106 (N-terminus extending from N+105 to N+1) significantly decreased DNA binding. Further deletion of the N-terminus region del_N137-101 (N-terminus extending from N+100 to N+1) did not lead to recovery of DNA binding. Thus, a fragment of the N-terminus of a TALE protein extending from N+115 to N+1 shows full activity. Mock/GFP is a negative control. The AAVS1/del_N137-116 data shows that an N1-115 TALEN monomer can be combined with a monomer comprising full-length N-terminus region of a TALE protein.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-104. (canceled)

105. A non-naturally occurring DNA-binding polypeptide comprising from N-terminus to C-terminus:
an N-terminus region comprises at least residues N+110 to N+1 of a Xanthomonas Transcription Activator-Like Effector (TALE) protein, wherein the N-terminus region does not include residues N+288 to N+116 of the TALE protein;
a plurality of Xanthomonas TALE-repeat units, the TALE repeat units comprising a repeat variable di-residue (RVD), wherein the TALE repeat units are ordered from N-terminus to C-terminus to specifically bind to a target nucleic acid in genomic DNA; and
a C-terminus region of the TALE protein.

106. The DNA binding polypeptide of claim 105, wherein the N-terminus region comprises residues N+1 up to N+115 of the TALE protein.

107. The DNA binding polypeptide of claim 105, wherein the N-terminus region comprises residues N+1 up to N+110 of the TALE protein.

108. The DNA binding polypeptide of claim 105, wherein the C-terminus region comprises residues C+1 to C+63 of the TALE protein.

109. The DNA binding polypeptide of claim 105, wherein the N-terminus region consists of residues N+1 to N+115 of the TALE protein.

110. The DNA binding polypeptide of claim 105, wherein a heterologous functional domain is conjugated to the N-terminus and/or C-terminus.

111. The DNA binding polypeptide of claim 110, wherein the functional domain comprises a fluorophore, a detectable tag, an enzyme, a transcriptional activator, a transcriptional repressor, or a DNA nucleotide modifier.

112. The DNA binding polypeptide of claim 111, wherein the enzyme is a DNA modifying protein or a chromatin modifying protein.

113. The DNA binding polypeptide of claim 112, wherein the chromatin modifying protein is lysine-specific histone demethylase 1 (LSD1), and the DNA nucleotide modifier is adenosine deaminase.

114. The DNA binding polypeptide of claim 111, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).

115. The DNA binding polypeptide of claim 111, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

116. The DNA binding polypeptide of claim 105, wherein the target nucleic acid is within a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEAl gene, a TTR gene, a NR3CI gene, a CD52 gene, an erythroid specific enhancer of the ECLIIA gene, a CELE gene, a TGFERI gene, a SERPINAI gene, a HEV genomic DNA in infected cells, a CEP290 gene, a DMD gene, a CFTR gene, or an IL2RG gene.

117. A nucleic acid encoding the polypeptide of claim 105 or a vector comprising a nucleic acid encoding the polypeptide of claim 105.

118. A host cell comprising the nucleic acid or the vector of claim 117.

119. A pharmaceutical composition comprising the polypeptide of claim 105 or the nucleic acid or vector of claim 117 and a pharmaceutically acceptable excipient.

120. A method of modulating expression of an endogenous gene in a cell, the method comprising:
introducing into the cell the polypeptide of claim 105,
wherein the DNA binding polypeptide binds to a target nucleic acid sequence present in the endogenous gene and the heterologous functional domain modulates expression of the endogenous gene.

121. The method of claim 120, wherein the polypeptide is introduced as a nucleic acid encoding the polypeptide.


SEQUENCE LISTING (451 sequences)

SEQ ID NO: 1 - protein, 196 aa, synthetic construct
FLVKGAMEIKKSELRHKLRHVPHEYIELIEIAQDSKQNRLLEFKVVEFFKKIYGYRGKHLGGSRKPDGALFTDGLVLNHGIILDTKAYKDGYRLPISQADEMQRYVDENNKRSQVINPNEWWEIYPTSITDFKFLFVSGFFQGDYRKQLERVSHLTKCQGAVMSVEQLLLGGEKIKEGSLTLEEVGKKFKNDEIVF

SEQ ID NO: 2 - protein, 192 aa, synthetic construct
QIVKSSIEMSKANMRDNLQMLPHDYIELIEISQDPYQNRIFEMKVMDLFINEYGFSGSHLGGSRKPDGAMYAHGFGVIVDTKAYKDGYNLPISQADEMERYVRENIDRNEHVNSNRWWNIFPEDTNEYKFLFVSGFFKGNFEKQLERISIDTGVQGGALSVEHLLLGAEYIKRGILTLYDFKNSFLNKEIQF

SEQ ID NO: 3 - protein, 196 aa, synthetic construct
QTIKSSIEELKSELRTQLNVISHDYLQLVDISQDSQQNRLFEMKVMDLFINEFGYNGSHLGGSRKPDGILYTEGLSKDYGIIVDTKAYKDGYNLPIAQADEMERYIRENIDRNEVVNPNRWWEVFPSKINDYKFLFVSAYFKGNFKEQLERISINTGILGGAISVEHLLLGAEYFKRGILSLEDVRDKFCNTEIEF

SEQ ID NO: 4 - protein, 195 aa, synthetic construct
GKSEVETIKEQMRGELTHLSHEYLGLLDLAYDSKQNRLFELKTMQLLTEECGFEGLHLGGSRKPDGIVYTKDENEQVGKENYGIIIDTKAYSGGYSLPISQADEMERYIGENQTRDIRINPNEWWKNFGDGVTEYYYLFVAGHFKGKYQEQIDRINCNKNIKGAAVSIQQLLRIVNDYKAGKLTHEDMKLKIFHY

SEQ ID NO: 5 - protein, 151 aa, synthetic construct
MKILELLINECGYKGLHLGGARKPDGIIYTEKEKYNYGVIIDTKAYSKGYNLPIGQIDEMIRYIIENNERNIKRNTNCWWNNFEKNVNEFYFSFISGEFTGNIEEKLNRIFISTNIKGNAMSVKTLLYLANEIKANRISYIELLNYFDNKV

SEQ ID NO: 6 - protein, 194 aa, synthetic construct
AKSSQSETKEKLREKLRNLPHEYLSLVDLAYDSKQNRLFEMKVIELLTEECGFQGLHLGGSRRPDGVLYTAGLTDNYGIILDTKAYSSGYSLPIAQADEMERYVRENQTRDELVNPNQWWENFENGLGTFYFLFVAGHFNGNVQAQLERISRNTGVLGAAASISQLLLLADAIRGGRMDRERLRHLMFQNEEFL

SEQ ID NO: 7 - protein, 193 aa, synthetic construct
NSEKSEFTQEKDNLREKLDTLSHEYLSLVDLAFDSQQNRLFEMKTVELLTKECNYKGVHLGGSRKPDGIIYTENSTDNYGVIIDTKAYSNGYNLPISQVDEMVRYVEENNKREKERNSNEWWKEFGDNINKFYFSFISGKFIGNIEEKLQRITIFTNVYGNAMTIITLLYLANEIKANRLKTMEVVKYFDNKV

SEQ ID NO: 8 - protein, 200 aa, synthetic construct
NLTCSDLTEIKEEVRNALTHLSHEYLALIDLAYDSTQNRLFEMKTLQLLVEECGYQGTHLGGSRKPDGICYSEEAKSEGLEANYGIIIDTKSYSGGYGLPISQADEMERYIRENQTRDAEVNRNKWWEAFPETIDIFYFMFVAGHFKGNYFNQLERLQRSTGIKGAAVDIKTLLLTANRCKTGELDHAGIESCFFNNCRL

SEQ ID NO: 9 - protein, 193 aa, synthetic construct
DNVKSNFNQEKDELREKLDTLSHEYLYLLDLAYDSKQNKLFEMKILELLINECGYRGLHLGGVRKPDGIIYTEKEKYNYGVIIDTKAYSKGYNLPIGQIDEMIRYIIENNERNIKRNTNCWWNNFEKNVNEFYFSFISGEFTGNIEEKLNRIFISTNIKGNAMSVKTLLYLANEIKANRISFLEMEKYFDNKV

SEQ ID NO: 10 - protein, 196 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFI

SEQ ID NO: 11 - protein, 191 aa, synthetic construct
ISKTNVLELKDKVRDKLKYVDNRYLALIDLAYDGTANRDFEIQTIDLLINELKFKGVRLGESRKPDGIISYDINGVIIDNKAYSSGYNLPINQADEMIRYIEENQTRDKKINPNKWWESFDDKVKDFNYLFVSSFFKGNFKNNLKHIANRTGVNGGVINVENLLYFAEELKSGRLSYVDLFKMYDNDEINI

SEQ ID NO: 12 - protein, 191 aa, synthetic construct
ISKTNVLELKDKVRDKLKYVDHRYLALIDLAYDGTANRDFEIQTIDLLINELKFKGVRLGESRKPDGIISYDINGVIIDNKAYSTGYNLPINQADEMIRYIEENQTRDKKINSNKWWESFDDKVKNFNYLFVSSFFKGNFKNNLKHIANRTGVNGGAINVENLLYFAEELKAGRLSYVDSFTMYDNDEIYV

SEQ ID NO: 13 - protein, 193 aa, synthetic construct
KAEKSEFLIEKDKLREKLDTLPHDYLSMVDLAYDSKQNRLFEMKTIELLINECNYKGLHLGGTRKPDGIVYTNNEVENYGIIIDTKAYSKGYNLPISQVDEMTRYVEENNKREKKRNPNEWWNNFDSNVKKFYFSFISGKFVGNIEEKLQRITLFTEIYGNAITVTTLLYIANEIKANRMKKSDIMEYFNDKV

SEQ ID NO: 14 - protein, 191 aa, synthetic construct
ISKTNVLELKDKVRDKLKYVDHRYLALIDLAYDGTANRDFEIQTIDLLINELKFKGVRLGESRKPDGIISYNINGVIIDNKAYSTGYNLPINQADEMIRYIEENQTRDEKINSNKWWESFDDEVKDFNYLFVSSFFKGNFKNNLKHIANRTGVNGGAINVENLLYFAEELKAGRLSYVDSFTMYDNDEIYV

SEQ ID NO: 15 - protein, 191 aa, synthetic construct
ISKTNILELKDKVRDKLKYVDHRYLALIDLAYDGTANRDFEIQTIDLLINELKFKGVRLGESRKPDGIISYNINGVIIDNKAYSTGYNLPINQADEMIRYIEENQTRDEKINSNKWWESFDEKVKDFNYLFVSSFFKGNFKNNLKHIANRTGVNGGAINVENLLYFAEELKAGRISYLDSFKMYNNDEIYL

SEQ ID NO: 16 - protein, 191 aa, synthetic construct
ISKTNVLELKDKVRDKLKYVDHRYLALIDLAYDGTANRDFEIQTIDLLINELKFKGVRLGESRKPDGIISYNINGVIIDNKAYSTGYNLPINQADEMIRYIEENQTRDEKINSNKWWESFDDKVKDFNYLFVSSFFKGNFKNNLKHIANRTGVSGGAINVENLLYFAEELKAGRLSYVDSFKMYDNDEIYV

SEQ ID NO: 17 - protein, 191 aa, synthetic construct
ISKTNVLELKDKVRNKLKYVDHRYLALIDLAYDGTANRDFEIQTIDLLINELKFKGVRLGESRKPDGIISYDINGVIIDNKSYSTGYNLPINQADEMIRYIEENQTRDEKINSNKWWESFDEKVKDFNYLFVSSFFKGNFKNNLKHIANRTGVNGGAINVENLLYFAEELKSGRLSYVDSFTMYDNDEIYV

SEQ ID NO: 18 - protein, 191 aa, synthetic construct
ISKTNVLELKDKVRDKLKYVDHRYLSLIDLAYDGNANRDFEIQTIDLLINELNFKGVRLGESRKPDGIISYNINGVIIDNKAYSTGYNLPINQADEMIRYIEENQTRDEKINSNKWWESFDDKVKDFNYLFVSSFFKGNFKNNLKHIANRTGVSGGAINVENLLYFAEELKAGRLSYADSFTMYDNDEIYV

SEQ ID NO: 19 - protein, 191 aa, synthetic construct
IAKTNVLGLKDKVRDRLKYVDHRYLALIDLAYDGTANRDFEIQTIDLLINELKFKGVRLGESRKPDGIISYNVNGVIIDNKAYSKGYNLPINQADEMIRYIEENQTRDEKINANKWWESFDDKVEEFSYLFVSSFFKGNFKNNLKHIANRTGVNGGAINVENLLYFAEELKSGRLSYMDSFSLYDNDEICV

SEQ ID NO: 20 - protein, 193 aa, synthetic construct
ELKDEQSEKRKAKFLKETKLPMKYIELLDIAYDGKRNRDFEIVTMELFREVYRLNSKLLGGGRKPDGLIYTDDFGVIVDTKAYGEGYSKSINQADEMIRYIEDNKRRDEKRNPIKWWESFPSSISQNNFYFLWVSSKFVGKFQEQLAYTANETQTKGGAINVEQILIGADLIMQKMLDINTIPSFFENQEIIF

SEQ ID NO: 21 - protein, 191 aa, synthetic construct
IFKTNVLELKDSIREKLDYIDHRYLSLVDLAYDSKANRDFEIQTIDLLINELDFKGLRLGESRKPDGIISYDINGVIIDNKAYSKGYNLPINQADEMIRYIQENQSRNEKINPNKWWENFEDKVIKFNYLFISSLFVGGFKKNLQHIANRTGVNGGAIDVENLLYFAEEIKSGRLTYKDSFSRYINDEIKM

SEQ ID NO: 22 - protein, 193 aa, synthetic construct
LPVKSEVSVFKDYLRTHLTHVDHRYLILVDLGFDGSSDRDYEMKTAELFTAELGFMGARLGDTRKPDVCVYHGANGLIIDNKAYGKGYSLPIKQADEIYRYIEENKERDARLNPNQWWKVFDESVTHFRFAFISGSFTGGFKDRIELISMRSGICGAAVNSVNLLLMAEELKSGRLDYEEWFQYFDCNDEISF

SEQ ID NO: 23 - protein, 193 aa, synthetic construct
ISVKSDMAVVKDSVRERLAHVSHEYLILIDLGFDGTSDRDYEIQTAELFTRELDFLGGRLGDTRKPDVCIYYGKDGMIIDNKAYGKGYSLPIKQADEMYRYLEENKERNEKINPNRWWKVFDEGVTDYRFAFVSGSFTGGFKDRLENIHMRSGLCGGAIDSVTLLLLAEELKAGRMEYSEFFRLFDCNDEVTF

SEQ ID NO: 24 - protein, 193 aa, synthetic construct
ELKDKAADAVKAKFLKLTGLSMKYIELLDIAYDSSRNRDFEILTADLFKNVYGLDAMHLGGGRKPDAIAQTSHFGIIIDTKAYGNGYSKSISQEDEMVRYIEDNQQRSITRNSVEWWKNFNSSIPSTAFYFLWVSSKFVGKFDDQLLATYNRTNTCGGALNVEQLLIGAYKVKAGLLGIGQIPSYFKNKEIAW

SEQ ID NO: 25 - protein, 193 aa, synthetic construct
ISVKSDMAVVKDSVRERLAHVSHEYLLLIDLGFDGTSDRDYEIQTAELLTRELDFLGGRLGDTRKPDVCIYYGKDGMIIDNKAYGKGYSLPIKQADEMYRYLEENKERNEKINPNRWWKVFDEGVTDYRFAFVSGSFTGGFKDRLENIHMRSGLCGGAIDSVTLLLLAEELKAGRMEYSEFFRLFDCNDEVTF

SEQ ID NO: 26 - protein, 193 aa, synthetic construct
ELKDEQAEKRKAKFLKETNLPMKYIELLDIAYDGKRNRDFEIVTMELFRNVYRLHSKLLGGGRKPDGLLYQDRFGVIVDTKAYGKGYSKSINQADEMIRYIEDNKRRDENRNPIKWWEAFPDTIPQEEFYFMWVSSKFIGKFQEQLDYTSNETQIKGAALNVEQLLLGADLVLKGQLHISDLPSYFQNKEIEF

SEQ ID NO: 27 - protein, 195 aa, synthetic construct
RNLDNVERDNRKAEFLAKTSLPPRFIELLSIAYESKSNRDFEMITAELFKDVYGLGAVHLGNAKKPDALAFNDDFGIIIDTKAYSNGYSKNINQEDEMVRYIEDNQIRSPDRNNNEWWLSFPPSIPENDFHFLWVSSYFTGRFEEQLQETSARTGGTTGGALDVEQLLIGGSLIQEGSLAPHEVPAYMQNRVIHF

SEQ ID NO: 28 - protein, 193 aa, synthetic construct
SPVKSEVSVFKDYLRTHLTHVDHRYLILVDLGFDGSSDRDYEMKTAELFTAELGFMGARLGDTRKPDVCVYHGAHGLIIDNKAYGKGYSLPIKQADEIYRYIEENKERAVRLNPNQWWKVFDESVAHFRFAFISGSFTGGFKDRIELISMRSGICGAAVNSVNLLLMAEELKSGRLNYEEWFQYFDCNDEISL

SEQ ID NO: 29 - protein, 193 aa, synthetic construct
TLVDIEKERKKAYFLKETSLSPRYIELLEIAFDPKRNRDFEVITAELLKAGYGLKAKVLGGGRRPDGIAYTKDYGLIVDTKAYSNGYGKNIGQADEMIRYIEDNQKRDNKRNPIEWWREFEVQIPANSYYYLWVSGRFTGRFDEQLVYTSSQTNTRGGALEVEQLLWGADAVMKGKLNVSDLPKYMNNSIIKL

SEQ ID NO: 30 - protein, 193 aa, synthetic construct
ELRDKVIEEQKAIFLQKTKLPLSYIELLEIARDGKRSRDFELITIELFKNIYKINARILGGARKPDGVLYMPEFGVIVDTKAYADGYSKSIAQADEMIRYIEDNKRRDPSRNSTKWWEHFPTSIPANNFYFLWVSSVFVNKFHEQLSYTAQETQTVGAALSVEQLLLGADSVLKGNLTTEKFIDSFKNQEIVF

SEQ ID NO: 31 - protein, 193 aa, synthetic construct
GATKSDLSLLKDDIRKKLNHINHKYLVLIDLGFDGTADRDYELQTADLLTSELAFKGARLGDSRKPDVCVYHDKNGLIIDNKAYGSGYSLPIKQADEMLRYIEENQKRDKALNPNEWWTIFDDAVSKFNFAFVSGEFTGGFKDRLENISRRSYTNGAAINSVNLLLLAEEIKSGRISYGDAFTKFECNDEIII

SEQ ID NO: 32 - protein, 193 aa, synthetic construct
ELRNAALDKQKVNFINKTGLPMKYIELLEIAFDGSRNRDFEMVTADLFKNVYGFNSILLGGGRKPDGLIFTDRFGVIIDTKAYGNGYSKSIGQEDEMVRYIEDNQLRDSNRNSVEWWKNFDEKIESENFYFMWISSKFIGQFSDQLQSTSDRTNTKGAALNVEQLLLGAAAARDGKLDINSLPIYMNNKEILW

SEQ ID NO: 33 - protein, 193 aa, synthetic construct
ELKDEQSEKRKAYFLKETNLPLKYIELLDIAYDGKRNRDFEIVTMELFRNVYRLQSKLLGGVRKPDGLLYKHRFGIIVDTKAYGEGYSKSISQADEMIRYIEDNKRRDENRNSTKWWEHFPDCIPKQSFYFMWVSSKFVGKFQEQLDYTANETKTNGAALNVEQLLWGADLVAKGKLDISQLPSYFQNKEIEF

SEQ ID NO: 34 - protein, 188 aa, synthetic construct
HNNKFKNYLRENSELSFKFIELIDIAYDGNRNRDMEIITAELLKEIYGLNVKLLGGGRKPDILAYTDDIGIIIDTKAYKDGYGKQINQADEMIRYIEDNQRRDLIRNPNEWWRYFPKSISKEKIYFMWISSYFKNNFYEQVQYTAQETKSIGAALNVRQLLLCADAIQKEVLSLDTFLGSFRNEEINL

SEQ ID NO: 35 - protein, 193 aa, synthetic construct
LPVKSEVSILKDYLRSHLTHIDHKYLILVDLGYDGTSDRDYEIQTAQLLTAELSFLGGRLGDTRKPDVCIYYEDNGLIIDNKAYGKGYSLPMKQADEMYRYIEENKERSELLNPNCWWNIFDKDVKTFHFAFLSGEFTGGFRDRLNHISMRSGMRGAAVNSANLLIMAEKLKAGTMEYEEFFRLFDTNDEILF

SEQ ID NO: 36 - protein, 193 aa, synthetic construct
LPVKSQVSILKDYLRSYLSHVDHKYLILLDLGFDGTSDRDYEIWTAQLLTAELSFLGGRLGDTRKPDVCIYYEDNGLIIDNKAYGKGYSLPIKQADEMYRYIEENKERSDLLNPNCWWNIFGEGVKTFRFAFLSGEFTGGFKDRLNHISMRSGIKGAAVNSANLLIMAEQLKSGTMSYEEFFQLFDYNDEIIF

SEQ ID NO: 37 - protein, 191 aa, synthetic construct
VSKTNILELKDNTREKLVYLDHRYLSLFDLAYDDKASRDFEIQTIDLLINELQFKGLRLGERRKPDGIISYGVNGVIIDNKAYSKGYNLPIRQADEMIRYIQENQSRDEKLNPNKWWENFEEETSKFNYLFISSKFISGFKKNLQYIADRTGVNGGAINVENLLCFAEMLKSGKLEYNDFFNQYNNDEIIM

SEQ ID NO: 38 - protein, 193 aa, synthetic construct
LPVKSQVSILKDYLRSCLSHVDHKYLILLDLGFDGTSDRDYEIQTAQLLTAELSFLGGRLGDTRKPDVCIYYEDNGLIIDNKAYGKGYSLPIKQADEMYRYIEENKERSELLNPNCWWNIFDEGVKTFRFAFLSGEFTGGFKDRLNHISMRSGIKGAAVNSANLLIIAEQLKSGTMSYEEFFQLFDQNDEITV

SEQ ID NO: 39 - protein, 193 aa, synthetic construct
MSSKSEISVIKDNIRKRLNHINHKYLVLIDLGFDGTADRDYELQTADLLTSELSFKGARLGDTRKPDVCVYHGTNGLIIDNKAYGKGYSLPIKQADEMLRYIEENQKRDKSLNPNEWWTIFDDAVSKFNFAFVSGEFTGGFKDRLENISRRSSVNGAAINSVNLLLLAEEIKSGRMSYSDAFKNFDCNKEITI

SEQ ID NO: 40 - protein, 195 aa, synthetic construct
RNLDKVERDSRKAEFLAKTSLPPRFIELLSIAYESKSNRDFEMITAEFFKDVYGLGAVHLGNARKPDALAFTDNFGIVIDTKAYSNGYSKNINQEDEMVRYIEDNQIRSPERNKNEWWLSFPPSIPENNFHFLWVSSYFTGYFEEQLQETSDRAGGMTGGALDIEQLLIGGSLVQEGKLAPHDIPEYMQNRVIHF

SEQ ID NO: 41 - protein, 193 aa, synthetic construct
APVKSEVSLCKDILRSHLTHVDHKYLILLDLGFDGTSDRDYEIQTAQLLTAELDFKGARLGDTRKPDVCVYYGEDGLILDNKAYGKGYSLPIKQADEMYRYIEENKERNERLNPNKWWEIFDKDVVRYHFAFVSGTFTGGFKERLDNIRMRSGICGAAVNSMNLLLMAEELKSGRLGYKECFALFDCNDEIAF

SEQ ID NO: 42 - protein, 201 aa, synthetic construct
SCVKDEVNDIVDRVRVKLKNIDHKYLILISLAYSDETERTKKNSDARDFEIQTAELFTKELGFNGIRLGESNKPDVLISFGANGTIIDNKSYKDGFNIPRVTSDQMIRYINENNQRTTQLNPNEWWKNFDSSVSNYTFLFVTSFLKGSFKNQIEYISNATNGTRGAAINVESLLYISEDIKSGKIKQSDFYSEFKNDEIVY

SEQ ID NO: 43 - protein, 193 aa, synthetic construct
SQGDKAREQLKAKFLAKTNLLPRYVELLDIAYDSKRNRDFEMVTAELFNFAYLLPAVHLGGVRKPDALVATKKFGIIVDTKAYANGYSRNANQADEMARYITENQKRDPKTNPNRWWDNFDARIPPNAYYFLWVSSFFTGQFDDQLSYTAHRTNTHGGALNVEQLLIGANMIQTGQLDRNKLPEYMQDKEITF

SEQ ID NO: 44 - protein, 193 aa, synthetic construct
KVQKSNILDVIEKCREKINNIPHEYLALIPMSFDENESTMFEIKTIELLTEHCKFDGLHCGGASKPDGLIYSEDYGVIIDTKSYKDGFNIQTPERDKMKRYIEENQNRNPQHNKTRWWDEFPHNISNFLFLFVSGKFGGNFKEQLRILSEQTNNTLGGALSSYVLLNIAEQIAINKIDHCDFKTRISCLDEVA

SEQ ID NO: 45 - protein, 193 aa, synthetic construct
VPVKSEVSLCKDYLRSYLTHVDHKYLILLDLGFDGTSDRDYEIQTAQLLTAELDFKGARLGDTRKPDVCVYYGEDGLIIDNKAYGKGYSLPIKQADEIYRYIEENKKRDEKLNPNKWWEIFDKGVVRYHFAFVSGAFTGGFKERLDNIRMRSGICGAAINSMNLLLMAEELKSGRLGYEECFALFDCNDEITF

SEQ ID NO: 46 - protein, 193 aa, synthetic construct
VPVKSEVSLCKDYLRSHLNHVDHRYLILLDLGFDGTSDRDYEIQTAQLLTGELNFKGARLGDTRKPDVCVYYGEDGLIIDNKAYGKGYSLPIKQADEMYRYIEENKERNEKLNPNKWWEIFDKDVIHYHFAFVSGAFTGGFKERLENIRMRSGIYGAAVNSMNLLLMAEELKSGRLDYKECFKLFDCNDEIVL

SEQ ID NO: 47 - protein, 193 aa, synthetic construct
VPVKSEVSLLKDYLRSHLVHVDHKYLVLLDLGFDGTSDRDYEIQTAQLLTGELNFKGARLGDTRKPDVCVYYGEDGLIIDNKAYGKGYSLPIKQADEMYRYIEENKERNEKLNPNKWWEIFGNDVIHYHFAFVSGAFTGGFKERLDNIRMRSGIYGAAVNSMNLLLLAEELKSGRLGYKECFKLFDCNDEIVL

SEQ ID NO: 48 - protein, 201 aa, synthetic construct
ECVKDNVVDIKDRVRNKLIHLDHKYLALIDLAYSDAASRAKKNADAREFEIQTADLFTKELSFNGQRLGDSRKPDVIISYGLDGTIVDNKSYKDGFNISRTCADEMSRYINENNLRQKSLNPNEWWKNFDSTITAYTFLFITSYLKGQFEDQLEYVSNANGGIKGAAIGVESLLYLSEGIKAGRISHADFYSNFNNKEMIY

SEQ ID NO: 49 - protein, 192 aa, synthetic construct
IAKSDFSIIKDNIRRKLQYVNHKYLLLIDLGFDSDSNRDYEIQTAELLTTELAFKGARLGDTRKPDVCVYYGENGLIIDNKAYSKGYSLPMSQADEMVRYIEENKARQSSINPNQWWKIFEDTVCNFNYAFVSGEFTGGFKDRLNNICERTRVSGGAINTINLLLLAEELKSGRMSYPKCFSYFDTNDEVHI

SEQ ID NO: 50 - protein, 166 aa, synthetic construct
LKYLGIKKQNRAFEIITAELFNTSYKLSATHLGGGRRPDVLVYNDNFGIIVDTKAYKDGYGRNVNQEDEMVRYITENNIRKQDINKNDWWKYFSKSIPSTSYYHLWISSQFVGMFSDQLRETSSRTGENGGAMNVEQLLIGANQVLNNVLDPNCLPKYMENKEIIF

SEQ ID NO: 51 - protein, 193 aa, synthetic construct
VPVKSEVSLCKDYLRSHLNHVDHKYLILLDLGFDGTSDRDYEIQTAQLLTGELNFKGARLGDTRKPDVCVYYGEDGLIIDNKAYGKGYSLPIKQADEMYRYIEENKERNEKLNPNKWWEIFDKDVIHYHFAFVSGAFTGGFRERLENIRMRSGIYGAAVNSMNLLLMAEELKSGRLGYKECFKLFDCNDEIVL

SEQ ID NO: 52 - protein, 193 aa, synthetic construct
VPVKSEVSLLKDYLRTHLLHVDHRYLILLDLGFDGTSDRDYEIQTAQLLTGELNFKGARLGDTRKPDVCVYYGEDGLIIDNKAYGKGYSLPIKQADEMYRYIEENKERNEKLNPNKWWEIFDNDVIHYHFAFISGAFTGGFKERLDNIRMRSGIYGAAVNSMNLLLMAEELKSGRLGYKECFKLFDCNDEIVL

SEQ ID NO: 53 - protein, 193 aa, synthetic construct
VPVKSEVSLCKDYLRSHLNHVDHKYLILLDLGFDGTSDRDYEIQTAQLLTGELNFKGARLGDTRKPDVCVYYGEDGLIIDNKAYGKGYSLPIKQADEMYRYIEENKERNEKLNPNKWWEIFDNDVIHYHFAFVSGAFTGGFRERLENIRMRSGIYGAAVNSMNLLLMAEELKSGRLGYKECFKLFDCNDEIVL

SEQ ID NO: 54 - protein, 193 aa, synthetic construct
VPVKSEMSLLKDYLRTHLLHVDHRYLILLDLGFDGASDRDYEIQTAQLLTGELNFKGARLGDTRKPDVCVYYGEDGLIIDNKAYGKGYSLPIKQADEMYRYIEENKERNEKLNPNKWWEIFDNDVIHYHFAFVSGAFTGGFKERLDNIRMRSGIYGAAVNSMNLLLMAEELKSGRLGYKECFKLFDCNDEIVL

SEQ ID NO: 55 - protein, 193 aa, synthetic construct
ILVDKEREMRKAKFLKETVLDSKFISLLDLAADATKSRDFEIVTAELFKEAYNLNSVLLGGSNKPDGLVFTDDFGILLDTKAYKNGFSIYAKDRDQMIRYVDDNNKRDKIRNPNEWWKSFSPLIPNDKFYYLWVSNFFKGQFKNQIEYVNRETNTYGAVLNVEQLLYGADAVIKGIINPNKLHEYFSNDEIKF

SEQ ID NO: 56 - protein, 193 aa, synthetic construct
TVDEKERLELKEYFISNTRIPSKYITLLDLAYDGNANRDFEIVTAELFKDIFKLQSKHMGGTRKPDILIWTDKFGVIADTKAYSKGYKKNISEADKMVRYVNENTNRNKVDNTNEWWNSFDSRIPKDAYYFLWISSEFVGKFDEQLTETSSRTGRNGASINVYQLLRGADLVQKSKFNIHDLPNLMQNNEIKF

SEQ ID NO: 57 - protein, 195 aa, synthetic construct
TLQKSDIEKFKNQLRTELTNIDHSYLKGIDIASKKTTTNVENTEFEAISTKVFTDELGFFGEHLGGSNKPDGLIWDNDCAIILDSKAYSEGFPLTASHTDAMGRYLRQFKERKEEIKPTWWDIAPDNLANTYFAYVSGSFSGNYKAQLQKFRQDTNHMGGALEFVKLLLLANNYKAHKMSINEVKESILDYNISY

SEQ ID NO: 58 - protein, 194 aa, synthetic construct
VKEKTDAALVKERVRLQLHNINHKYLALIDYAFSGKNNSRDFEVYTIDLLVNELTFGGLHLGGTRKPDGIFYHGSNGIIIDNKAYAKGFVITRNMADEMIRYVQENNDRNPERNPNCWWKGFPHDVTRYNYVFISSMFKGEVEHMLDNIRQSTGIDGCVLTIENLLYYADAIKGGTLSKATFINGFNANKEMVF

SEQ ID NO: 59 - protein, 195 aa, synthetic construct
VKETTDSVIIKDRVRLKLHHVNHKYLTLIDYAFSGKNNCMDFEVYTIDLLVNELAFNGVHLGGTRKPDGIFYHNRNGIIIDNKAYSHGFTLSRAMADEMIRYIQENNDRNPERNPNKWWENFDKGVNQFNFVFISSLFKGEIEHMLTNIKQSTDGVEGCVLSAENLLYFAEAMKSGVMPKTEFISYFGAGKEIQF

SEQ ID NO: 60 - protein, 201 aa, synthetic construct
SACKADITELKDKIRKSLKVLDHKYLVLVDLAYSDASTKSKKNSDAREFEIQTADLFTKELKFDGMRLGDSNRPDVIISHDNFGTIIDNKSYKDGFNIDKKCADEMSRYINENQRRIPELPKNEWWKNFDVNVDIFTFLFITSYLKGNFKDQLEYISKSQSDIKGAAISVEHLLYISEKVKNGSMDKADFFKLFNNDEIRV

SEQ ID NO: 61 - protein, 193 aa, synthetic construct
VLKDKHLEKIKEKFLENTSLDPRFISLIEISRDKKQNRAFEIITAELFNTSYNLSAIHLGGGRRPDVLAYNDNFGIIVDTKAYKNGYGRNVNQEDEMVRYITENKIRKQDISKNNWWKYFSKSIPSTSYYHLWISSEFVGMFSDQLRETSSRTGENGGAMNVEQLLIGANQVLNNVLDPNRLPEYMENKEIIF

SEQ ID NO: 62 - protein, 193 aa, synthetic construct
ALKDKHLEKIKEKFLENTSLDPRFISLIEISRDKKQNRAFEIITAELFNTSYKLSATHLGGGRRPDVLVYNDNFGIIVDTKAYKDGYGRNVNQEDEMVRYITENNIRKQDINKNDWWKYFSKSIPSTSYYHLWISSQFVGMFSDQLRETSSRTGENGGAMNVEQLLIGANQVLNNVLDPNCLPKYMENKEIIF

SEQ ID NO: 63 - protein, 195 aa, synthetic construct
VLEKSDIEKFKNQLRTELTNIDHSYLKGIDIASKKKTSNVENTEFEAISTKIFTDELGFSGKHLGGSNKPDGLLWDDDCAIILDSKAYSEGFPLTASHTDAMGRYLRQFTERKEEIKPTWWDIAPEHLDNTYFAYVSGSFSGNYKEQLQKFRQDTNHLGGALEFVKLLLLANNYKTQKMSKKEVKKSILDYNISY

SEQ ID NO: 64 - protein, 193 aa, synthetic construct
AEADVTSEKIKNHFRRVTELPERYLELLDIAFDHKRNRDFEMVTAGLFKDVYGLESVHLGGANKPDGVVYNDNFGIILDTKAYENGYGKHISQIDEMVRYIDDNRLRDTTRNPNKWWENFDADIPSDQFYYLWVSGKFLPNFAEQLKQTNYRSHANGGGLEVQQLLLGADAVKRRKLDVNTIPNYMKNEVITL

SEQ ID NO: 65 - protein, 193 aa, synthetic construct
AEADLNSEKIKNHYRKITNLPEKYIELLDIAFDHRRHQDFEIVTAGLFKDCYGLSSIHLGGQNKPDGVVFNNKFGIILDTKAYEKGYGMHIGQIDEMCRYIDDNKKRDIVRQPNEWWKNFGDNIPKDQFYYLWISGKFLPRFNEQLKQTHYRTSINGGGLEVSQLLLGANAAMKGKLDVNTLPKHMNNQVIKL

SEQ ID NO: 66 - protein, 203 aa, synthetic construct
VLKDAALQKTKNTLLNELTEIDPADIEVIEMSWKKATTRSQNTLEATLFEVKVVEIFKKYFELNGEHLGGQNRPDGAVYYNSTYGIILDTKAYSNGYNIPVDQQREMVDYITDVIDKNQNVTPNRWWEAFPATLLKNNIYYLWVAGGFTGKYLDQLTRTHNQTNMDGGAMTTEVLLRLANKVSSGNLKTTDIPKLMTNKLILS

SEQ ID NO: 67 - protein, 192 aa, synthetic construct
AEADLDSERIKNHYRKITNLPEKYIELLDIAFDHHRHQDFEIITAGLFKDCYGLSSIHLGGQNKPDGVVFNGKFGIILDTKAYEKGYGMHINQIDEMCRYIEDNKQRDKIRQPNEWWNNFGDNIPENKFYYLWVSGKFLPKFNEQLKQTHYRTGINGGGLEVSQLLLGADAVMKGALNVNILPTYMHNNVIQ

SEQ ID NO: 68 - protein, 193 aa, synthetic construct
EISDIALQKEKAYFYKNTALSKRHISILEIAFDGSKNRDLEILSAEVFKDYYQLESIHLGGGLKPDGIAFNQNFGIIVDTKAYKGVYSRSRAEADKMFRYIEDNKKRDPKRNQSLWWRSFNEHIPANNFYFLWISGKFQRNFDTQINQLNYETGYRGGALSARQFLIGADAIQKGKIDINDLPSYFNNSVISF

SEQ ID NO: 69 - protein, 193 aa, synthetic construct
TSREKSRLNLKEYFVSNTNLPNKFITLLDLAYDGKANRDFELITSELFREIYKLNTRHLGGTRKPDILIWNENFGIIADTKAYSKGYKKNISEEDKMVRYIDENIKRSKDYNPNEWWKVFDNEISSNNYFYLWISSEFIGKFEEQLQETAQRTNVKGASINVYQLLMGAHKVQTKELNVNSIPKYMNNTEIKF

SEQ ID NO: 70 - protein, 201 aa, synthetic construct
NCIKDSIIDIKDRVRTKLVHLDHKYLALIDLAFSDADTRTKKNSDAREFEIQTADLFTKELSFNGQRLGDSRKPDIIISFDKIGTIIDNKSYKDGFNISRPCADEMIRYINENNLRKKSLNANEWWNKFDPTITAYSFLFITSYLKGQFQEQLEYISNANGGIKGAAIGIENLLYLSEALKSGKISHKDFYQNFNNKEITY

SEQ ID NO: 71 - protein, 197 aa, synthetic construct
LPQKDQVQQQQDELRPMLKNVDHRYLQLVELALDSDQNSEYSQFEQLTMELVLKHLDFDGKPLGGSNKPDGIAWDNDGNFIIFDTKAYNKGYSLAGNTDKVKRYIDDVRDRDTSRTSTWWQLVPKSIDVHNLLRFVYVSGNFTGNYMKLLDSLRSWSNAQGGLASVEKLLLTSELYLRNMYSHQELIDSWTDNNVKH

SEQ ID NO: 72 - protein, 189 aa, synthetic construct
TTDAVVVKDRARVRLHNINHKYLTLIDYAFSGKNNCTEFEIYTIDLLVNELAFNGIHLGGTRKPDGIFDYNQQGIIIDNKAYSKGFTITRSMADEMVRYVQENNDRNPERNKTQWWLNFGDNVNHFNFVFISSMFKGEVRHMLNNIKQSTGVDGCVLTAENLLYFADAIKGGTVKRTDFINLFGKNDEL

SEQ ID NO: 73 - protein, 198 aa, synthetic construct
LPKKDNVQRQQDELRPLLKHVDHRYLQLVELALDSSQNSEYSMLESMTMELLLTHLDFDGASLGGASKPDGIAWDKDGNFLIVDTKAYDNGYSLAGNTDKVARYIDDVRAKDPNRASTWWTQVPESLNVDDNLSFMYVSGSFTGNYQRLLKDLRARTNARGGLTTVEKLLLTSEAYLAKSGYGHTQLLNDWTDDNIDH

SEQ ID NO: 74 - protein, 193 aa, synthetic construct
QIKDKYLEDLKLELYKKTNLPNKYYEMVDIAYDGKRNREFEIYTSDLMQEIYGFKTTLLGGTRKPDVVSYSDAHGYIIDTKAYANGYRKEIKQEDEMVRYIEDNQLKDVLRNPNKWWECFDDAEHKKEYYFLWISSKFVGEFSSQLQDTSRRTGIKGGAVNIVQLLLGAHLVYSGEISKDQFAAYMNNTEINF

SEQ ID NO: 75 - protein, 146 aa, synthetic construct
MNPRNEIVIAKHLSGGNRPEIVCYHPEDKPDHGLILDSKAYKSGFTIPSGERDKMVRYIEEYITKNQLQNPNEWWKNLKGAEYPGIVGFGFISNSFLGHYRKQLDYIMRRTKIKGSSITTEHLLKTVEDVLSEKGNVIDFFKYFLE

SEQ ID NO: 76 - protein, 195 aa, synthetic construct
EIKNQEIEELKQIALNKYTALPSEWVELIEISRDKDQSTIFEMKVAELFKTCYRIKSLHLGGASKPDCLLWDDSFSVIVDAKAYKDGFPFQASEKDKMVRYLRECERKDKAENATEWWNNFPPELNSNQLFFMFASSFFSSTAEKHLESVSIASKFSGCAWDVDNLLSGANFFLQNPQATLQYHLIRVFSNKVVD

SEQ ID NO: 77 - protein, 198 aa, synthetic construct
LPHKDNVIKQQDELRPMLKHVNHKYLQLVELAFESSRNSEYSQFETLTMELVLKYLDFSGKSLGGANKPDGIAWDPLGNFLIFDTKAYKHGYTLSNNTDRVARYINDVRDKDIQRISRWWQSIPTYIDVKNKLQFVYISGSFTGHYLRLLNDLRSRTRAKGGLVTVEKLLLTTERYLAEADYTHKELFDDWMDDNIEH

SEQ ID NO: 78 - protein, 200 aa, synthetic construct
RISPSNLEQTKQQLREELINLDHQYLDILDFSIAGNVGARQFEVRIVELLNEIIIAKHLSGGNRPEIIGFNPKENPEDCIIMDSKAYKEGFNIPANERDKMIRYVEEYNAKDNTLNNNKWWKNFESPNYPTNQVKFSFVSSSFIGQFTNQLTYINNRTNVNGSAITAETLLRKVENVMNVNTEYNLNNFFEELGSNTLVA

SEQ ID NO: 79 - protein, 194 aa, synthetic construct
TFDSTVADNLKNLILPKLKELDHKYLQAIDIAYKRSNTTNHENTLLEVLSADLFTKEMDYHGKHLGGANKPDGFVYDEETGWILDSKAYRDGFAVTAHTTDAMGRYIDQYRDRDDKSTWWEDFPKDLPQTYFAYVSGFYIGKYQEQLQDFENRKHMKGGLIEVAKLILLAEKYKENKITHDQITLQILNDHISQ

SEQ ID NO: 80 - protein, 186 aa, synthetic construct
PLDVVEQMKAELRPLLNHVNHRLLAIIDFSYNMSRGDDKRLEDYTAQIYKLISHDTHLLAGPSRPDVVSVINDLGIIIDSKAYKQGFNIPQAEEDKMVRYLDESIRRDPAINPTKWWEYLGASTEYVFQFVSSSFSSGASAKLRQIHRRSSIEGSIITAKNLLLLAENFLCTNTINIDLFRQNNEI

SEQ ID NO: 81 - protein, 201 aa, synthetic construct
QLVPSYITQTKLRLSGLINYIDHSYFDLIDLGFDGRQNRLYELRIVELLNLINSLKALHLSGGNRPEIIAYSPDVNPINGVIMDSKSYRGGFNIPNSERDKMIRYINEYNQKNPTLNSNRWWENFRAPDYPQSPLKYSFVSGNFIGQFLNQIQYILTQTGINGGAITSEKLIEKVNAVLNPNISYTINNFFNDLGCNRLVQ

SEQ ID NO: 82 - DNA, 588 bp, synthetic construct
  source 1..588: mol_type = other DNA
ttcctggtgaagggcgccatggagatcaagaagagcgagctgaggcacaagctgaggcacgtgccccacgagtacatcgagctgatcgagatcgcccaggacagcaagcagaacaggctgctggagttcaaggtggtggagttcttcaagaagatctacggctacaggggcaagcacctgggcggcagcaggaagcccgacggcgccctgttcaccgacggcctggtgctgaaccacggcatcatcctggacaccaaggcctacaaggacggctacaggctgcccatcagccaggccgacgagatgcagaggtacgtggacgagaacaacaagaggagccaggtgatcaaccccaacgagtggtgggagatctaccccaccagcatcaccgacttcaagttcctgttcgtgagcggcttcttccagggcgactacaggaagcagctggagagggtgagccacctgaccaagtgccagggcgccgtgatgagcgtggagcagctgctgctgggcggcgagaagatcaaggagggcagcctgaccctggaggaggtgggcaagaagttcaagaacgacgagatcgtgttc

SEQ ID NO: 83 - DNA, 576 bp, synthetic construct
  source 1..576: mol_type = other DNA
cagatcgtgaagagcagcatcgagatgagcaaggccaacatgagggacaacctgcagatgctgccccacgactacatcgagctgatcgagatcagccaggacccctaccagaacaggatcttcgagatgaaggtgatggacctgttcatcaacgagtacggcttcagcggcagccacctgggcggcagcaggaagcccgacggcgccatgtacgcccacggcttcggcgtgatcgtggacaccaaggcctacaaggacggctacaacctgcccatcagccaggccgacgagatggagaggtacgtgagggagaacatcgacaggaacgagcacgtgaacagcaacaggtggtggaacatcttccccgaggacaccaacgagtacaagttcctgttcgtgagcggcttcttcaagggcaacttcgagaagcagctggagaggatcagcatcgacaccggcgtgcagggcggcgccctgagcgtggagcacctgctgctgggcgccgagtacatcaagaggggcatcctgaccctgtacgacttcaagaacagcttcctgaacaaggagatccagttc

SEQ ID NO: 84 - DNA, 588 bp, synthetic construct
  source 1..588: mol_type = other DNA
cagaccatcaagagcagcatcgaggagctgaagagcgagctgaggacccagctgaacgtgatcagccacgactacctgcagctggtggacatcagccaggacagccagcagaacaggctgttcgagatgaaggtgatggacctgttcatcaacgagttcggctacaacggcagccacctgggcggcagcaggaagcccgacggcatcctgtacaccgagggcctgagcaaggactacggcatcatcgtggacaccaaggcctacaaggacggctacaacctgcccatcgcccaggccgacgagatggagaggtacatcagggagaacatcgacaggaacgaggtggtgaaccccaacaggtggtgggaggtgttccccagcaagatcaacgactacaagttcctgttcgtgagcgcctacttcaagggcaacttcaaggagcagctggagaggatcagcatcaacaccggcatcctgggcggcgccatcagcgtggagcacctgctgctgggcgccgagtacttcaagaggggcatcctgagcctggaggacgtgagggacaagttctgcaacaccgagatcgagttc

SEQ ID NO: 85 - DNA, 585 bp, synthetic construct
  source 1..585: mol_type = other DNA
ggcaagagcgaggtggagaccatcaaggagcagatgaggggcgagctgacccacctgagccacgagtacctgggcctgctggacctggcctacgacagcaagcagaacaggctgttcgagctgaagaccatgcagctgctgaccgaggagtgcggcttcgagggcctgcacctgggcggcagcaggaagcccgacggcatcgtgtacaccaaggacgagaacgagcaggtgggcaaggagaactacggcatcatcatcgacaccaaggcctacagcggcggctacagcctgcccatcagccaggccgacgagatggagaggtacatcggcgagaaccagaccagggacatcaggatcaaccccaacgagtggtggaagaacttcggcgacggcgtgaccgagtactactacctgttcgtggccggccacttcaagggcaagtaccaggagcagatcgacaggatcaactgcaacaagaacatcaagggcgccgccgtgagcatccagcagctgctgaggatcgtgaacgactacaaggccggcaagctgacccacgaggacatgaagctgaagatcttccactac

SEQ ID NO: 86 - DNA, 453 bp, synthetic construct
  source 1..453: mol_type = other DNA
atgaagatcctggagctgctgatcaacgagtgcggctacaagggcctgcacctgggcggcgccaggaagcccgacggcatcatctacaccgagaaggagaagtacaactacggcgtgatcatcgacaccaaggcctacagcaagggctacaacctgcccatcggccagatcgacgagatgatcaggtacatcatcgagaacaacgagaggaacatcaagaggaacaccaactgctggtggaacaacttcgagaagaacgtgaacgagttctacttcagcttcatcagcggcgagttcaccggcaacatcgaggagaagctgaacaggatcttcatcagcaccaacatcaagggcaacgccatgagcgtgaagaccctgctgtacctggccaacgagatcaaggccaacaggatcagctacatcgagctgctgaactacttcgacaacaaggtg

SEQ ID NO: 87 - DNA, 582 bp, synthetic construct
  source 1..582: mol_type = other DNA
gccaagagcagccagagcgagaccaaggagaagctgagggagaagctgaggaacctgccccacgagtacctgagcctggtggacctggcctacgacagcaagcagaacaggctgttcgagatgaaggtgatcgagctgctgaccgaggagtgcggcttccagggcctgcacctgggcggcagcaggaggcccgacggcgtgctgtacaccgccggcctgaccgacaactacggcatcatcctggacaccaaggcctacagcagcggctacagcctgcccatcgcccaggccgacgagatggagaggtacgtgagggagaaccagaccagggacgagctggtgaaccccaaccagtggtgggagaacttcgagaacggcctgggcaccttctacttcctgttcgtggccggccacttcaacggcaacgtgcaggcccagctggagaggatcagcaggaacaccggcgtgctgggcgccgccgccagcatcagccagctgctgctgctggccgacgccatcaggggcggcaggatggacagggagaggctgaggcacctgatgttccagaacgaggagttcctg

SEQ ID NO: 88 - DNA, 579 bp, synthetic construct
  source 1..579: mol_type = other DNA
aacagcgagaagagcgagttcacccaggagaaggacaacctgagggagaagctggacaccctgagccacgagtacctgagcctggtggacctggccttcgacagccagcagaacaggctgttcgagatgaagaccgtggagctgctgaccaaggagtgcaactacaagggcgtgcacctgggcggcagcaggaagcccgacggcatcatctacaccgagaacagcaccgacaactacggcgtgatcatcgacaccaaggcctacagcaacggctacaacctgcccatcagccaggtggacgagatggtgaggtacgtggaggagaacaacaagagggagaaggagaggaacagcaacgagtggtggaaggagttcggcgacaacatcaacaagttctacttcagcttcatcagcggcaagttcatcggcaacatcgaggagaagctgcagaggatcaccatcttcaccaacgtgtacggcaacgccatgaccatcatcaccctgctgtacctggccaacgagatcaaggccaacaggctgaagaccatggaggtggtgaagtacttcgacaacaaggtg

SEQ ID NO: 89 - DNA, 600 bp, synthetic construct
  source 1..600: mol_type = other DNA
aacctgacctgcagcgacctgaccgagatcaaggaggaggtgaggaacgccctgacccacctgagccacgagtacctggccctgatcgacctggcctacgacagcacccagaacaggctgttcgagatgaagaccctgcagctgctggtggaggagtgcggctaccagggcacccacctgggcggcagcaggaagcccgacggcatctgctacagcgaggaggccaagagcgagggcctggaggccaactacggcatcatcatcgacaccaagagctacagcggcggctacggcctgcccatcagccaggccgacgagatggagaggtacatcagggagaaccagaccagggacgccgaggtgaacaggaacaagtggtgggaggccttccccgagaccatcgacatcttctacttcatgttcgtggccggccacttcaagggcaactacttcaaccagctggagaggctgcagaggagcaccggcatcaagggcgccgccgtggacatcaagaccctgctgctgaccgccaacaggtgcaagaccggcgagctggaccacgccggcatcgagagctgcttcttcaacaactgcaggctg

SEQ ID NO: 90 - DNA, 579 bp, synthetic construct
  source 1..579: mol_type = other DNA
gacaacgtgaagagcaacttcaaccaggagaaggacgagctgagggagaagctggacaccctgagccacgagtacctgtacctgctggacctggcctacgacagcaagcagaacaagctgttcgagatgaagatcctggagctgctgatcaacgagtgcggctacaggggcctgcacctgggcggcgtgaggaagcccgacggcatcatctacaccgagaaggagaagtacaactacggcgtgatcatcgacaccaaggcctacagcaagggctacaacctgcccatcggccagatcgacgagatgatcaggtacatcatcgagaacaacgagaggaacatcaagaggaacaccaactgctggtggaacaacttcgagaagaacgtgaacgagttctacttcagcttcatcagcggcgagttcaccggcaacatcgaggagaagctgaacaggatcttcatcagcaccaacatcaagggcaacgccatgagcgtgaagaccctgctgtacctggccaacgagatcaaggccaacaggatcagcttcctggagatggagaagtacttcgacaacaaggtg

SEQ ID NO: 91 - DNA, 588 bp, synthetic construct
  source 1..588: mol_type = other DNA
gagggcatcaagagcaacatcagcctgctgaaggacgagctgaggggccagatcagccacatcagccacgagtacctgagcctgatcgacctggccttcgacagcaagcagaacaggctgttcgagatgaaggtgctggagctgctggtgaacgagtacggcttcaagggcaggcacctgggcggcagcaggaagcccgacggcatcgtgtacagcaccaccctggaggacaacttcggcatcatcgtggacaccaaggcctacagcgagggctacagcctgcccatcagccaggccgacgagatggagaggtacgtgagggagaacagcaacagggacgaggaggtgaaccccaacaagtggtgggagaacttcagcgaggaggtgaagaagtactacttcgtgttcatcagcggcagcttcaagggcaagttcgaggagcagctgaggaggctgagcatgaccaccggcgtgaacggcagcgccgtgaacgtggtgaacctgctgctgggcgccgagaagatcaggagcggcgagatgaccatcgaggagctggagagggccatgttcaacaacagcgagttcatc

SEQ ID NO: 92 - DNA, 573 bp, synthetic construct
  source 1..573: mol_type = other DNA
atcagcaagaccaacgtgctggagctgaaggacaaggtgagggacaagctgaagtacgtggacaacaggtacctggccctgatcgacctggcctacgacggcaccgccaacagggacttcgagatccagaccatcgacctgctgatcaacgagctgaagttcaagggcgtgaggctgggcgagagcaggaagcccgacggcatcatcagctacgacatcaacggcgtgatcatcgacaacaaggcctacagcagcggctacaacctgcccatcaaccaggccgacgagatgatcaggtacatcgaggagaaccagaccagggacaagaagatcaaccccaacaagtggtgggagagcttcgacgacaaggtgaaggacttcaactacctgttcgtgagcagcttcttcaagggcaacttcaagaacaacctgaagcacatcgccaacaggaccggcgtgaacggcggcgtgatcaacgtggagaacctgctgtacttcgccgaggagctgaagagcggcaggctgagctacgtggacctgttcaagatgtacgacaacgacgagatcaacatc

SEQ ID NO: 93 - DNA, 573 bp, synthetic construct
  source 1..573: mol_type = other DNA
atcagcaagaccaacgtgctggagctgaaggacaaggtgagggacaagctgaagtacgtggaccacaggtacctggccctgatcgacctggcctacgacggcaccgccaacagggacttcgagatccagaccatcgacctgctgatcaacgagctgaagttcaagggcgtgaggctgggcgagagcaggaagcccgacggcatcatcagctacgacatcaacggcgtgatcatcgacaacaaggcctacagcaccggctacaacctgcccatcaaccaggccgacgagatgatcaggtacatcgaggagaaccagaccagggacaagaagatcaacagcaacaagtggtgggagagcttcgacgacaaggtgaagaacttcaactacctgttcgtgagcagcttcttcaagggcaacttcaagaacaacctgaagcacatcgccaacaggaccggcgtgaacggcggcgccatcaacgtggagaacctgctgtacttcgccgaggagctgaaggccggcaggctgagctacgtggacagcttcaccatgtacgacaacgacgagatctacgtg

SEQ ID NO: 94 - DNA, 579 bp, synthetic construct
  source 1..579: mol_type = other DNA
aaggccgagaagagcgagttcctgatcgagaaggacaagctgagggagaagctggacaccctgccccacgactacctgagcatggtggacctggcctacgacagcaagcagaacaggctgttcgagatgaagaccatcgagctgctgatcaacgagtgcaactacaagggcctgcacctgggcggcaccaggaagcccgacggcatcgtgtacaccaacaacgaggtggagaactacggcatcatcatcgacaccaaggcctacagcaagggctacaacctgcccatcagccaggtggacgagatgaccaggtacgtggaggagaacaacaagagggagaagaagaggaaccccaacgagtggtggaacaacttcgacagcaacgtgaagaagttctacttcagcttcatcagcggcaagttcgtgggcaacatcgaggagaagctgcagaggatcaccctgttcaccgagatctacggcaacgccatcaccgtgaccaccctgctgtacatcgccaacgagatcaaggccaacaggatgaagaagagcgacatcatggagtacttcaacgacaaggtg

SEQ ID NO: 95 - DNA, 573 bp, synthetic construct
  source 1..573: mol_type = other DNA
atcagcaagaccaacgtgctggagctgaaggacaaggtgagggacaagctgaagtacgtggaccacaggtacctggccctgatcgacctggcctacgacggcaccgccaacagggacttcgagatccagaccatcgacctgctgatcaacgagctgaagttcaagggcgtgaggctgggcgagagcaggaagcccgacggcatcatcagctacaacatcaacggcgtgatcatcgacaacaaggcctacagcaccggctacaacctgcccatcaaccaggccgacgagatgatcaggtacatcgaggagaaccagaccagggacgagaagatcaacagcaacaagtggtgggagagcttcgacgacgaggtgaaggacttcaactacctgttcgtgagcagcttcttcaagggcaacttcaagaacaacctgaagcacatcgccaacaggaccggcgtgaacggcggcgccatcaacgtggagaacctgctgtacttcgccgaggagctgaaggccggcaggctgagctacgtggacagcttcaccatgtacgacaacgacgagatctacgtg

SEQ ID NO: 96 - DNA, 573 bp, synthetic construct
  source 1..573: mol_type = other DNA
atcagcaagaccaacatcctggagctgaaggacaaggtgagggacaagctgaagtacgtggaccacaggtacctggccctgatcgacctggcctacgacggcaccgccaacagggacttcgagatccagaccatcgacctgctgatcaacgagctgaagttcaagggcgtgaggctgggcgagagcaggaagcccgacggcatcatcagctacaacatcaacggcgtgatcatcgacaacaaggcctacagcaccggctacaacctgcccatcaaccaggccgacgagatgatcaggtacatcgaggagaaccagaccagggacgagaagatcaacagcaacaagtggtgggagagcttcgacgagaaggtgaaggacttcaactacctgttcgtgagcagcttcttcaagggcaacttcaagaacaacctgaagcacatcgccaacaggaccggcgtgaacggcggcgccatcaacgtggagaacctgctgtacttcgccgaggagctgaaggccggcaggatcagctacctggacagcttcaagatgtacaacaacgacgagatctacctg

SEQ ID NO: 97 - DNA, 573 bp, synthetic construct
  source 1..573: mol_type = other DNA
atcagcaagaccaacgtgctggagctgaaggacaaggtgagggacaagctgaagtacgtggaccacaggtacctggccctgatcgacctggcctacgacggcaccgccaacagggacttcgagatccagaccatcgacctgctgatcaacgagctgaagttcaagggcgtgaggctgggcgagagcaggaagcccgacggcatcatcagctacaacatcaacggcgtgatcatcgacaacaaggcctacagcaccggctacaacctgcccatcaaccaggccgacgagatgatcaggtacatcgaggagaaccagaccagggacgagaagatcaacagcaacaagtggtgggagagcttcgacgacaaggtgaaggacttcaactacctgttcgtgagcagcttcttcaagggcaacttcaagaacaacctgaagcacatcgccaacaggaccggcgtgagcggcggcgccatcaacgtggagaacctgctgtacttcgccgaggagctgaaggccggcaggctgagctacgtggacagcttcaagatgtacgacaacgacgagatctacgtg

SEQ ID NO: 98 - DNA, 573 bp, synthetic construct
  source 1..573: mol_type = other DNA
atcagcaagaccaacgtgctggagctgaaggacaaggtgaggaacaagctgaagtacgtggaccacaggtacctggccctgatcgacctggcctacgacggcaccgccaacagggacttcgagatccagaccatcgacctgctgatcaacgagctgaagttcaagggcgtgaggctgggcgagagcaggaagcccgacggcatcatcagctacgacatcaacggcgtgatcatcgacaacaagagctacagcaccggctacaacctgcccatcaaccaggccgacgagatgatcaggtacatcgaggagaaccagaccagggacgagaagatcaacagcaacaagtggtgggagagcttcgacgagaaggtgaaggacttcaactacctgttcgtgagcagcttcttcaagggcaacttcaagaacaacctgaagcacatcgccaacaggaccggcgtgaacggcggcgccatcaacgtggagaacctgctgtacttcgccgaggagctgaagagcggcaggctgagctacgtggacagcttcaccatgtacgacaacgacgagatctacgtg

SEQ ID NO: 99 - DNA, 573 bp, synthetic construct
  source 1..573: mol_type = other DNA
atcagcaagaccaacgtgctggagctgaaggacaaggtgagggacaagctgaagtacgtggaccacaggtacctgagcctgatcgacctggcctacgacggcaacgccaacagggacttcgagatccagaccatcgacctgctgatcaacgagctgaacttcaagggcgtgaggctgggcgagagcaggaagcccgacggcatcatcagctacaacatcaacggcgtgatcatcgacaacaaggcctacagcaccggctacaacctgcccatcaaccaggccgacgagatgatcaggtacatcgaggagaaccagaccagggacgagaagatcaacagcaacaagtggtgggagagcttcgacgacaaggtgaaggacttcaactacctgttcgtgagcagcttcttcaagggcaacttcaagaacaacctgaagcacatcgccaacaggaccggcgtgagcggcggcgccatcaacgtggagaacctgctgtacttcgccgaggagctgaaggccggcaggctgagctacgccgacagcttcaccatgtacgacaacgacgagatctacgtg

SEQ ID NO: 100 - DNA, 573 bp, synthetic construct
  source 1..573: mol_type = other DNA
atcgccaagaccaacgtgctgggcctgaaggacaaggtgagggacaggctgaagtacgtggaccacaggtacctggccctgatcgacctggcctacgacggcaccgccaacagggacttcgagatccagaccatcgacctgctgatcaacgagctgaagttcaagggcgtgaggctgggcgagagcaggaagcccgacggcatcatcagctacaacgtgaacggcgtgatcatcgacaacaaggcctacagcaagggctacaacctgcccatcaaccaggccgacgagatgatcaggtacatcgaggagaaccagaccagggacgagaagatcaacgccaacaagtggtgggagagcttcgacgacaaggtggaggagttcagctacctgttcgtgagcagcttcttcaagggcaacttcaagaacaacctgaagcacatcgccaacaggaccggcgtgaacggcggcgccatcaacgtggagaacctgctgtacttcgccgaggagctgaagagcggcaggctgagctacatggacagcttcagcctgtacgacaacgacgagatctgcgtg

SEQ ID NO: 101 - DNA, 579 bp, synthetic construct
  source 1..579: mol_type = other DNA
gagctgaaggacgagcagagcgagaagaggaaggccaagttcctgaaggagaccaagctgcccatgaagtacatcgagctgctggacatcgcctacgacggcaagaggaacagggacttcgagatcgtgaccatggagctgttcagggaggtgtacaggctgaacagcaagctgctgggcggcggcaggaagcccgacggcctgatctacaccgacgacttcggcgtgatcgtggacaccaaggcctacggcgagggctacagcaagagcatcaaccaggccgacgagatgatcaggtacatcgaggacaacaagaggagggacgagaagaggaaccccatcaagtggtgggagagcttccccagcagcatcagccagaacaacttctacttcctgtgggtgagcagcaagttcgtgggcaagttccaggagcagctggcctacaccgccaacgagacccagaccaagggcggcgccatcaacgtggagcagatcctgatcggcgccgacctgatcatgcagaagatgctggacatcaacaccatccccagcttcttcgagaaccaggagatcatcttc

SEQ ID NO: 102 - DNA, 573 bp, synthetic construct
  source 1..573: mol_type = other DNA
atcttcaagaccaacgtgctggagctgaaggacagcatcagggagaagctggactacatcgaccacaggtacctgagcctggtggacctggcctacgacagcaaggccaacagggacttcgagatccagaccatcgacctgctgatcaacgagctggacttcaagggcctgaggctgggcgagagcaggaagcccgacggcatcatcagctacgacatcaacggcgtgatcatcgacaacaaggcctacagcaagggctacaacctgcccatcaaccaggccgacgagatgatcaggtacatccaggagaaccagagcaggaacgagaagatcaaccccaacaagtggtgggagaacttcgaggacaaggtgatcaagttcaactacctgttcatcagcagcctgttcgtgggcggcttcaagaagaacctgcagcacatcgccaacaggaccggcgtgaacggcggcgccatcgacgtggagaacctgctgtacttcgccgaggagatcaagagcggcaggctgacctacaaggacagcttcagcaggtacatcaacgacgagatcaagatg

SEQ ID NO: 103 - DNA, 579 bp, synthetic construct
  source 1..579: mol_type = other DNA
ctgcccgtgaagagcgaggtgagcgtgttcaaggactacctgaggacccacctgacccacgtggaccacaggtacctgatcctggtggacctgggcttcgacggcagcagcgacagggactacgagatgaagaccgccgagctgttcaccgccgagctgggcttcatgggcgccaggctgggcgacaccaggaagcccgacgtgtgcgtgtaccacggcgccaacggcctgatcatcgacaacaaggcctacggcaagggctacagcctgcccatcaagcaggccgacgagatctacaggtacatcgaggagaacaaggagagggacgccaggctgaaccccaaccagtggtggaaggtgttcgacgagagcgtgacccacttcaggttcgccttcatcagcggcagcttcaccggcggcttcaaggacaggatcgagctgatcagcatgaggagcggcatctgcggcgccgccgtgaacagcgtgaacctgctgctgatggccgaggagctgaagagcggcaggctggactacgaggagtggttccagtacttcgactgcaacgacgagatcagcttc

SEQ ID NO: 104 - DNA, 579 bp, synthetic construct
  source 1..579: mol_type = other DNA
atcagcgtgaagagcgacatggccgtggtgaaggacagcgtgagggagaggctggcccacgtgagccacgagtacctgatcctgatcgacctgggcttcgacggcaccagcgacagggactacgagatccagaccgccgagctgttcaccagggagctggacttcctgggcggcaggctgggcgacaccaggaagcccgacgtgtgcatctactacggcaaggacggcatgatcatcgacaacaaggcctacggcaagggctacagcctgcccatcaagcaggccgacgagatgtacaggtacctggaggagaacaaggagaggaacgagaagatcaaccccaacaggtggtggaaggtgttcgacgagggcgtgaccgactacaggttcgccttcgtgagcggcagcttcaccggcggcttcaaggacaggctggagaacatccacatgaggagcggcctgtgcggcggcgccatcgacagcgtgaccctgctgctgctggccgaggagctgaaggccggcaggatggagtacagcgagttcttcaggctgttcgactgcaacgacgaggtgaccttc

SEQ ID NO: 105 - DNA, 579 bp, synthetic construct
  source 1..579: mol_type = other DNA
gagctgaaggacaaggccgccgacgccgtgaaggccaagttcctgaagctgaccggcctgagcatgaagtacatcgagctgctggacatcgcctacgacagcagcaggaacagggacttcgagatcctgaccgccgacctgttcaagaacgtgtacggcctggacgccatgcacctgggcggcggcaggaagcccgacgccatcgcccagaccagccacttcggcatcatcatcgacaccaaggcctacggcaacggctacagcaagagcatcagccaggaggacgagatggtgaggtacatcgaggacaaccagcagaggagcatcaccaggaacagcgtggagtggtggaagaacttcaacagcagcatccccagcaccgccttctacttcctgtgggtgagcagcaagttcgtgggcaagttcgacgaccagctgctggccacctacaacaggaccaacacctgcggcggcgccctgaacgtggagcagctgctgatcggcgcctacaaggtgaaggccggcctgctgggcatcggccagatccccagctacttcaagaacaaggagatcgcctgg

SEQ ID NO: 106 - DNA, 579 bp, synthetic construct
  source 1..579: mol_type = other DNA
atcagcgtgaagagcgacatggccgtggtgaaggacagcgtgagggagaggctggcccacgtgagccacgagtacctgctgctgatcgacctgggcttcgacggcaccagcgacagggactacgagatccagaccgccgagctgctgaccagggagctggacttcctgggcggcaggctgggcgacaccaggaagcccgacgtgtgcatctactacggcaaggacggcatgatcatcgacaacaaggcctacggcaagggctacagcctgcccatcaagcaggccgacgagatgtacaggtacctggaggagaacaaggagaggaacgagaagatcaaccccaacaggtggtggaaggtgttcgacgagggcgtgaccgactacaggttcgccttcgtgagcggcagcttcaccggcggcttcaaggacaggctggagaacatccacatgaggagcggcctgtgcggcggcgccatcgacagcgtgaccctgctgctgctggccgaggagctgaaggccggcaggatggagtacagcgagttcttcaggctgttcgactgcaacgacgaggtgaccttc

SEQ ID NO: 107 - DNA, 579 bp, synthetic construct
  source 1..579: mol_type = other DNA
gagctgaaggacgagcaggccgagaagaggaaggccaagttcctgaaggagaccaacctgcccatgaagtacatcgagctgctggacatcgcctacgacggcaagaggaacagggacttcgagatcgtgaccatggagctgttcaggaacgtgtacaggctgcacagcaagctgctgggcggcggcaggaagcccgacggcctgctgtaccaggacaggttcggcgtgatcgtggacaccaaggcctacggcaagggctacagcaagagcatcaaccaggccgacgagatgatcaggtacatcgaggacaacaagaggagggacgagaacaggaaccccatcaagtggtgggaggccttccccgacaccatcccccaggaggagttctacttcatgtgggtgagcagcaagttcatcggcaagttccaggagcagctggactacaccagcaacgagacccagatcaagggcgccgccctgaacgtggagcagctgctgctgggcgccgacctggtgctgaagggccagctgcacatcagcgacctgcccagctacttccagaacaaggagatcgagttc

SEQ ID NO: 108 - DNA, 585 bp, synthetic construct
  source 1..585: mol_type = other DNA
aggaacctggacaacgtggagagggacaacaggaaggccgagttcctggccaagaccagcctgccccccaggttcatcgagctgctgagcatcgcctacgagagcaagagcaacagggacttcgagatgatcaccgccgagctgttcaaggacgtgtacggcctgggcgccgtgcacctgggcaacgccaagaagcccgacgccctggccttcaacgacgacttcggcatcatcatcgacaccaaggcctacagcaacggctacagcaagaacatcaaccaggaggacgagatggtgaggtacatcgaggacaaccagatcaggagccccgacaggaacaacaacgagtggtggctgagcttcccccccagcatccccgagaacgacttccacttcctgtgggtgagcagctacttcaccggcaggttcgaggagcagctgcaggagaccagcgccaggaccggcggcaccaccggcggcgccctggacgtggagcagctgctgatcggcggcagcctgatccaggagggcagcctggccccccacgaggtgcccgcctacatgcagaacagggtgatccacttc

SEQ ID NO: 109 - DNA, 579 bp, synthetic construct
  source 1..579: mol_type = other DNA
agccccgtgaagagcgaggtgagcgtgttcaaggactacctgaggacccacctgacccacgtggaccacaggtacctgatcctggtggacctgggcttcgacggcagcagcgacagggactacgagatgaagaccgccgagctgttcaccgccgagctgggcttcatgggcgccaggctgggcgacaccaggaagcccgacgtgtgcgtgtaccacggcgcccacggcctgatcatcgacaacaaggcctacggcaagggctacagcctgcccatcaagcaggccgacgagatctacaggtacatcgaggagaacaaggagagggccgtgaggctgaaccccaaccagtggtggaaggtgttcgacgagagcgtggcccacttcaggttcgccttcatcagcggcagcttcaccggcggcttcaaggacaggatcgagctgatcagcatgaggagcggcatctgcggcgccgccgtgaacagcgtgaacctgctgctgatggccgaggagctgaagagcggcaggctgaactacgaggagtggttccagtacttcgactgcaacgacgagatcagcctg

SEQ ID NO: 110 - DNA, 579 bp, synthetic construct
  source 1..579: mol_type = other DNA
accctggtggacatcgagaaggagaggaagaaggcctacttcctgaaggagaccagcctgagccccaggtacatcgagctgctggagatcgccttcgaccccaagaggaacagggacttcgaggtgatcaccgccgagctgctgaaggccggctacggcctgaaggccaaggtgctgggcggcggcaggaggcccgacggcatcgcctacaccaaggactacggcctgatcgtggacaccaaggcctacagcaacggctacggcaagaacatcggccaggccgacgagatgatcaggtacatcgaggacaaccagaagagggacaacaagaggaaccccatcgagtggtggagggagttcgaggtgcagatccccgccaacagctactactacctgtgggtgagcggcaggttcaccggcaggttcgacgagcagctggtgtacaccagcagccagaccaacaccaggggcggcgccctggaggtggagcagctgctgtggggcgccgacgccgtgatgaagggcaagctgaacgtgagcgacctgcccaagtacatgaacaacagcatcatcaagctg

SEQ ID NO: 111 - DNA, 579 bp, synthetic construct
  source 1..579: mol_type = other DNA
gagctgagggacaaggtgatcgaggagcagaaggccatcttcctgcagaagaccaagctgcccctgagctacatcgagctgctggagatcgccagggacggcaagaggagcagggacttcgagctgatcaccatcgagctgttcaagaacatctacaagatcaacgccaggatcctgggcggcgccaggaagcccgacggcgtgctgtacatgcccgagttcggcgtgatcgtggacaccaaggcctacgccgacggctacagcaagagcatcgcccaggccgacgagatgatcaggtacatcgaggacaacaagaggagggaccccagcaggaacagcaccaagtggtgggagcacttccccaccagcatccccgccaacaacttctacttcctgtgggtgagcagcgtgttcgtgaacaagttccacgagcagctgagctacaccgcccaggagacccagaccgtgggcgccgccctgagcgtggagcagctgctgctgggcgccgacagcgtgctgaagggcaacctgaccaccgagaagttcatcgacagcttcaagaaccaggagatcgtgttc

SEQ ID NO: 112 - DNA, 579 bp, synthetic construct
  source 1..579: mol_type = other DNA
ggcgccaccaagagcgacctgagcctgctgaaggacgacatcaggaagaagctgaaccacatcaaccacaagtacctggtgctgatcgacctgggcttcgacggcaccgccgacagggactacgagctgcagaccgccgacctgctgaccagcgagctggccttcaagggcgccaggctgggcgacagcaggaagcccgacgtgtgcgtgtaccacgacaagaacggcctgatcatcgacaacaaggcctacggcagcggctacagcctgcccatcaagcaggccgacgagatgctgaggtacatcgaggagaaccagaagagggacaaggccctgaaccccaacgagtggtggaccatcttcgacgacgccgtgagcaagttcaacttcgccttcgtgagcggcgagttcaccggcggcttcaaggacaggctggagaacatcagcaggaggagctacaccaacggcgccgccatcaacagcgtgaacctgctgctgctggccgaggagatcaagagcggcaggatcagctacggcgacgccttcaccaagttcgagtgcaacgacgagatcatcatc

SEQ ID NO: 113 - DNA, 579 bp, synthetic construct
  source 1..579: mol_type = other DNA
gagctgaggaacgccgccctggacaagcagaaggtgaacttcatcaacaagaccggcctgcccatgaagtacatcgagctgctggagatcgccttcgacggcagcaggaacagggacttcgagatggtgaccgccgacctgttcaagaacgtgtacggcttcaacagcatcctgctgggcggcggcaggaagcccgacggcctgatcttcaccgacaggttcggcgtgatcatcgacaccaaggcctacggcaacggctacagcaagagcatcggccaggaggacgagatggtgaggtacatcgaggacaaccagctgagggacagcaacaggaacagcgtggagtggtggaagaacttcgacgagaagatcgagagcgagaacttctacttcatgtggatcagcagcaagttcatcggccagttcagcgaccagctgcagagcaccagcgacaggaccaacaccaagggcgccgccctgaacgtggagcagctgctgctgggcgccgccgccgccagggacggcaagctggacatcaacagcctgcccatctacatgaacaacaaggagatcctgtgg

SEQ ID NO: 114 - DNA, 579 bp, synthetic construct
  source 1..579: mol_type = other DNA
gagctgaaggacgagcagagcgagaagaggaaggcctacttcctgaaggagaccaacctgcccctgaagtacatcgagctgctggacatcgcctacgacggcaagaggaacagggacttcgagatcgtgaccatggagctgttcaggaacgtgtacaggctgcagagcaagctgctgggcggcgtgaggaagcccgacggcctgctgtacaagcacaggttcggcatcatcgtggacaccaaggcctacggcgagggctacagcaagagcatcagccaggccgacgagatgatcaggtacatcgaggacaacaagaggagggacgagaacaggaacagcaccaagtggtgggagcacttccccgactgcatccccaagcagagcttctacttcatgtgggtgagcagcaagttcgtgggcaagttccaggagcagctggactacaccgccaacgagaccaagaccaacggcgccgccctgaacgtggagcagctgctgtggggcgccgacctggtggccaagggcaagctggacatcagccagctgcccagctacttccagaacaaggagatcgagttc

SEQ ID NO: 115 - DNA, 564 bp, synthetic construct
  source 1..564: mol_type = other DNA
cacaacaacaagttcaagaactacctgagggagaacagcgagctgagcttcaagttcatcgagctgatcgacatcgcctacgacggcaacaggaacagggacatggagatcatcaccgccgagctgctgaaggagatctacggcctgaacgtgaagctgctgggcggcggcaggaagcccgacatcctggcctacaccgacgacatcggcatcatcatcgacaccaaggcctacaaggacggctacggcaagcagatcaaccaggccgacgagatgatcaggtacatcgaggacaaccagaggagggacctgatcaggaaccccaacgagtggtggaggtacttccccaagagcatcagcaaggagaagatctacttcatgtggatcagcagctacttcaagaacaacttctacgagcaggtgcagtacaccgcccaggagaccaagagcatcggcgccgccctgaacgtgaggcagctgctgctgtgcgccgacgccatccagaaggaggtgctgagcctggacaccttcctgggcagcttcaggaacgaggagatcaacctg

SEQ ID NO: 116 - DNA, 579 bp, synthetic construct
  source 1..579: mol_type = other DNA
ctgcccgtgaagagcgaggtgagcatcctgaaggactacctgaggagccacctgacccacatcgaccacaagtacctgatcctggtggacctgggctacgacggcaccagcgacagggactacgagatccagaccgcccagctgctgaccgccgagctgagcttcctgggcggcaggctgggcgacaccaggaagcccgacgtgtgcatctactacgaggacaacggcctgatcatcgacaacaaggcctacggcaagggctacagcctgcccatgaagcaggccgacgagatgtacaggtacatcgaggagaacaaggagaggagcgagctgctgaaccccaactgctggtggaacatcttcgacaaggacgtgaagaccttccacttcgccttcctgagcggcgagttcaccggcggcttcagggacaggctgaaccacatcagcatgaggagcggcatgaggggcgccgccgtgaacagcgccaacctgctgatcatggccgagaagctgaaggccggcaccatggagtacgaggagttcttcaggctgttcgacaccaacgacgagatcctgttc

SEQ ID NO: 117 - DNA, 579 bp, synthetic construct
  source 1..579: mol_type = other DNA
ctgcccgtgaagagccaggtgagcatcctgaaggactacctgaggagctacctgagccacgtggaccacaagtacctgatcctgctggacctgggcttcgacggcaccagcgacagggactacgagatctggaccgcccagctgctgaccgccgagctgagcttcctgggcggcaggctgggcgacaccaggaagcccgacgtgtgcatctactacgaggacaacggcctgatcatcgacaacaaggcctacggcaagggctacagcctgcccatcaagcaggccgacgagatgtacaggtacatcgaggagaacaaggagaggagcgacctgctgaaccccaactgctggtggaacatcttcggcgagggcgtgaagaccttcaggttcgccttcctgagcggcgagttcaccggcggcttcaaggacaggctgaaccacatcagcatgaggagcggcatcaagggcgccgccgtgaacagcgccaacctgctgatcatggccgagcagctgaagagcggcaccatgagctacgaggagttcttccagctgttcgactacaacgacgagatcatcttc

SEQ ID NO: 118 - DNA, 573 bp, synthetic construct
  source 1..573: mol_type = other DNA
gtgagcaagaccaacatcctggagctgaaggacaacaccagggagaagctggtgtacctggaccacaggtacctgagcctgttcgacctggcctacgacgacaaggccagcagggacttcgagatccagaccatcgacctgctgatcaacgagctgcagttcaagggcctgaggctgggcgagaggaggaagcccgacggcatcatcagctacggcgtgaacggcgtgatcatcgacaacaaggcctacagcaagggctacaacctgcccatcaggcaggccgacgagatgatcaggtacatccaggagaaccagagcagggacgagaagctgaaccccaacaagtggtgggagaacttcgaggaggagaccagcaagttcaactacctgttcatcagcagcaagttcatcagcggcttcaagaagaacctgcagtacatcgccgacaggaccggcgtgaacggcggcgccatcaacgtggagaacctgctgtgcttcgccgagatgctgaagagcggcaagctggagtacaacgacttcttcaaccagtacaacaacgacgagatcatcatg

SEQ ID NO: 119 - DNA, 579 bp, synthetic construct
  source 1..579: mol_type = other DNA
ctgcccgtgaagagccaggtgagcatcctgaaggactacctgaggagctgcctgagccacgtggaccacaagtacctgatcctgctggacctgggcttcgacggcaccagcgacagggactacgagatccagaccgcccagctgctgaccgccgagctgagcttcctgggcggcaggctgggcgacaccaggaagcccgacgtgtgcatctactacgaggacaacggcctgatcatcgacaacaaggcctacggcaagggctacagcctgcccatcaagcaggccgacgagatgtacaggtacatcgaggagaacaaggagaggagcgagctgctgaaccccaactgctggtggaacatcttcgacgagggcgtgaagaccttcaggttcgccttcctgagcggcgagttcaccggcggcttcaaggacaggctgaaccacatcagcatgaggagcggcatcaagggcgccgccgtgaacagcgccaacctgctgatcatcgccgagcagctgaagagcggcaccatgagctacgaggagttcttccagctgttcgaccagaacgacgagatcaccgtg

SEQ ID NO: 120 - DNA, 579 bp, synthetic construct
  source 1..579: mol_type = other DNA
atgagcagcaagagcgagatcagcgtgatcaaggacaacatcaggaagaggctgaaccacatcaaccacaagtacctggtgctgatcgacctgggcttcgacggcaccgccgacagggactacgagctgcagaccgccgacctgctgaccagcgagctgagcttcaagggcgccaggctgggcgacaccaggaagcccgacgtgtgcgtgtaccacggcaccaacggcctgatcatcgacaacaaggcctacggcaagggctacagcctgcccatcaagcaggccgacgagatgctgaggtacatcgaggagaaccagaagagggacaagagcctgaaccccaacgagtggtggaccatcttcgacgacgccgtgagcaagttcaacttcgccttcgtgagcggcgagttcaccggcggcttcaaggacaggctggagaacatcagcaggaggagcagcgtgaacggcgccgccatcaacagcgtgaacctgctgctgctggccgaggagatcaagagcggcaggatgagctacagcgacgccttcaagaacttcgactgcaacaaggagatcaccatc

SEQ ID NO: 121 - DNA, 585 bp, synthetic construct
  source 1..585: mol_type = other DNA
aggaacctggacaaggtggagagggacagcaggaaggccgagttcctggccaagaccagcctgccccccaggttcatcgagctgctgagcatcgcctacgagagcaagagcaacagggacttcgagatgatcaccgccgagttcttcaaggacgtgtacggcctgggcgccgtgcacctgggcaacgccaggaagcccgacgccctggccttcaccgacaacttcggcatcgtgatcgacaccaaggcctacagcaacggctacagcaagaacatcaaccaggaggacgagatggtgaggtacatcgaggacaaccagatcaggagccccgagaggaacaagaacgagtggtggctgagcttcccccccagcatccccgagaacaacttccacttcctgtgggtgagcagctacttcaccggctacttcgaggagcagctgcaggagaccagcgacagggccggcggcatgaccggcggcgccctggacatcgagcagctgctgatcggcggcagcctggtgcaggagggcaagctggccccccacgacatccccgagtacatgcagaacagggtgatccacttc

SEQ ID NO: 122 - DNA, 579 bp, synthetic construct
  source 1..579: mol_type = other DNA
gcccccgtgaagagcgaggtgagcctgtgcaaggacatcctgaggagccacctgacccacgtggaccacaagtacctgatcctgctggacctgggcttcgacggcaccagcgacagggactacgagatccagaccgcccagctgctgaccgccgagctggacttcaagggcgccaggctgggcgacaccaggaagcccgacgtgtgcgtgtactacggcgaggacggcctgatcctggacaacaaggcctacggcaagggctacagcctgcccatcaagcaggccgacgagatgtacaggtacatcgaggagaacaaggagaggaacgagaggctgaaccccaacaagtggtgggagatcttcgacaaggacgtggtgaggtaccacttcgccttcgtgagcggcaccttcaccggcggcttcaaggagaggctggacaacatcaggatgaggagcggcatctgcggcgccgccgtgaacagcatgaacctgctgctgatggccgaggagctgaagagcggcaggctgggctacaaggagtgcttcgccctgttcgactgcaacgacgagatcgccttc

SEQ ID NO: 123 - DNA, 603 bp, synthetic construct
  source 1..603: mol_type = other DNA
agctgcgtgaaggacgaggtgaacgacatcgtggacagggtgagggtgaagctgaagaacatcgaccacaagtacctgatcctgatcagcctggcctacagcgacgagaccgagaggaccaagaagaacagcgacgccagggacttcgagatccagaccgccgagctgttcaccaaggagctgggcttcaacggcatcaggctgggcgagagcaacaagcccgacgtgctgatcagcttcggcgccaacggcaccatcatcgacaacaagagctacaaggacggcttcaacatccccagggtgaccagcgaccagatgatcaggtacatcaacgagaacaaccagaggaccacccagctgaaccccaacgagtggtggaagaacttcgacagcagcgtgagcaactacaccttcctgttcgtgaccagcttcctgaagggcagcttcaagaaccagatcgagtacatcagcaacgccaccaacggcaccaggggcgccgccatcaacgtggagagcctgctgtacatcagcgaggacatcaagagcggcaagatcaagcagagcgacttctacagcgagttcaagaacgacgagatcgtgtac

SEQ ID NO: 124 - DNA, 579 bp, synthetic construct
  source 1..579: mol_type = other DNA
agccagggcgacaaggccagggagcagctgaaggccaagttcctggccaagaccaacctgctgcccaggtacgtggagctgctggacatcgcctacgacagcaagaggaacagggacttcgagatggtgaccgccgagctgttcaacttcgcctacctgctgcccgccgtgcacctgggcggcgtgaggaagcccgacgccctggtggccaccaagaagttcggcatcatcgtggacaccaaggcctacgccaacggctacagcaggaacgccaaccaggccgacgagatggccaggtacatcaccgagaaccagaagagggaccccaagaccaaccccaacaggtggtgggacaacttcgacgccaggatcccccccaacgcctactacttcctgtgggtgagcagcttcttcaccggccagttcgacgaccagctgagctacaccgcccacaggaccaacacccacggcggcgccctgaacgtggagcagctgctgatcggcgccaacatgatccagaccggccagctggacaggaacaagctgcccgagtacatgcaggacaaggagatcaccttc

SEQ ID NO: 125 - DNA, 579 bp, synthetic construct
  source 1..579: mol_type = other DNA
aaggtgcagaagagcaacatcctggacgtgatcgagaagtgcagggagaagatcaacaacatcccccacgagtacctggccctgatccccatgagcttcgacgagaacgagagcaccatgttcgagatcaagaccatcgagctgctgaccgagcactgcaagttcgacggcctgcactgcggcggcgccagcaagcccgacggcctgatctacagcgaggactacggcgtgatcatcgacaccaagagctacaaggacggcttcaacatccagacccccgagagggacaagatgaagaggtacatcgaggagaaccagaacaggaacccccagcacaacaagaccaggtggtgggacgagttcccccacaacatcagcaacttcctgttcctgttcgtgagcggcaagttcggcggcaacttcaaggagcagctgaggatcctgagcgagcagaccaacaacaccctgggcggcgccctgagcagctacgtgctgctgaacatcgccgagcagatcgccatcaacaagatcgaccactgcgacttcaagaccaggatcagctgcctggacgaggtggcc

SEQ ID NO: 126 - DNA, 579 bp, synthetic construct
  source 1..579: mol_type = other DNA
gtgcccgtgaagagcgaggtgagcctgtgcaaggactacctgaggagctacctgacccacgtggaccacaagtacctgatcctgctggacctgggcttcgacggcaccagcgacagggactacgagatccagaccgcccagctgctgaccgccgagctggacttcaagggcgccaggctgggcgacaccaggaagcccgacgtgtgcgtgtactacggcgaggacggcctgatcatcgacaacaaggcctacggcaagggctacagcctgcccatcaagcaggccgacgagatctacaggtacatcgaggagaacaagaagagggacgagaagctgaaccccaacaagtggtgggagatcttcgacaagggcgtggtgaggtaccacttcgccttcgtgagcggcgccttcaccggcggcttcaaggagaggctggacaacatcaggatgaggagcggcatctgcggcgccgccatcaacagcatgaacctgctgctgatggccgaggagctgaagagcggcaggctgggctacgaggagtgcttcgccctgttcgactgcaacgacgagatcaccttc

SEQ ID NO: 127 - DNA, 579 bp, synthetic construct
  source 1..579: mol_type = other DNA
gtgcccgtgaagagcgaggtgagcctgtgcaaggactacctgaggagccacctgaaccacgtggaccacaggtacctgatcctgctggacctgggcttcgacggcaccagcgacagggactacgagatccagaccgcccagctgctgaccggcgagctgaacttcaagggcgccaggctgggcgacaccaggaagcccgacgtgtgcgtgtactacggcgaggacggcctgatcatcgacaacaaggcctacggcaagggctacagcctgcccatcaagcaggccgacgagatgtacaggtacatcgaggagaacaaggagaggaacgagaagctgaaccccaacaagtggtgggagatcttcgacaaggacgtgatccactaccacttcgccttcgtgagcggcgccttcaccggcggcttcaaggagaggctggagaacatcaggatgaggagcggcatctacggcgccgccgtgaacagcatgaacctgctgctgatggccgaggagctgaagagcggcaggctggactacaaggagtgcttcaagctgttcgactgcaacgacgagatcgtgctg

SEQ ID NO: 128 - DNA, 579 bp, synthetic construct
  source 1..579: mol_type = other DNA
gtgcccgtgaagagcgaggtgagcctgctgaaggactacctgaggagccacctggtgcacgtggaccacaagtacctggtgctgctggacctgggcttcgacggcaccagcgacagggactacgagatccagaccgcccagctgctgaccggcgagctgaacttcaagggcgccaggctgggcgacaccaggaagcccgacgtgtgcgtgtactacggcgaggacggcctgatcatcgacaacaaggcctacggcaagggctacagcctgcccatcaagcaggccgacgagatgtacaggtacatcgaggagaacaaggagaggaacgagaagctgaaccccaacaagtggtgggagatcttcggcaacgacgtgatccactaccacttcgccttcgtgagcggcgccttcaccggcggcttcaaggagaggctggacaacatcaggatgaggagcggcatctacggcgccgccgtgaacagcatgaacctgctgctgctggccgaggagctgaagagcggcaggctgggctacaaggagtgcttcaagctgttcgactgcaacgacgagatcgtgctg

SEQ ID NO: 129 - DNA, 603 bp, synthetic construct
  source 1..603: mol_type = other DNA
gagtgcgtgaaggacaacgtggtggacatcaaggacagggtgaggaacaagctgatccacctggaccacaagtacctggccctgatcgacctggcctacagcgacgccgccagcagggccaagaagaacgccgacgccagggagttcgagatccagaccgccgacctgttcaccaaggagctgagcttcaacggccagaggctgggcgacagcaggaagcccgacgtgatcatcagctacggcctggacggcaccatcgtggacaacaagagctacaaggacggcttcaacatcagcaggacctgcgccgacgagatgagcaggtacatcaacgagaacaacctgaggcagaagagcctgaaccccaacgagtggtggaagaacttcgacagcaccatcaccgcctacaccttcctgttcatcaccagctacctgaagggccagttcgaggaccagctggagtacgtgagcaacgccaacggcggcatcaagggcgccgccatcggcgtggagagcctgctgtacctgagcgagggcatcaaggccggcaggatcagccacgccgacttctacagcaacttcaacaacaaggagatgatctac

SEQ ID NO: 130 - DNA, 576 bp, synthetic construct
  source 1..576: mol_type = other DNA
atcgccaagagcgacttcagcatcatcaaggacaacatcaggaggaagctgcagtacgtgaaccacaagtacctgctgctgatcgacctgggcttcgacagcgacagcaacagggactacgagatccagaccgccgagctgctgaccaccgagctggccttcaagggcgccaggctgggcgacaccaggaagcccgacgtgtgcgtgtactacggcgagaacggcctgatcatcgacaacaaggcctacagcaagggctacagcctgcccatgagccaggccgacgagatggtgaggtacatcgaggagaacaaggccaggcagagcagcatcaaccccaaccagtggtggaagatcttcgaggacaccgtgtgcaacttcaactacgccttcgtgagcggcgagttcaccggcggcttcaaggacaggctgaacaacatctgcgagaggaccagggtgagcggcggcgccatcaacaccatcaacctgctgctgctggccgaggagctgaagagcggcaggatgagctaccccaagtgcttcagctacttcgacaccaacgacgaggtgcacatc

SEQ ID NO: 131 - DNA, 498 bp, synthetic construct
  source 1..498: mol_type = other DNA
ctgaagtacctgggcatcaagaagcagaacagggccttcgagatcatcaccgccgagctgttcaacaccagctacaagctgagcgccacccacctgggcggcggcaggaggcccgacgtgctggtgtacaacgacaacttcggcatcatcgtggacaccaaggcctacaaggacggctacggcaggaacgtgaaccaggaggacgagatggtgaggtacatcaccgagaacaacatcaggaagcaggacatcaacaagaacgactggtggaagtacttcagcaagagcatccccagcaccagctactaccacctgtggatcagcagccagttcgtgggcatgttcagcgaccagctgagggagaccagcagcaggaccggcgagaacggcggcgccatgaacgtggagcagctgctgatcggcgccaaccaggtgctgaacaacgtgctggaccccaactgcctgcccaagtacatggagaacaaggagatcatcttc

SEQ ID NO: 132 - DNA, 579 bp, synthetic construct
  source 1..579: mol_type = other DNA
gtgcccgtgaagagcgaggtgagcctgtgcaaggactacctgaggagccacctgaaccacgtggaccacaagtacctgatcctgctggacctgggcttcgacggcaccagcgacagggactacgagatccagaccgcccagctgctgaccggcgagctgaacttcaagggcgccaggctgggcgacaccaggaagcccgacgtgtgcgtgtactacggcgaggacggcctgatcatcgacaacaaggcctacggcaagggctacagcctgcccatcaagcaggccgacgagatgtacaggtacatcgaggagaacaaggagaggaacgagaagctgaaccccaacaagtggtgggagatcttcgacaaggacgtgatccactaccacttcgccttcgtgagcggcgccttcaccggcggcttcagggagaggctggagaacatcaggatgaggagcggcatctacggcgccgccgtgaacagcatgaacctgctgctgatggccgaggagctgaagagcggcaggctgggctacaaggagtgcttcaagctgttcgactgcaacgacgagatcgtgctg

SEQ ID NO: 133 - DNA, 579 bp, synthetic construct
  source 1..579: mol_type = other DNA
gtgcccgtgaagagcgaggtgagcctgctgaaggactacctgaggacccacctgctgcacgtggaccacaggtacctgatcctgctggacctgggcttcgacggcaccagcgacagggactacgagatccagaccgcccagctgctgaccggcgagctgaacttcaagggcgccaggctgggcgacaccaggaagcccgacgtgtgcgtgtactacggcgaggacggcctgatcatcgacaacaaggcctacggcaagggctacagcctgcccatcaagcaggccgacgagatgtacaggtacatcgaggagaacaaggagaggaacgagaagctgaaccccaacaagtggtgggagatcttcgacaacgacgtgatccactaccacttcgccttcatcagcggcgccttcaccggcggcttcaaggagaggctggacaacatcaggatgaggagcggcatctacggcgccgccgtgaacagcatgaacctgctgctgatggccgaggagctgaagagcggcaggctgggctacaaggagtgcttcaagctgttcgactgcaacgacgagatcgtgctg

SEQ ID NO: 134 - DNA, 579 bp, synthetic construct
  source 1..579: mol_type = other DNA
gtgcccgtgaagagcgaggtgagcctgtgcaaggactacctgaggagccacctgaaccacgtggaccacaagtacctgatcctgctggacctgggcttcgacggcaccagcgacagggactacgagatccagaccgcccagctgctgaccggcgagctgaacttcaagggcgccaggctgggcgacaccaggaagcccgacgtgtgcgtgtactacggcgaggacggcctgatcatcgacaacaaggcctacggcaagggctacagcctgcccatcaagcaggccgacgagatgtacaggtacatcgaggagaacaaggagaggaacgagaagctgaaccccaacaagtggtgggagatcttcgacaacgacgtgatccactaccacttcgccttcgtgagcggcgccttcaccggcggcttcagggagaggctggagaacatcaggatgaggagcggcatctacggcgccgccgtgaacagcatgaacctgctgctgatggccgaggagctgaagagcggcaggctgggctacaaggagtgcttcaagctgttcgactgcaacgacgagatcgtgctg

SEQ ID NO: 135 - DNA, 579 bp, synthetic construct
  source 1..579: mol_type = other DNA
gtgcccgtgaagagcgagatgagcctgctgaaggactacctgaggacccacctgctgcacgtggaccacaggtacctgatcctgctggacctgggcttcgacggcgccagcgacagggactacgagatccagaccgcccagctgctgaccggcgagctgaacttcaagggcgccaggctgggcgacaccaggaagcccgacgtgtgcgtgtactacggcgaggacggcctgatcatcgacaacaaggcctacggcaagggctacagcctgcccatcaagcaggccgacgagatgtacaggtacatcgaggagaacaaggagaggaacgagaagctgaaccccaacaagtggtgggagatcttcgacaacgacgtgatccactaccacttcgccttcgtgagcggcgccttcaccggcggcttcaaggagaggctggacaacatcaggatgaggagcggcatctacggcgccgccgtgaacagcatgaacctgctgctgatggccgaggagctgaagagcggcaggctgggctacaaggagtgcttcaagctgttcgactgcaacgacgagatcgtgctg

SEQ ID NO: 136 - DNA, 579 bp, synthetic construct
  source 1..579: mol_type = other DNA
atcctggtggacaaggagagggagatgaggaaggccaagttcctgaaggagaccgtgctggacagcaagttcatcagcctgctggacctggccgccgacgccaccaagagcagggacttcgagatcgtgaccgccgagctgttcaaggaggcctacaacctgaacagcgtgctgctgggcggcagcaacaagcccgacggcctggtgttcaccgacgacttcggcatcctgctggacaccaaggcctacaagaacggcttcagcatctacgccaaggacagggaccagatgatcaggtacgtggacgacaacaacaagagggacaagatcaggaaccccaacgagtggtggaagagcttcagccccctgatccccaacgacaagttctactacctgtgggtgagcaacttcttcaagggccagttcaagaaccagatcgagtacgtgaacagggagaccaacacctacggcgccgtgctgaacgtggagcagctgctgtacggcgccgacgccgtgatcaagggcatcatcaaccccaacaagctgcacgagtacttcagcaacgacgagatcaagttc

SEQ ID NO: 137 - DNA, 579 bp, synthetic construct
  source 1..579: mol_type = other DNA
accgtggacgagaaggagaggctggagctgaaggagtacttcatcagcaacaccaggatccccagcaagtacatcaccctgctggacctggcctacgacggcaacgccaacagggacttcgagatcgtgaccgccgagctgttcaaggacatcttcaagctgcagagcaagcacatgggcggcaccaggaagcccgacatcctgatctggaccgacaagttcggcgtgatcgccgacaccaaggcctacagcaagggctacaagaagaacatcagcgaggccgacaagatggtgaggtacgtgaacgagaacaccaacaggaacaaggtggacaacaccaacgagtggtggaacagcttcgacagcaggatccccaaggacgcctactacttcctgtggatcagcagcgagttcgtgggcaagttcgacgagcagctgaccgagaccagcagcaggaccggcaggaacggcgccagcatcaacgtgtaccagctgctgaggggcgccgacctggtgcagaagagcaagttcaacatccacgacctgcccaacctgatgcagaacaacgagatcaagttc

SEQ ID NO: 138 - DNA, 585 bp, synthetic construct
  source 1..585: mol_type = other DNA
accctgcagaagagcgacatcgagaagttcaagaaccagctgaggaccgagctgaccaacatcgaccacagctacctgaagggcatcgacatcgccagcaagaagaccaccaccaacgtggagaacaccgagttcgaggccatcagcaccaaggtgttcaccgacgagctgggcttcttcggcgagcacctgggcggcagcaacaagcccgacggcctgatctgggacaacgactgcgccatcatcctggacagcaaggcctacagcgagggcttccccctgaccgccagccacaccgacgccatgggcaggtacctgaggcagttcaaggagaggaaggaggagatcaagcccacctggtgggacatcgcccccgacaacctggccaacacctacttcgcctacgtgagcggcagcttcagcggcaactacaaggcccagctgcagaagttcaggcaggacaccaaccacatgggcggcgccctggagttcgtgaagctgctgctgctggccaacaactacaaggcccacaagatgagcatcaacgaggtgaaggagagcatcctggactacaacatcagctac

SEQ ID NO: 139 - DNA, 582 bp, synthetic construct
  source 1..582: mol_type = other DNA
gtgaaggagaagaccgacgccgccctggtgaaggagagggtgaggctgcagctgcacaacatcaaccacaagtacctggccctgatcgactacgccttcagcggcaagaacaacagcagggacttcgaggtgtacaccatcgacctgctggtgaacgagctgaccttcggcggcctgcacctgggcggcaccaggaagcccgacggcatcttctaccacggcagcaacggcatcatcatcgacaacaaggcctacgccaagggcttcgtgatcaccaggaacatggccgacgagatgatcaggtacgtgcaggagaacaacgacaggaaccccgagaggaaccccaactgctggtggaagggcttcccccacgacgtgaccaggtacaactacgtgttcatcagcagcatgttcaagggcgaggtggagcacatgctggacaacatcaggcagagcaccggcatcgacggctgcgtgctgaccatcgagaacctgctgtactacgccgacgccatcaagggcggcaccctgagcaaggccaccttcatcaacggcttcaacgccaacaaggagatggtgttc

SEQ ID NO: 140 - DNA, 585 bp, synthetic construct
  source 1..585: mol_type = other DNA
gtgaaggagaccaccgacagcgtgatcatcaaggacagggtgaggctgaagctgcaccacgtgaaccacaagtacctgaccctgatcgactacgccttcagcggcaagaacaactgcatggacttcgaggtgtacaccatcgacctgctggtgaacgagctggccttcaacggcgtgcacctgggcggcaccaggaagcccgacggcatcttctaccacaacaggaacggcatcatcatcgacaacaaggcctacagccacggcttcaccctgagcagggccatggccgacgagatgatcaggtacatccaggagaacaacgacaggaaccccgagaggaaccccaacaagtggtgggagaacttcgacaagggcgtgaaccagttcaacttcgtgttcatcagcagcctgttcaagggcgagatcgagcacatgctgaccaacatcaagcagagcaccgacggcgtggagggctgcgtgctgagcgccgagaacctgctgtacttcgccgaggccatgaagagcggcgtgatgcccaagaccgagttcatcagctacttcggcgccggcaaggagatccagttc

SEQ ID NO: 141 - DNA, 603 bp, synthetic construct
  source 1..603: mol_type = other DNA
agcgcctgcaaggccgacatcaccgagctgaaggacaagatcaggaagagcctgaaggtgctggaccacaagtacctggtgctggtggacctggcctacagcgacgccagcaccaagagcaagaagaacagcgacgccagggagttcgagatccagaccgccgacctgttcaccaaggagctgaagttcgacggcatgaggctgggcgacagcaacaggcccgacgtgatcatcagccacgacaacttcggcaccatcatcgacaacaagagctacaaggacggcttcaacatcgacaagaagtgcgccgacgagatgagcaggtacatcaacgagaaccagaggaggatccccgagctgcccaagaacgagtggtggaagaacttcgacgtgaacgtggacatcttcaccttcctgttcatcaccagctacctgaagggcaacttcaaggaccagctggagtacatcagcaagagccagagcgacatcaagggcgccgccatcagcgtggagcacctgctgtacatcagcgagaaggtgaagaacggcagcatggacaaggccgacttcttcaagctgttcaacaacgacgagatcagggtg

SEQ ID NO: 142 - DNA, 579 bp, synthetic construct
  source 1..579: mol_type = other DNA
gtgctgaaggacaagcacctggagaagatcaaggagaagttcctggagaacaccagcctggaccccaggttcatcagcctgatcgagatcagcagggacaagaagcagaacagggccttcgagatcatcaccgccgagctgttcaacaccagctacaacctgagcgccatccacctgggcggcggcaggaggcccgacgtgctggcctacaacgacaacttcggcatcatcgtggacaccaaggcctacaagaacggctacggcaggaacgtgaaccaggaggacgagatggtgaggtacatcaccgagaacaagatcaggaagcaggacatcagcaagaacaactggtggaagtacttcagcaagagcatccccagcaccagctactaccacctgtggatcagcagcgagttcgtgggcatgttcagcgaccagctgagggagaccagcagcaggaccggcgagaacggcggcgccatgaacgtggagcagctgctgatcggcgccaaccaggtgctgaacaacgtgctggaccccaacaggctgcccgagtacatggagaacaaggagatcatcttc

SEQ ID NO: 143 - DNA, 579 bp, synthetic construct
  source 1..579: mol_type = other DNA
gccctgaaggacaagcacctggagaagatcaaggagaagttcctggagaacaccagcctggaccccaggttcatcagcctgatcgagatcagcagggacaagaagcagaacagggccttcgagatcatcaccgccgagctgttcaacaccagctacaagctgagcgccacccacctgggcggcggcaggaggcccgacgtgctggtgtacaacgacaacttcggcatcatcgtggacaccaaggcctacaaggacggctacggcaggaacgtgaaccaggaggacgagatggtgaggtacatcaccgagaacaacatcaggaagcaggacatcaacaagaacgactggtggaagtacttcagcaagagcatccccagcaccagctactaccacctgtggatcagcagccagttcgtgggcatgttcagcgaccagctgagggagaccagcagcaggaccggcgagaacggcggcgccatgaacgtggagcagctgctgatcggcgccaaccaggtgctgaacaacgtgctggaccccaactgcctgcccaagtacatggagaacaaggagatcatcttc

SEQ ID NO: 144 - DNA, 585 bp, synthetic construct
  source 1..585: mol_type = other DNA
gtgctggagaagagcgacatcgagaagttcaagaaccagctgaggaccgagctgaccaacatcgaccacagctacctgaagggcatcgacatcgccagcaagaagaagaccagcaacgtggagaacaccgagttcgaggccatcagcaccaagatcttcaccgacgagctgggcttcagcggcaagcacctgggcggcagcaacaagcccgacggcctgctgtgggacgacgactgcgccatcatcctggacagcaaggcctacagcgagggcttccccctgaccgccagccacaccgacgccatgggcaggtacctgaggcagttcaccgagaggaaggaggagatcaagcccacctggtgggacatcgcccccgagcacctggacaacacctacttcgcctacgtgagcggcagcttcagcggcaactacaaggagcagctgcagaagttcaggcaggacaccaaccacctgggcggcgccctggagttcgtgaagctgctgctgctggccaacaactacaagacccagaagatgagcaagaaggaggtgaagaagagcatcctggactacaacatcagctac

SEQ ID NO: 145 - DNA, 579 bp, synthetic construct
  source 1..579: mol_type = other DNA
gccgaggccgacgtgaccagcgagaagatcaagaaccacttcaggagggtgaccgagctgcccgagaggtacctggagctgctggacatcgccttcgaccacaagaggaacagggacttcgagatggtgaccgccggcctgttcaaggacgtgtacggcctggagagcgtgcacctgggcggcgccaacaagcccgacggcgtggtgtacaacgacaacttcggcatcatcctggacaccaaggcctacgagaacggctacggcaagcacatcagccagatcgacgagatggtgaggtacatcgacgacaacaggctgagggacaccaccaggaaccccaacaagtggtgggagaacttcgacgccgacatccccagcgaccagttctactacctgtgggtgagcggcaagttcctgcccaacttcgccgagcagctgaagcagaccaactacaggagccacgccaacggcggcggcctggaggtgcagcagctgctgctgggcgccgacgccgtgaagaggaggaagctggacgtgaacaccatccccaactacatgaagaacgaggtgatcaccctg

SEQ ID NO: 146 - DNA, 579 bp, synthetic construct
  source 1..579: mol_type = other DNA
gccgaggccgacctgaacagcgagaagatcaagaaccactacaggaagatcaccaacctgcccgagaagtacatcgagctgctggacatcgccttcgaccacaggaggcaccaggacttcgagatcgtgaccgccggcctgttcaaggactgctacggcctgagcagcatccacctgggcggccagaacaagcccgacggcgtggtgttcaacaacaagttcggcatcatcctggacaccaaggcctacgagaagggctacggcatgcacatcggccagatcgacgagatgtgcaggtacatcgacgacaacaagaagagggacatcgtgaggcagcccaacgagtggtggaagaacttcggcgacaacatccccaaggaccagttctactacctgtggatcagcggcaagttcctgcccaggttcaacgagcagctgaagcagacccactacaggaccagcatcaacggcggcggcctggaggtgagccagctgctgctgggcgccaacgccgccatgaagggcaagctggacgtgaacaccctgcccaagcacatgaacaaccaggtgatcaagctg

SEQ ID NO: 147 - DNA, 609 bp, synthetic construct
  source 1..609: mol_type = other DNA
gtgctgaaggacgccgccctgcagaagaccaagaacaccctgctgaacgagctgaccgagatcgaccccgccgacatcgaggtgatcgagatgagctggaagaaggccaccaccaggagccagaacaccctggaggccaccctgttcgaggtgaaggtggtggagatcttcaagaagtacttcgagctgaacggcgagcacctgggcggccagaacaggcccgacggcgccgtgtactacaacagcacctacggcatcatcctggacaccaaggcctacagcaacggctacaacatccccgtggaccagcagagggagatggtggactacatcaccgacgtgatcgacaagaaccagaacgtgacccccaacaggtggtgggaggccttccccgccaccctgctgaagaacaacatctactacctgtgggtggccggcggcttcaccggcaagtacctggaccagctgaccaggacccacaaccagaccaacatggacggcggcgccatgaccaccgaggtgctgctgaggctggccaacaaggtgagcagcggcaacctgaagaccaccgacatccccaagctgatgaccaacaagctgatcctgagc

SEQ ID NO: 148 - DNA, 576 bp, synthetic construct
  source 1..576: mol_type = other DNA
gccgaggccgacctggacagcgagaggatcaagaaccactacaggaagatcaccaacctgcccgagaagtacatcgagctgctggacatcgccttcgaccaccacaggcaccaggacttcgagatcatcaccgccggcctgttcaaggactgctacggcctgagcagcatccacctgggcggccagaacaagcccgacggcgtggtgttcaacggcaagttcggcatcatcctggacaccaaggcctacgagaagggctacggcatgcacatcaaccagatcgacgagatgtgcaggtacatcgaggacaacaagcagagggacaagatcaggcagcccaacgagtggtggaacaacttcggcgacaacatccccgagaacaagttctactacctgtgggtgagcggcaagttcctgcccaagttcaacgagcagctgaagcagacccactacaggaccggcatcaacggcggcggcctggaggtgagccagctgctgctgggcgccgacgccgtgatgaagggcgccctgaacgtgaacatcctgcccacctacatgcacaacaacgtgatccag

SEQ ID NO: 149 - DNA, 579 bp, synthetic construct
  source 1..579: mol_type = other DNA
gagatcagcgacatcgccctgcagaaggagaaggcctacttctacaagaacaccgccctgagcaagaggcacatcagcatcctggagatcgccttcgacggcagcaagaacagggacctggagatcctgagcgccgaggtgttcaaggactactaccagctggagagcatccacctgggcggcggcctgaagcccgacggcatcgccttcaaccagaacttcggcatcatcgtggacaccaaggcctacaagggcgtgtacagcaggagcagggccgaggccgacaagatgttcaggtacatcgaggacaacaagaagagggaccccaagaggaaccagagcctgtggtggaggagcttcaacgagcacatccccgccaacaacttctacttcctgtggatcagcggcaagttccagaggaacttcgacacccagatcaaccagctgaactacgagaccggctacaggggcggcgccctgagcgccaggcagttcctgatcggcgccgacgccatccagaagggcaagatcgacatcaacgacctgcccagctacttcaacaacagcgtgatcagcttc

SEQ ID NO: 150 - DNA, 579 bp, synthetic construct
  source 1..579: mol_type = other DNA
accagcagggagaagagcaggctgaacctgaaggagtacttcgtgagcaacaccaacctgcccaacaagttcatcaccctgctggacctggcctacgacggcaaggccaacagggacttcgagctgatcaccagcgagctgttcagggagatctacaagctgaacaccaggcacctgggcggcaccaggaagcccgacatcctgatctggaacgagaacttcggcatcatcgccgacaccaaggcctacagcaagggctacaagaagaacatcagcgaggaggacaagatggtgaggtacatcgacgagaacatcaagaggagcaaggactacaaccccaacgagtggtggaaggtgttcgacaacgagatcagcagcaacaactacttctacctgtggatcagcagcgagttcatcggcaagttcgaggagcagctgcaggagaccgcccagaggaccaacgtgaagggcgccagcatcaacgtgtaccagctgctgatgggcgcccacaaggtgcagaccaaggagctgaacgtgaacagcatccccaagtacatgaacaacaccgagatcaagttc

SEQ ID NO: 151 - DNA, 603 bp, synthetic construct
  source 1..603: mol_type = other DNA
aactgcatcaaggacagcatcatcgacatcaaggacagggtgaggaccaagctggtgcacctggaccacaagtacctggccctgatcgacctggccttcagcgacgccgacaccaggaccaagaagaacagcgacgccagggagttcgagatccagaccgccgacctgttcaccaaggagctgagcttcaacggccagaggctgggcgacagcaggaagcccgacatcatcatcagcttcgacaagatcggcaccatcatcgacaacaagagctacaaggacggcttcaacatcagcaggccctgcgccgacgagatgatcaggtacatcaacgagaacaacctgaggaagaagagcctgaacgccaacgagtggtggaacaagttcgaccccaccatcaccgcctacagcttcctgttcatcaccagctacctgaagggccagttccaggagcagctggagtacatcagcaacgccaacggcggcatcaagggcgccgccatcggcatcgagaacctgctgtacctgagcgaggccctgaagagcggcaagatcagccacaaggacttctaccagaacttcaacaacaaggagatcacctac

SEQ ID NO: 152 - DNA, 591 bp, synthetic construct
  source 1..591: mol_type = other DNA
ctgccccagaaggaccaggtgcagcagcagcaggacgagctgaggcccatgctgaagaacgtggaccacaggtacctgcagctggtggagctggccctggacagcgaccagaacagcgagtacagccagttcgagcagctgaccatggagctggtgctgaagcacctggacttcgacggcaagcccctgggcggcagcaacaagcccgacggcatcgcctgggacaacgacggcaacttcatcatcttcgacaccaaggcctacaacaagggctacagcctggccggcaacaccgacaaggtgaagaggtacatcgacgacgtgagggacagggacaccagcaggaccagcacctggtggcagctggtgcccaagagcatcgacgtgcacaacctgctgaggttcgtgtacgtgagcggcaacttcaccggcaactacatgaagctgctggacagcctgaggagctggagcaacgcccagggcggcctggccagcgtggagaagctgctgctgaccagcgagctgtacctgaggaacatgtacagccaccaggagctgatcgacagctggaccgacaacaacgtgaagcac

SEQ ID NO: 153 - DNA, 567 bp, synthetic construct
  source 1..567: mol_type = other DNA
accaccgacgccgtggtggtgaaggacagggccagggtgaggctgcacaacatcaaccacaagtacctgaccctgatcgactacgccttcagcggcaagaacaactgcaccgagttcgagatctacaccatcgacctgctggtgaacgagctggccttcaacggcatccacctgggcggcaccaggaagcccgacggcatcttcgactacaaccagcagggcatcatcatcgacaacaaggcctacagcaagggcttcaccatcaccaggagcatggccgacgagatggtgaggtacgtgcaggagaacaacgacaggaaccccgagaggaacaagacccagtggtggctgaacttcggcgacaacgtgaaccacttcaacttcgtgttcatcagcagcatgttcaagggcgaggtgaggcacatgctgaacaacatcaagcagagcaccggcgtggacggctgcgtgctgaccgccgagaacctgctgtacttcgccgacgccatcaagggcggcaccgtgaagaggaccgacttcatcaacctgttcggcaagaacgacgagctg

SEQ ID NO: 154 - DNA, 594 bp, synthetic construct
  source 1..594: mol_type = other DNA
ctgcccaagaaggacaacgtgcagaggcagcaggacgagctgaggcccctgctgaagcacgtggaccacaggtacctgcagctggtggagctggccctggacagcagccagaacagcgagtacagcatgctggagagcatgaccatggagctgctgctgacccacctggacttcgacggcgccagcctgggcggcgccagcaagcccgacggcatcgcctgggacaaggacggcaacttcctgatcgtggacaccaaggcctacgacaacggctacagcctggccggcaacaccgacaaggtggccaggtacatcgacgacgtgagggccaaggaccccaacagggccagcacctggtggacccaggtgcccgagagcctgaacgtggacgacaacctgagcttcatgtacgtgagcggcagcttcaccggcaactaccagaggctgctgaaggacctgagggccaggaccaacgccaggggcggcctgaccaccgtggagaagctgctgctgaccagcgaggcctacctggccaagagcggctacggccacacccagctgctgaacgactggaccgacgacaacatcgaccac

SEQ ID NO: 155 - DNA, 579 bp, synthetic construct
  source 1..579: mol_type = other DNA
cagatcaaggacaagtacctggaggacctgaagctggagctgtacaagaagaccaacctgcccaacaagtactacgagatggtggacatcgcctacgacggcaagaggaacagggagttcgagatctacaccagcgacctgatgcaggagatctacggcttcaagaccaccctgctgggcggcaccaggaagcccgacgtggtgagctacagcgacgcccacggctacatcatcgacaccaaggcctacgccaacggctacaggaaggagatcaagcaggaggacgagatggtgaggtacatcgaggacaaccagctgaaggacgtgctgaggaaccccaacaagtggtgggagtgcttcgacgacgccgagcacaagaaggagtactacttcctgtggatcagcagcaagttcgtgggcgagttcagcagccagctgcaggacaccagcaggaggaccggcatcaagggcggcgccgtgaacatcgtgcagctgctgctgggcgcccacctggtgtacagcggcgagatcagcaaggaccagttcgccgcctacatgaacaacaccgagatcaacttc

SEQ ID NO: 156 - DNA, 438 bp, synthetic construct
  source 1..438: mol_type = other DNA
atgaaccccaggaacgagatcgtgatcgccaagcacctgagcggcggcaacaggcccgagatcgtgtgctaccaccccgaggacaagcccgaccacggcctgatcctggacagcaaggcctacaagagcggcttcaccatccccagcggcgagagggacaagatggtgaggtacatcgaggagtacatcaccaagaaccagctgcagaaccccaacgagtggtggaagaacctgaagggcgccgagtaccccggcatcgtgggcttcggcttcatcagcaacagcttcctgggccactacaggaagcagctggactacatcatgaggaggaccaagatcaagggcagcagcatcaccaccgagcacctgctgaagaccgtggaggacgtgctgagcgagaagggcaacgtgatcgacttcttcaagtacttcctggag

SEQ ID NO: 157 - DNA, 585 bp, synthetic construct
  source 1..585: mol_type = other DNA
gagatcaagaaccaggagatcgaggagctgaagcagatcgccctgaacaagtacaccgccctgcccagcgagtgggtggagctgatcgagatcagcagggacaaggaccagagcaccatcttcgagatgaaggtggccgagctgttcaagacctgctacaggatcaagagcctgcacctgggcggcgccagcaagcccgactgcctgctgtgggacgacagcttcagcgtgatcgtggacgccaaggcctacaaggacggcttccccttccaggccagcgagaaggacaagatggtgaggtacctgagggagtgcgagaggaaggacaaggccgagaacgccaccgagtggtggaacaacttcccccccgagctgaacagcaaccagctgttcttcatgttcgccagcagcttcttcagcagcaccgccgagaagcacctggagagcgtgagcatcgccagcaagttcagcggctgcgcctgggacgtggacaacctgctgagcggcgccaacttcttcctgcagaacccccaggccaccctgcagtaccacctgatcagggtgttcagcaacaaggtggtggac

SEQ ID NO: 158 - DNA, 594 bp, synthetic construct
  source 1..594: mol_type = other DNA
ctgccccacaaggacaacgtgatcaagcagcaggacgagctgaggcccatgctgaagcacgtgaaccacaagtacctgcagctggtggagctggccttcgagagcagcaggaacagcgagtacagccagttcgagaccctgaccatggagctggtgctgaagtacctggacttcagcggcaagagcctgggcggcgccaacaagcccgacggcatcgcctgggaccccctgggcaacttcctgatcttcgacaccaaggcctacaagcacggctacaccctgagcaacaacaccgacagggtggccaggtacatcaacgacgtgagggacaaggacatccagaggatcagcaggtggtggcagagcatccccacctacatcgacgtgaagaacaagctgcagttcgtgtacatcagcggcagcttcaccggccactacctgaggctgctgaacgacctgaggagcaggaccagggccaagggcggcctggtgaccgtggagaagctgctgctgaccaccgagaggtacctggccgaggccgactacacccacaaggagctgttcgacgactggatggacgacaacatcgagcac

SEQ ID NO: 159 - DNA, 600 bp, synthetic construct
  source 1..600: mol_type = other DNA
aggatcagccccagcaacctggagcagaccaagcagcagctgagggaggagctgatcaacctggaccaccagtacctggacatcctggacttcagcatcgccggcaacgtgggcgccaggcagttcgaggtgaggatcgtggagctgctgaacgagatcatcatcgccaagcacctgagcggcggcaacaggcccgagatcatcggcttcaaccccaaggagaaccccgaggactgcatcatcatggacagcaaggcctacaaggagggcttcaacatccccgccaacgagagggacaagatgatcaggtacgtggaggagtacaacgccaaggacaacaccctgaacaacaacaagtggtggaagaacttcgagagccccaactaccccaccaaccaggtgaagttcagcttcgtgagcagcagcttcatcggccagttcaccaaccagctgacctacatcaacaacaggaccaacgtgaacggcagcgccatcaccgccgagaccctgctgaggaaggtggagaacgtgatgaacgtgaacaccgagtacaacctgaacaacttcttcgaggagctgggcagcaacaccctggtggcc

SEQ ID NO: 160 - DNA, 582 bp, synthetic construct
  source 1..582: mol_type = other DNA
accttcgacagcaccgtggccgacaacctgaagaacctgatcctgcccaagctgaaggagctggaccacaagtacctgcaggccatcgacatcgcctacaagaggagcaacaccaccaaccacgagaacaccctgctggaggtgctgagcgccgacctgttcaccaaggagatggactaccacggcaagcacctgggcggcgccaacaagcccgacggcttcgtgtacgacgaggagaccggctggatcctggacagcaaggcctacagggacggcttcgccgtgaccgcccacaccaccgacgccatgggcaggtacatcgaccagtacagggacagggacgacaagagcacctggtgggaggacttccccaaggacctgccccagacctacttcgcctacgtgagcggcttctacatcggcaagtaccaggagcagctgcaggacttcgagaacaggaagcacatgaagggcggcctgatcgaggtggccaagctgatcctgctggccgagaagtacaaggagaacaagatcacccacgaccagatcaccctgcagatcctgaacgaccacatcagccag

SEQ ID NO: 161 - DNA, 558 bp, synthetic construct
  source 1..558: mol_type = other DNA
cccctggacgtggtggagcagatgaaggccgagctgaggcccctgctgaaccacgtgaaccacaggctgctggccatcatcgacttcagctacaacatgagcaggggcgacgacaagaggctggaggactacaccgcccagatctacaagctgatcagccacgacacccacctgctggccggccccagcaggcccgacgtggtgagcgtgatcaacgacctgggcatcatcatcgacagcaaggcctacaagcagggcttcaacatcccccaggccgaggaggacaagatggtgaggtacctggacgagagcatcaggagggaccccgccatcaaccccaccaagtggtgggagtacctgggcgccagcaccgagtacgtgttccagttcgtgagcagcagcttcagcagcggcgccagcgccaagctgaggcagatccacaggaggagcagcatcgagggcagcatcatcaccgccaagaacctgctgctgctggccgagaacttcctgtgcaccaacaccatcaacatcgacctgttcaggcagaacaacgagatc

SEQ ID NO: 162 - DNA, 603 bp, synthetic construct
  source 1..603: mol_type = other DNA
cagctggtgcccagctacatcacccagaccaagctgaggctgagcggcctgatcaactacatcgaccacagctacttcgacctgatcgacctgggcttcgacggcaggcagaacaggctgtacgagctgaggatcgtggagctgctgaacctgatcaacagcctgaaggccctgcacctgagcggcggcaacaggcccgagatcatcgcctacagccccgacgtgaaccccatcaacggcgtgatcatggacagcaagagctacaggggcggcttcaacatccccaacagcgagagggacaagatgatcaggtacatcaacgagtacaaccagaagaaccccaccctgaacagcaacaggtggtgggagaacttcagggcccccgactacccccagagccccctgaagtacagcttcgtgagcggcaacttcatcggccagttcctgaaccagatccagtacatcctgacccagaccggcatcaacggcggcgccatcaccagcgagaagctgatcgagaaggtgaacgccgtgctgaaccccaacatcagctacaccatcaacaacttcttcaacgacctgggctgcaacaggctggtgcag

SEQ ID NO: 163 - protein, 196 aa, synthetic construct
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 164 - skipped

SEQ ID NO: 165 - DNA, 45 bp, synthetic construct
  source 1..45: mol_type = other DNA
ggcggtggcggagggatggatgctaagtcactaactgcctggtcc

SEQ ID NO: 166 - protein, 15 aa, synthetic construct
GGGGGMDAKSLTAWS

SEQ ID NO: 167 - skipped

SEQ ID NO: 168 - protein, 35 aa, synthetic construct
LDTEQVVAIASHNGGKQALEAVKADLLDLLGAPYV

SEQ ID NO: 169 - protein, 35 aa, synthetic construct
LDTEQVVAIASHNGGKQALEAVKADLLDLRGAPYA

SEQ ID NO: 170 - protein, 35 aa, synthetic construct
LDTEQVVAIASHNGGKQALEAVKADLLELRGAPYA

SEQ ID NO: 171 - protein, 35 aa, synthetic construct
LDTEQVVAIASHNGGKQALEAVKAHLLDLRGAPYA

SEQ ID NO: 172 - protein, 35 aa, synthetic construct
LNTEQVVAIASHNGGKQALEAVKADLLDLRGAPYA

SEQ ID NO: 173 - protein, 35 aa, synthetic construct
LNTEQVVAIASNNGGKQALEAVKTHLLDLRGARYA

SEQ ID NO: 174 - protein, 35 aa, synthetic construct
LNTEQVVAIASNPGGKQALEAVRALFPDLRAAPYA

SEQ ID NO: 175 - protein, 35 aa, synthetic construct
LNTEQVVAIASSHGGKQALEAVRALFPDLRAAPYA

SEQ ID NO: 176 - protein, 35 aa, synthetic construct
LNTEQVVAVASNKGGKQALEAVGAQLLALRAVPYA

SEQ ID NO: 177 - protein, 35 aa, synthetic construct
LNTEQVVAVASNKGGKQALEAVGAQLLALRAVPYE

SEQ ID NO: 178 - protein, 35 aa, synthetic construct
LSAAQVVAIASHDGGKQALEAVGTQLVALRAAPYA

SEQ ID NO: 179 - protein, 35 aa, synthetic construct
LSIAQVVAVASRSGGKQALEAVRAQLLALRAAPYG

SEQ ID NO: 180 - protein, 35 aa, synthetic construct
LSPEQVVAIASNHGGKQALEAVRALFRGLRAAPYG

SEQ ID NO: 181 - protein, 35 aa, synthetic construct
LSPEQVVAIASNNGGKQALEAVKAQLLELRAAPYE

SEQ ID NO: 182 - protein, 35 aa, synthetic construct
LSTAQLVAIASNPGGKQALEAIRALFRELRAAPYA

SEQ ID NO: 183 - protein, 35 aa, synthetic construct
LSTAQLVAIASNPGGKQALEAVRALFRELRAAPYA

SEQ ID NO: 184 - protein, 35 aa, synthetic construct
LSTAQLVAIASNPGGKQALEAVRAPFREVRAAPYA

SEQ ID NO: 185 - protein, 35 aa, synthetic construct
LSTAQLVSIASNPGGKQALEAVRALFRELRAAPYA

SEQ ID NO: 186 - protein, 35 aa, synthetic construct
LSTAQVAAIASHDGGKQALEAVGTQLVVLRAAPYA

SEQ ID NO: 187 - protein, 35 aa, synthetic construct
LSTAQVATIASSIGGRQALEALKVQLPVLRAAPYG

SEQ ID NO: 188 - protein, 35 aa, synthetic construct
LSTAQVATIASSIGGRQALEAVKVQLPVLRAAPYG

SEQ ID NO: 189 - protein, 35 aa, synthetic construct
LSTAQVVAIAANNGGKQALEAVRALLPVLRVAPYE

SEQ ID NO: 190 - protein, 35 aa, synthetic construct
LSTAQVVAIAGNGGGKQALEGIGEQLLKLRTAPYG

SEQ ID NO: 191 - protein, 35 aa, synthetic construct
LSTAQVVAIASHDGGKQALEAAGTQLVALRAAPYA

SEQ ID NO: 192 - protein, 35 aa, synthetic construct
LSTAQVVAIASHDGGKQALEAVGAQLVELRAAPYA

SEQ ID NO: 193 - protein, 35 aa, synthetic construct
LSTAQVVAIASHDGGKQALEAVGTQLVALRAAPYA

SEQ ID NO: 194 - protein, 35 aa, synthetic construct
LSTAQVVAIASHDGGNQALEAVGTQLVALRAAPYA

SEQ ID NO: 195 - protein, 35 aa, synthetic construct
LSTAQVVAIASHNGGKQALEAVKAQLLDLRGAPYA

SEQ ID NO: 196 - protein, 35 aa, synthetic construct
LSTAQVVAIASNDGGKQALEEVEAQLLALRAAPYE

SEQ ID NO: 197 - protein, 35 aa, synthetic construct
LSTAQVVAIASNGGGKQALEGIGEQLLKLRTAPYG

SEQ ID NO: 198 - protein, 35 aa, synthetic construct
LSTAQVVAIASNGGGKQALEGIGEQLRKLRTAPYG

SEQ ID NO: 199 - protein, 35 aa, synthetic construct
LSTAQVVAIASNPGGKQALEAVRALFRELRAAPYA

SEQ ID NO: 200 - protein, 35 aa, synthetic construct
LSTAQVVAIASQNGGKQALEAVKAQLLDLRGAPYA

SEQ ID NO: 201 - protein, 35 aa, synthetic construct
LSTAQVVAIASSHGGKQALEAVRALFRELRAAPYG

SEQ ID NO: 202 - protein, 35 aa, synthetic construct
LSTAQVVAIASSNGGKQALEAVWALLPVLRATPYD

SEQ ID NO: 203 - protein, 35 aa, synthetic construct
LSTAQVVAIATRSGGKQALEAVRAQLLDLRAAPYG

SEQ ID NO: 204 - protein, 35 aa, synthetic construct
LSTAQVVAVAGRNGGKQALEAVRAQLPALRAAPYG

SEQ ID NO: 205 - protein, 35 aa, synthetic construct
LSTAQVVAVASSNGGKQALEAVWALLPVLRATPYD

SEQ ID NO: 206 - protein, 35 aa, synthetic construct
LSTAQVVTIASSNGGKQALEAVWALLPVLRATPYD

SEQ ID NO: 207 - protein, 35 aa, synthetic construct
LSTEQVVAIAGHDGGKQALEAVGAQLVALRAAPYA

SEQ ID NO: 208 - protein, 35 aa, synthetic construct
LSTEQVVAIASHDGGKQALEAVGAQLVALLAAPYA

SEQ ID NO: 209 - protein, 35 aa, synthetic construct
LSTEQVVAIASHDGGKQALEAVGAQLVALRAAPYA

SEQ ID NO: 210 - protein, 35 aa, synthetic construct
LSTEQVVAIASHDGGKQALEAVGGQLVALRAAPYA

SEQ ID NO: 211 - protein, 35 aa, synthetic construct
LSTEQVVAIASHDGGKQALEAVGTQLVALRAAPYA

SEQ ID NO: 212 - protein, 35 aa, synthetic construct
LSTEQVVAIASHDGGKQALEAVGVQLVALRAAPYA

SEQ ID NO: 213 - protein, 35 aa, synthetic construct
LSTEQVVAIASHDGGKQALEAVVAQLVALRAAPYA

SEQ ID NO: 214 - protein, 35 aa, synthetic construct
LSTEQVVAIASHDGGKQPLEAVGAQLVALRAAPYA

SEQ ID NO: 215 - protein, 35 aa, synthetic construct
LSTEQVVAIASHGGGKQVLEGIGEQLLKLRAAPYG

SEQ ID NO: 216 - protein, 35 aa, synthetic construct
LSTEQVVAIASHKGGKQALEGIGEQLLKLRAAPYG

SEQ ID NO: 217 - protein, 35 aa, synthetic construct
LSTEQVVAIASHNGGKQALEAVKADLLDLRGAPYA

SEQ ID NO: 218 - protein, 35 aa, synthetic construct
LSTEQVVAIASHNGGKQALEAVKADLLELRGAPYA

SEQ ID NO: 219 - protein, 35 aa, synthetic construct
LSTEQVVAIASHNGGKQALEAVKAHLLDLRGAPYA

SEQ ID NO: 220 - protein, 35 aa, synthetic construct
LSTEQVVAIASHNGGKQALEAVKAHLLDLRGVPYA

SEQ ID NO: 221 - protein, 35 aa, synthetic construct
LSTEQVVAIASHNGGKQALEAVKAHLLELRGAPYA

SEQ ID NO: 222 - protein, 35 aa, synthetic construct
LSTEQVVAIASHNGGKQALEAVKAQLLDLRGAPYA

SEQ ID NO: 223 - protein, 35 aa, synthetic construct
LSTEQVVAIASHNGGKQALEAVKAQLLELRGAPYA

SEQ ID NO: 224 - protein, 35 aa, synthetic construct
LSTEQVVAIASHNGGKQALEAVKAQLPVLRRAPYG

SEQ ID NO: 225 - protein, 35 aa, synthetic construct
LSTEQVVAIASHNGGKQALEAVKTQLLELRGAPYA

SEQ ID NO: 226 - protein, 35 aa, synthetic construct
LSTEQVVAIASHNGGKQALEAVRAQLPALRAAPYG

SEQ ID NO: 227 - protein, 35 aa, synthetic construct
LSTEQVVAIASHNGSKQALEAVKAQLLDLRGAPYA

SEQ ID NO: 228 - protein, 35 aa, synthetic construct
LSTEQVVAIASNGGGKQALEGIGKQLQELRAAPHG

SEQ ID NO: 229 - protein, 35 aa, synthetic construct
LSTEQVVAIASNGGGKQALEGIGKQLQELRAAPYG

SEQ ID NO: 230 - protein, 35 aa, synthetic construct
LSTEQVVAIASNHGGKQALEAVRALFRELRAAPYA

SEQ ID NO: 231 - protein, 35 aa, synthetic construct
LSTEQVVAIASNHGGKQALEAVRALFRGLRAAPYG

SEQ ID NO: 232 - protein, 35 aa, synthetic construct
LSTEQVVAIASNKGGKQALEAVKADLLDLRGAPYV

SEQ ID NO: 233 - protein, 35 aa, synthetic construct
LSTEQVVAIASNKGGKQALEAVKAHLLDLLGAPYV

SEQ ID NO: 234 - protein, 35 aa, synthetic construct
LSTEQVVAIASNKGGKQALEAVKAQLLALRAAPYA

SEQ ID NO: 235 - protein, 35 aa, synthetic construct
LSTEQVVAIASNKGGKQALEAVKAQLLELRGAPYA

SEQ ID NO: 236 - protein, 35 aa, synthetic construct
LSTEQVVAIASNNGGKQALEAVKALLLELRAAPYE

SEQ ID NO: 237 - protein, 35 aa, synthetic construct
LSTEQVVAIASNNGGKQALEAVKAQLLALRAAPYE

SEQ ID NO: 238 - protein, 35 aa, synthetic construct
LSTEQVVAIASNNGGKQALEAVKAQLLDLRGAPYA

SEQ ID NO: 239 - protein, 35 aa, synthetic construct
LSTEQVVAIASNNGGKQALEAVKAQLLVLRAAPYG

SEQ ID NO: 240 - protein, 35 aa, synthetic construct
LSTEQVVAIASNNGGKQALEAVKAQLPALRAAPYE

SEQ ID NO: 241 - protein, 35 aa, synthetic construct
LSTEQVVAIASNNGGKQALEAVKAQLPVLRRAPCG

SEQ ID NO: 242 - protein, 35 aa, synthetic construct
LSTEQVVAIASNNGGKQALEAVKAQLPVLRRAPYG

SEQ ID NO: 243 - protein, 35 aa, synthetic construct
LSTEQVVAIASNNGGKQALEAVKARLLDLRGAPYA

SEQ ID NO: 244 - protein, 35 aa, synthetic construct
LSTEQVVAIASNNGGKQALEAVKTQLLALRTAPYE

SEQ ID NO: 245 - protein, 35 aa, synthetic construct
LSTEQVVAIASNPGGKQALEAVRALFPDLRAAPYA

SEQ ID NO: 246 - protein, 35 aa, synthetic construct
LSTEQVVAIASSHGGKQALEAVRALFPDLRAAPYA

SEQ ID NO: 247 - protein, 35 aa, synthetic construct
LSTEQVVAIASSHGGKQALEAVRALLPVLRATPYD

SEQ ID NO: 248 - protein, 35 aa, synthetic construct
LSTEQVVAVASHNGGKQALEAVRAQLLDLRAAPYE

SEQ ID NO: 249 - protein, 35 aa, synthetic construct
LSTEQVVAVASNKGGKQALAAVEAQLLRLRAAPYE

SEQ ID NO: 250 - protein, 35 aa, synthetic construct
LSTEQVVAVASNKGGKQALEEVEAQLLRLRAAPYE

SEQ ID NO: 251 - protein, 35 aa, synthetic construct
LSTEQVVAVASNKGGKQVLEAVGAQLLALRAVPYE

SEQ ID NO: 252 - protein, 35 aa, synthetic construct
LSTEQVVAVASNNGGKQALKAVKAQLLALRAAPYE

SEQ ID NO: 253 - protein, 35 aa, synthetic construct
LSTEQVVVIANSIGGKQALEAVKVQLPVLRAAPYE

SEQ ID NO: 254 - protein, 35 aa, synthetic construct
LSTGQVVAIASNGGGRQALEAVREQLLALRAVPYE

SEQ ID NO: 255 - protein, 35 aa, synthetic construct
LSVAQVVTIASHNGGKQALEAVRAQLLALRAAPYG

SEQ ID NO: 256 - protein, 35 aa, synthetic construct
LTIAQVVAVASHNGGKQALEAIGAQLLALRAAPYA

SEQ ID NO: 257 - protein, 35 aa, synthetic construct
LTIAQVVAVASHNGGKQALEVIGAQLLALRAAPYA

SEQ ID NO: 258 - protein, 35 aa, synthetic construct
LTPQQVVAIAANTGGKQALGAITTQLPILRAAPYE

SEQ ID NO: 259 - protein, 35 aa, synthetic construct
LTPQQVVAIASNTGGKQALEAVTVQLRVLRGARYG

SEQ ID NO: 260 - protein, 35 aa, synthetic construct
LTPQQVVAIASNTGGKRALEAVCVQLPVLRAAPYR

SEQ ID NO: 261 - protein, 35 aa, synthetic construct
LTPQQVVAIASNTGGKRALEAVRVQLPVLRAAPYE

SEQ ID NO: 262 - protein, 35 aa, synthetic construct
LTTAQVVAIASNDGGKQALEAVGAQLLVLRAVPYE

SEQ ID NO: 263 - protein, 35 aa, synthetic construct
LTTAQVVAIASNDGGKQTLEVAGAQLLALRAVPYE

SEQ ID NO: 264 - protein, 137 aa, synthetic construct
FGKLVALGYSREQIRKLKQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLH

SEQ ID NO: 265 - protein, 19 aa, synthetic construct
LSTAQVVAIACISGQQALE

SEQ ID NO: 266 - protein, 62 aa, synthetic construct
AIEAHMPTLRQASHSLSPERVAAIACIGGRSAVEAVRQGLPVKAIRRIRREKAPVAGPPPAS

SEQ ID NO: 267 - protein, 35 aa, synthetic construct
  VARIANT 2: note = X at position 2 can be selected from any one of D, N, S, T.
  VARIANT 3: note = X at position 3 can be selected from any one of I, T, V.
  VARIANT 4: note = X at position 4 can be selected from any one of A, E.
  VARIANT 8: note = X at position 8 can be selected from any one of A, T.
  VARIANT 9: note = X at position 9 can be selected from any one of I, V.
  VARIANT 15: note = X at position 15 can be selected from any one of G, S.
  VARIANT 21: note = X at position 21 can be selected from any one of A, V.
  VARIANT 22: note = X at position 22 can be selected from any one of I, V.
  VARIANT 23: note = X at position 23 can be selected from any one of G, K, R.
  VARIANT 24: note = X at position 24 can be selected from any one of A, T.
  VARIANT 25: note = X at position 25 can be selected from any one of D, H, Q.
  VARIANT 27: note = X at position 27 can be selected from any one of L, P.
  VARIANT 28: note = X at position 28 can be selected from any one of A, D, E, V.
  VARIANT 30: note = X at position 30 can be selected from any one of L, R.
  VARIANT 31: note = X at position 31 can be selected from any one of A, G, R.
  VARIANT 32: note = X at position 32 can be selected from any one of A, V.
  VARIANT 35: note = X at position 35 can be selected from any one of A, E, G, V.
LXXXQVVXXASHNGXKQALEXXXXXLXXLXXXPYX

SEQ ID NO: 268 - protein, 35 aa, synthetic construct
  VARIANT 2: note = X at position 2 can be selected from any one of N, S.
  VARIANT 3: note = X at position 3 can be selected from any one of P, T.
  VARIANT 4: note = X at position 4 can be selected from any one of A, E.
  VARIANT 9: note = X at position 9 can be selected from any one of I, V.
  VARIANT 11: note = X at position 11 can be selected from any one of A, S.
  VARIANT 20: note = X at position 20 can be selected from any one of E, K.
  VARIANT 23: note = X at position 23 can be selected from any one of K, R.
  VARIANT 24: note = X at position 24 can be selected from any one of A, T.
  VARIANT 25: note = X at position 25 can be selected from any one of H, L, Q, R.
  VARIANT 27: note = X at position 27 can be selected from any one of L, P.
  VARIANT 28: note = X at position 28 can be selected from any one of A, D, E, V.
  VARIANT 31: note = X at position 31 can be selected from any one of A, G, R, T, V.
  VARIANT 33: note = X at position 33 can be selected from any one of P, R.
  VARIANT 34: note = X at position 34 can be selected from any one of C, Y.
  VARIANT 35: note = X at position 35 can be selected from any one of A, E, G.
LXXXQVVAXAXNNGGKQALXAVXXXLXXLRXAXXX

SEQ ID NO: 269 - protein, 35 aa, synthetic construct
  VARIANT 2: note = X at position 2 can be selected from any one of N, S.
  VARIANT 4: note = X at position 4 can be selected from any one of A, E.
  VARIANT 6: note = X at position 6 can be selected from any one of L, V.
  VARIANT 8: note = X at position 8 can be selected from any one of A, S.
  VARIANT 22: note = X at position 22 can be selected from any one of I, V.
  VARIANT 25: note = X at position 25 can be selected from any one of L, P.
  VARIANT 27: note = X at position 27 can be selected from any one of P, R.
  VARIANT 28: note = X at position 28 can be selected from any one of D, E.
  VARIANT 29: note = X at position 29 can be selected from any one of L, V.
LXTXQXVXIASNPGGKQALEAXRAXFXXXRAAPYA

SEQ ID NO: 270 - protein, 35 aa, synthetic construct
  VARIANT 2: note = X at position 2 can be selected from any one of N, S.
  VARIANT 4: note = X at position 4 can be selected from any one of A, E.
  VARIANT 26: note = X at position 26 can be selected from any one of F, L.
  VARIANT 27: note = X at position 27 can be selected from any one of P, R.
  VARIANT 28: note = X at position 28 can be selected from any one of D, E, V.
  VARIANT 32: note = X at position 32 can be selected from any one of A, T.
  VARIANT 35: note = X at position 35 can be selected from any one of A, D, G.
LXTXQVVAIASSHGGKQALEAVRALXXXLRAXPYX

SEQ ID NO: 271 - protein, 35 aa, synthetic construct
  VARIANT 2: note = X at position 2 can be selected from any one of N or S.
  VARIANT 9: note = X at position 9 can be selected from any one of I or V.
  VARIANT 18: note = X at position 18 can be selected from any one of A or V.
  VARIANT 20: note = X at position 20 can be selected from any one of A or E.
  VARIANT 21: note = X at position 21 can be selected from any one of A or E.
  VARIANT 23: note = X at position 23 can be selected from any one of E, G, or K.
  VARIANT 25: note = X at position 25 can be selected from any one of D, H, or Q.
  VARIANT 28: note = X at position 28 can be selected from any one of A, D, E, or R.
  VARIANT 30: note = X at position 30 can be selected from any one of L or R.
  VARIANT 31: note = X at position 31 can be selected from any one of A or G.
  VARIANT 32: note = X at position 32 can be selected from any one of A or V.
  VARIANT 35: note = X at position 35 can be selected from any one of A, E, or V.
LXTEQVVAXASNKGGKQXLXXVXAXLLXLXXXPYX

SEQ ID NO: 272 - protein, 35 aa, synthetic construct
  VARIANT 3: note = X at position 3 can be selected from any one of A, T.
  VARIANT 4: note = X at position 4 can be selected from any one of A, E.
  VARIANT 7: note = X at position 7 can be selected from any one of A, V.
  VARIANT 11: note = X at position 11 can be selected from any one of G, S.
  VARIANT 16: note = X at position 16 can be selected from any one of K, N.
  VARIANT 18: note = X at position 18 can be selected from any one of A, P.
  VARIANT 22: note = X at position 22 can be selected from any one of A, V.
  VARIANT 23: note = X at position 23 can be selected from any one of G, V.
  VARIANT 24: note = X at position 24 can be selected from any one of A, G, T, V.
  VARIANT 28: note = X at position 28 can be selected from any one of A, E, V.
  VARIANT 30: note = X at position 30 can be selected from any one of L, R.
LSXXQVXAIAXHDGGXQXLEAXXXQLVXLXAAPYA

SEQ ID NO: 273 - protein, 35 aa, synthetic construct
  VARIANT 3: note = X at position 3 can be selected from any one of I, T.
  VARIANT 9: note = X at position 9 can be selected from any one of I, V.
  VARIANT 11: note = X at position 11 can be selected from any one of S, T.
  VARIANT 28: note = X at position 28 can be selected from any one of A, D.
LSXAQVVAXAXRSGGKQALEAVRAQLLXLRAAPYG

SEQ ID NO: 274 - protein, 35 aa, synthetic construct
  VARIANT 3: note = X at position 3 can be selected from any one of P, T.
  VARIANT 28: note = X at position 28 can be selected from any one of E, G.
  VARIANT 35: note = X at position 35 can be selected from any one of A, G.
LSXEQVVAIASNHGGKQALEAVRALFRXLRAAPYX

SEQ ID NO: 275 - protein, 35 aa, synthetic construct
  VARIANT 4: note = X at position 4 can be selected from any one of A, E.
  VARIANT 7: note = X at position 7 can be selected from any one of A, V.
  VARIANT 8: note = X at position 8 can be selected from any one of T, V.
  VARIANT 11: note = X at position 11 can be selected from any one of N, S.
  VARIANT 16: note = X at position 16 can be selected from any one of K, R.
  VARIANT 22: note = X at position 22 can be selected from any one of L, V.
  VARIANT 35: note = X at position 35 can be selected from any one of E, G.
LSTXQVXXIAXSIGGXQALEAXKVQLPVLRAAPYX

SEQ ID NO: 276 - protein, 35 aa, synthetic construct
  VARIANT 2: note = X at position 2 can be selected from any one of S, T.
  VARIANT 18: note = X at position 18 can be selected from any one of A, T.
  VARIANT 21: note = X at position 21 can be selected from any one of A, E, V.
  VARIANT 22: note = X at position 22 can be selected from any one of A, V.
  VARIANT 23: note = X at position 23 can be selected from any one of E, G.
  VARIANT 28: note = X at position 28 can be selected from any one of A, V.
  VARIANT 32: note = X at position 32 can be selected from any one of A, V.
LXTAQVVAIASNDGGKQXLEXXXAQLLXLRAXPYE

SEQ ID NO: 277 - protein, 35 aa, synthetic construct
  VARIANT 7: note = X at position 7 can be selected from any one of A, T.
  VARIANT 8: note = X at position 8 can be selected from any one of I, V.
  VARIANT 9: note = X can be any naturally occurring amino acid
LSTAQVVXXASSNGGKQALEAVWALLPVLRATPYD

SEQ ID NO: 278 - protein, 35 aa, synthetic construct
  VARIANT 4: note = X at position 4 can be selected from any one of A, E, G.
  VARIANT 11: note = X at position 11 can be selected from any one of G, S.
  VARIANT 16: note = X at position 16 can be selected from any one of K, R.
  VARIANT 21: note = X at position 21 can be selected from any one of A, G.
  VARIANT 22: note = X at position 22 can be selected from any one of I, V.
  VARIANT 23: note = X at position 23 can be selected from any one of G, R.
  VARIANT 24: note = X at position 24 can be selected from any one of E, K.
  VARIANT 27: note = X at position 27 can be selected from any one of L, Q, R.
  VARIANT 28: note = X at position 28 can be selected from any one of A, E, K.
  VARIANT 31: note = X at position 31 can be selected from any one of A, T.
  VARIANT 32: note = X at position 32 can be selected from any one of A, V.
  VARIANT 34: note = X at position 34 can be selected from any one of H, Y.
  VARIANT 35: note = X at position 35 can be selected from any one of E, G.
LSTXQVVAIAXNGGGXQALEXXXXQLXXLRXXPXX

SEQ ID NO: 279 - protein, 35 aa, synthetic construct
  VARIANT 11: note = X at position 11 can be selected from any one of A or S.
  VARIANT 17: note = X at position 17 can be selected from any one of Q or R.
  VARIANT 20: note = X at position 20 can be selected from any one of E or G.
  VARIANT 22: note = X at position 22 can be selected from any one of I or V.
  VARIANT 23: note = X at position 23 can be selected from any one of C, R, or T.
  VARIANT 24: note = X at position 24 can be selected from any one of T or V.
  VARIANT 27: note = X at position 27 can be selected from any one of P or R.
  VARIANT 28: note = X at position 28 can be selected from any one of I or V.
  VARIANT 31: note = X at position 31 can be selected from any one of A or G.
  VARIANT 33: note = X at position 33 can be selected from any one of P or R.
  VARIANT 35: note = X at position 35 can be selected from any one of E, G, or R.
LTPQQVVAIAXNTGGKXALXAXXXQLXXLRXAXYX

SEQ ID NO: 280 - protein, 22 aa, synthetic construct
GGKQALEAVRAQLLDLRAAPYG

SEQ ID NO: 281 - protein, 635 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDGLGHKELIKIAARNGGGNNLIAVLSCYAKLKEMGFSSQQIIRMVSHAGGANNLKAVTANHDDLQNMGFNVEQIVRMVSHNGGSKNLKAVTDNHDDLKNMGFNAEQIVRMVSHGGGSKNLKAVTDNHDDLKNMGFNAEQIVSMVSNNGGSKNLKAVTDNHDDLKNMGFNAEQIVSMVSNGGGSLNLKAVKKYHDALKDRGFNTEQIVRMVSHDGGSLNLKAVKKYHDALRERKFNVEQIVSIVSHGGGSLNLKAVKKYHDVLKDREFNAEQIVRMVSHDGGSLNLKAVTDNHDDLKNMGFNAEQIVRMVSHKGGSKNLALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 282 - protein, 33 aa, synthetic construct
FSSQQIIRMVSHAGGANNLKAVTANHDDLQNMG

SEQ ID NO: 283 - protein, 33 aa, synthetic construct
FNVEQIVRMVSHNGGSKNLKAVTDNHDDLKNMG

SEQ ID NO: 284 - protein, 33 aa, synthetic construct
FNAEQIVRMVSHGGGSKNLKAVTDNHDDLKNMG

SEQ ID NO: 285 - protein, 33 aa, synthetic construct
FNAEQIVSMVSNNGGSKNLKAVTDNHDDLKNMG

SEQ ID NO: 286 - protein, 33 aa, synthetic construct
FNAEQIVSMVSNGGGSLNLKAVKKYHDALKDRG

SEQ ID NO: 287 - protein, 33 aa, synthetic construct
FNTEQIVRMVSHDGGSLNLKAVKKYHDALRERK

SEQ ID NO: 288 - protein, 33 aa, synthetic construct
FNVEQIVSIVSHGGGSLNLKAVKKYHDVLKDRE

SEQ ID NO: 289 - protein, 33 aa, synthetic construct
FNAEQIVRMVSHDGGSLNLKAVTDNHDDLKNMG

SEQ ID NO: 290 - protein, 19 aa, synthetic construct
FNAEQIVRMVSHKGGSKNL

SEQ ID NO: 291 - protein, 143 aa, synthetic construct
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 292-296 - skipped (5 entries)

SEQ ID NO: 297 - protein, 20 aa, synthetic construct
LTPQQVVAIASNGGGRPALE

SEQ ID NO: 298 - protein, 63 aa, synthetic construct
SIVAQLSRPDPALAALTNDHLVALACLGGRPALDAVKKGLPHAPALIKRTNRRIPERTSHRVA

SEQ ID NO: 299 - protein, 1164 aa, synthetic construct
MDPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNSGGKQALETVQRLLPVLCQAHGLTPEQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNSGGKQALETVQALLPVLCQAHGLTPEQVVAIASNSGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLSWRVKRPRTSIGGGLPDPGTPTAADLAASSTVMREQDEDPFAGAADDFPAFNEEELAWLMELLPQ

SEQ ID NO: 300 - protein, 137 aa, synthetic construct
MVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 301 - protein, 120 aa, synthetic construct
KPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 302 - protein, 278 aa, synthetic construct
SIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPTHEGDQRRASSRKRSRSDRAVTGPSAQQSFEVRAPEQRDALHLPLSWRVKRPRTSIGGGLPDPGTPTAADLAASSTVMREQDEDPFAGAADDFPAFNEEELAWLMELLPQ

SEQ ID NO: 303 - protein, 120 aa, synthetic construct
KQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLH

SEQ ID NO: 304 - protein, 120 aa, synthetic construct
DATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 305 - protein, 176 aa, synthetic construct
ALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 306 - protein, 63 aa, synthetic construct
ALVKEYFPVFSSFHFTADQIVALICQSKQCFRNLKKNHQQWKNKGLSAEQIVDLILQETPPKP

SEQ ID NO: 307 - protein, 575 aa, synthetic construct
  REGION 35: note = X at position 35 represents a linker comprising a recognition site and can comprise 1-40 amino acid residues. An amino acid residue of the linker can comprise a glycine, an alanine, a threonine, or a histidine.
  REGION 70: note = X at position 70 represents a linker comprising a recognition site and can comprise 1-40 amino acid residues. An amino acid residue of the linker can comprise a glycine, an alanine, a threonine, or a histidine.
  REGION 105: note = X at position 105 represents a linker comprising a recognition site and can comprise 1-40 amino acid residues. An amino acid residue of the linker can comprise a glycine, an alanine, a threonine, or a histidine.
  REGION 140: note = X at position 140 represents a linker comprising a recognition site and can comprise 1-40 amino acid residues. An amino acid residue of the linker can comprise a glycine, an alanine, a threonine, or a histidine.
  REGION 175: note = X at position 175 represents a linker comprising a recognition site and can comprise 1-40 amino acid residues. An amino acid residue of the linker can comprise a glycine, an alanine, a threonine, or a histidine.
  REGION 210: note = X at position 210 represents a linker comprising a recognition site and can comprise 1-40 amino acid residues. An amino acid residue of the linker can comprise a glycine, an alanine, a threonine, or a histidine.
  REGION 245: note = X at position 245 represents a linker comprising a recognition site and can comprise 1-40 amino acid residues. An amino acid residue of the linker can comprise a glycine, an alanine, a threonine, or a histidine.
  REGION 280: note = X at position 280 represents a linker comprising a recognition site and can comprise 1-40 amino acid residues. An amino acid residue of the linker can comprise a glycine, an alanine, a threonine, or a histidine.
  REGION 315: note = X at position 315 represents a linker comprising a recognition site and can comprise 1-40 amino acid residues. An amino acid residue of the linker can comprise a glycine, an alanine, a threonine, or a histidine.
  REGION 350: note = X at position 350 represents a linker comprising a recognition site and can comprise 1-40 amino acid residues. An amino acid residue of the linker can comprise a glycine, an alanine, a threonine, or a histidine.
  REGION 385: note = X at position 385 represents a linker comprising a recognition site and can comprise 1-40 amino acid residues. An amino acid residue of the linker can comprise a glycine, an alanine, a threonine, or a histidine.
  REGION 420: note = X at position 420 represents a linker comprising a recognition site and can comprise 1-40 amino acid residues. An amino acid residue of the linker can comprise a glycine, an alanine, a threonine, or a histidine.
  REGION 455: note = X at position 455 represents a linker comprising a recognition site and can comprise 1-40 amino acid residues. An amino acid residue of the linker can comprise a glycine, an alanine, a threonine, or a histidine.
  REGION 490: note = X at position 490 represents a linker comprising a recognition site and can comprise 1-40 amino acid residues. An amino acid residue of the linker can comprise a glycine, an alanine, a threonine, or a histidine.
  REGION 525: note = X at position 525 represents a linker comprising a recognition site and can comprise 1-40 amino acid residues. An amino acid residue of the linker can comprise a glycine, an alanine, a threonine, or a histidine.
  REGION 560: note = X at position 560 represents a linker comprising a recognition site and can comprise 1-40 amino acid residues. An amino acid residue of the linker can comprise a glycine, an alanine, a threonine, or a histidine.
LTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASNHGGKQALETVQRLLPVLCQDHGXLTPDQVVAIASNGGG

SEQ ID NO: 308 - protein, 594 aa, synthetic construct
  REGION 35: note = X at position 35 represents a linker comprising a recognition site and can comprise 1-40 amino acid residues. An amino acid residue of the linker can comprise a glycine, an alanine, a threonine, or a histidine.
  REGION 70: note = X at position 70 represents a linker comprising a recognition site and can comprise 1-40 amino acid residues. An amino acid residue of the linker can comprise a glycine, an alanine, a threonine, or a histidine.
  REGION 105: note = X at position 105 represents a linker comprising a recognition site and can comprise 1-40 amino acid residues. An amino acid residue of the linker can comprise a glycine, an alanine, a threonine, or a histidine.
  REGION 140: note = X at position 140 represents a linker comprising a recognition site and can comprise 1-40 amino acid residues. An amino acid residue of the linker can comprise a glycine, an alanine, a threonine, or a histidine.
  REGION 175: note = X at position 175 represents a linker comprising a recognition site and can comprise 1-40 amino acid residues. An amino acid residue of the linker can comprise a glycine, an alanine, a threonine, or a histidine.
  REGION 210: note = X at position 210 represents a linker comprising a recognition site and can comprise 1-40 amino acid residues. An amino acid residue of the linker can comprise a glycine, an alanine, a threonine, or a histidine.
  REGION 245: note = X at position 245 represents a linker comprising a recognition site and can comprise 1-40 amino acid residues. An amino acid residue of the linker can comprise a glycine, an alanine, a threonine, or a histidine.
  REGION 280: note = X at position 280 represents a linker comprising a recognition site and can comprise 1-40 amino acid residues. An amino acid residue of the linker can comprise a glycine, an alanine, a threonine, or a histidine.
  REGION 315: note = X at position 315 represents a linker comprising a recognition site and can comprise 1-40 amino acid residues. An amino acid residue of the linker can comprise a glycine, an alanine, a threonine, or a histidine.
  REGION 350: note = X at position 350 represents a linker comprising a recognition site and can comprise 1-40 amino acid residues. An amino acid residue of the linker can comprise a glycine, an alanine, a threonine, or a histidine.
  REGION 385: note = X at position 385 represents a linker comprising a recognition site and can comprise 1-40 amino acid residues. An amino acid residue of the linker can comprise a glycine, an alanine, a threonine, or a histidine.
  REGION 420: note = X at position 420 represents a linker comprising a recognition site and can comprise 1-40 amino acid residues. An amino acid residue of the linker can comprise a glycine, an alanine, a threonine, or a histidine.
  REGION 455: note = X at position 455 represents a linker comprising a recognition site and can comprise 1-40 amino acid residues. An amino acid residue of the linker can comprise a glycine, an alanine, a threonine, or a histidine.
  REGION 490: note = X at position 490 represents a linker comprising a recognition site and can comprise 1-40 amino acid residues. An amino acid residue of the linker can comprise a glycine, an alanine, a threonine, or a histidine.
  REGION 525: note = X at position 525 represents a linker comprising a recognition site and can comprise 1-40 amino acid residues. An amino acid residue of the linker can comprise a glycine, an alanine, a threonine, or a histidine.
  REGION 560: note = X at position 560 represents a linker comprising a recognition site and can comprise 1-40 amino acid residues. An amino acid residue of the linker can comprise a glycine, an alanine, a threonine, or a histidine.
LTPDQVVAIASNGGGKQALETVQRLLPVLCQDHGXLTPDQVVAIASNGGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASNHGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASNIGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNGGGKQALESIVAQLSRPDPALA

SEQ ID NO: 309 - protein, 646 aa, synthetic construct
  REGION 35: note = X at position 35 represents a linker comprising a recognition site and can comprise 1-40 amino acid residues. An amino acid residue of the linker can comprise a glycine, an alanine, a threonine, or a histidine.
  REGION 70: note = X at position 70 represents a linker comprising a recognition site and can comprise 1-40 amino acid residues. An amino acid residue of the linker can comprise a glycine, an alanine, a threonine, or a histidine.
  REGION 105: note = X at position 105 represents a linker comprising a recognition site and can comprise 1-40 amino acid residues. An amino acid residue of the linker can comprise a glycine, an alanine, a threonine, or a histidine.
  REGION 140: note = X at position 140 represents a linker comprising a recognition site and can comprise 1-40 amino acid residues. An amino acid residue of the linker can comprise a glycine, an alanine, a threonine, or a histidine.
  REGION 175: note = X at position 175 represents a linker comprising a recognition site and can comprise 1-40 amino acid residues. An amino acid residue of the linker can comprise a glycine, an alanine, a threonine, or a histidine.
  REGION 210: note = X at position 210 represents a linker comprising a recognition site and can comprise 1-40 amino acid residues. An amino acid residue of the linker can comprise a glycine, an alanine, a threonine, or a histidine.
  REGION 245: note = X at position 245 represents a linker comprising a recognition site and can comprise 1-40 amino acid residues. An amino acid residue of the linker can comprise a glycine, an alanine, a threonine, or a histidine.
  REGION 280: note = X at position 280 represents a linker comprising a recognition site and can comprise 1-40 amino acid residues. An amino acid residue of the linker can comprise a glycine, an alanine, a threonine, or a histidine.
  REGION 315: note = X at position 315 represents a linker comprising a recognition site and can comprise 1-40 amino acid residues. An amino acid residue of the linker can comprise a glycine, an alanine, a threonine, or a histidine.
  REGION 350: note = X at position 350 represents a linker comprising a recognition site and can comprise 1-40 amino acid residues. An amino acid residue of the linker can comprise a glycine, an alanine, a threonine, or a histidine.
  REGION 385: note = X at position 385 represents a linker comprising a recognition site and can comprise 1-40 amino acid residues. An amino acid residue of the linker can comprise a glycine, an alanine, a threonine, or a histidine.
  REGION 420: note = X at position 420 represents a linker comprising a recognition site and can comprise 1-40 amino acid residues. An amino acid residue of the linker can comprise a glycine, an alanine, a threonine, or a histidine.
  REGION 455: note = X at position 455 represents a linker comprising a recognition site and can comprise 1-40 amino acid residues. An amino acid residue of the linker can comprise a glycine, an alanine, a threonine, or a histidine.
  REGION 490: note = X at position 490 represents a linker comprising a recognition site and can comprise 1-40 amino acid residues. An amino acid residue of the linker can comprise a glycine, an alanine, a threonine, or a histidine.
  REGION 525: note = X at position 525 represents a linker comprising a recognition site and can comprise 1-40 amino acid residues. An amino acid residue of the linker can comprise a glycine, an alanine, a threonine, or a histidine.
  REGION 560: note = X at position 560 represents a linker comprising a recognition site and can comprise 1-40 amino acid residues. An amino acid residue of the linker can comprise a glycine, an alanine, a threonine, or a histidine.
  REGION 595: note = X at position 595 represents a linker comprising a recognition site and can comprise 1-40 amino acid residues. An amino acid residue of the linker can comprise a glycine, an alanine, a threonine, or a histidine.
  REGION 630: note = X at position 630 represents a linker comprising a recognition site and can comprise 1-40 amino acid residues. An amino acid residue of the linker can comprise a glycine, an alanine, a threonine, or a histidine.
LTPDQVVAIASNHGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASNHGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASNHGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASNHGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNGGGK

SEQ ID NO: 310 - protein, 734 aa, synthetic construct
  REGION 35: note = X at position 35 represents a linker comprising a recognition site and can comprise 1-40 amino acid residues. An amino acid residue of the linker can comprise a glycine, an alanine, a threonine, or a histidine.
  REGION 70: note = X at position 70 represents a linker comprising a recognition site and can comprise 1-40 amino acid residues. An amino acid residue of the linker can comprise a glycine, an alanine, a threonine, or a histidine.
  REGION 105: note = X at position 105 represents a linker comprising a recognition site and can comprise 1-40 amino acid residues. An amino acid residue of the linker can comprise a glycine, an alanine, a threonine, or a histidine.
  REGION 140: note = X at position 140 represents a linker comprising a recognition site and can comprise 1-40 amino acid residues. An amino acid residue of the linker can comprise a glycine, an alanine, a threonine, or a histidine.
  REGION 175: note = X at position 175 represents a linker comprising a recognition site and can comprise 1-40 amino acid residues. An amino acid residue of the linker can comprise a glycine, an alanine, a threonine, or a histidine.
  REGION 210: note = X at position 210 represents a linker comprising a recognition site and can comprise 1-40 amino acid residues. An amino acid residue of the linker can comprise a glycine, an alanine, a threonine, or a histidine.
  REGION 245: note = X at position 245 represents a linker comprising a recognition site and can comprise 1-40 amino acid residues. An amino acid residue of the linker can comprise a glycine, an alanine, a threonine, or a histidine.
  REGION 280: note = X at position 280 represents a linker comprising a recognition site and can comprise 1-40 amino acid residues. An amino acid residue of the linker can comprise a glycine, an alanine, a threonine, or a histidine.
  REGION 315: note = X at position 315 represents a linker comprising a recognition site and can comprise 1-40 amino acid residues. An amino acid residue of the linker can comprise a glycine, an alanine, a threonine, or a histidine.
  REGION 350: note = X at position 350 represents a linker comprising a recognition site and can comprise 1-40 amino acid residues. An amino acid residue of the linker can comprise a glycine, an alanine, a threonine, or a histidine.
  REGION 385: note = X at position 385 represents a linker comprising a recognition site and can comprise 1-40 amino acid residues. An amino acid residue of the linker can comprise a glycine, an alanine, a threonine, or a histidine.
  REGION 420: note = X at position 420 represents a linker comprising a recognition site and can comprise 1-40 amino acid residues. An amino acid residue of the linker can comprise a glycine, an alanine, a threonine, or a histidine.
  REGION 455: note = X at position 455 represents a linker comprising a recognition site and can comprise 1-40 amino acid residues. An amino acid residue of the linker can comprise a glycine, an alanine, a threonine, or a histidine.
  REGION 490: note = X at position 490 represents a linker comprising a recognition site and can comprise 1-40 amino acid residues. An amino acid residue of the linker can comprise a glycine, an alanine, a threonine, or a histidine.
  REGION 525: note = X at position 525 represents a linker comprising a recognition site and can comprise 1-40 amino acid residues. An amino acid residue of the linker can comprise a glycine, an alanine, a threonine, or a histidine.
  REGION 560: note = X at position 560 represents a linker comprising a recognition site and can comprise 1-40 amino acid residues. An amino acid residue of the linker can comprise a glycine, an alanine, a threonine, or a histidine.
  REGION 595: note = X at position 595 represents a linker comprising a recognition site and can comprise 1-40 amino acid residues. An amino acid residue of the linker can comprise a glycine, an alanine, a threonine, or a histidine.
  REGION 630: note = X at position 630 represents a linker comprising a recognition site and can comprise 1-40 amino acid residues. An amino acid residue of the linker can comprise a glycine, an alanine, a threonine, or a histidine.
  REGION 665: note = X at position 665 represents a linker comprising a recognition site and can comprise 1-40 amino acid residues. An amino acid residue of the linker can comprise a glycine, an alanine, a threonine, or a histidine.
  REGION 700: note = X at position 700 represents a linker comprising a recognition site and can comprise 1-40 amino acid residues. An amino acid residue of the linker can comprise a glycine, an alanine, a threonine, or a histidine.
LTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVAIASNGGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASHDGGKQALETVQRLLPVLCQDHGXLTPDQVVAIASNGGGKQALETVQRLLPVLCQDHGXLTPDQVVAIASNIGGKQALETVQRLLPVLCQDHGXLTPDQVVAIASHDGGKQALETVQRLLPVLCQDHGXLTPDQVVAIASHDGGKQALETVQRLLPVLCQDHGXLTPDQVVAIASNIGGKQALETVQRLLPVLCQDHGXLTPDQVVASASNGGGKQALESIVAQLSRPDPALA

SEQ ID NO: 311 - protein, 883 aa, synthetic construct
FGKLVALGYSREQIRKLKQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLHLTPQQVVAIASNTGGKRALEAVCVQLPVLRAAPYRLSTEQVVAIASHDGGKQALEAVGAQLVALRAAPYALSTEQVVAIASHDGGKQALEAVGAQLVALRAAPYALSTAQVVAIASNGGGKQALEGIGEQLLKLRTAPYGLSTEQVVAIASNKGGKQALEAVKAHLLDLLGAPYVLSTEQVVAIASNKGGKQALEAVKAHLLDLLGAPYVLSTEQVVAIASNKGGKQALEAVKAHLLDLLGAPYVLSTEQVVVIANSIGGKQALEAVKVQLPVLRAAPYELSTEQVVAIASHDGGKQALEAVGAQLVALRAAPYALSTEQVVVIANSIGGKQALEAVKVQLPVLRAAPYELSTEQVVAIASNKGGKQALEAVKAHLLDLLGAPYVLSTAQVVAIASNGGGKQALEGIGEQLLKLRTAPYGLSTAQVVAIASNGGGKQALEGIGEQLLKLRTAPYGLSTAQVVAIASNGGGKQALEGIGEQLLKLRTAPYGLSTEQVVAIASHDGGKQALEAVGAQLVALRAAPYALSTEQVVAIASHDGGKQALEAVGAQLVALRAAPYALSTEQVVAIASHDGGKQALEAVGAQLVALRAAPYALSTAQVVAIASNGGGKQALEGIGEQLLKLRTAPYGLSTAQVVAIASNGGGKQALEGIGEQLLKLRTAPYGLSTAQVVAIACISGQQALEAIEAHMPTLRQASHSLSPERVAAIACIGGRSAVEAVRQGLPVKAIRRIRREKAPVAGPPPAS

SEQ ID NO: 312 - DNA, 20 bp, synthetic construct
  source 1..20: mol_type = other DNA
gacctgggacagtttccctt

SEQ ID NO: 313 - protein, 813 aa, synthetic construct
FGKLVALGYSREQIRKLKQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLHLTPQQVVAIASNTGGKRALEAVCVQLPVLRAAPYRLSTAQVVAIASNGGGKQALEGIGEQLLKLRTAPYGLSTEQVVAIASHDGGKQALEAVGAQLVALRAAPYALSTAQVVAIASNGGGKQALEGIGEQLLKLRTAPYGLSTEQVVAIASHNGGKQALEAVKADLLELRGAPYALSTEQVVAIASHDGGKQALEAVGAQLVALRAAPYALSTEQVVVIANSIGGKQALEAVKVQLPVLRAAPYELSTAQVVAIASNGGGKQALEGIGEQLLKLRTAPYGLSTEQVVAIASHNGGKQALEAVKADLLELRGAPYALSTEQVVAIASHDGGKQALEAVGAQLVALRAAPYALSTEQVVAIASHDGGKQALEAVGAQLVALRAAPYALSTAQVVAIASNGGGKQALEGIGEQLLKLRTAPYGLSTEQVVAIASHNGGKQALEAVKADLLELRGAPYALSTEQVVAIASHNGGKQALEAVKADLLELRGAPYALSTEQVVVIANSIGGKQALEAVKVQLPVLRAAPYELSTEQVVAIASHNGGKQALEAVKADLLELRGAPYALSTEQVVAIASHDGGKQALEAVGAQLVALRAAPYALSTAQVVAIACISGQQALEAIEAHMPTLRQASHSLSPERVAAIACIGGRSAVEAVRQGLPVKAIRRIRREKAPVAGPPPAS

SEQ ID NO: 314 - DNA, 18 bp, synthetic construct
  source 1..18: mol_type = other DNA
gatctgcatgcctggagc

SEQ ID NO: 315 - protein, 813 aa, synthetic construct
FGKLVALGYSREQIRKLKQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLHLTPQQVVAIASNTGGKRALEAVCVQLPVLRAAPYRLSTAQVVAIASNGGGKQALEGIGEQLLKLRTAPYGLSTEQVVAIASHDGGKQALEAVGAQLVALRAAPYALSTAQVVAIASNGGGKQALEGIGEQLLKLRTAPYGLSTEQVVAIASHNGGKQALEAVKADLLELRGAPYALSTEQVVAIASHDGGKQALEAVGAQLVALRAAPYALSTAQVVAIATRSGGKQALEAVRAQLLDLRAAPYGLSTAQVVAIASNGGGKQALEGIGEQLLKLRTAPYGLSTEQVVAIASHNGGKQALEAVKADLLELRGAPYALSTEQVVAIASHDGGKQALEAVGAQLVALRAAPYALSTEQVVAIASHDGGKQALEAVGAQLVALRAAPYALSTAQVVAIASNGGGKQALEGIGEQLLKLRTAPYGLSTEQVVAIASHNGGKQALEAVKADLLELRGAPYALSTEQVVAIASHNGGKQALEAVKADLLELRGAPYALSTAQVVAIATRSGGKQALEAVRAQLLDLRAAPYGLSTEQVVAIASHNGGKQALEAVKADLLELRGAPYALSTEQVVAIASHDGGKQALEAVGAQLVALRAAPYALSTAQVVAIACISGQQALEAIEAHMPTLRQASHSLSPERVAAIACIGGRSAVEAVRQGLPVKAIRRIRREKAPVAGPPPAS

SEQ ID NO: 316 - protein, 202 aa, synthetic construct
  VARIANT 1: note = X is F, Q, N, D, or absent
  VARIANT 2: note = X is L, I, T, S, N, or absent
  VARIANT 3: note = X is V, I, G, A, E, T, or absent
  VARIANT 4: note = X is K, C, or absent
  VARIANT 5: note = X is G, S, or absent
  VARIANT 6: note = X is A, S, E, D, N, or absent
  VARIANT 7: note = X is M, I, V, Q, F, L, or absent
  VARIANT 8: note = X is E, S, T, N, or absent
  VARIANT 9: note = X is I, M, E, T, Q, or absent
  VARIANT 10: note = X is K, S, L, I, T, E, or absent
  VARIANT 11: note = X is K or absent
  VARIANT 12: note = X is S, A, E, D, or absent
  VARIANT 13: note = X is E, N, Q, K, or absent
  VARIANT 14: note = X is L, M, V, or absent
  VARIANT 15: note = X is R or absent
  VARIANT 16: note = X is H, D, T, G, E, N, or absent
  VARIANT 17: note = X is K, N, Q, E, A,
  VARIANT 18: note = X is L or absent
  VARIANT 19: note = X is R, Q, N, T, D, or absent
  VARIANT 20: note = X is H, M, V, N, T, or absent
  VARIANT 21: note = X is V, L, I, or absent
  VARIANT 22: note = X is P, S, or absent
  VARIANT 23: note = X is H or absent
  VARIANT 24: note = X is E, D, or absent
  VARIANT 25: note = X is Y or absent
  VARIANT 26: note = X is I, L, or absent
  VARIANT 27: note = X is E, Q, G, S, A, Y, or absent
  VARIANT 28: note = X is L or absent
  VARIANT 29: note = X is I, V, L, or absent
  VARIANT 30: note = X is E, D, or absent
  VARIANT 31: note = X is I, L, or absent
  VARIANT 32: note = X is A, S, or absent
  VARIANT 33: note = X is Q, Y, F, or absent
  VARIANT 34: note = X is D or absent
  VARIANT 35: note = X is S, P, or absent
  VARIANT 36: note = X is K, Y, Q, T, or absent
  VARIANT 37: note = X is Q or absent
  VARIANT 38: note = X is N or absent
  VARIANT 39: note = X is R, K, or absent
  VARIANT 40: note = X is L, I, or absent
  VARIANT 41: note = X is L, F, or absent
  VARIANT 42: note = X is E or absent
  VARIANT 43: note = X is F, M, L, or absent
  VARIANT 45: note = X is V, T, or I
  VARIANT 46: note = X is V, M, L, or I
  VARIANT 47: note = X is E, D, or Q
  VARIANT 48: note = X is F or L
  VARIANT 49: note = X is F or L
  VARIANT 50: note = X is K, I, T, or V
  VARIANT 51: note = X is K, N, or E,
  VARIANT 52: note = X is I or E
  VARIANT 53: note = X is Y, F, or C
  VARIANT 54: note = X is G, or N
  VARIANT 55: note = X is Y, or F
  VARIANT 56: note = X is R, S, N, E, K, or Q
  VARIANT 58: note = X is K, S, L, V, or T
  VARIANT 63: note = X is S, A,or V
  VARIANT 65: note = X is K or R
  VARIANT 69: note = X is A, I, or V
  VARIANT 70: note = X is L, M, V, I, or C
  VARIANT 71: note = X is F or Y
  VARIANT 72: note = X is T, A, or S
  VARIANT 73: note = X is K, E, or absent
  VARIANT 74: note = X is D, E, or absent
  VARIANT 75: note = X is E, A, or absent
  VARIANT 76: note = X is N, K, or absent
  VARIANT 77: note = X is E, S, or absent
  VARIANT 78: note = X is D, E, Q, A, or absent
  VARIANT 79: note = X is G, V, K, N, or absent
  VARIANT 80: note = X is L, G, E, S, or absent
  VARIANT 81: note = X is V, S, K, T, E, or absent
  VARIANT 82: note = X is L, H, K, E, Y, D, or A
  VARIANT 83: note = X is N, G, or D
  VARIANT 84: note = X is H, F, or Y
  VARIANT 86: note = X is I, or V
  VARIANT 87: note = X is L, V, or I
  VARIANT 88: note = X is A or S
  VARIANT 92: note = X is K or S
  VARIANT 94: note = X is D, G, K, S, or N
  VARIANT 95: note = X is R, N, S, or G
  VARIANT 98: note = X is S, A, or G
  VARIANT 102: note = X is A, I, or V
  VARIANT 104: note = X is Q, E, I, or V
  VARIANT 108: note = X is V or I
  VARIANT 112: note = X is D, R, G, I, or E, X is N, I, or Q
  VARIANT 115: note = X is K, D, T, E, or K
  VARIANT 116: note = X is S, N, D, or E
  VARIANT 118: note = X is Q, E, I, K, or A
  VARIANT 119: note = X is V, H, R, K, L, or E
  VARIANT 120: note = X is I, V, or R
  VARIANT 121: note = X is P, S, T, or R
  VARIANT 123: note = X is E, R, C, Q, or K
  VARIANT 125: note = X is E, N, or K
  VARIANT 128: note = X is I, V, N, E, or A
  VARIANT 129: note = X is Y or F
  VARIANT 130: note = X is P, G, or E
  VARIANT 131: note = X is T, E, S, D, K, or N
  VARIANT 132: note = X is S, D, K, G, N, or T
  VARIANT 133: note = X is I, T, V, or L
  VARIANT 134: note = X is T, N, G, or D
  VARIANT 135: note = X is D, E, T, K, or I
  VARIANT 136: note = X is F or Y
  VARIANT 137: note = X is K or Y
  VARIANT 138: note = X is F or Y
  VARIANT 139: note = X is L, S, or M
  VARIANT 140: note = X is V or I
  VARIANT 142: note = X is S or A
  VARIANT 143: note = X is G or A
  VARIANT 144: note = X is F, Y, H, E, or K
  VARIANT 145: note = X is Q, K, T, N, or I
  VARIANT 147: note = X is D, N, or K
  VARIANT 149: note = X is Y, F, I, or V
  VARIANT 150: note = X is R, E, K, Q, or F
  VARIANT 151: note = X is K, E, A, or N
  VARIANT 152: note = X is Q or K
  VARIANT 153: note = X is L or I
  VARIANT 154: note = X is E, D, N, or Q
  VARIANT 155: note = X is V, I, or L
  VARIANT 157: note = X is S, N, F, T, or Q
  VARIANT 158: note = X is H, I, C, or R
  VARIANT 159: note = X is L, D, N, S, or F
  VARIANT 160: note = X is T or K
  VARIANT 161: note = X is K, G, or N
  VARIANT 162: note = X is C, V, or I
  VARIANT 163: note = X is Q, L, K, or Y
  VARIANT 164: note = X is A, G, or N
  VARIANT 166: note = X is V or A
  VARIANT 167: note = X is M, L, I, V, or A
  VARIANT 168: note = X is S, T, or D
  VARIANT 169: note = X is V or I
  VARIANT 170: note = X is E, Q, K, S, or I
  VARIANT 171: note = X is Q, H, or T
  VARIANT 172: note = X is L, R, or Y
  VARIANT 175: note = X is G, I, L, or T
  VARIANT 176: note = X is G, A, or V
  VARIANT 177: note = X is E, N, or D
  VARIANT 178: note = X is K, Y, D, E, A, or R
  VARIANT 179: note = X is I, F, Y, or C
  VARIANT 180: note = X is K or R
  VARIANT 181: note = X is E, R, A, G, or T
  VARIANT 182: note = X is G or N
  VARIANT 183: note = X is S, I, K, R, or E
  VARIANT 184: note = X is L, I, or M,
  VARIANT 185: note = X is T, S, D, or K
  VARIANT 186: note = X is L, H, Y, R, T, or F
  VARIANT 187: note = X is E, Y, I, M, A, or L
  VARIANT 188: note = X is E, D, R, or G
  VARIANT 189: note = X is V, F, M, L, or I
  VARIANT 190: note = X is G, K, R, L, V, or E
  VARIANT 191: note = X is K, N, D, L, H, or S
  VARIANT 192: note = X is K, L, C, or absent
  VARIANT 193: note = X is K, S, I, Y, M, or F
  VARIANT 194: note = X is K, L, C, H, D, Q, or N
  VARIANT 196: note = X is N or Y
  VARIANT 197: note = X is D, K, T, E, C, or absent
  VARIANT 198: note = X is E, V, R, or absent
  VARIANT 199: note = X is I, F, L, or absent
  VARIANT 200: note = X is V, Q, E, L, or absent
  VARIANT 201: note = X is F or absent
XXXXXXXXXXXXXXXXXXXXXXXXXXXXXXXXXXXXXXXXXXXKXXXXXXXXXXXXGXHLGGXRXPDGXXXXXXXXXXXXXXXXGXIXDTKXYXXGYXLPIXQXDEMXRYXXENXXRXXXXNXNXWWXXXXXXXXXXXXXFXXXXFXGXXXXXXXRXXXXXXXXGXXXXXXXLLXXXXXXXXXXXXXXXXXXXXFXXXXXXX

SEQ ID NO: 317 - protein, 202 aa, synthetic construct; identical to SEQ ID NO: 316
  VARIANT 1: note = X is F, Q, N, or absent
  VARIANT 2: note = X is L, I, T, S, or absent
  VARIANT 3: note = X is V, I, G, A, E, T, or absent
  VARIANT 4: note = X is K, C, or absent
  VARIANT 5: note = X is G, S, or absent
  VARIANT 6: note = X is A, S, E, D, or absent
  VARIANT 7: note = X is M, I, V, Q, F, L, or absent
  VARIANT 8: note = X is E, S, T, or absent
  VARIANT 9: note = X is I, M, E, T, Q, or absent
  VARIANT 10: note = X is K, S, L, I, T, E, or absent
  VARIANT 11: note = X is K or absent
  VARIANT 12: note = X is S, A, E, D, or absent
  VARIANT 13: note = X is E, N, Q, K, or absent
  VARIANT 14: note = X is L, M, V, or absent
  VARIANT 15: note = X is R or absent
  VARIANT 16: note = X is H, D, T, G, E, N, or absent
  VARIANT 17: note = X is K, N, Q, E, A, or absent
  VARIANT 18: note = X is L or absent
  VARIANT 19: note = X is R, Q, N, T, D, or absent
  VARIANT 20: note = X is H, M, V, N, T, or absent
  VARIANT 21: note = X is V, L, I, or absent
  VARIANT 22: note = X is P, S, or absent
  VARIANT 23: note = X is H or absent
  VARIANT 24: note = X is E, D, or absent
  VARIANT 25: note = X is Y or absent
  VARIANT 26: note = X is I, L, or absent
  VARIANT 27: note = X is E, Q, G, S, A, Y, or absent
  VARIANT 28: note = X is L or absent
  VARIANT 29: note = X is I, V, L, or absent
  VARIANT 30: note = X is E, D, or absent
  VARIANT 31: note = X is I, L, or absent
  VARIANT 32: note = X is A, S, or absent
  VARIANT 33: note = X is Q, Y, F, or absent
  VARIANT 34: note = X is D or absent
  VARIANT 35: note = X is S, P, or absent
  VARIANT 36: note = X is K, Y, Q, T, or absent
  VARIANT 37: note = X is Q or absent
  VARIANT 38: note = X is N or absent
  VARIANT 39: note = X is R, K, or absent
  VARIANT 40: note = X is L, I, or absent
  VARIANT 41: note = X is L, F, or absent
  VARIANT 42: note = X is E or absent
  VARIANT 43: note = X is F, M, L, or absent
  VARIANT 45: note = X is V, T, or I
  VARIANT 46: note = X is V, M, L, or I
  VARIANT 47: note = X is E, D, or Q
  VARIANT 48: note = X is F or L
  VARIANT 49: note = X is F or L
  VARIANT 50: note = X is K, I, T, or V
  VARIANT 51: note = X is K, N, or E,
  VARIANT 52: note = X is I or E
  VARIANT 53: note = X is Y, F, or C
  VARIANT 54: note = X is G, or N
  VARIANT 55: note = X is Y, or F
  VARIANT 56: note = X is R, S, N, E, K, or Q
  VARIANT 58: note = X is K, S, L, V, or T
  VARIANT 63: note = X is S, A,or V
  VARIANT 65: note = X is K or R
  VARIANT 69: note = X is A, I, or V
  VARIANT 70: note = X is L, M, V, I, or C
  VARIANT 71: note = X is F or Y
  VARIANT 72: note = X is T, A, or S
  VARIANT 73: note = X is K, E, or absent
  VARIANT 74: note = X is D, E, or absent
  VARIANT 75: note = X is E, A, or absent
  VARIANT 76: note = X is N, K, or absent
  VARIANT 77: note = X is E, S, or absent
  VARIANT 78: note = X is D, E, Q, A, or absent
  VARIANT 79: note = X is G, V, K, N, or absent
  VARIANT 80: note = X is L, G, E, S, or absent
  VARIANT 81: note = X is V, S, K, T, E, or absent
  VARIANT 82: note = X is L, H, K, E, Y, D, or A
  VARIANT 83: note = X is N, G, or D
  VARIANT 84: note = X is H, F, or Y
  VARIANT 86: note = X is I, or V
  VARIANT 87: note = X is L, V, or I
  VARIANT 88: note = X is A or S
  VARIANT 92: note = X is K or S
  VARIANT 94: note = X is D, G, K, S, or N
  VARIANT 95: note = X is R, N, S, or G
  VARIANT 98: note = X is S, A, or G
  VARIANT 102: note = X is A, I, or V
  VARIANT 104: note = X is Q, E, I, or V
  VARIANT 108: note = X is V or I
  VARIANT 112: note = X is D, R, G, I, or E, X is N, I, or Q
  VARIANT 115: note = X is K, D, T, E, or K
  VARIANT 116: note = X is S, N, D, or E
  VARIANT 118: note = X is Q, E, I, K, or A
  VARIANT 119: note = X is V, H, R, K, L, or E
  VARIANT 120: note = X is I, V, or R
  VARIANT 121: note = X is P, S, T, or R
  VARIANT 123: note = X is E, R, C, Q, or K
  VARIANT 125: note = X is E, N, or K
  VARIANT 128: note = X is I, V, N, E, or A
  VARIANT 129: note = X is Y or F
  VARIANT 130: note = X is P, G, or E
  VARIANT 131: note = X is T, E, S, D, K, or N
  VARIANT 132: note = X is S, D, K, G, N, or T
  VARIANT 133: note = X is I, T, V, or L
  VARIANT 134: note = X is T, N, G, or D
  VARIANT 135: note = X is D, E, T, K, or I
  VARIANT 136: note = X is F or Y
  VARIANT 137: note = X is K or Y
  VARIANT 138: note = X is F or Y
  VARIANT 139: note = X is L, S, or M
  VARIANT 140: note = X is V or I
  VARIANT 142: note = X is S or A
  VARIANT 143: note = X is G or A
  VARIANT 144: note = X is F, Y, H, E, or K
  VARIANT 145: note = X is Q, K, T, N, or I
  VARIANT 147: note = X is D, N, or K
  VARIANT 149: note = X is Y, F, I, or V
  VARIANT 150: note = X is R, E, K, Q, or F
  VARIANT 151: note = X is K, E, A, or N
  VARIANT 152: note = X is Q or K
  VARIANT 153: note = X is L or I
  VARIANT 154: note = X is E, D, N, or Q
  VARIANT 155: note = X is V, I, or L
  VARIANT 157: note = X is S, N, F, T, or Q
  VARIANT 158: note = X is H, I, C, or R
  VARIANT 159: note = X is L, D, N, S, or F
  VARIANT 160: note = X is T or K
  VARIANT 161: note = X is K, G, or N
  VARIANT 162: note = X is C, V, or I
  VARIANT 163: note = X is Q, L, K, or Y
  VARIANT 164: note = X is A, G, or N
  VARIANT 166: note = X is V or A
  VARIANT 167: note = X is M, L, I, V, or A
  VARIANT 168: note = X is S, T, or D
  VARIANT 169: note = X is V or I
  VARIANT 170: note = X is E, Q, K, S, or I
  VARIANT 171: note = X is Q, H, or T
  VARIANT 172: note = X is L, R, or Y
  VARIANT 175: note = X is G, I, L, or T
  VARIANT 176: note = X is G, A, or V
  VARIANT 177: note = X is E, N, or D
  VARIANT 178: note = X is K, Y, D, E, A, or R
  VARIANT 179: note = X is I, F, Y, or C
  VARIANT 180: note = X is K or R
  VARIANT 181: note = X is E, R, A, G, or T
  VARIANT 182: note = X is G or N
  VARIANT 183: note = X is S, I, K, R, or E
  VARIANT 184: note = X is L, I, or M,
  VARIANT 185: note = X is T, S, D, or K
  VARIANT 186: note = X is L, H, Y, R, T, or F
  VARIANT 187: note = X is E, Y, I, M, A, or L
  VARIANT 188: note = X is E, D, R, or G
  VARIANT 189: note = X is V, F, M, L, or I
  VARIANT 190: note = X is G, K, R, L, V, or E
  VARIANT 191: note = X is K, N, D, L, H, or S
  VARIANT 192: note = X is K, L, C, or absent
  VARIANT 193: note = X is K, S, I, Y, M, or F
  VARIANT 194: note = X is K, L, C, H, D, Q, or N
  VARIANT 196: note = X is N or Y
  VARIANT 197: note = X is D, K, T, E, C, or absent
  VARIANT 198: note = X is E, V, R, or absent
  VARIANT 199: note = X is I, F, L, or absent
  VARIANT 200: note = X is V, Q, E, L, or absent
  VARIANT 201: note = X is F or absent

SEQ ID NO: 318 - protein, 196 aa, synthetic construct
  VARIANT 1: note = X at position 1 can be selected from any one of F, Q, N, D, or absent.
  VARIANT 7: note = X at position 7 can be selected from any one of M, I, V, Q, F, L, or absent.
  VARIANT 10: note = X at position 10 can be selected from any one of K, S, L, I, T, E, or absent.
  VARIANT 19: note = X at position 19 can be selected from any one of R, Q, N, T, D, or absent.
  VARIANT 27: note = X at position 27 can be selected from any one of E, Q, G, S, A, Y, or absent.
  VARIANT 29: note = X at position 29 can be selected from any one of I, V, L, or absent.
  VARIANT 46: note = X at position 46 can be selected from any one of V, M, L, or I.
  VARIANT 56: note = X at position 56 can be selected from any one of R, S, N, E, K, or Q.
  VARIANT 70: note = X at position 70 can be selected from any one of L, M, V, I, or C.
  VARIANT 77: note = X at position 77 can be selected from any one of L, H, K, E, Y, D, or A.
  VARIANT 114: note = X at position 114 can be selected from any one of Q, E, I, K, or A.
  VARIANT 115: note = X at position 115 can be selected from any one of V, H, R, K, L, or E.
  VARIANT 127: note = X at position 127 can be selected from any one of T, E, S, D, K, or N.
  VARIANT 145: note = X at position 145 can be selected from any one of Y, F, I, or V.
  VARIANT 155: note = X at position 155 can be selected from any one of L, D, N, S, or F.
  VARIANT 157: note = X at position 157 can be selected from any one of K, G, or N.
  VARIANT 166: note = X at position 166 can be selected from any one of E, Q, K, S, or I.
  VARIANT 181: note = X at position 181 can be selected from any one of T, S, D, or K.
  VARIANT 186: note = X at position 186 can be selected from any one of G, K, R, L, V, or E.
  VARIANT 195: note = X at position 195 can be selected from any one of V, Q, E, L, or absent.
XLVKSSXEEXKEELREKLXHLSHEYLXLXDLAYDSKQNRLFEMKVXELLINECGYXGLHLGGSRKPDGIXYTEGLKXNYGIIIDTKAYSDGYNLPISQADEMERYIRENNTRNXXVNPNEWWENFPXNINEFYFLFVSGHFKGNXEEQLERISIXTXIKGAAMSVXTLLLLANEIKAGRLXLEEVXKYFDNKEIXF

SEQ ID NO: 319 - protein, 196 aa, synthetic construct; identical to SEQ ID NO: 318
  VARIANT 1: note = X at position 1 can be selected from any one of F, Q, N, or absent.
  VARIANT 7: note = X at position 7 can be selected from any one of M, I, V, Q, F, L, or absent.
  VARIANT 10: note = X at position 10 can be selected from any one of K, S, L, I, T, E, or absent.
  VARIANT 19: note = X at position 19 can be selected from any one of R, Q, N, T, D, or absent.
  VARIANT 27: note = X at position 27 can be selected from any one of E, Q, G, S, A, or absent.
  VARIANT 29: note = X at position 29 can be selected from any one of I, V, L, or absent.
  VARIANT 46: note = X at position 46 can be selected from any one of V, M, L, or I.
  VARIANT 56: note = X at position 56 can be selected from any one of R, S, N, E, K, or Q.
  VARIANT 70: note = X at position 70 can be selected from any one of L, M, V, I, or C.
  VARIANT 77: note = X at position 77 can be selected from any one of L, H, K, E, Y, D, or A.
  VARIANT 114: note = X at position 114 can be selected from any one of Q, E, I, K, or A.
  VARIANT 115: note = X at position 115 can be selected from any one of V, H, R, K, L, or E.
  VARIANT 127: note = X at position 127 can be selected from any one of T, E, S, D, K, or N.
  VARIANT 145: note = X at position 145 can be selected from any one of Y, F, I, or V.
  VARIANT 155: note = X at position 155 can be selected from any one of L, D, N, S, or F.
  VARIANT 157: note = X at position 157 can be selected from any one of K, G, or N.
  VARIANT 166: note = X at position 166 can be selected from any one of E, Q, K, S, or I.
  VARIANT 181: note = X at position 181 can be selected from any one of T, S, D, or K.
  VARIANT 186: note = X at position 186 can be selected from any one of G, K, R, L, V, or E.
  VARIANT 195: note = X at position 195 can be selected from any one of V, Q, E, L, or absent.

SEQ ID NO: 320 - protein, 115 aa, synthetic construct
SEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLH

SEQ ID NO: 321 - protein, 115 aa, synthetic construct
STVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 322 - protein, 115 aa, synthetic construct
NSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 323 - protein, 34 aa, synthetic construct
LTPDQVVAIASNHGGKQALETVQRLLPVLCQDHG

SEQ ID NO: 324 - protein, 34 aa, synthetic construct
LTPDQVVAIASNGGGKQALETVQRLLPVLCQDHG

SEQ ID NO: 325 - protein, 34 aa, synthetic construct
LTPDQVVAIASNIGGKQALETVQRLLPVLCQDHG

SEQ ID NO: 326 - protein, 34 aa, synthetic construct
LTPDQVVAIASHDGGKQALETVQRLLPVLCQDHG

SEQ ID NO: 327 - protein, 20 aa, synthetic construct
LTPEQVVAIASNGGGRPALE

SEQ ID NO: 328 - protein, 20 aa, synthetic construct
LTPDQVVAIASNGGGRPALE

SEQ ID NO: 329 - protein, 20 aa, synthetic construct
LTPEQVVAIASNIGGRPALE

SEQ ID NO: 330 - protein, 20 aa, synthetic construct
LTPDQVVAIASNIGGRPALE

SEQ ID NO: 331 - protein, 20 aa, synthetic construct
LTPEQVVAIASHDGGRPALE

SEQ ID NO: 332 - protein, 20 aa, synthetic construct
LTPDQVVAIASHDGGRPALE

SEQ ID NO: 333 - protein, 20 aa, synthetic construct
LTPEQVVAIASNHGGRPALE

SEQ ID NO: 334 - protein, 20 aa, synthetic construct
LTPDQVVAIASNHGGRPALE

SEQ ID NO: 335 - protein, 137 aa, synthetic construct
NFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 336 - protein, 35 aa, synthetic construct
LSTAQVVAVASGSGGKPALEAVRAQLLALRAAPYG

SEQ ID NO: 337 - protein, 35 aa, synthetic construct; identical to SEQ ID NO: 336

SEQ ID NO: 338 - protein, 35 aa, synthetic construct
LNTAQIVAIASHDGGKPALEAVWAKLPVLRGAPYA

SEQ ID NO: 339 - protein, 35 aa, synthetic construct
LNTAQVVAIASHDGGKPALEAVRAKLPVLRGVPYA

SEQ ID NO: 340 - protein, 35 aa, synthetic construct
LNTAQVVAIASHDGGKPALEAVWAKLPVLRGVPYA

SEQ ID NO: 341 - protein, 35 aa, synthetic construct
LNTAQVVAIASHDGGKPALEAVWAKLPVLRGVPYE

SEQ ID NO: 342 - protein, 35 aa, synthetic construct
LSTAQVVAIASHDGGKPALEAVWAKLPVLRGAPYA

SEQ ID NO: 343 - protein, 35 aa, synthetic construct
LSTAQVVAVASHDGGKPALEAVRKQLPVLRGVPHQ

SEQ ID NO: 344 - protein, 35 aa, synthetic construct; identical to SEQ ID NO: 343

SEQ ID NO: 345 - protein, 35 aa, synthetic construct; identical to SEQ ID NO: 340

SEQ ID NO: 346 - protein, 35 aa, synthetic construct
LSTEQVVAIASHNGGKLALEAVKAHLLDLRGAPYA

SEQ ID NO: 347 - protein, 35 aa, synthetic construct
LSTEQVVAIASHNGGKPALEAVKAHLLALRAAPYA

SEQ ID NO: 348 - protein, 35 aa, synthetic construct
LNTAQVVAIASHYGGKPALEAVWAKLPVLRGVPYA

SEQ ID NO: 349 - protein, 35 aa, synthetic construct
LNTEQVVAIASNNGGKPALEAVKAQLLELRAAPYE

SEQ ID NO: 350 - protein, 35 aa, synthetic construct
LSPEQVVAIASNNGGKPALEAVKALLLALRAAPYE

SEQ ID NO: 351 - protein, 35 aa, synthetic construct
LSPEQVVAIASNNGGKPALEAVKAQLLELRAAPYE

SEQ ID NO: 352 - protein, 35 aa, synthetic construct
LSTEQVVAIASNNGGKPALEAVKALLLALRAAPYE

SEQ ID NO: 353 - protein, 35 aa, synthetic construct
LSTEQVVAIASNNGGKPALEAVKALLLELRAAPYE

SEQ ID NO: 354 - protein, 35 aa, synthetic construct; identical to SEQ ID NO: 350

SEQ ID NO: 355 - protein, 35 aa, synthetic construct; identical to SEQ ID NO: 351

SEQ ID NO: 356 - protein, 35 aa, synthetic construct; identical to SEQ ID NO: 353

SEQ ID NO: 357 - protein, 33 aa, synthetic construct
LGHKELIKIAARNGGGNNLIAVLSCYAKLKEMG

SEQ ID NO: 358 - protein, 33 aa, synthetic construct
FSAEQIVRIAAHDGGSRNIEAVQQAQHVLKELG

SEQ ID NO: 359 - protein, 33 aa, synthetic construct
FSAEQIVSIVAHDGGSRNIEAVQQAQHILKELG

SEQ ID NO: 360 - protein, 35 aa, synthetic construct
FSRQQILRIASHDGGSKNIAAVQKFLPKLMNFGFN

SEQ ID NO: 361 - protein, 33 aa, synthetic construct
FSAEQIVRIAAHDGGSLNIDAVQQAQQALKELG

SEQ ID NO: 362 - protein, 33 aa, synthetic construct
FSTEQIVCIAGHGGGSLNIKAVLLAQQALKDLG

SEQ ID NO: 363 - protein, 33 aa, synthetic construct
FSSEQIVRVAAHGGGSLNIKAVLQAHQALKELD

SEQ ID NO: 364 - protein, 33 aa, synthetic construct
FSAEQIVHIAAHGGGSLNIKAILQAHQTLKELN

SEQ ID NO: 365 - protein, 33 aa, synthetic construct
FSAEQIVRIAAHIGGSRNIEAIQQAHHALKELG

SEQ ID NO: 366 - protein, 33 aa, synthetic construct
FSAEQIVRIAAHIGGSHNLKAVLQAQQALKELD

SEQ ID NO: 367 - protein, 33 aa, synthetic construct
FSAKHIVRIAAHIGGSLNIKAVQQAQQALKELG

SEQ ID NO: 368 - protein, 33 aa, synthetic construct
FNAEQIVRMVSHKGGSKNLALVKEYFPVFSSFH

SEQ ID NO: 369 - protein, 35 aa, synthetic construct
FNAEQIVRMVSHKGGSKNLALVKEYFPVFSSFHFT

SEQ ID NO: 370 - protein, 33 aa, synthetic construct
FSADQIVRIAAHKGGSHNIVAVQQAQQALKELD

SEQ ID NO: 371 - protein, 35 aa, synthetic construct
FNVEQIVRMVSHNGGSKNLKAVTDNHDDLKNMGFN

SEQ ID NO: 372 - protein, 35 aa, synthetic construct
FSADQVVKIAGHSGGSNNIAVMLAVFPRLRDFGFK

SEQ ID NO: 373 - protein, 33 aa, synthetic construct
FSAEQIVSIAAHVGGSHNIEAVQKAHQALKELD

SEQ ID NO: 374 - protein, 35 aa, synthetic construct
FNAEQIVSMVSNNGGSKNLKAVTDNHDDLKNMGFN

SEQ ID NO: 375 - protein, 33 aa, synthetic construct
FSHKELIKIAARNGGGNNLIAVLSCYAKLKEMG

SEQ ID NO: 376 - protein, 35 aa, synthetic construct
FSHKELIKIAARNGGGNNLIAVLSCYAKLKEMGFS

SEQ ID NO: 377 - protein, 35 aa, synthetic construct
FSSGETVGATVGAGGTETVAQGGTASNTTVSSGGY

SEQ ID NO: 378 - protein, 35 aa, synthetic construct
FSGGMATSTTVGSGGTQDVLAGGAAVGGTVGTGGV

SEQ ID NO: 379 - protein, 35 aa, synthetic construct
FSAADIVKIAGKIGGAQALQAFITHRAALIQAGFS

SEQ ID NO: 380 - protein, 35 aa, synthetic construct
FNPTDIVKIAGNDGGAQALQAVLELEPALRERGFS

SEQ ID NO: 381 - protein, 35 aa, synthetic construct
FNPTDIVRMAGNDGGAQALQAVFELEPAFRERSFS

SEQ ID NO: 382 - protein, 35 aa, synthetic construct
FNPTDIVRMAGNDGGAQALQAVLELEPAFRERGFS

SEQ ID NO: 383 - protein, 35 aa, synthetic construct
FSQVDIVKIASNDGGAQALYSVLDVEPTFRERGFS

SEQ ID NO: 384 - protein, 35 aa, synthetic construct
FSRADIVKIAGNDGGAQALYSVLDVEPPLRERGFS

SEQ ID NO: 385 - protein, 35 aa, synthetic construct
FSRGDIVKIAGNDGGAQALYSVLDVEPPLRERGFS

SEQ ID NO: 386 - protein, 35 aa, synthetic construct
FNRADIVRIAGNGGGAQALYSVRDAGPTLGKRGFS

SEQ ID NO: 387 - protein, 35 aa, synthetic construct
FRQADIVKIASNGGSAQALNAVIKLGPTLRQRGFS

SEQ ID NO: 388 - protein, 35 aa, synthetic construct
FRQADIVKMASNGGSAQALNAVIKLGPTLRQRGFS

SEQ ID NO: 389 - protein, 35 aa, synthetic construct
FSRADIVKIAGNGGGAQALQAVLELEPTFRERGFS

SEQ ID NO: 390 - protein, 35 aa, synthetic construct
FSRADIVRIAGNGGGAQALYSVLDVGPTLGKRGFS

SEQ ID NO: 391 - protein, 35 aa, synthetic construct
FSRGDIVRIAGNGGGAQALQAVLELEPTLGERGFS

SEQ ID NO: 392 - protein, 35 aa, synthetic construct
FSRADIVKIAGNGGGAQALQAVITHRAALTQAGFS

SEQ ID NO: 393 - protein, 35 aa, synthetic construct
FSRGDTVKIAGNIGGAQALQAVLELEPTLRERGFS

SEQ ID NO: 394 - protein, 35 aa, synthetic construct
FNPTDIVKIAGNIGGAQALQAVLELEPAFRERGFS

SEQ ID NO: 395 - protein, 35 aa, synthetic construct
FSAADIVKIAGNIGGAQALQAIFTHRAALIQAGFS

SEQ ID NO: 396 - protein, 35 aa, synthetic construct
FSAADIVKIAGNIGGAQALQAVITHRATLTQAGFS

SEQ ID NO: 397 - protein, 35 aa, synthetic construct
FSATDIVKIASNIGGAQALQAVISRRAALIQAGFS

SEQ ID NO: 398 - protein, 35 aa, synthetic construct
FSQPDIVKIAGNIGGAQALQAVLELEPAFRERGFS

SEQ ID NO: 399 - protein, 35 aa, synthetic construct
FSRADIVKIAGNIGGAQALQAVLELESTFRERSFN

SEQ ID NO: 400 - protein, 35 aa, synthetic construct
FSRADIVKIAGNIGGAQALQAVLELESTLRERSFN

SEQ ID NO: 401 - protein, 35 aa, synthetic construct
FSRGDIVKMAGNIGGAQALQAGLELEPAFRERGFS

SEQ ID NO: 402 - protein, 35 aa, synthetic construct
FSRGDIVKMAGNIGGAQALQAVLELEPAFHERSFC

SEQ ID NO: 403 - protein, 35 aa, synthetic construct
FTLTDIVKMAGNIGGAQALKAVLEHGPTLRQRDLS

SEQ ID NO: 404 - protein, 35 aa, synthetic construct
FTLTDIVKMAGNIGGAQALKVVLEHGPTLRQRDLS

SEQ ID NO: 405 - protein, 35 aa, synthetic construct
FNPTDIVKIAGNNGGAQALQAVLELEPALRERGFS

SEQ ID NO: 406 - protein, 35 aa, synthetic construct
FNPTDIVKIAGNNGGAQALQAVLELEPALRERSFS

SEQ ID NO: 407 - protein, 35 aa, synthetic construct
FNPTDMVKIAGNNGGAQALQAVLELEPALRERGFS

SEQ ID NO: 408 - protein, 35 aa, synthetic construct
FSAADIVKIASNNGGAQALQALIDHWSTLSGKTKA

SEQ ID NO: 409 - protein, 35 aa, synthetic construct
FSAADIVKIASNNGGAQALQAVISRRAALIQAGFS

SEQ ID NO: 410 - protein, 35 aa, synthetic construct
FSAADIVKIASNNGGAQALQAVITHRAALAQAGFS

SEQ ID NO: 411 - protein, 35 aa, synthetic construct
FSAADIVKIASNNGGARALQALIDHWSTLSGKTKA

SEQ ID NO: 412 - protein, 35 aa, synthetic construct
FTLTDIVEMAGNNGGAQALKAVLEHGSTLDERGFT

SEQ ID NO: 413 - protein, 35 aa, synthetic construct
FTLTDIVKMAGNNGGAQALKAVLEHGPTLDERGFT

SEQ ID NO: 414 - protein, 35 aa, synthetic construct
FTLTDIVKMAGNNGGAQALKVVLEHGPTLRQRGFS

SEQ ID NO: 415 - protein, 35 aa, synthetic construct
FTLTDIVKMASNNGGAQALKAVLEHGPTLDERGFT

SEQ ID NO: 416 - protein, 35 aa, synthetic construct
FSAADIVKIAGNSGGAQALQAVISHRAALTQAGFS

SEQ ID NO: 417 - protein, 35 aa, synthetic construct
FSGGDAVSTVVRSGGAQSVASGGTASGTTVSAGAT

SEQ ID NO: 418 - protein, 35 aa, synthetic construct
FRQTDIVKMAGSGGSAQALNAVIKHGPTLRQRGFS

SEQ ID NO: 419 - protein, 34 aa, synthetic construct
FSLIDIVEIASNGGAQALKAVLKYGPVLTQAGRS

SEQ ID NO: 420 - protein, 35 aa, synthetic construct
FSGGDAAGTVVSSGGAQNVTGGLASGTTVASGGAA

SEQ ID NO: 421 - protein, 35 aa, synthetic construct
FNLTDIVEMAANSGGAQALKAVLEHGPTLRQRGLS

SEQ ID NO: 422 - protein, 35 aa, synthetic construct
FNRASIVKIAGNSGGAQALQAVLKHGPTLDERGFN

SEQ ID NO: 423 - protein, 35 aa, synthetic construct
FSQANIVKMAGNSGGAQALQAVLDLELVFRERGFS

SEQ ID NO: 424 - protein, 35 aa, synthetic construct
FSQPDIVKMAGNSGGAQALQAVLDLELAFRERGFS

SEQ ID NO: 425 - protein, 34 aa, synthetic construct
FSLIDIVEIASNGGAQALKAVLKYGPVLMQAGRS

SEQ ID NO: 426 - protein, 33 aa, synthetic construct
YKSEDIIRLASHDGGSVNLEAVLRLHSQLTRLG

SEQ ID NO: 427 - protein, 33 aa, synthetic construct
YKPEDIIRLASHGGGSVNLEAVLRLNPQLIGLG

SEQ ID NO: 428 - protein, 33 aa, synthetic construct
YKSEDIIRLASHGGGSVNLEAVLRLHSQLTRLG

SEQ ID NO: 429 - protein, 33 aa, synthetic construct
YKSEDIIRLASHGGGSVNLEAVLRLNPQLIGLG

SEQ ID NO: 430 - protein, 35 aa, synthetic construct
FNLTDIVEMAGKGGGAQALKAVLEHGPTLRQRGFN

SEQ ID NO: 431 - protein, 35 aa, synthetic construct
FRQADIIKIAGNDGGAQALQAVIEHGPTLRQHGFN

SEQ ID NO: 432 - protein, 35 aa, synthetic construct
FSQADIVKIAGNDGGTQALHAVLDLERMLGERGFS

SEQ ID NO: 433 - protein, 35 aa, synthetic construct
FSRADIVKIAGNGGGAQALKAVLEHEATLDERGFS

SEQ ID NO: 434 - protein, 35 aa, synthetic construct
FSRADIVRIAGNGGGAQALYSVLDVEPTLGKRGFS

SEQ ID NO: 435 - protein, 35 aa, synthetic construct
FSQPDIVKMASNIGGAQALQAVLELEPALRERGFS

SEQ ID NO: 436 - protein, 35 aa, synthetic construct
FSQPDIVKMAGNIGGAQALQAVLSLGPALRERGFS

SEQ ID NO: 437 - protein, 35 aa, synthetic construct
FSQPEIVKIAGNIGGAQALHTVLELEPTLHKRGFN

SEQ ID NO: 438 - protein, 35 aa, synthetic construct
FSQSDIVKIAGNIGGAQALQAVLDLESMLGKRGFS

SEQ ID NO: 439 - protein, 35 aa, synthetic construct
FSQSDIVKIAGNIGGAQALQAVLELEPTLRESDFR

SEQ ID NO: 440 - protein, 35 aa, synthetic construct
FNPTDIVKIAGNKGGAQALQAVLELEPALRERGFN

SEQ ID NO: 441 - protein, 35 aa, synthetic construct
FSPTDIIKIAGNNGGAQALQAVLDLELMLRERGFS

SEQ ID NO: 442 - protein, 35 aa, synthetic construct
FSQADIVKIAGNNGGAQALYSVLDVEPTLGKRGFS

SEQ ID NO: 443 - protein, 35 aa, synthetic construct
FSRGDIVTIAGNNGGAQALQAVLELEPTLRERGFN

SEQ ID NO: 444 - protein, 35 aa, synthetic construct
FSRIDIVKIAANNGGAQALHAVLDLGPTLRECGFS

SEQ ID NO: 445 - protein, 35 aa, synthetic construct
FSQADIVKIVGNNGGAQALQAVFELEPTLRERGFN

SEQ ID NO: 446 - protein, 35 aa, synthetic construct
FSQPDIVRITGNRGGAQALQAVLALELTLRERGFS

SEQ ID NO: 447 - protein, 110 aa, synthetic construct
HHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 448 - DNA, 17 bp, synthetic construct
  source 1..17: mol_type = other DNA
tttctgtcaccaatcct

SEQ ID NO: 449 - DNA, 18 bp, synthetic construct
  source 1..18: mol_type = other DNA
tcccctccaccccacagt

SEQ ID NO: 450 - protein, 32 aa, synthetic construct
  SITE 11..12: note = An RVD is present between the amino acids at positions 11 and 12.
LTPDQVVAIASGGKQALETVQRLLPVLCQDHG

SEQ ID NO: 451 - skipped